(19) United States
(12) Patent Application Publication
(10) Pub. No.: US 2025/0205363 A1
(43) Pub. Date: Jun. 26, 2025

(54) FUNCTIONAL AAV CAPSIDS FOR SYSTEMIC ADMINISTRATION

(71) Applicant: SHAPE THERAPEUTICS INC., Seattle, WA (US)

(72) Inventors: Thomas PACKARD, Seattle, WA (US); David Jeffrey HUSS, Seattle, WA (US); Francois VIGNEAULT, Seattle, WA (US); Adrian Wrangham BRIGGS, Seattle, WA (US); Ronald James HAUSE, JR., Seattle, WA (US); Kevin Christopher STEIN, Kenmore, WA (US); Yue JIANG, Los Angeles, CA (US)

(21) Appl. No.: 18/714,511

(22) PCT Filed: Nov. 30, 2022
(86) PCT No.: PCT/US2022/051452

§ 371 Date: May 29, 2024

Related U.S. Application Data
(60) Provisional application No. 63/284,977, filed on Dec. 1, 2021.
(60) Provisional application No. 63/342,032, filed on May 13, 2022.
(60) Provisional application No. 63/354,635, filed on Jun. 22, 2022.
(60) Provisional application No. 63/399,164, filed on Aug. 18, 2022.

(51) Int. Cl. A61K 48/00; C07K 14/005; C12N 9/22; C12N 9/80; C12N 15/11; C12N 15/86

(52) U.S. Cl. CPC A61K 48/005; A61K 48/0075; A61K 48/0083; C07K 14/005; C12N 9/22; C12N 9/80; C12N 15/111; C12N 15/86; C12N 2310/20; C12N 2750/14122; C12N 2750/14143; C12N 2750/14145; C12Y 305/04004

(57) ABSTRACT

Disclosed herein are engineered AAV VP capsid polypeptides with the ability to assemble into virus particles and having improved tissue tropism to, for example, CNS tissues or muscle tissues. The capsids are engineered using the high throughput discovery system described herein. In certain embodiments, provided herein are recombinant adeno-associated virus (AAV) VP capsid polypeptides having at least one mutation in a 581-589 region of the VP capsid polypeptide, corresponding to residues 581 to residue 589 of a VP1 polypeptide of SEQ ID NO: 1.

### CROSS-REFERENCE

The present application claims the benefit of U.S. Provisional Application No. 63/284,977, entitled “FUNCTIONAL AAV CAPSIDS FOR SYSTEMIC ADMINISTRATION,” filed on Dec. 1, 2021, U.S. Provisional Application No. 63/342,032, entitled “FUNCTIONAL AAV CAPSIDS FOR SYSTEMIC ADMINISTRATION,” filed on May 13, 2022, U.S. Provisional Application No. 63/354,635, entitled “FUNCTIONAL AAV CAPSIDS FOR SYSTEMIC ADMINISTRATION,” filed on Jun. 22, 2022, and U.S. Provisional Application No. 63/399,164, entitled “FUNCTIONAL AAV CAPSIDS FOR SYSTEMIC ADMINISTRATION,” filed on Aug. 18, 2022, each of which applications are herein incorporated by reference in their entireties for all purposes.

### SEQUENCE LISTING

The instant application contains a Sequence Listing which has been submitted electronically in eXtensible Markup Language (XML) format and is hereby incorporated by reference in its entirety. Said XML copy, created on Nov. 28, 2022, is named “421688-705021_SL.xml” and is 5 megabytes in size.

### BACKGROUND

Recombinant adeno-associated viruses (rAAV) provide the leading platform for in vivo delivery of gene therapies. Current clinical trials employ a limited number of AAV capsids, primarily from naturally occurring human or primate serotypes such as AAV1, AAV2, AAV5, AAV6, AAV8, AAV9, AAVrh.10, AAV4rh.74, and AAVhu.67. These capsids often provide suboptimal targeting to tissues of interest, both due to poor infectivity of the tissue of interest and competing liver tropism. Increasing the dose to ensure infection of desired tissues can lead to dose-dependent liver toxicity. In addition, use of naturally-occurring capsids presents an immunological memory challenge—pre-immune patient populations are excluded from treatment and repeat dosing in a previously immune naïve patient is often not possible. Thus, there is a need for additional AAV capsids for use in gene therapy, in particular capsids that confer upon the rAAV high infectivity for specific tissues, such as muscle tissue and tissues in the central nervous system, and low liver tropism

### SUMMARY OF THE INVENTION

Described herein is a system for high throughput engineering of functional AAV capsids with altered tropism for various tissues and capsid variants that have increased tropism for target tissues, such as muscle or central nervous system (CNS) tissues.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a central nervous system (CNS) tissue at a DNA enrichment of at least 100-fold greater than a wild type AAV9.

In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3846, SEQ ID NO: 3283, SEQ ID NO: 1956, SEQ ID NO: 3297, SEQ ID NO: 4545, SEQ ID NO: 2661, SEQ ID NO: 1576, SEQ ID NO: 425, SEQ ID NO: 709, SEQ ID NO: 2168, SEQ ID NO: 54, SEQ ID NO: 429, SEQ ID NO: 708, SEQ ID NO: 428, SEQ ID NO: 4119, SEQ ID NO: 3906, SEQ ID NO: 2456, SEQ ID NO: 2278, SEQ ID NO: 5006, SEQ ID NO: 426, SEQ ID NO: 307, SEQ ID NO: 5155, SEQ ID NO: 2640, SEQ ID NO: 4317, SEQ ID NO: 1145, SEQ ID NO: 430, and SEQ ID NO: 885. In some aspects, the recombinant viral capsid is capable of preferentially targeting the CNS tissue at a DNA enrichment of at least 200-fold greater than the wild type AAV9. In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3846, SEQ ID NO: 3283, SEQ ID NO: 1956, and SEQ ID NO: 3297. In some aspects, the recombinant viral capsid is capable of preferentially targeting the CNS tissue at a DNA enrichment of at least 1000-fold greater than the wild type AAV9. In some aspects, the 581-589 region has a sequence of SEQ ID NO: 18.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a central nervous system (CNS) tissue at an RNA enrichment of at least 100-fold greater than a wild type AAV9.

In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 3297, SEQ ID NO: 4545, SEQ ID NO: 2661, SEQ ID NO: 1576, SEQ ID NO: 425, SEQ ID NO: 709, SEQ ID NO: 54, SEQ ID NO: 429, SEQ ID NO: 428, SEQ ID NO: 426, and SEQ ID NO: 430. In some aspects, the recombinant viral capsid is capable of preferentially targeting the CNS tissue at an RNA enrichment of at least 200-fold or greater than the wild type AAV9. In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 2661, SEQ ID NO: 1576, SEQ ID NO: 425, SEQ ID NO: 709, SEQ ID NO: 54, SEQ ID NO: 430, SEQ ID NO: 569, and SEQ ID NO: 719. In some aspects, the recombinant viral capsid is capable of preferentially targeting the CNS tissue at an RNA enrichment of at least 500-fold or greater than the wild type AAV9. In some aspects, the 581-589 region has a sequence selected from the group consisting of SEQ ID NO: 18, SEQ ID NO: 3472, SEQ ID NO: 262, and SEQ ID NO: 2028.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a central nervous system (CNS) tissue at a DNA enrichment of at least 5-fold greater than a wild type AAV5.

In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3846, SEQ ID NO: 3283, SEQ ID NO: 1956, SEQ ID NO: 3297, SEQ ID NO: 4545, SEQ ID NO: 2661, and SEQ ID NO: 1576. In some aspects, the recombinant viral capsid is capable of preferentially targeting the CNS tissue at a DNA enrichment of at least 10-fold greater than the wild type AAV5. In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, and SEQ ID NO: 424.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a central nervous system (CNS) tissue at an RNA enrichment of at least 5-fold greater than a wild type AAV5.

In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 3297, SEQ ID NO: 4545, SEQ ID NO: 2661, SEQ ID NO: 1576, SEQ ID NO: 425, SEQ ID NO: 709, SEQ ID NO: 54, SEQ ID NO: 429, SEQ ID NO: 708, SEQ ID NO: 428, SEQ ID NO: 426, SEQ ID NO: 307, SEQ ID NO: 4317, SEQ ID NO: 430, and SEQ ID NO: 885. In some aspects, the recombinant viral capsid is capable of preferentially targeting the CNS tissue at an RNA enrichment of at least 20-fold greater than the wild type AAV5. In some aspects, the 581-589 region has a sequence selected from the group consisting of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 2661, SEQ ID NO: 1576, SEQ ID NO: 425, SEQ ID NO: 709, SEQ ID NO: 54, SEQ ID NO: 430, SEQ ID NO: 569, and SEQ ID NO: 719.

In various aspects, the present disclosure provides a viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 18, (b) SEQ ID NO: 3472, (c) SEQ ID NO: 262, (d) SEQ ID NO: 3306, (e) SEQ ID NO: 2028, (f) SEQ ID NO: 2791, (g) SEQ ID NO: 424, (h) SEQ ID NO: 2536, (i) SEQ ID NO: 1971, (j) SEQ ID NO: 415, (k) SEQ ID NO: 3846, (l) SEQ ID NO: 3283, (m) SEQ ID NO: 1956, (n) SEQ ID NO: 3297, (o) SEQ ID NO: 4545, (p) SEQ ID NO: 2661, (q) SEQ ID NO: 1576, (r) SEQ ID NO: 425, (s) SEQ ID NO: 709, (t) SEQ ID NO: 2168, (u) SEQ ID NO: 54, (v) SEQ ID NO: 429, (w) SEQ ID NO: 708, (x) SEQ ID NO: 428, (y) SEQ ID NO: 4119, (z) SEQ ID NO: 3906, (aa) SEQ ID NO: 2456, (bb) SEQ ID NO: 2278, (cc) SEQ ID NO: 5006, or (dd) SEQ ID NO: 426.

In some aspects, the 581-589 region has a sequence of: (a) SEQ ID NO: 18, (b) SEQ ID NO: 3472, (c) SEQ ID NO: 262, (d) SEQ ID NO: 3306, (e) SEQ ID NO: 2028, (f) SEQ ID NO: 2791, (g) SEQ ID NO: 424, (h) SEQ ID NO: 2536, (i) SEQ ID NO: 1971, (j) SEQ ID NO: 415, (k) SEQ ID NO: 3846, (l) SEQ ID NO: 3283, (m) SEQ ID NO: 1956, (n) SEQ ID NO: 3297, (o) SEQ ID NO: 4545, (p) SEQ ID NO: 2661, (q) SEQ ID NO: 1576, (r) SEQ ID NO: 425, (s) SEQ ID NO: 709, (t) SEQ ID NO: 2168, (u) SEQ ID NO: 54, (v) SEQ ID NO: 429, (w) SEQ ID NO: 708, (x) SEQ ID NO: 428, (y) SEQ ID NO: 4119, (z) SEQ ID NO: 3906, (aa) SEQ ID NO: 2456, (bb) SEQ ID NO: 2278, (cc) SEQ ID NO: 5006, or (dd) SEQ ID NO: 426.

In various aspects, the present disclosure provides a viral protein (VP) capsid polypeptide, 581-589 wherein the VP capsid polypeptide comprises a 581-589 region corresponding to residues 581 to 589 of a VP1 polypeptide; wherein the 581-589 region has a sequence that is at least 85% identical to any one of any one of SEQ ID NO: 12-SEQ ID NO: 3937 or SEQ ID NO: 3938-SEQ ID NO: 4237.

In some aspects, the 581-589 region has a sequence of any one of any one of SEQ ID NO: 12-SEQ ID NO: 3937 or SEQ ID NO: 3938-SEQ ID NO: 4237. In some aspects, the VP capsid polypeptide is capable of assembling into a recombinant viral capsid.

In various aspects, the present disclosure provides a viral protein (VP) capsid polypeptide, wherein the VP capsid polypeptide is capable of assembling into a recombinant viral capsid, and wherein the VP capsid polypeptide comprises a 581-589 region corresponding to residues 581 to 589 of a VP1 polypeptide; wherein the 581-589 region: comprises at least one amino acid substitution as compared to SEQ ID NO: 9, and confers on the recombinant viral capsid an infection rate for central nervous system (CNS) tissue with at least 3-fold higher CNS tissue tropism than a wild type VP capsid polypeptide of SEQ ID NO: 1; wherein the VP capsid polypeptide comprises at least one amino acid substitution as compared to each of SEQ ID NO: 3, SEQ ID NO: 4, SEQ ID NO: 5, SEQ ID NO: 6, SEQ ID NO: 7, and SEQ ID NO: 8.

In some aspects, the 581-589 region comprises a sequence of X¹X²X³X⁴X⁵X⁶X⁷X⁸X⁹, wherein X¹, X², X³, X⁴, X⁵, X⁶, X⁷, X⁸, and X⁹ are each individually selected from A, R, N, D, C, E, Q, G, H, I, L, K, M, F, P, S, T, W, Y, and V. In some aspects, the VP capsid polypeptide has a sequence of SEQ ID NO: 2, wherein residues 581 to 589 of SEQ ID NO: 2 (X¹X²X³X⁴X⁵X⁶X⁷X⁸X⁹) correspond to the 581-589 region. In some aspects, the 581-589 region has a sequence that is at least 70%, at least 77%, at least 80%, at least 88%, at least 90%, or at least 95% identical to any one of SEQ ID NO: 12-SEQ ID NO: 3937. In some aspects, the 581-589 region has a sequence of any one of SEQ ID NO: 12-SEQ ID NO: 3937. In some aspects, the 581-589 region has a sequence that is at least 70%, at least 77%, at least 80%, at least 88%, at least 90%, or at least 95% identical to any one of SEQ ID NO: 3938-SEQ ID NO: 4237. In some aspects, the 581-589 region has a sequence of any one of SEQ ID NO: 3938-SEQ ID NO: 4237.

In some aspects, the VP capsid polypeptide comprises an amino acid sequence at least 70%, at least 80%, at least 85%, at least 90%, at least 95%, at least 97%, at least 98%, at least 98.5%, at least 99%, or at least 99.5% identical to SEQ ID NO: 1. In some aspects, the infection rate for CNS tissue is at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold, or at least 1000-fold higher than an infection rate of the wild type VP capsid polypeptide for CNS tissue. In some aspects, the 581-589 region confers on a recombinant viral capsid assembled from the VP capsid polypeptide improved stability compared to a wild type AAV5 capsid comprising a peptide of SEQ ID NO: 1. In some aspects, the 581-589 region confers lower toxicity upon administration to a subject compared to a wild type VP capsid polypeptide of SEQ ID NO: 1. In some aspects, the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.

In various aspects, the present disclosure provides a pharmaceutical composition comprising a VP capsid polypeptide as described herein.

In some aspects, the VP capsid polypeptide is assembled into a recombinant viral capsid. In some aspects, the pharmaceutical composition further comprises a payload encapsidated by the recombinant viral capsid. In some aspects, the payload encodes a therapeutic polynucleotide or a therapeutic peptide. In some aspects, the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. In some aspects, the payload encodes a component of a CRISPR/Cas system, an adenosine deaminase acting on RNA (ADAR) enzyme, a transcriptional activator, or a transcriptional repressor.

In various aspects, the present disclosure provides a recombinant adeno-associated virus (rAAV), comprising a VP capsid polypeptide as described herein assembled into a recombinant viral capsid and a payload encapsidated by the recombinant viral capsid.

In some aspects, the rAAV further comprises a VP2 polypeptide comprising the 581-589 region and a VP3 polypeptide comprising the 581-589 region. In some aspects, the payload encodes a therapeutic polynucleotide or a therapeutic peptide. In some aspects, the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. In some aspects, the payload encodes a component of a CRISPR/Cas system, an adenosine deaminase acting on RNA (ADAR) enzyme, a transcriptional activator, or a transcriptional repressor.

In various aspects, the present disclosure provides a method of delivering a payload to a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the CNS tissue with the rAAV; and preferentially delivering the payload to the CNS tissue infected by the rAAV at a DNA enrichment of at least 100-fold greater than a wild type AAV9.

In some aspects, the DNA enrichment is at least 200-fold greater than the wild type AAV9. In some aspects, the DNA enrichment is at least 1000-fold greater than the wild type AAV9.

In various aspects, the present disclosure provides a method of transcribing a payload in a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the CNS tissue with the rAAV; and preferentially transcribing the payload to the CNS tissue infected by the rAAV at an RNA enrichment of at least 100-fold greater than a wild type AAV9.

In some aspects, the DNA enrichment is at least 200-fold greater than the wild type AAV9. In some aspects, the DNA enrichment is at least 500-fold greater than the wild type AAV9.

In various aspects, the present disclosure provides a method of delivering a payload to a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the CNS tissue with the rAAV; and preferentially delivering the payload to the CNS tissue infected by the rAAV at a DNA enrichment of at least 5-fold greater than a wild type AAV5.

In some aspects, the DNA enrichment is at least 10-fold greater than the wild type AAV5.

In various aspects, the present disclosure provides a method of transcribing a payload in a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the CNS tissue with the rAAV; and preferentially transcribing the payload to the CNS tissue infected by the rAAV at an RNA enrichment of at least 5-fold greater than a wild type AAV5.

In some aspects, the DNA enrichment is at least 20-fold greater than the wild type AAV5.

In various aspects, the present disclosure provides a method of delivering a payload to a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 18, (b) SEQ ID NO: 3472, (c) SEQ ID NO: 262, (d) SEQ ID NO: 3306, (e) SEQ ID NO: 2028, (f) SEQ ID NO: 2791, (g) SEQ ID NO: 424, (h) SEQ ID NO: 2536, (i) SEQ ID NO: 1971, (j) SEQ ID NO: 415, (k) SEQ ID NO: 3846, (l) SEQ ID NO: 3283, (m) SEQ ID NO: 1956, (n) SEQ ID NO: 3297, (o) SEQ ID NO: 4545, (p) SEQ ID NO: 2661, (q) SEQ ID NO: 1576, (r) SEQ ID NO: 425, (s) SEQ ID NO: 709, (t) SEQ ID NO: 2168, (u) SEQ ID NO: 54, (v) SEQ ID NO: 429, (w) SEQ ID NO: 708, (x) SEQ ID NO: 428, (y) SEQ ID NO: 4119, (z) SEQ ID NO: 3906, (aa) SEQ ID NO: 2456, (bb) SEQ ID NO: 2278, (cc) SEQ ID NO: 5006, or (dd) SEQ ID NO: 426; infecting the CNS tissue with the rAAV with at least 3-fold higher CNS tissue tropism as compared to a wild type AAV5 capsid comprising a peptide of SEQ ID NO: 1; and delivering the payload to the CNS tissue infected by the rAAV.

In various aspects, the present disclosure provides a method of delivering a payload to a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to residues 581 to 589 of SEQ ID NO: 1, and wherein the VP capsid polypeptide comprises at least one amino acid substitution as compared to each of SEQ ID NO: 3, SEQ ID NO: 4, SEQ ID NO: 5, SEQ ID NO: 6, SEQ ID NO: 7, and SEQ ID NO: 8; infecting the CNS tissue with the rAAV with at least 3-fold higher CNS tissue tropism as compared to a wild type AAV5 capsid comprising a peptide of SEQ ID NO: 1; and delivering the payload to the CNS tissue infected by the rAAV.

In various aspects, the present disclosure provides a method of delivering a payload to a central nervous system (CNS) tissue of a subject, the method comprising: administering a recombinant adeno-associated virus (rAAV) to the subject, wherein the rAAV encapsidates the payload, and wherein the rAAV comprises a VP capsid polypeptide as described herein; infecting the CNS tissue with the rAAV; and delivering the payload to the CNS tissue infected by the rAAV.

In some aspects, the method further comprises expressing a therapeutic polypeptide or a therapeutic polynucleotide encoded by the payload in the CNS tissue infected by the rAAV. In some aspects, the method further comprises producing a therapeutic effect in the CNS tissue of the subject. In some aspects, the therapeutic effect is produced upon administration of from 1×10⁵ to 5×10¹⁴ rAAVs per kg subject weight. In some aspects, the method comprises administering an amount of the rAAV sufficient to produce the therapeutic effect without producing a toxicity in the subject. In some aspects, the CNS tissue comprises forebrain cortex, occipital cortex, temporal cortex, thalamus, hypothalamus, substantia nigra, hippocampus DG, hippocampus CA1, hippocampus CA3, cerebellum, or a combination thereof.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a central nervous system (CNS) tissue of the subject with the rAAV; preferentially delivering the payload to the CNS tissue infected by the rAAV at a DNA enrichment of at least 100-fold greater than a wild type AAV9; and producing a therapeutic effect in the CNS tissue, thereby treating the condition.

In some aspects, the DNA enrichment is at least 200-fold greater than the wild type AAV9. In some aspects, the DNA enrichment is at least 1000-fold greater than the wild type AAV9.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting central nervous system (CNS) tissue of the subject with the rAAV; preferentially transcribing the payload to the CNS tissue infected by the rAAV at an RNA enrichment of at least 100-fold greater than a wild type AAV9; and producing a therapeutic effect in the CNS tissue, thereby treating the condition.

In some aspects, the DNA enrichment is at least 200-fold greater than the wild type AAV9. In some aspects, the DNA enrichment is at least 500-fold greater than the wild type AAV9.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting central nervous system (CNS) tissue of the subject with the rAAV; preferentially delivering the payload to the CNS tissue infected by the rAAV at a DNA enrichment of at least 5-fold greater than a wild type AAV5; and producing a therapeutic effect in the CNS tissue, thereby treating the condition.

In some aspects, the DNA enrichment is at least 10-fold greater than the wild type AAV5.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a central nervous system (CNS) tissue of the subject with the rAAV; preferentially transcribing the payload to the CNS tissue infected by the rAAV at an RNA enrichment of at least 5-fold greater than a wild type AAV5; and producing a therapeutic effect in the CNS tissue, thereby treating the condition.

In some aspects, the DNA enrichment is at least 20-fold greater than the wild type AAV5.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 18, (b) SEQ ID NO: 3472, (c) SEQ ID NO: 262, (d) SEQ ID NO: 3306, (e) SEQ ID NO: 2028, (f) SEQ ID NO: 2791, (g) SEQ ID NO: 424, (h) SEQ ID NO: 2536, (i) SEQ ID NO: 1971, (j) SEQ ID NO: 415, (k) SEQ ID NO: 3846, (l) SEQ ID NO: 3283, (m) SEQ ID NO: 1956, (n) SEQ ID NO: 3297, (o) SEQ ID NO: 4545, (p) SEQ ID NO: 2661, (q) SEQ ID NO: 1576, (r) SEQ ID NO: 425, (s) SEQ ID NO: 709, (t) SEQ ID NO: 2168, (u) SEQ ID NO: 54, (v) SEQ ID NO: 429, (w) SEQ ID NO: 708, (x) SEQ ID NO: 428, (y) SEQ ID NO: 4119, (z) SEQ ID NO: 3906, (aa) SEQ ID NO: 2456, (bb) SEQ ID NO: 2278, (cc) SEQ ID NO: 5006, or (dd) SEQ ID NO: 426; infecting a CNS tissue of the subject with the rAAV; delivering the payload to the CNS tissue infected by the rAAV; and producing a therapeutic effect in the CNS tissue, thereby treating the condition.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide a 581-589 region corresponding to residues 581 to 589 of a VP1 polypeptide, wherein the 581-589 region comprises at least one amino acid substitution as compared to residues 581 to 589 of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8; infecting a CNS tissue of the subject with the rAAV with at least 3-fold higher CNS tissue tropism as compared to a wild type AAV5 capsid comprising a peptide of SEQ ID NO: 1; delivering the payload to the CNS tissue infected by the rAAV; and producing a therapeutic effect in the CNS tissue, thereby treating the condition.

In various aspects, the present disclosure provides a method of treating a condition in a subject, the method comprising: administering a recombinant adeno-associated virus (rAAV) to the subject, wherein the rAAV encapsidates a payload, and wherein the rAAV comprises a VP capsid polypeptide as described herein; infecting a CNS tissue of the subject with the rAAV; delivering the payload to the CNS tissue infected by the rAAV; and producing a therapeutic effect in the CNS tissue, thereby treating the condition.

In some aspects, the condition is a neurological condition. In some aspects, the neurological condition is an aromatic l-amino acid decarboxylase (AADC) deficiency, Alzheimer's disease, a tauopathy, a synucleinopathy, Batten disease, mucopolysaccharidosis type III, frontotemporal dementia, Parkinson's disease, corticobasal degeneration, progressive supranuclear palsy, chronic traumatic encephalopathy, Gaucher disease, Canavan disease, Tay-Sachs disease, Huntington's disease, Protocki-Lupski syndrome, amyotrophic lateral sclerosis, Down syndrome, Sanfilippo disease type A, Sanfilippo disease type B, or Rett syndrome. In some aspects, the condition is Rett syndrome. In some aspects, the payload encodes a guide RNA that targets an mRNA encoding by MECP2 or a tRNA that targets a premature stope codon in MECP2. In some aspects, the condition is Parkinson's disease. In some aspects, the payload encodes a guide RNA targeting an mRNA encoding LRRK2, GBA, α-synuclein, AADC, GDNF, Neurturin, GAD, FOXG1, Kᵥ7.2, fragile X mental retardation protein, tau, or laforin. In some aspects, the condition is Alzheimer's disease. In some aspects, the payload encodes a guide RNA targeting an mRNA encoding amyloid precursor protein (APP), alpha-synuclein (SNCA), Tau protein (MAPT), or Apolipoprotein E (ApoE). In some aspects, the payload encodes a transgene encoding amyloid precursor protein (APP), alpha-synuclein (SNCA), Tau protein (MAPT), or Apolipoprotein E (ApoE).

In some aspects, the method comprises administering from 1×10⁵ to 5×10¹⁴ rAAVs per kg subject weight. In some aspects, the method comprises infecting the CNS tissue with at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold, or at least 1000-fold higher tissue tropism than a wild type AAV5 capsid comprising a peptide of SEQ ID NO: 1.

In some aspects, the payload encodes a therapeutic protein, therapeutic polynucleotide, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof. In some aspects, the guide RNA is a CRISPR/Cas guide RNA or an ADAR guide RNA. In some aspects, the therapeutic protein is selected from the group consisting of aromatic l-amino acid decarboxylase (AADC), amyloid precursor protein (APP), α-synuclein, microtubule associated protein tau (MAPT), ApoE, nerve growth factor (NGF), telomerase reverse transcriptase (TERT), CLN2, CLN3, CLN6, N-acetyl-α-glucosaminidase (NAGLU), granulin, glucosylceramidase 8 (GBA), aspartoacylase, GDNF, neurturin (NTN), glutamate decarboxylase (GAD), FOXG1, Kᵥ7.2, laforin, leucine rich repeat kinase 2 (LRRK2), hexosaminidase A (HEXA), hexosaminidase B (HEXB), huntingtin, cholesterol 24-hydroxylase, C9orf72, superoxide dismutase 1 (SOD1), dual specificity tyrosine-phosphorylation-regulated kinase 1A (DYRKA1), N-sulfoglucosamine sulfohydrolase (SGSH), surfeit locus protein 1 (SURF1), GABA transporter 1 (GAT1), and methyl-CpG binding protein 2 (MECP2). In some aspects, the therapeutic polynucleotide targets an mRNA encoding a protein selected from the group consisting of aromatic l-amino acid decarboxylase (AADC), amyloid precursor protein (APP), α-synuclein, microtubule associated protein tau (MAPT), ApoE, nerve growth factor (NGF), telomerase reverse transcriptase (TERT), CLN2, CLN3, CLN6, N-acetyl-α-glucosaminidase (NAGLU), granulin, glucosylceramidase 8 (GBA), aspartoacylase, GDNF, neurturin (NTN), glutamate decarboxylase (GAD), FOXG1, Kᵥ7.2, laforin, leucine rich repeat kinase 2 (LRRK2), hexosaminidase A (HEXA), hexosaminidase B (HEXB), huntingtin, cholesterol 24-hydroxylase, C9orf72, superoxide dismutase 1 (SOD1), dual specificity tyrosine-phosphorylation-regulated kinase 1A (DYRKA1), N-sulfoglucosamine sulfohydrolase (SGSH), surfeit locus protein 1 (SURF1), GABA transporter 1 (GAT1), and methyl-CpG binding protein 2 (MECP2). In some aspects, the payload encodes a component of a CRISPR/Cas system, an adenosine deaminase acting on RNA (ADAR) enzyme, a transcriptional activator, or a transcriptional repressor. In some aspects, the component of the CRISPR/Cas system comprises a Cas3, a Cas8, a Cas10, a Cas9, a Cas4, a Cas12, a Cas13, a guide RNA, or a combination thereof. In some aspects, the ADAR enzyme is ADAR1 or ADAR2. In some aspects, the transcriptional activator is VP64. In some aspects, the transcriptional repressor is KRAB.

In some aspects, the method comprises systemically administering the rAAV to the subject. In some aspects, the method comprises administering the rAAV via intravenous administration, intramuscular administration, intraperitoneal administration, or oral administration. In some aspects, the method further comprises expressing a therapeutic polypeptide or a therapeutic polynucleotide encoded by the payload in the CNS tissue infected by the rAAV. In some aspects, the method comprises expressing the therapeutic polypeptide or the therapeutic polynucleotide with higher CNS tissue tropism compared to a wild type AAV5 capsid comprising a peptide of SEQ ID NO: 1. In some aspects, the method comprises expressing the therapeutic polypeptide or the therapeutic polynucleotide with at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher tropism compared to the wild type AAV5 capsid.

In some aspects, the method comprises producing a toxicity in the subject that is lower than a toxicity produced upon systemic administration of a comparable number of wild type AAV5 capsids comprising a VP capsid polypeptide of SEQ ID NO: 1. In some aspects, the method comprises producing a toxicity in the subject that is lower than a toxicity produced upon systemic administration of a number of wild type AAV5 capsids comprising a VP capsid polypeptide of SEQ ID NO: 1 sufficient to deliver a comparable number of payloads to the CNS tissue. In some aspects, the method comprises producing a concentration of neutralizing antibodies in the subject that is lower than a concentration of neutralizing antibodies produced upon systemic administration of a comparable number of wild type AAV5 capsids comprising a VP capsid polypeptide of SEQ ID NO: 1. In some aspects, the method comprises producing a concentration of neutralizing antibodies in the subject that is lower than a concentration of neutralizing antibodies produced upon intravitreal administration of a comparable number of wild type AAV2 capsids. In some aspects, the method comprises producing a concentration of neutralizing antibodies in the subject that is lower than a concentration of neutralizing antibodies produced upon intravitreal administration of a comparable number of wild type AAV8 capsids. In some aspects, the method comprises producing a concentration of neutralizing antibodies in the subject that is lower than a concentration of neutralizing antibodies produced upon intravitreal administration of a comparable number of wild type AAV9 capsids. In some aspects, the VP capsid polypeptide further comprises one or more mutations outside of the 581-589 region that contributes to reduced production of neutralizing antibodies relative to a wild type AAV capsid. In some aspects, the method comprises producing a concentration of neutralizing antibodies in the subject that is lower than a concentration of neutralizing antibodies produced upon systemic administration of a number of wild type AAV5 capsids comprising a VP capsid polypeptide of SEQ ID NO: 1 sufficient to deliver a comparable number of payloads to the CNS tissue.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting central nervous system tissue and a cardiac muscle tissue.

In some aspects, the 581-589 region has a sequence having at least 85% sequence identity to a sequence selected from the group consisting of SEQ ID NO: 18, SEQ ID NO: 2536, SEQ ID NO: 3846, SEQ ID NO: 4545, SEQ ID NO: 2661, SEQ ID NO: 1576, SEQ ID NO: 4317, SEQ ID NO: 23, SEQ ID NO: 22, SEQ ID NO: 964, SEQ ID NO: 4288, SEQ ID NO: 4338, SEQ ID NO: 5037, and SEQ ID NO: 4936. In some aspects, the 581-589 region has a sequence selected from the group consisting of SEQ ID NO: 18, SEQ ID NO: 2536, SEQ ID NO: 3846, SEQ ID NO: 4545, SEQ ID NO: 2661, SEQ ID NO: 1576, SEQ ID NO: 4317, SEQ ID NO: 23, SEQ ID NO: 22, SEQ ID NO: 964, SEQ ID NO: 4288, SEQ ID NO: 4338, SEQ ID NO: 5037, and SEQ ID NO: 4936.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting central nervous system tissue and a skeletal muscle tissue.

In some aspects, the 581-589 region has a sequence having at least 85% sequence identity to a sequence selected from the group consisting of SEQ ID NO: 18, SEQ ID NO: 3472, SEQ ID NO: 262, SEQ ID NO: 3306, SEQ ID NO: 2536, SEQ ID NO: 1971, SEQ ID NO: 3283, SEQ ID NO: 3297, SEQ ID NO: 4545, SEQ ID NO: 2661, SEQ ID NO: 307, SEQ ID NO: 4317, SEQ ID NO: 1268, SEQ ID NO: 4288, SEQ ID NO: 724, and SEQ ID NO: 5037. In some aspects, the 581-589 region has a sequence selected from the group consisting of SEQ ID NO: 18, SEQ ID NO: 3472, SEQ ID NO: 262, SEQ ID NO: 3306, SEQ ID NO: 2536, SEQ ID NO: 1971, SEQ ID NO: 3283, SEQ ID NO: 3297, SEQ ID NO: 4545, SEQ ID NO: 2661, SEQ ID NO: 307, SEQ ID NO: 4317, SEQ ID NO: 1268, SEQ ID NO: 4288, SEQ ID NO: 724, and SEQ ID NO: 5037.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting central nervous system tissue and a muscle tissue.

In some aspects, the 581-589 region has a sequence having at least 85% sequence identity to a sequence selected from the group consisting of SEQ ID NO: 18, SEQ ID NO: 3472, SEQ ID NO: 262, SEQ ID NO: 2536, SEQ ID NO: 3846, SEQ ID NO: 3297, SEQ ID NO: 4545, SEQ ID NO: 2661, SEQ ID NO: 1576, SEQ ID NO: 4317, SEQ ID NO: 23, SEQ ID NO: 22, SEQ ID NO: 964, SEQ ID NO: 4288, SEQ ID NO: 4290, SEQ ID NO: 4338, and SEQ ID NO: 5037. In some aspects, the 581-589 region has a sequence selected from the group consisting of SEQ ID NO: 18, SEQ ID NO: 3472, SEQ ID NO: 262, SEQ ID NO: 2536, SEQ ID NO: 3846, SEQ ID NO: 3297, SEQ ID NO: 4545, SEQ ID NO: 2661, SEQ ID NO: 1576, SEQ ID NO: 4317, SEQ ID NO: 23, SEQ ID NO: 22, SEQ ID NO: 964, SEQ ID NO: 4288, SEQ ID NO: 4290, SEQ ID NO: 4338, and SEQ ID NO: 5037.

In various aspects, the present disclosure provides a recombinant adeno-associated virus (rAAV) for use in a method of treating a condition in a subject, the method comprising: administering an rAAV to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a central nervous system (CNS) tissue of the subject with the rAAV; and preferentially delivering the payload to the CNS tissue infected by the rAAV at a DNA enrichment of at least 100-fold greater than a wild type AAV9.

In various aspects, the present disclosure provides a recombinant adeno-associated virus (rAAV) for use in a method of treating a condition in a subject, the method comprising: administering an rAAV to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting central nervous system (CNS) tissue of the subject with the rAAV; and preferentially transcribing the payload to the CNS tissue infected by the rAAV at an RNA enrichment of at least 100-fold greater than a wild type AAV9.

In various aspects, the present disclosure provides a recombinant adeno-associated virus (rAAV) for use in a method of treating a condition in a subject, the method comprising: administering an rAAV to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting central nervous system (CNS) tissue of the subject with the rAAV; and preferentially delivering the payload to the CNS tissue infected by the rAAV at a DNA enrichment of at least 5-fold greater than a wild type AAV5.

In various aspects, the present disclosure provides a recombinant adeno-associated virus (rAAV) for use in a method of treating a condition in a subject, the method comprising: administering an rAAV to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a central nervous system (CNS) tissue of the subject with the rAAV; and preferentially transcribing the payload to the CNS tissue infected by the rAAV at an RNA enrichment of at least 5-fold greater than a wild type AAV5.

In various aspects, the present disclosure provides a recombinant adeno-associated virus (rAAV) for use in a method of treating a condition in a subject, the method comprising: administering an rAAV to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 18, (b) SEQ ID NO: 3472, (c) SEQ ID NO: 262, (d) SEQ ID NO: 3306, (e) SEQ ID NO: 2028, (f) SEQ ID NO: 2791, (g) SEQ ID NO: 424, (h) SEQ ID NO: 2536, (i) SEQ ID NO: 1971, (j) SEQ ID NO: 415, (k) SEQ ID NO: 3846, (l) SEQ ID NO: 3283, (m) SEQ ID NO: 1956, (n) SEQ ID NO: 3297, (o) SEQ ID NO: 4545, (p) SEQ ID NO: 2661, (q) SEQ ID NO: 1576, (r) SEQ ID NO: 425, (s) SEQ ID NO: 709, (t) SEQ ID NO: 2168, (u) SEQ ID NO: 54, (v) SEQ ID NO: 429, (w) SEQ ID NO: 708, (x) SEQ ID NO: 428, (y) SEQ ID NO: 4119, (z) SEQ ID NO: 3906, (aa) SEQ ID NO: 2456, (bb) SEQ ID NO: 2278, (cc) SEQ ID NO: 5006, or (dd) SEQ ID NO: 426; infecting a CNS tissue of the subject with the rAAV; and delivering the payload to the CNS tissue infected by the rAAV.

In various aspects, the present disclosure provides a recombinant adeno-associated virus (rAAV) for use in a method of treating a condition in a subject, the method comprising: administering an rAAV to the subject, wherein the rAAV encapsidates a payload, and herein the rAAV comprises a VP capsid polypeptide a 581-589 region corresponding to residues 581 to 589 of a VP1 polypeptide, wherein the 581-589 region comprises at least one amino acid substitution as compared to residues 581 to 589 of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8; infecting a CNS tissue of the subject with the rAAV with at least 3-fold higher CNS tissue tropism as compared to a wild type AAV5 capsid comprising a peptide of SEQ ID NO: 1; and delivering the payload to the CNS tissue infected by the rAAV.

In various aspects, the present disclosure provides a recombinant adeno-associated virus (rAAV) for use in a method of treating a condition in a subject, the method comprising: administering an rAAV to the subject, wherein the rAAV encapsidates a payload, and wherein the rAAV comprises a VP capsid polypeptide as described herein; infecting a CNS tissue of the subject with the rAAV; and delivering the payload to the CNS tissue infected by the rAAV

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a cardiac muscle tissue at a DNA enrichment of at least 25-fold greater than a wild type AAV9.

In some aspects, the 581-589 region has a sequence selected from the group consisting of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5092, SEQ ID NO: 4059, SEQ ID NO: 4295, SEQ ID NO: 5030, SEQ ID NO: 4938, SEQ ID NO: 2661, SEQ ID NO: 5215, SEQ ID NO: 4960, SEQ ID NO: 4969, SEQ ID NO: 5023, SEQ ID NO: 4934, SEQ ID NO: 4545, SEQ ID NO: 4963, SEQ ID NO: 4363, SEQ ID NO: 5159, and SEQ ID NO: 4973.

In various aspects, the present disclosure provides a viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 348, (b) SEQ ID NO: 2536, (c) SEQ ID NO: 1576, (d) SEQ ID NO: 4955, (e) SEQ ID NO: 5017, (f) SEQ ID NO: 4349, (g) SEQ ID NO: 4964, (h) SEQ ID NO: 293, (i) SEQ ID NO: 4314, (j) SEQ ID NO: 4995, (k) SEQ ID NO: 4366, (l) SEQ ID NO: 4961, (m) SEQ ID NO: 4952, (n) SEQ ID NO: 5075, (o) SEQ ID NO: 4354, (p) SEQ ID NO: 5060, (q) SEQ ID NO: 5138, (r) SEQ ID NO: 5206, (s) SEQ ID NO: 4288, (t) SEQ ID NO: 5092, (u) SEQ ID NO: 4059, (v) SEQ ID NO: 4295, (w) SEQ ID NO: 5030, (x) SEQ ID NO: 4938, (y) SEQ ID NO: 2661, (z) SEQ ID NO: 5215, (aa) SEQ ID NO: 4960, (bb) SEQ ID NO: 4969, (cc) SEQ ID NO: 5023, or (dd) SEQ ID NO: 4934.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a skeletal muscle tissue at a DNA enrichment of at least 15-fold greater than a wild type AAV9.

In some aspects, the 581-589 region has a sequence selected from the group consisting of SEQ ID NO: 3472, SEQ ID NO: 3297, SEQ ID NO: 2661, SEQ ID NO: 4545, SEQ ID NO: 348, SEQ ID NO: 18, SEQ ID NO: 4349, SEQ ID NO: 293, SEQ ID NO: 5017, SEQ ID NO: 4955, SEQ ID NO: 4366, SEQ ID NO: 5092, SEQ ID NO: 210, SEQ ID NO: 4059, SEQ ID NO: 2536, SEQ ID NO: 5206, and SEQ ID NO: 1971.

In various aspects, the present disclosure provides a viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 3472, (b) SEQ ID NO: 3297, (c) SEQ ID NO: 2661, (d) SEQ ID NO: 4545, (e) SEQ ID NO: 348, (f) SEQ ID NO: 18, (g) SEQ ID NO: 4349, (h) SEQ ID NO: 293, (i) SEQ ID NO: 5017, (j) SEQ ID NO: 4955, (k) SEQ ID NO: 4366, (l) SEQ ID NO: 5092, (m) SEQ ID NO: 210, (n) SEQ ID NO: 4059, (o) SEQ ID NO: 2536, (p) SEQ ID NO: 5206, (q) SEQ ID NO: 1971, (r) SEQ ID NO: 262, (s) SEQ ID NO: 4343, (t) SEQ ID NO: 4995, (u) SEQ ID NO: 4009, (v) SEQ ID NO: 5190, (w) SEQ ID NO: 724, (x) SEQ ID NO: 4288, (y) SEQ ID NO: 4973, (z) SEQ ID NO: 4314, (aa) SEQ ID NO: 1561, (bb) SEQ ID NO: 5147, (cc) SEQ ID NO: 4238, or (dd) SEQ ID NO: 4961.

In various aspects, the present disclosure provides a viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a muscle tissue at a DNA enrichment of at least 20-fold greater than a wild type AAV9.

In some aspects, the 581-589 region has a sequence selected from the group consisting of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, SEQ ID NO: 4366, SEQ ID NO: 4545, SEQ ID NO: 4961, SEQ ID NO: 5206, SEQ ID NO: 5092, SEQ ID NO: 3472, SEQ ID NO: 5075, SEQ ID NO: 4059, SEQ ID NO: 4354, SEQ ID NO: 5060, SEQ ID NO: 4288, SEQ ID NO: 4952, SEQ ID NO: 5138, SEQ ID NO: 18, SEQ ID NO: 5030, SEQ ID NO: 3297, SEQ ID NO: 4295, SEQ ID NO: 4363, SEQ ID NO: 4963, SEQ ID NO: 4973, SEQ ID NO: 5159, SEQ ID NO: 4960, SEQ ID NO: 4938, SEQ ID NO: 5157, SEQ ID NO: 5023, and SEQ ID NO: 4969.

In various aspects, the present disclosure provides a viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 348, (b) SEQ ID NO: 2536, (c) SEQ ID NO: 4955, (d) SEQ ID NO: 5017, (e) SEQ ID NO: 4349, (f) SEQ ID NO: 293, (g) SEQ ID NO: 1576, (h) SEQ ID NO: 2661, (i) SEQ ID NO: 4964, (j) SEQ ID NO: 4314, (k) SEQ ID NO: 4995, (l) SEQ ID NO: 4366, (m) SEQ ID NO: 4545, (n) SEQ ID NO: 4961, (o) SEQ ID NO: 5206, (p) SEQ ID NO: 5092, (q) SEQ ID NO: 3472, (r) SEQ ID NO: 5075, (s) SEQ ID NO: 4059, (t) SEQ ID NO: 4354, (u) SEQ ID NO: 5060, (v) SEQ ID NO: 4288, (w) SEQ ID NO: 4952, (x) SEQ ID NO: 5138, (y) SEQ ID NO: 18, (z) SEQ ID NO: 5030, (aa) SEQ ID NO: 3297, (bb) SEQ ID NO: 4295, (cc) SEQ ID NO: 4363, or (dd) SEQ ID NO: 4963.

In various aspects, the present disclosure provides a viral protein (VP) capsid polypeptide, wherein the VP capsid polypeptide comprises a 581-589 region corresponding to residues 581 to 589 of a VP1 polypeptide, wherein the 581-589 region comprises a sequence having at least 85% identity to any one of any one of SEQ ID NO: 4238-SEQ ID NO: 4933 or SEQ ID NO: 4934-SEQ ID NO: 5233.

In some aspects, the 581-589 region has a sequence of any one of any one of SEQ ID NO: 4238-SEQ ID NO: 4933 or SEQ ID NO: 4934-SEQ ID NO: 5233.

### INCORPORATION BY REFERENCE

All references cited herein are incorporated by reference to the same extent as if each individual publication, database entry (e.g., Genbank sequences or GeneID entries), patent application, or patent, was specifically and individually indicated incorporated by reference in its entirety, for all purposes. This statement of incorporation by reference is intended by Applicants, pursuant to 37 C.F.R. § 1.57(b)(1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

### BRIEF DESCRIPTION OF THE DRAWINGS

These and other features, aspects, and advantages of the present invention will become better understood with regard to the following description, and accompanying drawings where:

FIG. 1 is a schematic of the high throughput AAV capsid engineering system.

FIG. 2A provides a side view (top panel) and top view (bottom panel) of key residues of known AAV capsids that have been shown to interact with target cells.

FIG. 2B illustrates salient features of the AAV capsid library described in EXAMPLE 1, showing the region of introduced diversity, with residue numbering corresponding to the numbering of amino acids in AAV5 VP1.

FIG. 3A shows a next generation sequencing (NGS) analysis of purified secondary screen library virus containing engineered AAV5 variants of the present disclosure and barcoded control serotypes (AAV1, AAV2, AAV5, AAV9, or AAV PHP.B) spiked in at 1×, 10×, and 100× stoichiometric ratios.

FIG. 3B is a schematic highlighting identification of AAV spike-in control DNA in CNS, liver, heart, and skeletal muscle by NGS analysis of NHP tissues post-selection.

FIG. 3C demonstrates that DNA from 94% of capsid variants predicted to target the CNS were found in the CNS.

FIG. 3D shows a comparison of DNA abundance in the CNS for three engineered AAV5 variants, SEQ ID NO: 18 (AAGRFNYFG), SEQ ID NO: 54 (AEDYWDLGA), and SEQ ID NO: 2028 (MDDICEFYA), of the present disclosure compared with wild type AAV5 and AAV9 spike-ins shows much greater infection than the highest (100×) controls.

FIG. 3E shows that engineered AAV5 variants of the present disclosure exhibit decreased liver infection compared to wild type (parental) AAV5 control.

FIG. 4 is a bar graph showing relative accumulation, expressed as log 10 fold change in brain accumulation relative to other tissues, of variant AAV capsids comprising 581-589 regions of SEQ ID NO: 415 (DARVYRALD), SEQ ID NO: 569 (DSASPIMGM), SEQ ID NO: 428 (DAWQMLSGN), SEQ ID NO: 430 (DAWSYQCYH), SEQ ID NO: 708 (EAWMYHQFH), SEQ ID NO: 3906 (YSGVRVTGY), SEQ ID NO: 709 (EAWSKLEQP), SEQ ID NO: 3297 (TAGRFNYFD), SEQ ID NO: 1956 (LVGWTLQHV), SEQ ID NO: 885 (ESWMKLEWQ), SEQ ID NO: 3283 (TAAFAYKYE), SEQ ID NO: 3472 (TSHYITFTP), SEQ ID NO: 426 (DAWMMMWGS), SEQ ID NO: 3306 (TANVYRSGQ), SEQ ID NO: 1971 (LWGWTLQHQ), SEQ ID NO: 425 (DAWLQLKDN), SEQ ID NO: 262 (CATRFNIGG), SEQ ID NO: 424 (DAWCFISSY), SEQ ID NO: 18 (AAGRFNYFG), SEQ ID NO: 54 (AEDYWDLGA), or SEQ ID NO: 2028 (MDDICEFYA). Accumulation was compared to wild type AAV capsids of AAV9, AAV1, AAV PHP.B, AAV2, and AAV5.

FIG. 5 is a bar graph showing relative accumulation, expressed as log 2 fold change in muscle accumulation relative to other tissues, of variant AAV capsids comprising 581-589 regions of SEQ ID NO: 4318 (CKEWYVRDR), SEQ ID NO: 4960 (CWREFSFQN), SEQ ID NO: 4371 (CVSLHSISM), SEQ ID NO: 3975 (QAHHYNILN), SEQ ID NO: 5215 (CVRTLQSPD), SEQ ID NO: 4598 (LAWSQLLTP), SEQ ID NO: 4359 (CVATKSRML), SEQ ID NO: 5665 (RQTTYDCLD), SEQ ID NO: 257 (CAHYAQWGK), SEQ ID NO: 344 (CSSGFHLFH), SEQ ID NO: 5607 (QCGFIRLNE), SEQ ID NO: 3528 (TVTHMSQHC), SEQ ID NO: 5155 (CVIWYHKYE), SEQ ID NO: 5363 (GCMCIRHAY), SEQ ID NO: 4633 (MACWKNATE), SEQ ID NO: 2525 (QAGSSFHQA), SEQ ID NO: 386 (CYQFWSQYS), SEQ ID NO: 4970 (CINFEIKLG), SEQ ID NO: 5143 (CWRHNIISP), SEQ ID NO: 5178 (CIKWVDIFP), SEQ ID NO: 2505 (PTQFYGPAF), SEQ ID NO: 3709 (VSASFQQHV), SEQ ID NO: 5056 (CVLNYFQFG), SEQ ID NO: 5017 (CFYKIQHKG), SEQ ID NO: 5040 (CVLRMGTFC), SEQ ID NO: 5161 (CTHMMGKSP), SEQ ID NO: 1128 (GFANFMMNF), SEQ ID NO: 2204 (MTMTATTFG), SEQ ID NO: 4316 (CIRHAQDCY), SEQ ID NO: 5463 (KISPWDGFY), SEQ ID NO: 4944 (CWKWQMMFG), SEQ ID NO: 4955 (CVWSQILGG), SEQ ID NO: 4952 (CVILSTRDK), SEQ ID NO: 3260 (SVSPCFCNS), SEQ ID NO: 384 (CYMPFKMQH), SEQ ID NO: 5038 (CVVWPVLGQ), SEQ ID NO: 2371 (NMTMAHQAS), SEQ ID NO: 289 (CIHSVHFAA), SEQ ID NO: 2985 (RVDAFNRGT), SEQ ID NO: 4508 (GSCLKIAQE), SEQ ID NO: 4292 (CAMLLQAVQ), SEQ ID NO: 4995 (CVGYRVHSP), SEQ ID NO: 4949 (CYHQVFSQG), SEQ ID NO: 5077 (CVGSQLHAM), SEQ ID NO: 2370 (NMNTFGKMN), SEQ ID NO: 5193 (CWSHLYFQT), SEQ ID NO: 4568 (KANEDLKQF), SEQ ID NO: 5206 (CFFYPMSMS), SEQ ID NO: 4215 (LYAFYMSSE), SEQ ID NO: 4841 (SSDQYEEMN), SEQ ID NO: 5092 (CISNYLKQG), SEQ ID NO: 4335 (CPMKHIQDR), SEQ ID NO: 5029 (CSFNIHKHT), SEQ ID NO: 3258 (SVRMFDAQY), SEQ ID NO: 4349 (CSSYLVTAN), SEQ ID NO: 348 (CTASWMSWD), or SEQ ID NO: 2729 (QQQQTNFQN). AAV variants that were generated using machine learning (ML) are denoted with arrows. Accumulation was compared to wild type AAV capsids of AAV9, AAV PHP.B, AAV1, AAV2, and AAV5.

FIG. 6 is a Circos plot depicting variant AAV5 capsids identified in a primary screen performed as illustrated in FIG. 1. Venn diagrams in the figure are not to scale. Over 1 million variant capsids were identified as unique to cardiac tissue, over 100,000 variants were identified as unique to skeletal muscle tissue, and over 1,000 variants were identified as shared between cardiac tissue and skeletal muscle tissue. The variants identified in cardiac tissue, skeletal muscle tissue, or both cardiac and skeletal muscle tissue exhibited low shared identity with liver (about 0.7% overlap) and other tissues (e.g., non-muscle tissues).

FIG. 7A illustrates the normalized counts of plasmid library DNA compared to assembled virus DNA for wild type AAV controls (textured points) and for variant AAV candidates (gray circles). Levels of assembled virus for variant AAV candidates were comparable to wild type AAV controls at 1× spike-in frequency.

FIG. 7B shows DNA levels in liver versus CNS for variant AAV candidates (gray circles), wild type controls (textured points), and select AAV candidates with CNS tropism (open circles). Circle size for the select AAV candidates corresponds to amount of functional transduction in CNS tissue, with larger circles representing higher RNA expression in CNS tissue. CNS-tropic AAV candidates were enriched in CNS tissue relative to liver tissue.

FIG. 7C shows DNA levels in muscle versus CNS for variant AAV candidates (gray circles), wild type controls (textured points), and select AAV candidates with CNS tropism (open circles). Circle size for the select AAV candidates corresponds to amount of functional transduction in CNS tissue, with larger circles representing higher RNA expression in CNS tissue. CNS-tropic AAV candidates were enriched in CNS tissue relative to muscle tissue.

FIG. 8A shows a bar graph of the proportion of candidates from different library sub-sets that were identified as muscle-specific in a secondary screen. Muscle candidates generated using machine learning (“Muscle ML Synthetic”) contained a higher proportion of muscle-specific sequences than candidates identified in muscle in a primary screen (“Muscle Observed”), CNS targeted sequences, or negative control sequences. Muscle candidate sequences generated using machine learning were five times more likely to be heart- and/or skeletal muscle-specific than muscle candidates identified in the primary screen and were 20 times more likely to be heart- and/or skeletal muscle-specific than CNS targeted variants. Candidates were classified as muscle-specific if they were >2-fold enriched in heart and/or skeletal muscle relative to all other tissues and viral input (FDR <0.1, permutations with α-RRA to account for consistency across multiple barcodes for each capsid).

FIG. 8B shows violin and box and whisker plots comparing the predicted probability of muscle specificity from primary screen for candidates that were (“yes”) or were not (“no”) identified as muscle-specific in the secondary screen. Candidates were classified as muscle-specific if they were >2-fold enriched in heart and/or skeletal muscle relative to all other tissues and viral input (FDR <0.1, permutations with α-RRA to account for consistency across multiple barcodes for each capsid).

FIG. 9 shows the CNS prediction score for CNS targeted candidates identified from multiple non-human primates (NHPs), multiple samples, observational enrichment, frequency enrichment, or sequence similarity, or generated by machine learning. CNS targeted candidates generated using machine learning had, on average, higher CNS prediction scores than candidates identified from other sources.

FIG. 10 is a plot showing relative accumulation and functional transduction of wild type AAV9 capsids at varying concentrations (top) and three identified CNS tissue-tropic AAV variants (SEQ ID NO: 18, SEQ ID NO: 424, and SEQ ID NO: 2028, bottom) in different tissues following systemic administration. RNA expression, representing functional transduction in each tissue, is shown on the x-axis. Circle size represents relative accumulation of AAV virions in each tissue, as measured by DNA. The AAV variants are highly selective for CNS tissue.

FIG. 11 is a plot showing relative accumulation and functional transduction of three identified CNS tissue-tropic AAV variants (SEQ ID NO: 18, SEQ ID NO: 424, and SEQ ID NO: 2028) in different CNS tissues following systemic administration. DNA quantification of a given AAV variant is shown on the x-axis. Circle size corresponds to amount of functional transduction of a given AAV variant in each tissue, with larger circles representing higher RNA expression in CNS tissue. The AAV variants show broad functional transduction in CNS tissues.

FIG. 12 illustrates the relative functional transduction of RNA expression in different regions of the brain of wild type AAV9 (left) and a CNS tissue-tropic AAV variant of SEQ ID NO: 2028 (right). Brain regions sampled include cortex (forebrain), cortex (occipital), cortex (temporal), thalamus, hypothalamus, hippocampus, cerebellum, caudate, putamen, pons, medulla, midbrain, and substantia nigra. Darker shading indicates higher transduction, as measured by RNA expression. The AAV variant shows broad functional transduction in CNS tissues.

FIG. 13 shows that three AAV variants of the present disclosure (SEQ ID NO: 18, SEQ ID NO: 424, and SEQ ID NO: 2028, also shown in FIG. 11) functionally transduce neurons. CAG promoter-driven RNA expression highlights functional transduction of any cell type while SYN promoter-driven RNA expression highlights functional transduction of neurons.

FIG. 14 shows that some AAV variants of the present disclosure that target CNS also demonstrate dorsal root ganglion (DRG) depletion.

FIG. 15 shows that promoter usage differentiates CNS AAV variants and heart muscle AAV variants. For example, certain AAV CNS variants of the present disclosure show CAG and SYN promoter-driven RNA expression, while certain AAV heart muscle variants show only CAG promoter-driven RNA expression.

FIG. 16A is bar plot of group intersection size for capsids enriched in cardiac muscle tissue in non-human primates (NHPs), mice, or both NHPs and mice.

FIG. 16B is a scatter plot showing log 2-fold change in enrichment in cardiac tissue in NHPs versus mice for capsid variants or wild type AAV capsids. Seventeen variants enriched in cardiac tissue in both NHPs and mice are shown in dark circles.

FIG. 17A is bar plot of group intersection size for capsids enriched in CNS tissue in non-human primates (NHPs) or mice.

FIG. 17B is a scatter plot showing log 2-fold change in enrichment in CNS tissue in NHPs versus mice for capsid variants or wild type AAV capsids.

FIG. 18 is a scatter plot of the average Z-score of CNS tissue enrichment for screened variant AAV capsids, ordered by average variant capsid rank for twelve methods of differential expression analysis. The high scoring variant capsid, corresponding to the outlined points in the upper left quadrant, were ranked consistently highly across multiple analysis strategies and ranked consistently higher than wild type AAV5 (wtAAV5) and wild type AAV9 (wtAAV9).

### DETAILED DESCRIPTION OF THE INVENTION

Unless described otherwise, all technical and scientific terms used herein have the meaning commonly understood by one of ordinary skill in the art to which the invention pertains.

Unless otherwise stated, whenever a range is recited, the range is inclusive of the recited endpoints. For example, the region from amino acid residue 581 to amino acid residue 589 of SEQ ID NO: 1 includes amino acid residues 581 and 589.

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 77.7%, 80%, 85%, 88.8%, 90%, 92%, 95%, 96%, 97%, 98% or 99% identical to any sequence described herein (which can correspond with a particular nucleic acid or amino acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed. The term percent “identity” or percent “homology,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. For purposes herein, determination of percent identity and sequence similarity is performed using the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/). For example, where an amino acid sequence consisting of 9 residues is compared with another 9 residue amino acid sequence, if 8 residues match then the sequences have 88.8% identity, if 7 residues match then the sequences have 77.7% identity.

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

Peptide sequences for use in the present invention may include one or more conservative amino acid substitutions, such that the resulting sequence has a similar amino acid sequence and/or retains the same function. The skilled person is aware that various amino acids have similar biochemical properties and thus are “conservative”. One or more such amino acids of a protein, polypeptide or peptide can often be substituted by one or more other such amino acids without eliminating a desired activity of that protein, polypeptide or peptide. Thus, the amino acids glycine, alanine, valine, leucine, and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulfur containing side chains). It should be appreciated that amino acid substitutions within the scope of the present invention can be made using naturally occurring or non-naturally occurring amino acids. For example, the methyl group on an alanine may be replaced with an ethyl group, and/or minor changes may be made to the peptide backbone. Whether or not natural or synthetic amino acids are used, it is preferred that only L-amino acids are present. Substitutions of this nature are often referred to as “conservative substitutions”.

As used herein, “tropism” of a rAAV for a tissue may refer to the ability of a given rAAV to preferentially infect a given cell type or tissue. A degree of tropism may be determined by a ratio of an infection rate in a targeted tissue to an infection rate in a different, non-targeted tissue. As used herein, increased tropism for a given cell type or tissue, such as increased tropism conferred by a 581-589 region, is determined relative to a wild type AAV5 capsid. As used herein, “detargeting” of a rAAV to a tissue may refer to the ability of a given rAAV to avoid infecting a detargeted tissue or cell type while infecting one or more other tissues or cell types. A degree of detargeting may be determined by a ratio of an infection rate in a detargeted tissue to an infection rate of a different, non-detargeted tissue. As used herein, increased detargeting for a given cell type or tissue, such as increased detargeting conferred by a 581-589 region, is determined relative to a wild type AAV5 capsid.

As used herein, “tissue tropism” refers to a preference of a virus having an engineered VP capsid polypeptide of the present disclosure to infect a given tissue or be enriched in or accumulate in a given tissue. A “tissue-tropic” rAAV may specifically target or infect a first tissue or set of tissues and may not target or infect a second tissue or set of tissues. For example, a “CNS-tropic” rAAV may specifically target or infect CNS tissue and may not target or infect muscle, skin, bone, or other tissue or tissues. A “tissue-detargeted” rAAV may specifically avoid targeting or avoid infection of the detargeted tissue or set of tissues while infecting a second tissue or set of tissues. For example, a “liver-detargeted” rAAV may not target or infect liver tissue but may infect one or more other tissues, such as nervous, muscle, skin, bone, and/or other tissue. Tissue tropism or tissue detargeting, when used as a relative term and depending on the context in which it is described herein, refers to an increase or decrease in tissue tropism of a given rAAV virion having a first capsid polypeptide in a first tissue as compared to a second tissue and/or refers to an increase or decrease in tissue tropism of a given rAAV virion having a first capsid polypeptide to an rAAV virion having a second capsid polypeptide. In some embodiments, the first tissue can be a group of tissues. In some embodiments, the second tissue can be a group of tissues. For example, the first tissue may be CNS tissues, which comprise cortex forebrain, cortex occipital, cortex temporal, thalamus, hypothalamus, substantia nigra, hippocampus DG, hippocampus CA1, hippocampus CA3, and cerebellum and the second tissue may be a non-CNS tissue consisting collectively of liver, skeletal muscle, heart, lung, spleen, lymph node, bone marrow, mammary gland, skin, adrenal gland, thyroid, colon, sciatic nerve, and spinal cord tissues. As another example, the first tissue may be liver tissue and the second tissue may be non-liver tissue consisting collectively of CNS tissues, skeletal muscle, heart, lung, spleen, lymph node, bone marrow, mammary gland, skin, adrenal gland, thyroid, colon, sciatic nerve, and spinal cord tissues.

In some embodiments, the rAAV virions of the present disclosure may also be referred to as preferentially targeting a given tissue or having tissue selectivity for a given tissue. For example, an rAAV virion that preferentially targets CNS tissue may specifically target or infect CNS tissue and may not target or infect skeletal muscle, cardiac muscle, or other tissues or may target other tissues to a lesser degree than CNS tissue. As another example, an rAAV virion that has retinal tissue selectivity may specifically target or infect CNS tissue and may not target or infect skeletal muscle, cardiac muscle, or other tissues or may target other tissues to a lesser degree than CNS tissue.

For simplicity throughout this disclosure, viral capsid protein is generally referred to as “VP.” Viral capsid protein is referred to as VP1 when referencing AAV5 VP1 positional notation. In all cases, viral capsid sequences and mutations disclosed herein should be understood as pertaining to all isoforms of the capsid protein (VP1, VP2, and VP3), as a mixture of these isoforms assemble to form virions. The positional amino acid residue designations “581 to 589” are relative to the translational start of the VP1 polypeptide and should be adjusted accordingly to the relative start sites of VP2 and VP3. It should be understood that the present disclosure, when describing any particular VP1 sequence with mutations at particular amino acid residue positions, necessarily also encompasses corresponding mutations in VP2 and VP3. For example, any consensus sequence or specific sequence of a VP1 capsid protein having one or more mutations in the 581-589 region, corresponding to amino acid residues 581 to 589 of VP1, also encompasses VP2 and VP3 capsid proteins having said one or more mutations in an amino acid residue region in VP2 and VP3 corresponding to the amino acid residues of the VP1 581 to 589 region. For example, the amino acid residues of the 581 to 589 region of VP1 (SEQ ID NO: 1; MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPST SSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTYNLQEIVPGSVWMERDVYLQGPIWAKIPETGAHFHPSPAMGGFGLKHPPPM MLIKNTPVPGNIT SFSDVPVSSFITQYSTGQVTVEMEWELKKENSKRWNPEIQYTNNYN DPQFVDFAPDSTGEYRTTRPIGTRYLTRPL) correspond to the amino acid residues of the 445 to 453 region of VP2 (SEQ ID NO: 10; TAPTGKRIDDHFPKRKKARTEEDSKPSTSSDAEAGPSGSQQLQIPAQPASSLGADTMSAG GGGPLGDNNQGADGVGNASGDWHCDSTWMGDRVVTKSTRTWVLPSYNNHQYREIKS GSVDGSNANAYFGYSTPWGYFDFNRFHSHWSPRDWQRLINNYWGFRPRSLRVKIFNIQ VKEVTVQDSTTTIANNLTSTVQVFTDDDYQLPYVVGNGTEGCLPAFPPQVFTLPQYGY ATLNRDNTENPTERSSFFCLEYFPSKMLRTGNNFEFTYNFEEVPFHSSFAPSQNLFKLAN PLVDQYLYRFVSTNNTGGVQFNKNLAGRYANTYKNWFPGPMGRTQGWNLGSGVNRA SVSAFATTNRMELEGASYQVPPQPNGMTNNLQGSNTYALENTMIFNSQPANPGTTATY LEGNMLITSESETQPVNRVAYNVGGQMATNNQSSTTAPATGTYNLQEIVPGSVWMERD VYLQGPIWAKIPETGAHFHPSPAMGGFGLKHPPPMMLIKNTPVPGNITSFSDVPVSSFITQ YSTGQVTVEMEWELKKENSKRWNPEIQYTNNYNDPQFVDFAPDSTGEYRTTRPIGTRY LTRPL) and to the amino acid residues of 389 to 397 region of VP3 (SEQ ID NO: 11; MSAGGGGPLGDNNQGADGVGNASGDWHCDSTWMGDRVVTKSTRTWVLPSYNNHQY REIKSGSVDGSNANAYFGYSTPWGYFDFNRFHSHWSPRDWQRLINNYWGFRPRSLRVK IFNIQVKEVTVQDSTTTIANNLTSTVQVFTDDDYQLPYVVGNGTEGCLPAFPPQVFTLPQ YGYATLNRDNTENPTERSSFFCLEYFPSKMLRTGNNFEFTYNFEEVPFHSSFAPSQNLFK LANPLVDQYLYRFVSTNNTGGVQFNKNLAGRYANTYKNWFPGPMGRTQGWNLGSGV NRASVSAFATTNRMELEGASYQVPPQPNGMTNNLQGSNTYALENTMIFNSQPANPGTT ATYLEGNMLITSESETQPVNRVAYNVGGQMATNNQSSTTAPATGTYNLQEIVPGSVWM ERDVYLQGPIWAKIPETGAHFHPPSPAMGGFGLKHPPPMMLIKNTPVPGNITSFSDVPVSS FITQYSTGQVTVEMEWELKKENSKRWNPEIQYTNNYNDPQFVDFAPDSTGEYRTTRPIG TRYLTRPL). As used herein, “wild type AAV5” or “wild type AAV5 capsid polypeptide” refers to a VP1 capsid polypeptide of SEQ ID NO: 1, a VP2 capsid polypeptide of SEQ ID NO: 10, a VP3 capsid polypeptide of SEQ ID NO: 11, or a combination thereof. Also as used herein, a “wild type 581-589 region” refers to a 581 to 589 region of VP1 having a sequence of

(SEQ ID NO: 9)
ATGTYNLQE.

As used herein, “581-589 region” refers to a region or fragment of VP1 corresponding to amino acid residues 581 to 589 relative to the translational start of the VP1 polypeptide. The 581-589 region may also be referred to as a “variant region.” The 581-589 region corresponds to amino acid residues 445 to 453 of VP2 and to amino acid residues 389 to 397 of VP3. The 581-589 region may confer tissue tropism to an AAV, and defined variants (e.g., SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811) may be engineered to confer tissue tropism to an rAAV formed from viral capsid polypeptides (VP1, VP2, and VP3) comprising the 581-589 region. The VP1 with a generalized 581-589 region is provided in SEQ ID NO: 2 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPX¹X²X³X⁴X⁵X⁶X⁷X⁸X⁹IVPGSVWMERDVYLQGPIWAKIPETGAHFHPSPAMGGFGLK HPPPMMLIKNTPVPGNITSFSDVPVSSFITQYSTGQVTVEMEWELKKENSKRWNPEIQYT NNYNDPQFVDFAPDSTGEYRTTRPIGTRYLTRPL), in which the 581-589 region has a sequence of X¹X²X³X⁴X⁵X⁶X⁷X⁸X⁹; wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V.

It should be understood that the present disclosure includes polynucleotide sequences encoding for any sequence disclosed herein. For example, if an amino acid sequence is provided, the present disclosure also encompasses a polynucleotide sequence encoding for said amino acid sequence.

It should be understood that further embodiments include mutations in VP1, VP2, VP3, or any combination thereof that do not alter the desired properties (e.g., a particular tissue tropism) or affect viral assembly, as described herein.

An rAAV virion is made of a capsid that may include the engineered AAV5 VP capsid polypeptides disclosed herein (e.g., VP1, VP2, and VP3 capsid polypeptides).

### Engineered Capsids and Capsid Polypeptides for Tissue Tropism

Described herein are engineered capsids, engineered capsid polypeptides, and 581-589 regions of capsid polypeptides that confer tissue tropism (e.g., tissue-specific accumulation, tissue-specific infection, or both) to a viral capsid. In some embodiments, an engineered capsid may comprise one or more engineered capsid polypeptides assembled into a recombinant adeno-associated virus (rAAV) viral capsid. In some embodiments, an engineered capsid polypeptide may comprise a variation in the 581-589 region. The 581-589 region of viral capsid polypeptides, corresponding to amino acid residues 581 to 589 of the VP1 polypeptide, amino acid residues 445 to 453 of the VP2 polypeptide, and amino acid residues 389 to 397 of the VP3 polypeptide, is located at the AAV interface that interacts with host cells and tissues.

Also described herein are methods of using engineered capsids comprising engineered capsid polypeptides with 581-589 regions for tissue-specific delivery of a payload (e.g., a polynucleotide, such as a transgene) encapsidated by the engineered capsid. Recombinant AAVs comprising VP capsid polypeptides with 581-589 regions engineered for tissue specificity may be used to specifically infect a target tissue. Using tissue-tropic rAAV viral capsids for payload delivery provides numerous advantages over using adeno-associated virus (AAV) viral capsids that lack tissue tropism including reduced toxicity, lower dose needed to produce a therapeutic effect, wider therapeutic window, and reduced immune response. Furthermore, tissue-specific payload delivery may enable targeted therapies even when administering systemically. For example, a central nervous (CNS) tissue-tropic rAAV viral capsid may be systemically administered to specifically deliver a payload to the CNS for treatment of a neurological disease. In another example, a muscle-tropic rAAV viral capsid may be systemically administered to specifically deliver a payload to muscle for treatment of a muscular disease, including cardiac muscle and vascular diseases.

In some embodiments, a tissue-tropic capsid of the present disclosure may be CNS tissue-tropic. For example, a CNS tissue-tropic capsid polypeptide may confer tropism for one or more CNS tissues (e.g., hippocampus (dentate gyrus, CA1, or CA3), cerebellum, hypothalamus, cortex (occipital, temporal, or forebrain), substantia nigra, thalamus, or combinations thereof). In some embodiments, a tissue-tropic capsid, engineered capsid polypeptide, or 581-589 region of a capsid polypeptide may be muscle-tropic. For example, a muscle-tropic capsid polypeptide may confer tropism for one or more muscle tissues (e.g., aorta, esophagus, heart (including atrium, ventricle, valves), skeletal muscle (including biceps, brachii, femoris, diaphragm, gastrocnemius, tibialis anterior, triceps, quadriceps, including vastus lateralis)), or muscle fibers (e.g., type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof).

An engineered capsid polypeptide of the present disclosure may comprise one or more amino acid substitutions relative to an AAV5 viral protein (VP) polypeptide (e.g., a VP1 polypeptide of SEQ ID NO: 1, a VP2 polypeptide of SEQ ID NO: 10, or a VP3 polypeptide of SEQ ID NO: 11). The engineered capsid polypeptide may comprise one or more amino acid substitutions relative to a VP1 polypeptide of any or all of SEQ ID NO: 3-SEQ ID NO: 8. In some embodiments, the engineered capsid polypeptide may comprise a 581-589 region comprising one or more amino acid substitutions in a region of a VP polypeptide (e.g., a 581-589 region of VP1, VP2, VP3, or a combination thereof) corresponding to amino acid residues 581 to 589 of VP1 (e.g., SEQ ID NO: 1), amino acid residues 445 to 453 of VP2 (e.g., SEQ ID NO: 10), or amino acid residues 389 to 397 of VP3 (e.g., SEQ ID NO: 11). In some embodiments, the 581-589 region may be present in VP1, VP2, and VP3. An engineered viral capsid may be assembled from VP1, VP2, and VP3 polypeptides comprising a 581-589 region with one or more amino acid substitutions. In some embodiments, the 581-589 region may confer or increase the likelihood of conferring CNS tissue-tropism. For example, a 581-589 region with increased likelihood of conferring CNS tissue-tropism may comprise a sequence of any one of SEQ ID NO: 12-SEQ ID NO: 3937, SEQ ID NO: 3938-SEQ ID NO: 4237, or SEQ ID NO: 5234-SEQ ID NO: 5807. In some embodiments, the 581-589 region may confer or increase the likelihood of conferring muscle-tropism. For example, a 581-589 region with increased likelihood of conferring muscle-tropism may comprise a sequence of any one of SEQ ID NO: 4238-SEQ ID NO: 4933, SEQ ID NO: 4934-SEQ ID NO: 5233, or SEQ ID NO: 5808-SEQ ID NO: 5811.

In some embodiments, the 581-589 region may confer or increase the likelihood of conferring CNS tissue-tropism. For example, a 581-589 region with increased likelihood of conferring CNS tissue-tropism may comprise a sequence of any one of SEQ ID NO: 12-SEQ ID NO: 3937 or SEQ ID NO: 3938-SEQ ID NO: 4237. In some embodiments, the 581-589 region may confer or increase the likelihood of conferring muscle-tropism. For example, a 581-589 region with increased likelihood of conferring muscle-tropism may comprise a sequence of any one of SEQ ID NO: 4238-SEQ ID NO: 4933 or SEQ ID NO: 4934-SEQ ID NO: 5233.

### Capsid Engineering Methods

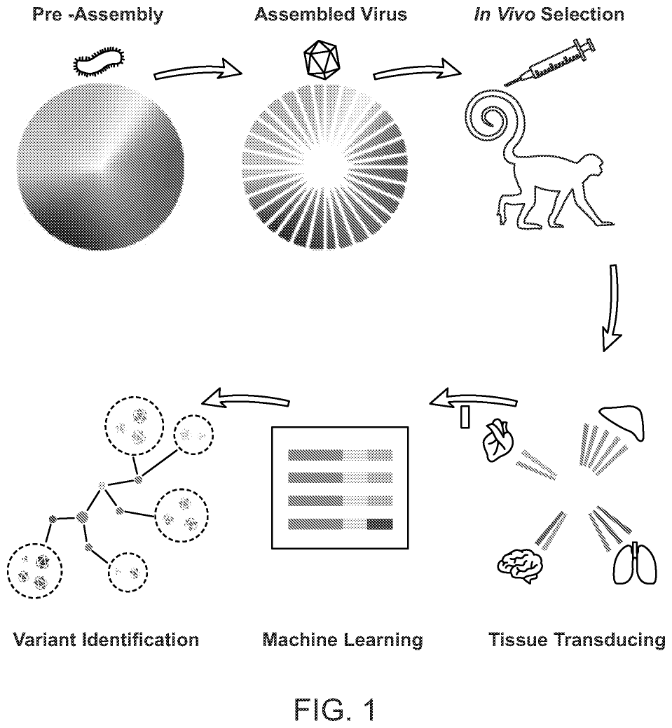

Disclosed herein is a system for high-throughput engineering of engineered AAV capsids with modified function, including increased or decreased infectivity of desired tissues, such as increased targeting of the central nervous system (CNS), increased targeting of muscle, decreased targeting of non-CNS tissue, or decreased targeting of non-muscle tissue relative to a wild type AAV5 capsid (e.g., comprising a VP capsid polypeptide of SEQ ID NO: 1). A general schematic of the process is shown in FIG. 1. However, it should be understood that the present disclosure also encompasses reasonable variations or extensions to the method that are understood to those of ordinary skill in the art. As shown in FIG. 1, the method may begin with production of a capsid library with theoretical diversity of 5×10¹¹ (5e11) unique sequence variants. Higher or lower theoretical diversities are also encompassed herein. For example, a capsid library may have a theoretical diversity of from about 1×10³ (1e3) to about 1×10²⁰ (1e20). The library may then be cloned into plasmids, transformed into bacteria, and subsequently, library plasmids are screened for productive virion assembly in a production cell line. The assembled virions may then be administered intravenously into non-human primates (NHP). After a period of time sufficient for distribution, infection, and stable transduction, the NHP may be sacrificed, organs harvested, and sequences of AAV capsids in each tissue may be determined by deep sequencing.

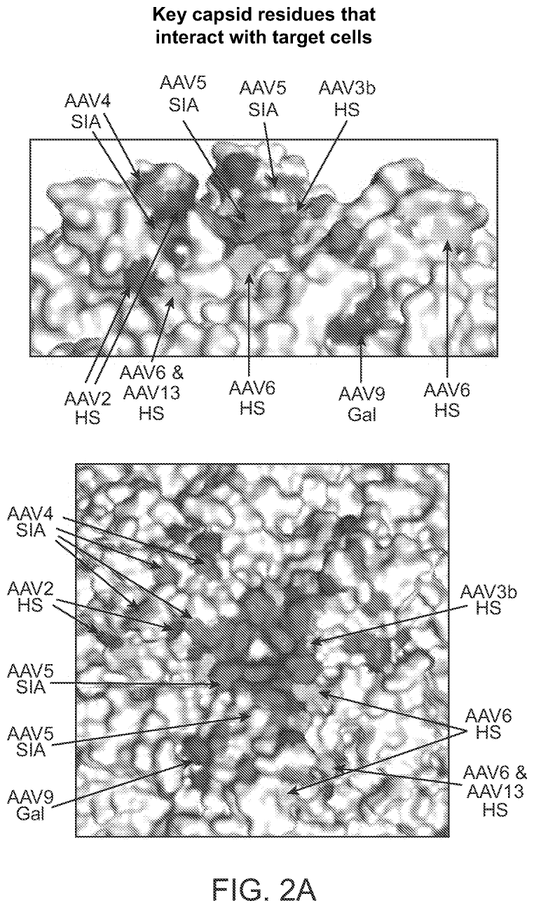

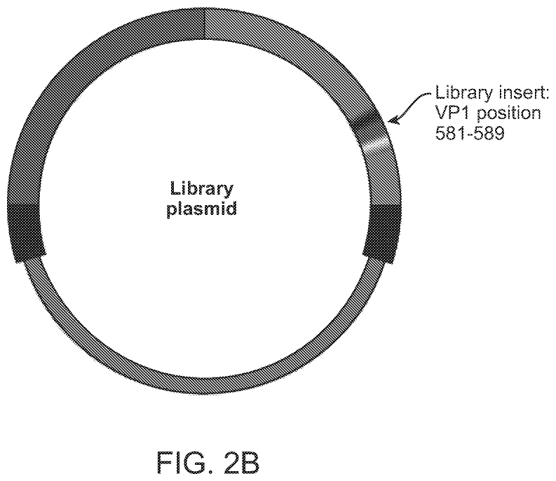

FIG. 2A provides a side view (top panel) and top view (bottom panel) of the surface of a prototype AAV virion, identifying residues of known AAV capsids, including AAV2, AAV5, AAV6, and AAV9, that have been shown in the research literature to interact with target cells. These target-interacting residues correspond to amino acids 581 to 589 in the AAV5 VP1 capsid protein.

FIG. 2B shows the salient elements of the library plasmid, illustrating rep and cap coding sequences positioned between AAV ITRs. In the illustrated embodiment, further described in EXAMPLE 1, variation is introduced into each of residues 581 to 589 of the AAV5 cap protein (“Library variant region”) that is present in VP1, VP2, and VP3. Each of the 20 natural amino acids is introduced at each of the 9 positions of the 581-589 region, providing a theoretical library diversity of 20⁹ (20\hat{ }9; approximately 5×10¹¹ (5e11)) unique sequence variants.

The 581-589 region targeted for engineering is the most likely to interact with target cell receptors, and relatively tolerant to changes without disrupting virion assembly. Unlike earlier approaches that add unstructured peptides that protrude above the virion 3-fold axis of symmetry, the current approach introduces sequence diversity that alters the characteristics of the binding pocket. In addition, this approach may change the overall structure of the receptor-binding trimer, allowing for altered allosteric interactions outside the binding pocket (e.g., AAVR PKD1). Introduced diversity is non-random, thereby reducing missense and frameshifts of randomized libraries.

By cloning the polynucleotide encoding the capsid variants into the packaged viral genome (between the ITRs), the recombinant virions with variant capsids carry polynucleotides having their cognate mutation, so the unique variant providing the desired function can be identified by sequencing packaged virus or infected cells.

In some embodiments, the capsid is a capsid selected from AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, or AAVhu68 (for example, as described in WO2020/033842, incorporated herein by reference in its entirety). For example, the capsid may be an AAV5 capsid. The hu68 capsid is described in WO 2018/160582, incorporated herein by reference in its entirety.

Such capsids may comprise a 581-589 region corresponding amino acid residues 581 to 589 of the AAV5 VP1, and as such analogous engineered VP capsids with desired tissue tropism or preference, ability to assemble, and exhibit various other desired traits are encompassed herein. Thus, any one of the engineered AAV5 VP capsid polypeptides disclosed herein having a mutation or substitution in the 581-589 region corresponding to the 581 to 589 region of AAV5 VP1 may be inserted into the corresponding region in any one of the other AAV capsids described herein and the present disclosure encompasses such variants.

In some embodiments, the capsid is a derivative, modification, or pseudotype of AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, or AAVhu68. For example, the capsid may be a derivative of AAV5.

In some embodiments, capsid protein is a chimera of capsid proteins from two or more serotype selected from AAV1, AAV2, AAV3, AAV4, AAV5, AAV6, AAV7, AAV8, AAV9, AAV 10, AAV11, AAV12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.Rh74, AAV.v66, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, or AAVhu68 (for example, as described in WO2020/033842, incorporated herein by reference in its entirety). In certain embodiments, the capsid is an rh32.33 capsid, described in U.S. Pat. No. 8,999,678, incorporated herein by reference in its entirety.

Such capsids may comprise a 581-589 region corresponding to 581 to 589 of the AAV5 VP1, and as such analogous engineered VP capsids with desired tissue tropism or preference, ability to assemble, and exhibit various other desired traits are encompassed herein.

### VP-Encoding Polynucleotides, Vectors, and Vector Libraries

Accordingly, in a first aspect, polynucleotides are provided. The polynucleotides encode an adeno-associated virus (AAV) VP1 capsid polypeptide having the amino acid sequence of SEQ ID NO: 2, wherein X¹, X², X³, X⁴, X⁵, X⁶, X⁷, X⁸, and X⁹ are each independently selected from the 20 naturally occurring amino acids, using standard one letter codes, from A, R, N, D, C, E, Q, G, H, I, L, K, M, F, P, S, T, W, Y, and V. The sequence X¹X²X³X⁴X⁵X⁶X⁷X⁸X⁹ of SEQ ID NO: 2 corresponds to the 581-589 region of VP1. The polynucleotide encodes a polypeptide that includes at least one mutation of the native AAV5 capsid and thus does not have the sequence of SEQ ID NO: 1. In addition, the polypeptide does not have the sequence of SEQ ID NO: 3 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTVNLQEIVPGSVWMERDVYLQGPIWAKIPETGAHFHPSPAMGGFGLKHPPPM MLIKNTPVPGNIT SFSDVPVSSFITQYSTGQVTVEMEWELKKENSKRWNPEIQYTNNYN DPQFVDFAPDSTGEYRTTRPIGTRYLTRPL), SEQ ID NO: 4 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTYNTQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), SEQ ID NO: 5 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPTTGTYNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), SEQ ID NO: 6 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCD STWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPAAGTYNLQEIVPGSVWMERDVYLQGPIWAKIPETGAHFHPSPAMGGFGLKHPPPM MLIKNTPVPGNIT SFSDVPVSSFITQYSTGQVTVEMEWELKKENSKRWNPEIQYTNNYN DPQFVDFAPDSTGEYRTTRPIGTRYLTRPL), SEQ ID NO: 7 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGAYNLQEIVPGSVWMERDVYLQGPIWAKIPETGAHFHPSPAMGGFGLKHPPPM MLIKNTPVPGNITSFSDVPVSSFITQYSTGQVTVEMEWELKKENSKRWNPEIQYTNNYN DPQFVDFAPDSTGEYRTTRPIGTRYLTRPL), or SEQ ID NO: 8 (MSFVDHPPDWLEEVGEGLREFLGLEAGPPKPKPNQQHQDQARGLVLPGYNYLGPGNG LDRGEPVNRADEVAREHDISYNEQLEAGDNPYLKYNHADAEFQEKLADDTSFGGNLG KAVFQAKKRVLEPFGLVEEGAKTAPTGKRIDDHFPKRKKARTEEDSKPSTSSDAEAGPS GSQQLQIPAQPASSLGADTMSAGGGGPLGDNNQGADGVGNASGDWHCDSTWMGDRV VTKSTRTWVLPSYNNHQYREIKSGSVDGSNANAYFGYSTPWGYFDFNRFHSHWSPRD WQRLINNYWGFRPRSLRVKIFNIQVKEVTVQDSTTTIANNLTSTVQVFTDDDYQLPYVV GNGTEGCLPAFPPQVFTLPQYGYATLNRDNTENPTERSSFFCLEYFPSKMLRTGNNFEFT YNFEEVPFHSSFAPSQNLFKLANPLVDQYLYRFVSTNNTGGVQFNKNLAGRYANTYKN WFPGPMGRTQGWNLGSGVNRASVSAFATTNRMELEGASYQVPPQPNGMTNNLQGSN TYALENTMIFNSQPANPGTTATYLEGNMLITSESETQPVNRVAYNVGGQMATNNQSST TAPATGTVNTQEIVPGSVWMERDVYLQGPIWAKIPETGAHFHPSPAMGGFGLKHPPPM MLIKNTPVPGNIT SFSDVPVSSFITQYSTGQVTVEMEWELKKENSKRWNPEIQYTNNYN DPQFVDFAPDSTGEYRTTRPIGTRYLTRPL). In some embodiments, the VP1 capsid polypeptide comprises a variation in the 581-589 region. For example, a VP1 capsid polypeptide comprising a variant 581-589 region may comprise a sequence of SEQ ID NO: 1 in which residues 581 to residue 589 are replaced with a sequence of X¹X²X³X⁴X⁵X⁶X⁷X⁸X⁹, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V, and wherein the VP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, the polynucleotide encodes an AAV VP1 capsid polypeptide that further comprises one or more mutations at an amino acid residue outside of the 581-589 region, with reference to SEQ ID NO: 1, wherein the resulting recombinant capsid is capable of forming an assembled virion that exhibits desired tissue targeting as compared to wild type AAV5 (SEQ ID NO: 1). The one or more mutations may confer increased tissue tropism (e.g., increased CNS tissue tropism or increased muscle tissue tropism) or tissue preference (e.g., increased CNS tissue preference or increased muscle tissue preference) on the assembled virion as compared to a wild type AAV5 capsid polypeptide.

In another aspect, a vector capable of replication in prokaryotic cells is provided, wherein the vector comprises the polynucleotide described immediately above. In typical embodiments, the vector is a plasmid encoding a replication competent AAV genome.

In a further aspect, a library is provided. The library comprises a plurality of vectors comprising the AAV capsid-encoding polynucleotides. In some embodiments, the vectors are plasmids, and the plurality of plasmids comprise a plurality of different AAV VP-encoding polynucleotides. The library may comprise a plurality of vectors encoding AAV VP1 capsid polypeptides having the amino acid sequence of SEQ ID NO: 2 comprising one or more amino acid variations in the 581-589 region.

In various embodiments, the library encodes at least 1×10⁹ (1e9) different AAV VP capsid polypeptides, at least 2.5×10⁹ (2.5e9) different AAV VP capsid polypeptides, at least 5×10⁹ (5e9) different AAV VP capsid polypeptides, at least 7.5×10⁹ (7.5e9) different AAV VP capsid polypeptides, at least 1×10¹⁰ (1e10) different AAV VP capsid polypeptides, at least 2.5×10¹⁰ (2.5e10) different AAV VP capsid polypeptides, at least 5×10¹⁰ (5e10) different AAV VP capsid polypeptides, at least 7.5×10¹⁰ (7.5e10) different AAV VP capsid polypeptides, at least 1×10¹¹ (1e11) different AAV VP capsid polypeptides, at least 2.5×10¹¹ (2.5e11) different AAV VP capsid polypeptides, or at least 5×10¹¹ (5e11) different AAV VP capsid polypeptides. The library may encode VP capsid polypeptides comprising 581-589 region variants.

In another aspect, prokaryotic cells comprising the vectors are provided. In some embodiments, the prokaryotic cell is an E. coli cell and the vector is a plasmid.

In a related aspect, libraries are provided, the library comprising a plurality of E. coli cells, wherein the plurality of cells comprise a plurality of plasmids, wherein the plurality of plasmids comprise a plurality of different AAV VP-encoding polynucleotides.

In some embodiments, the library encodes at least 1×10⁹ (1e9) different AAV VP capsid polypeptides, at least 2.5×10⁹ (2.5e9) different AAV VP capsid polypeptides, at least 5×10⁹ (5e9) different AAV VP capsid polypeptides, at least 7.5×10⁹ (7.5e9) different AAV VP capsid polypeptides, at least 1×10¹⁰ (1e10) different AAV VP capsid polypeptides, at least 5×10¹⁰ (5e10) different AAV VP capsid polypeptides, at least 7.5×10¹⁰ (7.5e10) different AAV VP capsid polypeptides, at least 1×10¹¹ (1e11) different AAV VP capsid polypeptides, at least 2.5×10¹¹ (2.5e11) different AAV VP capsid polypeptides, or at least 5×10¹¹ (5e1l) different AAV VP capsid polypeptides. The library may encode VP capsid polypeptides comprising 581-589 region variants.

### VP Polypeptides, Peptide Libraries

In another aspect, AAV VP1 capsid polypeptides are provided. The polypeptide has the amino acid sequence of SEQ ID NO: 2, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V. The polypeptide includes at least one mutation as compared to native AAV VP1, and thus does not have the sequence of SEQ ID NO: 1. In addition, the polypeptide does not have the sequence of SEQ ID NO: 3, SEQ ID NO: 4, SEQ ID NO: 5, SEQ ID NO: 6, SEQ ID NO: 7, or SEQ ID NO: 8.

In some embodiments, the polypeptide further comprises one or more mutations at an amino acid residue outside of the 581-589 region, with reference to SEQ ID NO: 1, wherein the resulting recombinant capsid is capable of forming an assembled virion that exhibits desired tissue targeting as compared to wild type AAV5 (SEQ ID NO: 1).

In a further aspect, libraries are provided, the libraries comprising a plurality of polypeptides as described immediately above, the plurality having different primary amino acid sequences. A library may comprise a plurality of polypeptides of SEQ ID NO: 2 comprising one or more amino acid variations in the 581-589 region.

In some embodiments, library comprises at least 1×10⁹ (1e9) different AAV VP capsid polypeptides, at least 2.5×10⁹ (2.5e9) different AAV VP capsid polypeptides, at least 5×10⁹ (5e9) different AAV VP capsid polypeptides, at least 7.5×10⁹ (7.5e9) different AAV VP capsid polypeptides, at least 1×10¹⁰ (1e10) different AAV VP capsid polypeptides, at least 2.5×10¹⁰ (2.5e10) different AAV VP capsid polypeptides, at least 5×10¹⁰ (5e10) different AAV VP capsid polypeptides, at least 7.5×10¹⁰ (7.5e10) different AAV VP capsid polypeptides, at least 1×10¹¹ (1e11) different AAV VP capsid polypeptides, at least 2.5×10¹¹ (2.5e11) different AAV VP capsid polypeptides, or at least 5×10¹¹ (5e11) different AAV VP capsid polypeptides. The library may comprise VP capsid polypeptides comprising 581-589 region variants.

In certain embodiments, the library comprises at least from about 1×10⁵ (1e5) to at least about 5×10¹¹ (5e11) different AAV VP capsid polypeptides. In certain embodiments, the library comprises at least about 1×10⁵ (1e5), at least about 2×10⁵ (2e5), at least about 3×10⁵ (3e5), at least about 4×10⁵ (4e5), at least about 5×10⁵ (5e5), at least about 6×10⁵ (6e5), at least about 7×10⁵ (7e5), at least about 8×10⁵ (8e5), at least about 9×10⁵ (9e5), at least about 1×10⁶ (1e6), at least about 2×10⁶ (2e6), at least about 3×10⁶ (3e6), at least about 4×10⁶ (4e6), at least about 5×10⁶ (5e6), at least about 6×10⁶ (6e6), at least about 7×10⁶ (7e6), at least about 8×10⁶ (8e6), at least about 9×10⁶ (9e6), at least about 1×10⁷ (1e7), at least about 2×10⁷ (2e7), at least about 3×10⁷ (3e7), at least about 4×10⁷ (4e7), at least about 5×10⁷ (5e7), at least about 6×10⁷ (6e7), at least about 7×10⁷ (7e7), at least about 8×10⁷ (8e7), at least about 9×10⁷ (9e7), at least about 1×10⁸ (1e8), at least about 2×10⁸ (2e8), at least about 3×10⁸ (3e8), at least about 4×10⁸ (4e8), at least about 5×10⁸ (5e8), at least about 6×10⁸ (6e8), at least about 7×10⁸ (7e8), at least about 8×10⁸ (8e8), at least about 9×10⁸ (9e8), at least about 1×109 (1e9), at least about 2×10⁹ (2e9), at least about 3×10⁹ (3e9), at least about 4×10⁹ (4e9), at least about 5×10⁹ (5e9), at least about 6×10⁹ (6e9), at least about 7×10⁹ (7e9), at least about 8×10⁹ (8e9), at least about 9×10⁹ (9e9), at least about 1×10¹⁰ (1e10), at least about 2×10¹⁰ (2e10), at least about 3×10¹⁰ (3e10), at least about 4×10¹⁰ (4e10), at least about 5×10¹⁰ (5e10), at least about 6×10¹⁰ (6e10), at least about 7×10¹⁰ (7e10), at least about 8×10¹⁰ (8e10), at least about 9×10¹⁰ (9e10), at least about 1×10¹¹ (1e11), at least about 2×10¹¹ (2e11), at least about 3×10¹¹ (3e11), at least about 4×10¹¹ (4e11), or at least about 5×10¹¹ (5e1l) AAV VP capsid polypeptides.

In certain embodiments, provided herein is a recombinant adeno-associated virus AAV VP1 capsid polypeptide having at least one mutation in a residue of region 581 to residue 589 in SEQ ID NO: 1, inclusive, wherein the mutation confers at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold increased accumulation of an AAV virion having said AAV VP1 capsid polypeptide in a target tissue (e.g., a CNS tissue or a muscle tissue) relative to a non-target tissue, as compared to wild type AAV virion having a wild type AAV5 VP1 capsid polypeptide, and wherein the AAVVP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. In some embodiments, a VP1 capsid polypeptide comprises a variation in the 581-589 region. For example, a VP1 capsid polypeptide comprising a 581-589 region may comprise a sequence of SEQ ID NO: 1 in which residues 581 to residue 589 are replaced with a sequence of X¹X²X³X⁴X⁵X⁶X⁷X⁸X⁹, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V, and wherein the VP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. rAAV virions, virion libraries

In another aspect, recombinant AAV virions (rAAV) are provided. The virion comprises an AAV VP capsid polypeptide as described above. In some embodiments, the rAAV has increased tropism for primate and human CNS tissue as compared to a rAAV having the native AAV5 VP1 capsid polypeptide (SEQ ID NO:1). In some embodiments, the rAAV has increased tropism for primate and human muscle tissue as compared to a rAAV having the native AAV5 VP1 capsid polypeptide (SEQ ID NO:1). In some embodiments, the rAAV has increased ability to assemble, or exhibits greater virion stability, as compared to a rAAV having the native AAV5 VP1 capsid polypeptide (SEQ ID NO: 1).

In some embodiments, the rAAV has increased ability to cross the blood-brain barrier following intravenous administration as compared to a rAAV having the native AAV5 VP1 capsid polypeptide (SEQ ID NO: 1).

In certain of these embodiments, the rAAV has increased ability to infect one or more brain regions selected from hippocampus, dentate gyrus, cerebral cortex, temporal cortex, occipital cortex, thalamus, forebrain, substantia nigra, hypothalamus, cerebellum, and combinations thereof following systemic administration, as compared to a rAAV having the native AAV5 VP1 capsid polypeptide (SEQ ID NO: 1).

In some embodiments, the rAAV has increased ability to infect one or more brain regions selected from hippocampus, dentate gyrus, cerebral cortex, temporal cortex, occipital cortex, thalamus, forebrain, substantia nigra, hypothalamus, cerebellum, and combinations thereof following systemic administration, and also has reduced tropism for non-CNS tissues, as compared to a rAAV having the native AAV5 VP1 capsid polypeptide (SEQ ID NO: 1).

In some embodiments, the rAAV has increased ability to infect muscle tissue, including aorta, esophagus, heart (e.g., atrium, ventricle, valves), skeletal muscle (e.g., biceps, brachii, femoris, diaphragm, gastrocnemius, tibialis anterior, triceps, quadriceps, including vastus lateralis)), or muscle fibers, including type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof, following systemic administration, as compared to a rAAV having the native AAV5 VP1 capsid polypeptide (SEQ ID NO: 1).

In some embodiments, the rAAV has increased ability to infect muscle tissue, including aorta, esophagus, heart (e.g., atrium, ventricle, valves), skeletal muscle (e.g., biceps, brachii, femoris, diaphragm, gastrocnemius, tibialis anterior, triceps, quadriceps, including vastus lateralis)), or muscle fibers, including type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof, following systemic administration, and also has reduced tropism for non-muscle tissues, as compared to a rAAV having the native AAV5 VP1 capsid polypeptide (SEQ ID NO: 1).

Additionally, provided are polynucleotide sequences encoding the rAAV capsid VP proteins described herein.

In a further aspect, libraries are provided that comprise a plurality of rAAV as described above. The plurality of rAAVs comprise a plurality of VP capsid polypeptides having different primary amino acid sequences. An rAAV of the plurality of rAAVs may comprise a polypeptide of SEQ ID NO: 2 comprising one or more amino acid variations in the 581-589 region.

In some embodiments, the library comprises at least 1×10⁹ (1e5) different AAV VP capsid polypeptides, at least 2.5×10⁹ (2.5e9) different AAV VP capsid polypeptides, at least 5×10⁹ (5e9) different AAV VP capsid polypeptides, at least 7.5×10⁹ (7.5e9) different AAV VP capsid polypeptides, at least 1×10¹⁰ (1e10) different AAV VP capsid polypeptides, at least 2.5×10¹⁰ (2.5e10) different AAV VP capsid polypeptides, at least 5×10¹⁰ (5e10) different AAV VP capsid polypeptides, at least 7.5×10¹⁰ (7.5e10) different AAV VP capsid polypeptides, at least 1×10¹¹ (1e11) different AAV VP capsid polypeptides, at least 2.5×10¹¹ (2.5e11) different AAV VP capsid polypeptides, or at least 5×10¹¹ (5e11) different AAV VP capsid polypeptides. The library may comprise AAV VP capsid polypeptides comprising 581-589 region variants. For example, a VP1 capsid polypeptide comprising a 581-589 region may comprise a sequence of SEQ ID NO: 1 in which residues 581 to residue 589 are replaced with a sequence of X¹X²X³X⁴X⁵X⁶X⁷X⁸X⁹, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V, and wherein the VP1 capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

### Pharmaceutical Compositions

In another aspect, pharmaceutical compositions are provided. The pharmaceutical composition comprises a rAAV as described above and a pharmaceutically acceptable carrier.

A pharmaceutical composition can comprise a first active ingredient. The first active ingredient can comprise a viral vector as described herein and/or any payload as described herein. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient—such as to facilitate enhanced gene replacement, RNA editing, DNA editing, or imaging.

A pharmaceutical composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof Δn excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerol, xanthan rubber, soy isoflavones, polysorbate 80, ethyl alcohol, water, teprenone, or any combination thereof.

Compositions and methods provided herein can utilize pharmaceutical compositions. The compositions described throughout can be formulated into a pharmaceutical and be used to treat a human or mammal, in need thereof, diagnosed with a disease. In some cases, pharmaceutical compositions can be used prophylactically.

The compositions provided herein can be utilized in methods provided herein. Any of the provided compositions provided herein can be utilized in methods provided herein. In some cases, a method comprises at least partially preventing, reducing, ameliorating, and/or treating a disease or condition, or a symptom of a disease or condition. A subject can be a human or non-human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

### Methods of Treatment or Detection

In some aspects, the present disclosure provides for methods of treatment using an rAAV virion having any one of the engineered AAV VP capsid polypeptide sequences disclosed herein. In some aspects, the present disclosure provides for methods of detection using an rAAV virion having any one of the engineered AAV VP capsid polypeptide sequences disclosed herein. A method of treatment may comprise administering to a subject an effective amount of a pharmaceutical composition comprising rAAV virions assembled from VP polypeptides comprising a 581-589 region that convers tissue tropism (e.g., CNS tissue tropism or muscle tropism) to the rAAV. The rAAV virions may comprise a VP polypeptide 581-589 region described herein (e.g., comprising a variant 581-589 region that confers tissue tropism or tissue preference). In some embodiments, the rAAV may be assembled from VP capsid polypeptides comprising a 581-589 region of any one of SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811. The rAAV virions may encapsidate any payload, including those payloads disclosed herein.

A method of treatment may comprise administering an rAAV encapsidating a DNA molecule and enriching the DNA in a tissue of interest (e.g., a CNS tissue, a muscle tissue, a cardiac muscle tissue, or a skeletal muscle tissue) with a DNA enrichment that is at least 1-fold, at least 2-fold, at least 3-fold, at least 4-fold, at least 5-fold, at least 10-fold, at least 15-fold, at least 25-fold, at least 30-fold, at least 40-fold, at least 50-fold, at least 100-fold, at least 200-fold, at least 300-fold, at least 500-fold, or at least 1000-fold a DNA enrichment of a wild type AAV9.

A method of treatment may comprise administering an rAAV encapsidating a DNA molecule and enriching the DNA in a tissue of interest (e.g., a CNS tissue, a muscle tissue, a cardiac muscle tissue, or a skeletal muscle tissue) with a DNA enrichment that is at least 1-fold, at least 2-fold, at least 3-fold, at least 4-fold, at least 5-fold, at least 10-fold, at least 15-fold, at least 25-fold, at least 30-fold, at least 40-fold, at least 50-fold, at least 100-fold, at least 200-fold, at least 300-fold, at least 500-fold, or at least 1000-fold a DNA enrichment of a wild type AAV5.

A method of treatment may comprise administering an rAAV encapsidating a DNA molecule and expressing an RNA encoded by the DNA in a tissue of interest (e.g., a CNS tissue, a muscle tissue, a cardiac muscle tissue, or a skeletal muscle tissue) with an RNA enrichment that is at least 1-fold, at least 2-fold, at least 3-fold, at least 4-fold, at least 5-fold, at least 10-fold, at least 15-fold, at least 25-fold, at least 30-fold, at least 40-fold, at least 50-fold, at least 100-fold, at least 200-fold, at least 300-fold, at least 500-fold, or at least 1000-fold an RNA enrichment of a wild type AAV9.

A method of treatment may comprise administering an rAAV encapsidating a DNA molecule and expressing an RNA encoded by the DNA in a tissue of interest (e.g., a CNS tissue, a muscle tissue, a cardiac muscle tissue, or a skeletal muscle tissue) with an RNA enrichment that is at least 1-fold, at least 2-fold, at least 3-fold, at least 4-fold, at least 5-fold, at least 10-fold, at least 15-fold, at least 25-fold, at least 30-fold, at least 40-fold, at least 50-fold, at least 100-fold, at least 200-fold, at least 300-fold, at least 500-fold, or at least 1000-fold an RNA enrichment of a wild type AAV5.

In some embodiments, the effective amount is at least 1×10⁸ (1e8) viral genomes per dose. In some embodiments, the effective amount is at least 5×10⁸ (5e8) viral genomes/dose, 7.5×10⁸ (7.5e8) viral genomes/dose, at least 1×10⁹ (1e9) viral genomes/dose, at least 2.5×10⁹ (2.5e9) viral genomes/dose, at least 5×10⁹ (5e9) viral genomes/dose. In some embodiments, an effective amount of an rAAV assembled from VP polypeptides comprising a 581-589 region of any one of SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811 may be lower than an effective amount of an AAV assembled from VP1, VP2, and VP3 polypeptides of SEQ ID NO: 1, SEQ ID NO: 10, and SEQ ID NO: 11, respectively.

In some embodiments, the effective amount is at least 1×10¹¹ (1e11) viral genomes/kg patient weight, at least 5×10¹¹ (5e1l) viral genomes/kg, at least 1×10¹² (1e12) viral genomes/kg, at least 5×10¹² (5e12) viral genomes/kg, at least 1×10¹³ (1e13) viral genomes/kg, at least 1×10¹⁴ (1e14) viral genomes/kg, or at least 5×10¹⁴ (5e14). In some embodiments, an effective amount of an rAAV assembled from VP polypeptides comprising a 581-589 region of any one of SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811 may be lower than an effective amount of an AAV assembled from VP1, VP2, and VP3 polypeptides of SEQ ID NO: 1, SEQ ID NO: 10, and SEQ ID NO: 11, respectively.

In some embodiments, the rAAV virion is administered via a systemic administration route including enteral routes of administration and parenteral routes of administration. The rAAV virion may be administered intravenously. In some embodiments, the rAAV may be administered intramuscularly. In some embodiments, the rAAV may be administered intraperitoneally. In some embodiments, the rAAV may be administered topically. In some embodiments, the rAAV may be administered orally. In particular embodiments, the rAAV virion is administered intravenously. In some embodiments, the rAAV is administered intrathecally. In some embodiments, the rAAV is administered by intracerebroventricular injection. In some embodiments, the rAAV is administered by intracerebral ventricular injection. In some embodiments, the rAAV is administered by intracisternal magna administration. In some embodiments, the rAAV is administered by intravitreal injection. In some embodiments, the rAAV is administered by parenchymal injection. In some embodiments, the rAAV is administered by intraparenchymal injection. In some embodiments, the rAAV is administered by intramyocardial injection. In some embodiments, the rAAV is administered by intracoronary injection. In some embodiments, the rAAV is administered by intrapericardial injection. For example, an rAAV virion with CNS tissue tropism may be administered via intrathecal injection, intracisternal magna injection, intracerebroventricular injection, intraparenchymal injection, or intravenous injection. In another example, an rAAV virion with muscle tissue tropism may be administered via intramuscular injection, intracoronary injection, intrapericardial injection, intramyocardial injection, or intravenous injection. In another example, an rAAV virion with cardiac tissue tropism may be administered via intramyocardial injection, intrapericardial injection, intracoronary injection, or intravenous injection.

In various embodiments, the patient suffers from one of the conditions listed in TABLE 1, below. In particular embodiments, the patient suffers from one of the conditions listed in TABLE 1 and the rAAV includes a payload comprising the transgene product associated therewith in TABLE 1. In some embodiments, an rAAV may be selected to specifically target the primary gene delivery target (e.g., CNS or muscle). For example, an rAAV selected to target the CNS may comprise VP capsid polypeptides comprising a 581-589 region with increased likelihood of conferring CNS tissue tropism (e.g., any one of SEQ ID NO: 12-SEQ ID NO: 3937, SEQ ID NO: 3938-SEQ ID NO: 4237, or SEQ ID NO: 5234-SEQ ID NO: 5807). In another example, an rAAV selected to target muscle may comprise VP capsid polypeptides comprising a 581-589 region with increased likelihood of conferring muscle tropism (e.g., any one of SEQ ID NO: 4238-SEQ ID NO: 4933, SEQ ID NO: 4934-SEQ ID NO: 5233, or SEQ ID NO: 5808-SEQ ID NO: 5811).

In some embodiments, an rAAV virion of the present disclosure, having any of the engineered AAV VP capsid polypeptide sequences disclosed herein, comprises a vector genome, the vector genome comprising a therapeutic polynucleotide or payload. In further embodiments, said payload may be under control of regulatory sequences that direct expression in infected human cells. In some embodiments, the payload comprises a therapeutic polynucleotide encoding any genetically encodable payload, such as an RNA (e.g., a guide RNA), a suppressor tRNA, a transgene, or a genome modifying entity. In some embodiments, the payload may be under control of a promoter. The promoter may be a ubiquitous promoter, or the promoter may be cell or tissue specific. For example, a payload may be under control of a neuronal promoter for expression in neurons. In another example, a payload may be under control of a muscle promoter for expression in muscle cells.

In some embodiments, the therapeutic polynucleotide encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof. In some embodiments, the therapeutic polynucleotide encodes a linear therapeutic polynucleotide or a circular therapeutic polynucleotide.

In some embodiments, the therapeutic polynucleotide is a transgene, encoding a therapeutic protein. In particular embodiments, the transgene encodes a protein selected from the targets suitable for modification or transgene products of TABLE 1. In some embodiments, a transgene encoding a CNS target is encapsidated by an rAAV comprising VP capsid polypeptides comprising a 581-589 region with increased likelihood of conferring CNS tissue tropism or preference (e.g., any one of SEQ ID NO: 12-SEQ ID NO: 3937, SEQ ID NO: 3938-SEQ ID NO: 4237, or SEQ ID NO: 5234-SEQ ID NO: 5807). In some embodiments, a transgene encoding a muscle target is encapsidated by an rAAV comprising VP capsid polypeptides comprising a 581-589 region with increased likelihood of conferring muscle tropism or preference (e.g., any one of SEQ ID NO: 4238-SEQ ID NO: 4933, SEQ ID NO: 4934-SEQ ID NO: 5233, or SEQ ID NO: 5808-SEQ ID NO: 5811).

TABLE 1
Suitable Therapeutic Targets

| Primary Gene | | Target of a Therapeutic |
|---|---|---|
| Delivery Target | Condition | Polynucleotide |
| Brain/CNS | AADC deficiency | AADC |
| | Alzheimer's Disease | Multiple, including APP, SNCA, |
| | | MAPT, ApoE, NGF, TERT |
| | Tauopathies | MAPT |
| | Synucleinopathies | SNCA |
| | Batten disease (CLN2) | CLN2 |
| | Batten disease (CLN3) | CLN3 |
| | Batten disease (CLN6) | CLN6 |
| | MPS-IIIB | NAGLU |
| | Frontotemporal dementia with GRN | GRN |
| | mutations (FTD-GRN) | |
| | Parkinson's Disease with GBA1 mutations | GBA1 |
| | (PD-GBA) and neuronpathic Gaucher's | |
| | disease | |
| | Corticobasal Degeneration (CBD) | MAPT |
| | Progressive Supranuclear Palsy (PSP) | MAPT |
| | Chronic Traumatic Encephalopathy (CTE) | MAPT |
| | Synucleinopathies | GBA1 + alpha-synuclein |
| | Gaucher disease type 2 | GBA |
| | Canavan Disease | ASPA |
| | Parkinson disease | AADC |
| | Parkinson disease | GDNF |
| | Parkinson disease | Neurturin |
| | Parkinson disease | GAD |
| | Parkinson disease | NTN |
| | Parkinson disease | hFOXG1 |
| | Parkinson disease | hKCNQ2 |
| | Parkinson disease | hFMR1 |
| | Parkinson disease | anti-Tau/miRNA |
| | Parkinson disease | EPM2A or EPM2B |
| | Parkinson disease | LRRK2 |
| | Parkinson's Disease | LRRK2 |
| | Parkinson's Disease | SNCA |
| | Tay-Sachs Disease | HEXA |
| | Huntington's disease | IT15 |
| | Huntington's disease | CYP46A1 |
| | Huntington's disease | HTT |
| | Protocki-Lupski Syndrome | IT15 |
| | Amyotrophic lateral sclerosis | C9orf72 |
| | Amyotrophic lateral sclerosis | SOD1 |
| | Down syndrome | DYRK1A |
| | Sanfilippo disease type A | SGSH |
| | Sanfilippo disease type B | hNAGLU |
| | (Nervous system) | HEXB and HEXA |
| | (Nervous system) | human codon-optimized CLN1 |
| | | complementary DNA |
| | (Nervous system) | SURF1 |
| | (Nervous system) | anti-UBE3A-ATS shRNA |
| | (Nervous system) | hSLC6A1 |
| | Rett syndrome | MECP2 |
| Muscle | Charcot-Marie-Tooth disease type 1A | NTF3 |
| | (CMT1A) | |
| | Duchenne muscular dystrophy (DMD) | Micro-dystrophin |
| | Duchenne muscular dystrophy (DMD) | Mini-dystrophin |
| | Facioscapulohumeral muscular dystrophy- | DUX4 |
| | 1 (FSHD) | |
| | Dysferlinopathy | DYSF |
| | Pompe disease | GAA |
| | Limb-girdle muscular dystrophies (LGMD) | FKRP |
| | (2i/R9) | |
| | Duchenne muscular dystrophy (DMD) | DMD |
| | Facioscapulohumeral Dystrophy | DUX4 |
| | Myotonic Dystrophy | DMPK |
| | Glycogen storage disorders | anti-GYS1 miRNA |
| | X-linked myotubular myopathy (X-linked | MTM1 |
| | MTM) | |
| | euchromatic histone-lysine N- | anti-EHMT2 shRNA |
| | methyltransferase 2 | |

In some embodiments, the therapeutic polynucleotide encodes a therapeutic RNA. In some embodiments the therapeutic polynucleotide encodes an RNA, such as a guide RNA (including an engineered or synthetic guide RNA) for genome editing or for RNA editing. The guide RNA may target a gene, such as those provided in TABLE 1 or encoding a protein nucleotide provided in TABLE 1, to promote editing of the target gene. In some embodiments, editing of the target gene may treat a condition in a subject, such as a condition provided in TABLE 1.

In some embodiments, the therapeutic polynucleotide encodes a tRNA or a modified tRNA (engineered or synthetic tRNA). For example, the tRNA or modified tRNA can be a suppressor tRNA. The suppressor tRNA can be engineered to have an anticodon region that recognizes a stop codon, such as any premature stop codon (opal, ochre, or amber stop codons). A suppressor tRNA may target a premature stop codon in a gene, such as those provided in TABLE 1 or encoding a protein nucleotide provided in TABLE 1, to promote readthrough of the gene. In some embodiments, suppressing the premature stop codon may treat a condition in a subject, such as a condition provided in TABLE 1.

In some embodiments, the therapeutic polynucleotide (e.g., a therapeutic RNA, a tRNA, or a genome modifying entity) can target a gene listed in TABLE 1 or any gene associated with a neurologic disease, Parkinson's disease, Alzheimer's disease, a Tauopathy, Stargardt disease, alpha-1 antitrypsin deficiency, Duchenne's muscular dystrophy, Rett syndrome, cystic fibrosis, or any genetic disease. In some embodiments, the targeted gene may be ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some embodiments, the therapeutic polynucleotide is a gene therapy payload (e.g., a transgene) and, thus, may itself be one of the genes listed in TABLE 1 or any gene associated with a neurologic disease, Parkinson's disease, Alzheimer's disease, a Tauopathy, Stargardt disease, alpha-1 antitrypsin deficiency, Duchenne's muscular dystrophy, Rett syndrome, cystic fibrosis, or any genetic disease. In some embodiments, the transgene may be ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof.

An engineered AAV comprising a VP capsid polypeptide comprising a CNS tissue-tropic 581-589 region may be used to deliver a payload to treat a neurological condition, or a condition of the central nervous system. For example, an engineered AAV comprising a VP capsid polypeptide comprising a 581-589 region of any one of SEQ ID NO: 12-SEQ ID NO: 3937 or SEQ ID NO: 3938-SEQ ID NO: 4237 may be used to treat a neurological condition (e.g., an aromatic l-amino acid decarboxylase (AADC) deficiency, Alzheimer's disease, a tauopathy, a synucleinopathy, Batten disease, mucopolysaccharidosis type III, frontotemporal dementia, Parkinson's disease, Gaucher disease, Canavan disease, Tay-Sachs disease, Huntington's disease, Protocki-Lupski syndrome, amyotrophic lateral sclerosis, Down syndrome, Sanfilippo disease type A, Sanfilippo disease type B, or Rett syndrome). In some embodiments, the engineered AAV comprising a VP capsid polypeptide comprising a CNS tissue-tropic 581-589 region may be used to deliver a transgene encoding a protein associated with a neurological condition (e.g., aromatic l-amino acid decarboxylase (AADC), amyloid precursor protein (APP), α-synuclein, microtubule associated protein tau (MAPT), ApoE, nerve growth factor (NGF), telomerase reverse transcriptase (TERT), CLN2, CLN3, CLN6, N-acetyl-α-glucosaminidase (NAGLU), granulin, glucosylceramidase 13 (GBA), aspartoacylase, GDNF, neurturin (NTN), glutamate decarboxylase (GAD), FOXG1, Kᵥ7.2, laforin, leucine rich repeat kinase 2 (LRRK2), hexosaminidase A (HEXA), hexosaminidase B (HEXB), huntingtin, cholesterol 24-hydroxylase, C9orf72, superoxide dismutase 1 (SOD1), dual specificity tyrosine-phosphorylation-regulated kinase 1A (DYRKA1), N-sulfoglucosamine sulfohydrolase (SGSH), surfeit locus protein 1 (SURF1), GABA transporter 1 (GAT1), and methyl-CpG binding protein 2 (MECP2)). In some embodiments, the engineered AAV comprising a VP capsid polypeptide comprising a CNS tissue-tropic 581-589 region may be used to deliver a therapeutic polynucleotide that alters expression or promotes editing of a gene encoding a protein associated with a neurological condition (e.g., aromatic l-amino acid decarboxylase (AADC), amyloid precursor protein (APP), α-synuclein, microtubule associated protein tau (MAPT), ApoE, nerve growth factor (NGF), telomerase reverse transcriptase (TERT), CLN2, CLN3, CLN6, N-acetyl-α-glucosaminidase (NAGLU), granulin, glucosylceramidase 13 (GBA), aspartoacylase, GDNF, neurturin (NTN), glutamate decarboxylase (GAD), FOXG1, Kᵥ7.2, laforin, leucine rich repeat kinase 2 (LRRK2), hexosaminidase A (HEXA), hexosaminidase B (HEXB), huntingtin, cholesterol 24-hydroxylase, C9orf72, superoxide dismutase 1 (SOD1), dual specificity tyrosine-phosphorylation-regulated kinase 1A (DYRKA1), N-sulfoglucosamine sulfohydrolase (SGSH), surfeit locus protein 1 (SURF1), GABA transporter 1 (GAT1), and methyl-CpG binding protein 2 (MECP2)).

An engineered AAV comprising a VP capsid polypeptide comprising a muscle-tropic 581-589 region may be used to deliver a payload to treat a muscular condition. For example, an engineered AAV comprising a VP capsid polypeptide comprising a 581-589 region of any one of SEQ ID NO: 4238-SEQ ID NO: 4933 or SEQ ID NO: 4934-SEQ ID NO: 5233 may be used to treat a muscular condition (e.g., Charcot-Marie-Tooth disease, Duchenne muscular dystrophy, dysferlinopathy, Pompe disease, Limb-girdle muscular dystrophies, facioscapulohumeral dystrophy, myotonic dystrophy, a glycogen storage disorder, X-linked myotubular myopathy, or euchromatic histone-lysine N-methyltransferase 2). In some embodiments, the engineered AAV comprising a VP capsid polypeptide comprising a muscle-tropic 581-589 region may be used to deliver a transgene encoding a protein associated with a muscular condition (e.g., neurotrophin 3 (NFT3), dystrophin, mini-dystrophin, micro-dystrophin, dysferlin, acid α-glucosidase (GAA), fukutin-related protein (FKRP), double homeobox 4 (DUX4), glycogen synthase 1 (GYS1), myotubularin, or euchromatic histone-lysine N-methyltransferase 2 (EHMT2)). In some embodiments, the engineered AAV comprising a VP capsid polypeptide comprising a muscle-tropic 581-589 region may be used to deliver a therapeutic polynucleotide that alters expression or promotes editing of a gene encoding a protein associated with a muscular condition (e.g., neurotrophin 3 (NFT3), dystrophin, mini-dystrophin, micro-dystrophin, dysferlin, acid α-glucosidase (GAA), fukutin-related protein (FKRP), double homeobox 4 (DUX4), glycogen synthase 1 (GYS1), myotubularin, or euchromatic histone-lysine N-methyltransferase 2 (EHMT2)).

In some embodiments, the therapeutic polynucleotide encodes genome modifying entities. For example, a genome modifying entity may be a DNA editing enzyme, an RNA editing enzyme, a transcriptional activator, or a transcriptional repressor. The DNA editing enzyme may be any DNA editing enzyme, including any CRISPR/Cas systems, meganucleases, zinc-finger nucleases, (ZFNs), TALE Nucleases (TALENs and megaTALENS). The CRISPR/Cas system can be a Cas3, Cas8, Cas10, Cas9, Cas4, Cas12, or Cas13. The RNA editing enzyme may be ADAR. In some embodiments, the ADAR is a human ADAR1 or human ADAR2. The transcriptional activator may be VP64. A transcriptional repressor may be KRAB. Such genome modifying entities may target any gene listed in TABLE 1 for editing.

In some embodiments, the present disclosure provides for rAAV virions having an engineered AAV VP capsid polypeptide (e.g., comprising a 581-589 region variant), where the virion encapsidates any one of or any combination of the therapeutic payloads disclosed herein. In some embodiments, multiple copies of the therapeutic payload are encapsidated.

In some embodiments, the therapeutic polynucleotide is a polynucleotide capable of serving as a homology template for homology-directed repair. In some embodiments, the therapeutic polynucleotide may be a guide polynucleotide for a CRISPR/Cas system or an ADAR enzyme. For example, the therapeutic polynucleotide may be a CRISPR/Cas guide RNA or an ADAR guide RNA.

In some embodiments, an rAAV virion of the present disclosure, having any of the engineered AAV VP capsid polypeptide sequences disclosed herein, comprises a vector genome, the vector genome comprising a detectable polynucleotide or payload. In further embodiments, said payload may be under control of regulatory sequences that direct expression in infected human cells. Examples of detectable polynucleotides include, but are not limited to, any genetically encodable detectable moiety. For example, a genetically encodable detectable moiety may be a fluorescent protein such as EGFP, GFP, YFP, RFP, CFP, or any variants thereof. In some embodiments, the present disclosure provides for rAAV virions having an engineered AAV VP capsid polypeptide, where the virion encapsidates any one of or any combination of the detectable payloads disclosed herein. In some embodiments, multiple copies of the detectable payload are encapsidated.

In some embodiments, the present disclosure provides for rAAV virions having an engineered AAV VP capsid polypeptide, where the virion encapsidates any one of or any combination of the therapeutic payloads and detectable payloads disclosed herein. For example, an rAAV of the present disclosure having an engineered AAV VP capsid polypeptide may encapsidate a transgene and a fluorescent protein. As another example, an rAAV of the present disclosure having an engineered AAV VP capsid polypeptide may encapsidate a therapeutic RNA (e.g., a guide RNA) and a fluorescent protein.

Delivering a payload (e.g., a payload encoding a therapeutic polypeptide or a therapeutic polynucleotide) using the CNS specific or muscle specific AAVs described herein may produce lower toxicity in a subject compared to non-specific AAVs (e.g., AAVs comprising a VP1 polypeptide of SEQ ID NO: 1). Tissue specific AAVs (e.g., CNS specific AAVs or muscle specific AAVs) may be effective at lower doses compared to non-specific AAVs since a larger fraction of the administered AAVs infect the relevant tissue (e.g., CNS tissue or muscle tissue). As a result, a therapeutically effective dose of tissue specific AAVs may be lower than a therapeutically effect dose of non-specific AAVs, leading to lower toxicity due administering a lower dose. For example, administration of a therapeutically effective dose of tissue specific AAVs may result in lower liver toxicity than administration of a therapeutically effective dose of non-specific AAVs. Administration of a lower dose may also lead to lower production of neutralizing antibodies in the subject. Neutralizing antibodies may decrease the efficacy of the AAV therapy by inhibiting infection of the target tissue or may cause severe side effects in the subject due to the immune response. Additionally, tissue specific AAVs (e.g., CNS specific AAVs or muscle specific AAVs) may produce fewer off-target effects compared to non-specific AAVs when administered at the same dose since fewer of the AAVs infect off target tissues (e.g., non-CNS tissues or non-muscle tissues). Off-target effects may include increased gene expression in an off-target tissue, decreased gene expression in an off-target tissue, gene editing in an off-target tissue, immune response, or liver toxicity.

### In Vivo Selected VP Polypeptides

In a further aspect, engineered (synonymously, recombinant) adeno-associated virus (AAV) VP capsid polypeptides identified using the methods described herein are provided.

In some embodiments, the engineered adeno-associated virus (AAV) viral protein (VP) capsid polypeptide has an amino acid sequence at least 70% identical to SEQ ID NO: 1, wherein the engineered AAV VP capsid polypeptide has at least one substitution as compared to SEQ ID NO: 1 in the 581-589 region, corresponding to residue 581 to residue 589 of SEQ ID NO: 1, inclusive, wherein the capsid polypeptide is capable of assembling into a recombinant AAV virion (rAAV), and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8. In some embodiments, the VP capsid polypeptide comprises a variant 581-589 region (e.g., comprising one or more amino acid substitutions relative to SEQ ID NO: 1 in the region from residue 581 to residue 589, inclusive). The VP capsid polypeptide may comprise a sequence of SEQ ID NO: 2, wherein the 581-589 region has a sequence of any one of SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811.

In some embodiments, the engineered adeno-associated virus (AAV) viral protein (VP) capsid polypeptide is CNS tissue-tropic and has an amino acid sequence at least 70% identical to SEQ ID NO: 1, wherein the engineered AAV VP capsid polypeptide has at least one substitution as compared to SEQ ID NO: 1 in the 581-589 region, corresponding to residue 581 to residue 589 of SEQ ID NO: 1, inclusive, wherein the capsid polypeptide is capable of assembling into a recombinant AAV virion (rAAV), wherein the at least one substitution confers higher tropism for a central nervous system (CNS) tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8. In some embodiments, the CNS-tropic VP capsid polypeptide comprises a variant 581-589 region (e.g., comprising one or more amino acid substitutions relative to SEQ ID NO: 1 in the region from residue 581 to residue 589, inclusive). The CNS-tropic VP capsid polypeptide may comprise a sequence of SEQ ID NO: 2, wherein the 581-589 region has a sequence of any one of SEQ ID NO: 12-SEQ ID NO: 3937, SEQ ID NO: 3938-SEQ ID NO: 4237, or SEQ ID NO: 5234-SEQ ID NO: 5807.

In some embodiments, the engineered adeno-associated virus (AAV) viral protein (VP) capsid polypeptide is muscle-tropic and has an amino acid sequence at least 70% identical to SEQ ID NO: 1, wherein the engineered AAV VP capsid polypeptide has at least one substitution as compared to SEQ ID NO: 1 in the 581-589 region, corresponding to residue 581 to residue 589 of SEQ ID NO: 1, inclusive, wherein the capsid polypeptide is capable of assembling into a recombinant AAV virion (rAAV), wherein the at least one substitution confers higher tropism for a muscle tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8. In some embodiments, the muscle-tropic VP capsid polypeptide comprises a variant 581-589 region (e.g., comprising one or more amino acid substitutions relative to SEQ ID NO: 1 in the region from residue 581 to residue 589, inclusive). The muscle-tropic VP capsid polypeptide may comprise a sequence of SEQ ID NO: 2, wherein the 581-589 region has a sequence of any one of SEQ ID NO: 4238-SEQ ID NO: 4933, SEQ ID NO: 4934-SEQ ID NO: 5233, or SEQ ID NO: 5808-SEQ ID NO: 5811.

In particular embodiments, the engineered adeno-associated virus (AAV) viral protein (VP) capsid polypeptide (e.g., a CNS tissue-tropic VP capsid polypeptide or a muscle-tropic VP capsid polypeptide) has an amino acid sequence at least 75%, 80%, 85%, 90%, 91%, 92%, 93%, 94%, 95%, 96%, 97%, 98% identical to the sequence of SEQ ID NO: 1.

In some embodiments, the AAV VP capsid polypeptide has an amino acid sequence of SEQ ID NO: 2, wherein X¹, X², X³, X⁴, X⁵, X⁶, X⁷, X⁸, and X⁹ are each independently selected from any amino acid, wherein the capsid polypeptide is capable of assembling into a recombinant AAV virion (rAAV), and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8, optionally with further mutations elsewhere in the VP capsid polypeptide. In some embodiments, the X¹X²X³X⁴X⁵X⁶X⁷X⁸X⁹ portion corresponds to a sequence selected from any one of SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811.

In some embodiments, the AAV VP capsid polypeptide is a CNS tissue-tropic capsid polypeptide and has an amino acid sequence of SEQ ID NO: 2, wherein X¹, X², X³, X⁴, X⁵, X⁶, X⁷, X⁸, and X⁹ are each independently selected from any amino acid, wherein the capsid polypeptide is capable of assembling into a recombinant AAV virion (rAAV), wherein the at least one substitution confers higher tropism for a central nervous system (CNS) tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8, optionally with further mutations elsewhere in the VP capsid polypeptide. In some embodiments, X¹X²X³X⁴X⁵X⁶X⁷X⁸X⁹ of the CNS tissue-tropic VP capsid polypeptide correspond to a sequence of any one of SEQ ID NO: 12-SEQ ID NO: 3937, SEQ ID NO: 3938-SEQ ID NO: 4237, or SEQ ID NO: 5234-SEQ ID NO: 5807.

In some embodiments, the AAV VP capsid polypeptide is a muscle-tropic capsid polypeptide and has an amino acid sequence of SEQ ID NO: 2, wherein X¹, X², X³, X⁴, X⁵, X⁶, X⁷, X⁸, and X⁹ are each independently selected from any amino acid, wherein the capsid polypeptide is capable of assembling into a recombinant AAV virion (rAAV), wherein the at least one substitution confers higher tropism for a muscle tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8, optionally with further mutations elsewhere in the VP capsid polypeptide. In some embodiments, X¹X²X³X⁴X⁵X⁶X⁷X⁸X⁹ of the muscle-tropic VP capsid polypeptide correspond to a sequence of any one of SEQ ID NO: 4238-SEQ ID NO: 4933, SEQ ID NO: 4934-SEQ ID NO: 5233, or SEQ ID NO: 5808-SEQ ID NO: 5811.

In some embodiments, the engineered adeno-associated virus (AAV) viral protein (VP) capsid polypeptide (e.g., a CNS tissue-tropic VP capsid polypeptide or a muscle-tropic VP capsid polypeptide) has an amino acid sequence of SEQ ID NO: 2, wherein amino acid residues X¹, X², X³, X⁴, X⁵, X⁶, X⁷, X⁸, and X⁹ are each independently selected from A, R, N, D, C, E, Q, G, H, I, L, K, M, F, P, S, T, W, Y, and V; wherein the engineered AAV VP capsid polypeptide is capable of assembling into a recombinant AAV virion (rAAV); and wherein the rAAV VP capsid polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, the 581-589 region of the engineered VP capsid polypeptide, corresponding to residues 581 to 589, inclusive, with reference to SEQ ID NO: 1, has a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to any one of SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811. In some embodiments, the 581-589 region of the engineered VP capsid polypeptide comprises 1 amino acid substitution relative to any one of SEQ ID NO: SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811. In some embodiments, the 581-589 region of the engineered VP capsid polypeptide comprises 2 amino acid substitutions relative to any one of SEQ ID NO: SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811. In particular embodiments, the 581-589 region of the engineered VP capsid polypeptide has a sequence that is identical to any one of SEQ ID NO: SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811.

In some embodiments, the engineered adeno-associated virus (AAV) viral protein (VP) capsid polypeptide is an engineered AAV5 viral capsid protein, wherein the engineered AAV VP5 capsid polypeptide has at least one substitution as compared to SEQ ID NO: 1 in the 581-589 region, corresponding to residues 581 to 589, inclusive, of SEQ ID NO: 1, inclusive; wherein the capsid polypeptide is capable of assembling into a recombinant AAV virion (rAAV); wherein the at least one substitution confers higher tropism for a central nervous system (CNS) tissue on the rAAV as compared to an rAAV virion having an AAV5 VP capsid polypeptide of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8, optionally with further mutations elsewhere in the VP protein.

In some embodiments, the AAV VP capsid polypeptides have an amino acid sequence of SEQ ID NO: 2, wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V; and wherein the polypeptide does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8.

In some embodiments, the engineered AAV VP capsid polypeptide comprises a polypeptide sequence represented by the formula: (A)-(X)—(B), wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence comprising amino acid residues X¹, X², X³, X⁴, X⁵, X⁶, X⁷, X⁸, and X⁹ of SEQ ID NO: 2; and (B) is the polypeptide sequence of SEQ ID NO: 5813 (IVPGSVWMERDVYLQGPIWA, corresponding to residues 590 to 609 of SEQ ID NO: 2); wherein X¹, X², X³, X⁴, X⁵, X⁶, X⁷, X⁸, and X⁹ are each independently selected from A, R, N, D, C, E, Q, G, H, I, L, K, M, F, P, S, T, W, Y, and V; and wherein the capsid polypeptide is capable of assembling into a recombinant AAV virion (rAAV); and wherein the polypeptide does not have the sequence of any of SEQ ID NO: 1, SEQ ID NO: 3, SEQ ID NO: 4, SEQ ID NO: 5, SEQ ID NO: 6, SEQ ID NO: 7, and SEQ ID NO: 8.

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 9 that is expected to confer CNS tissue tropism or preference on a recombinant AAV virion (rAAV), based on a secondary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 9 at the region corresponding to amino acid residues 445 to 453 in AAV5 VP2 or the region corresponding to amino acid residues 389 to 397 in AAV5 VP3.

In some embodiments, a 581-589 region of an AAV5 VP capsid polypeptide has a sequence that is at least 75%, at least 77.7%, at least 85%, at least 88.8%, or 100% identical to a sequence provided in TABLE 9. In some embodiments, a 581-589 region of an AAV5 VP capsid polypeptide has a sequence that is at least 75%, at least 77.7%, at least 85%, at least 88.8% or 100% identical to SEQ ID NO: 18. In some embodiments, a 581-589 region of an AAV5 VP capsid polypeptide has a sequence that is at least 75%, at least 77.7%, at least 85%, at least 88.8% or 100% identical to SEQ ID NO: 3472. In some embodiments, a 581-589 region of an AAV5 VP capsid polypeptide has a sequence that is at least 75%, at least 77.7%, at least 85%, at least 88.8% or 100% identical to SEQ ID NO: 262. In some embodiments, a 581-589 region of an AAV5 VP capsid polypeptide has a sequence that is at least 75%, at least 77.7%, at least 85%, at least 88.8% or 100% identical to SEQ ID NO: 3306. In some embodiments, a 581-589 region of an AAV5 VP capsid polypeptide has a sequence that is at least 75%, at least 77.7%, at least 85%, at least 88.8% or 100% identical to SEQ ID NO: 2028. In some embodiments, a 581-589 region of an AAV5 VP capsid polypeptide has a sequence that is at least 75%, at least 77.7%, at least 85%, at least 88.8% or 100% identical to SEQ ID NO: 2791. In some embodiments, a 581-589 region of an AAV5 VP capsid polypeptide has a sequence that is at least 75%, at least 77.7%, at least 85%, at least 88.8% or 100% identical to SEQ ID NO: 424. In some embodiments, a 581-589 region of an AAV5 VP capsid polypeptide has a sequence that is at least 75%, at least 77.7%, at least 85%, at least 88.8% or 100% identical to SEQ ID NO: 2536. In some embodiments, a 581-589 region of an AAV5 VP capsid polypeptide has a sequence that is at least 75%, at least 77.7%, at least 85%, at least 88.8% or 100% identical to SEQ ID NO: 1971. In some embodiments, a 581-589 region of an AAV5 VP capsid polypeptide has a sequence that is at least 75%, at least 77.7%, at least 85%, at least 88.8% or 100% identical to SEQ ID NO: 415. In some embodiments, a 581-589 region of an AAV5 VP capsid polypeptide has a sequence that is at least 75%, at least 77.7%, at least 85%, at least 88.8% or 100% identical to SEQ ID NO: 3846. In some embodiments, a 581-589 region of an AAV5 VP capsid polypeptide has a sequence that is at least 75%, at least 77.7%, at least 85%, at least 88.8% or 100% identical to SEQ ID NO: 3283. In some embodiments, a 581-589 region of an AAV5 VP capsid polypeptide has a sequence that is at least 75%, at least 77.7%, at least 85%, at least 88.8% or 100% identical to SEQ ID NO: 1956. In some embodiments, a 581-589 region of an AAV5 VP capsid polypeptide has a sequence that is at least 75%, at least 77.7%, at least 85%, at least 88.8% or 100% identical to SEQ ID NO: 3297. In some embodiments, a 581-589 region of an AAV5 VP capsid polypeptide has a sequence that is at least 75%, at least 77.7%, at least 85%, at least 88.8% or 100% identical to SEQ ID NO: 4545. In some embodiments, a 581-589 region of an AAV5 VP capsid polypeptide has a sequence that is at least 75%, at least 77.7%, at least 85%, at least 88.8% or 100% identical to SEQ ID NO: 2661. In some embodiments, a 581-589 region of an AAV5 VP capsid polypeptide has a sequence that is at least 75%, at least 77.7%, at least 85%, at least 88.8% or 100% identical to SEQ ID NO: 1576. In some embodiments, a 581-589 region of an AAV5 VP capsid polypeptide has a sequence that is at least 75%, at least 77.7%, at least 85%, at least 88.8% or 100% identical to SEQ ID NO: 425. In some embodiments, a 581-589 region of an AAV5 VP capsid polypeptide has a sequence that is at least 75%, at least 77.7%, at least 85%, at least 88.8% or 100% identical to SEQ ID NO: 709. In some embodiments, a 581-589 region of an AAV5 VP capsid polypeptide has a sequence that is at least 75%, at least 77.7%, at least 85%, at least 88.8% or 100% identical to SEQ ID NO: 2168. In some embodiments, a 581-589 region of an AAV5 VP capsid polypeptide has a sequence that is at least 75%, at least 77.7%, at least 85%, at least 88.8% or 100% identical to SEQ ID NO: 54. In some embodiments, a 581-589 region of an AAV5 VP capsid polypeptide has a sequence that is at least 75%, at least 77.7%, at least 85%, at least 88.8% or 100% identical to SEQ ID NO: 429. In some embodiments, a 581-589 region of an AAV5 VP capsid polypeptide has a sequence that is at least 75%, at least 77.7%, at least 85%, at least 88.8% or 100% identical to SEQ ID NO: 708. In some embodiments, a 581-589 region of an AAV5 VP capsid polypeptide has a sequence that is at least 75%, at least 77.7%, at least 85%, at least 88.8% or 100% identical to SEQ ID NO: 428. In some embodiments, a 581-589 region of an AAV5 VP capsid polypeptide has a sequence that is at least 75%, at least 77.7%, at least 85%, at least 88.8% or 100% identical to SEQ ID NO: 4119. In some embodiments, a 581-589 region of an AAV5 VP capsid polypeptide has a sequence that is at least 75%, at least 77.7%, at least 85%, at least 88.8% or 100% identical to SEQ ID NO: 3906. In some embodiments, a 581-589 region of an AAV5 VP capsid polypeptide has a sequence that is at least 75%, at least 77.7%, at least 85%, at least 88.8% or 100% identical to SEQ ID NO: 2456. In some embodiments, a 581-589 region of an AAV5 VP capsid polypeptide has a sequence that is at least 75%, at least 77.7%, at least 85%, at least 88.8% or 100% identical to SEQ ID NO: 2278. In some embodiments, a 581-589 region of an AAV5 VP capsid polypeptide has a sequence that is at least 75%, at least 77.7%, at least 85%, at least 88.8% or 100% identical to SEQ ID NO: 5006. In some embodiments, a 581-589 region of an AAV5 VP capsid polypeptide has a sequence that is at least 75%, at least 77.7%, at least 85%, at least 88.8% or 100% identical to SEQ ID NO: 426.

In some embodiments, a 581-589 region of an AAV5 VP capsid polypeptide has a sequence of SEQ ID NO: 18. In some embodiments, a 581-589 region of an AAV5 VP capsid polypeptide has a sequence of SEQ ID NO: 3472. In some embodiments, a 581-589 region of an AAV5 VP capsid polypeptide has a sequence of SEQ ID NO: 262. In some embodiments, a 581-589 region of an AAV5 VP capsid polypeptide has a sequence of SEQ ID NO: 3306. In some embodiments, a 581-589 region of an AAV5 VP capsid polypeptide has a sequence of SEQ ID NO: 2028. In some embodiments, a 581-589 region of an AAV5 VP capsid polypeptide has a sequence of SEQ ID NO: 2791. In some embodiments, a 581-589 region of an AAV5 VP capsid polypeptide has a sequence of SEQ ID NO: 424. In some embodiments, a 581-589 region of an AAV5 VP capsid polypeptide has a sequence of SEQ ID NO: 2536. In some embodiments, a 581-589 region of an AAV5 VP capsid polypeptide has a sequence of SEQ ID NO: 1971. In some embodiments, a 581-589 region of an AAV5 VP capsid polypeptide has a sequence of SEQ ID NO: 415. In some embodiments, a 581-589 region of an AAV5 VP capsid polypeptide has a sequence of SEQ ID NO: 3846. In some embodiments, a 581-589 region of an AAV5 VP capsid polypeptide has a sequence of SEQ ID NO: 3283. In some embodiments, a 581-589 region of an AAV5 VP capsid polypeptide has a sequence of SEQ ID NO: 1956. In some embodiments, a 581-589 region of an AAV5 VP capsid polypeptide has a sequence of SEQ ID NO: 3297. In some embodiments, a 581-589 region of an AAV5 VP capsid polypeptide has a sequence of SEQ ID NO: 4545. In some embodiments, a 581-589 region of an AAV5 VP capsid polypeptide has a sequence of SEQ ID NO: 2661. In some embodiments, a 581-589 region of an AAV5 VP capsid polypeptide has a sequence of SEQ ID NO: 1576. In some embodiments, a 581-589 region of an AAV5 VP capsid polypeptide has a sequence of SEQ ID NO: 425. In some embodiments, a 581-589 region of an AAV5 VP capsid polypeptide has a sequence of SEQ ID NO: 709. In some embodiments, a 581-589 region of an AAV5 VP capsid polypeptide has a sequence of SEQ ID NO: 2168. In some embodiments, a 581-589 region of an AAV5 VP capsid polypeptide has a sequence of SEQ ID NO: 54. In some embodiments, a 581-589 region of an AAV5 VP capsid polypeptide has a sequence of SEQ ID NO: 429. In some embodiments, a 581-589 region of an AAV5 VP capsid polypeptide has a sequence of SEQ ID NO: 708. In some embodiments, a 581-589 region of an AAV5 VP capsid polypeptide has a sequence of SEQ ID NO: 428. In some embodiments, a 581-589 region of an AAV5 VP capsid polypeptide has a sequence of SEQ ID NO: 4119. In some embodiments, a 581-589 region of an AAV5 VP capsid polypeptide has a sequence of SEQ ID NO: 3906. In some embodiments, a 581-589 region of an AAV5 VP capsid polypeptide has a sequence of SEQ ID NO: 2456. In some embodiments, a 581-589 region of an AAV5 VP capsid polypeptide has a sequence of SEQ ID NO: 2278. In some embodiments, a 581-589 region of an AAV5 VP capsid polypeptide has a sequence of SEQ ID NO: 5006. In some embodiments, a 581-589 region of an AAV5 VP capsid polypeptide has a sequence of SEQ ID NO: 426.

In some embodiments, a 581-589 region of an AAV5 VP capsid polypeptide has a sequence of SEQ ID NO: 18. In some embodiments, a 581-589 region of an AAV5 VP capsid polypeptide has a sequence of SEQ ID NO: 2028. In some embodiments, a 581-589 region of an AAV5 VP capsid polypeptide has a sequence of SEQ ID NO: 424. In some embodiments, a 581-589 region of an AAV5 VP capsid polypeptide has a sequence of SEQ ID NO: 3306. In some embodiments, a 581-589 region of an AAV5 VP capsid polypeptide has a sequence of SEQ ID NO: 3472. In some embodiments, a 581-589 region of an AAV5 VP capsid polypeptide has a sequence of SEQ ID NO: 415. In some embodiments, a 581-589 region of an AAV5 VP capsid polypeptide has a sequence of SEQ ID NO: 709. In some embodiments, a 581-589 region of an AAV5 VP capsid polypeptide has a sequence of SEQ ID NO: 1971. In some embodiments, a 581-589 region of an AAV5 VP capsid polypeptide has a sequence of SEQ ID NO: 2791. In some embodiments, a 581-589 region of an AAV5 VP capsid polypeptide has a sequence of SEQ ID NO: 1956. In some embodiments, a 581-589 region of an AAV5 VP capsid polypeptide has a sequence of SEQ ID NO: 262. In some embodiments, a 581-589 region of an AAV5 VP capsid polypeptide has a sequence of SEQ ID NO: 425. In some embodiments, a 581-589 region of an AAV5 VP capsid polypeptide has a sequence of SEQ ID NO: 2536. In some embodiments, a 581-589 region of an AAV5 VP capsid polypeptide has a sequence of SEQ ID NO: 708. In some embodiments, a 581-589 region of an AAV5 VP capsid polypeptide has a sequence of SEQ ID NO: 54. In some embodiments, a 581-589 region of an AAV5 VP capsid polypeptide has a sequence of SEQ ID NO: 3283. In some embodiments, a 581-589 region of an AAV5 VP capsid polypeptide has a sequence of SEQ ID NO: 430. In some embodiments, a 581-589 region of an AAV5 VP capsid polypeptide has a sequence of SEQ ID NO: 3297. In some embodiments, a 581-589 region of an AAV5 VP capsid polypeptide has a sequence of SEQ ID NO: 2661. In some embodiments, a 581-589 region of an AAV5 VP capsid polypeptide has a sequence of SEQ ID NO: 428. In some embodiments, a 581-589 region of an AAV5 VP capsid polypeptide has a sequence of SEQ ID NO: 885. In some embodiments, a 581-589 region of an AAV5 VP capsid polypeptide has a sequence of SEQ ID NO: 429. In some embodiments, a 581-589 region of an AAV5 VP capsid polypeptide has a sequence of SEQ ID NO: 569. In some embodiments, a 581-589 region of an AAV5 VP capsid polypeptide has a sequence of SEQ ID NO: 724. In some embodiments, a 581-589 region of an AAV5 VP capsid polypeptide has a sequence of SEQ ID NO: 1576. In some embodiments, a 581-589 region of an AAV5 VP capsid polypeptide has a sequence of SEQ ID NO: 4545. In some embodiments, a 581-589 region of an AAV5 VP capsid polypeptide has a sequence of SEQ ID NO: 426. In some embodiments, a 581-589 region of an AAV5 VP capsid polypeptide has a sequence of SEQ ID NO: 1887. In some embodiments, a 581-589 region of an AAV5 VP capsid polypeptide has a sequence of SEQ ID NO: 3906. In some embodiments, a 581-589 region of an AAV5 VP capsid polypeptide has a sequence of SEQ ID NO: 3935.

In some embodiments, a 581-589 region of an AAV5 VP capsid polypeptide has a sequence of SEQ ID NO: 18 with 1 or 2 conservative substitutions. In some embodiments, a 581-589 region of an AAV5 VP capsid polypeptide has a sequence of SEQ ID NO: 3472 with 1 or 2 conservative substitutions. In some embodiments, a 581-589 region of an AAV5 VP capsid polypeptide has a sequence of SEQ ID NO: 262 with 1 or 2 conservative substitutions. In some embodiments, a 581-589 region of an AAV5 VP capsid polypeptide has a sequence of SEQ ID NO: 3306 with 1 or 2 conservative substitutions. In some embodiments, a 581-589 region of an AAV5 VP capsid polypeptide has a sequence of SEQ ID NO: 2028 with 1 or 2 conservative substitutions. In some embodiments, a 581-589 region of an AAV5 VP capsid polypeptide has a sequence of SEQ ID NO: 2791 with 1 or 2 conservative substitutions. In some embodiments, a 581-589 region of an AAV5 VP capsid polypeptide has a sequence of SEQ ID NO: 424 with 1 or 2 conservative substitutions. In some embodiments, a 581-589 region of an AAV5 VP capsid polypeptide has a sequence of SEQ ID NO: 2536 with 1 or 2 conservative substitutions. In some embodiments, a 581-589 region of an AAV5 VP capsid polypeptide has a sequence of SEQ ID NO: 1971 with 1 or 2 conservative substitutions. In some embodiments, a 581-589 region of an AAV5 VP capsid polypeptide has a sequence of SEQ ID NO: 415 with 1 or 2 conservative substitutions. In some embodiments, a 581-589 region of an AAV5 VP capsid polypeptide has a sequence of SEQ ID NO: 3846 with 1 or 2 conservative substitutions. In some embodiments, a 581-589 region of an AAV5 VP capsid polypeptide has a sequence of SEQ ID NO: 3283 with 1 or 2 conservative substitutions. In some embodiments, a 581-589 region of an AAV5 VP capsid polypeptide has a sequence of SEQ ID NO: 1956 with 1 or 2 conservative substitutions. In some embodiments, a 581-589 region of an AAV5 VP capsid polypeptide has a sequence of SEQ ID NO: 3297 with 1 or 2 conservative substitutions. In some embodiments, a 581-589 region of an AAV5 VP capsid polypeptide has a sequence of SEQ ID NO: 4545 with 1 or 2 conservative substitutions. In some embodiments, a 581-589 region of an AAV5 VP capsid polypeptide has a sequence of SEQ ID NO: 2661 with 1 or 2 conservative substitutions. In some embodiments, a 581-589 region of an AAV5 VP capsid polypeptide has a sequence of SEQ ID NO: 1576 with 1 or 2 conservative substitutions. In some embodiments, a 581-589 region of an AAV5 VP capsid polypeptide has a sequence of SEQ ID NO: 425 with 1 or 2 conservative substitutions. In some embodiments, a 581-589 region of an AAV5 VP capsid polypeptide has a sequence of SEQ ID NO: 709 with 1 or 2 conservative substitutions. In some embodiments, a 581-589 region of an AAV5 VP capsid polypeptide has a sequence of SEQ ID NO: 2168 with 1 or 2 conservative substitutions. In some embodiments, a 581-589 region of an AAV5 VP capsid polypeptide has a sequence of SEQ ID NO: 54 with 1 or 2 conservative substitutions. In some embodiments, a 581-589 region of an AAV5 VP capsid polypeptide has a sequence of SEQ ID NO: 429 with 1 or 2 conservative substitutions. In some embodiments, a 581-589 region of an AAV5 VP capsid polypeptide has a sequence of SEQ ID NO: 708 with 1 or 2 conservative substitutions. In some embodiments, a 581-589 region of an AAV5 VP capsid polypeptide has a sequence of SEQ ID NO: 428 with 1 or 2 conservative substitutions. In some embodiments, a 581-589 region of an AAV5 VP capsid polypeptide has a sequence of SEQ ID NO: 4119 with 1 or 2 conservative substitutions. In some embodiments, a 581-589 region of an AAV5 VP capsid polypeptide has a sequence of SEQ ID NO: 3906 with 1 or 2 conservative substitutions. In some embodiments, a 581-589 region of an AAV5 VP capsid polypeptide has a sequence of SEQ ID NO: 2456 with 1 or 2 conservative substitutions. In some embodiments, a 581-589 region of an AAV5 VP capsid polypeptide has a sequence of SEQ ID NO: 2278 with 1 or 2 conservative substitutions. In some embodiments, a 581-589 region of an AAV5 VP capsid polypeptide has a sequence of SEQ ID NO: 5006 with 1 or 2 conservative substitutions. In some embodiments, a 581-589 region of an AAV5 VP capsid polypeptide has a sequence of SEQ ID NO: 426 with 1 or 2 conservative substitutions. The conservative substitutions may be at any position in the 581-589 region.

In some embodiments, an AAV VP capsid polypeptide preferentially targets a CNS tissue at a DNA enrichment of at least 100-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3846, SEQ ID NO: 3283, SEQ ID NO: 1956, SEQ ID NO: 3297, SEQ ID NO: 4545, SEQ ID NO: 2661, SEQ ID NO: 1576, SEQ ID NO: 425, SEQ ID NO: 709, SEQ ID NO: 2168, SEQ ID NO: 54, SEQ ID NO: 429, SEQ ID NO: 708, SEQ ID NO: 428, SEQ ID NO: 4119, SEQ ID NO: 3906, SEQ ID NO: 2456, SEQ ID NO: 2278, SEQ ID NO: 5006, SEQ ID NO: 426, SEQ ID NO: 307, SEQ ID NO: 5155, SEQ ID NO: 2640, SEQ ID NO: 4317, SEQ ID NO: 1145, SEQ ID NO: 430, or SEQ ID NO: 885 may preferentially target a CNS tissue at a DNA enrichment of at least 100-fold greater than a wild type AAV9.

In some embodiments, an AAV VP capsid polypeptide preferentially targets a CNS tissue at a DNA enrichment of at least 200-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3846, SEQ ID NO: 3283, SEQ ID NO: 1956, or SEQ ID NO: 3297 may preferentially target a CNS tissue at a DNA enrichment of at least 200-fold greater than a wild type AAV9.

In some embodiments, an AAV VP capsid polypeptide preferentially targets a CNS tissue at a DNA enrichment of at least 1000-fold greater than a wild type AAV9. For example, an AAV VP capsid polypeptide comprising a 581-589 region of SEQ ID NO: 18 may preferentially target a CNS tissue at a DNA enrichment of at least 1000-fold greater than a wild type AAV9.

In some embodiments, an AAV VP capsid polypeptide preferentially targets a CNS tissue at an RNA enrichment of at least 100-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 3297, SEQ ID NO: 4545, SEQ ID NO: 2661, SEQ ID NO: 1576, SEQ ID NO: 425, SEQ ID NO: 709, SEQ ID NO: 54, SEQ ID NO: 429, SEQ ID NO: 428, SEQ ID NO: 426, or SEQ ID NO: 430 may preferentially target a CNS tissue at an RNA enrichment of at least 100-fold greater than a wild type AAV9.

In some embodiments, an AAV VP capsid polypeptide preferentially targets a CNS tissue at an RNA enrichment of at least 200-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 2661, SEQ ID NO: 1576, SEQ ID NO: 425, SEQ ID NO: 709, SEQ ID NO: 54, SEQ ID NO: 430, SEQ ID NO: 569, and SEQ ID NO: 719 may preferentially target a CNS tissue at an RNA enrichment of at least 200-fold greater than a wild type AAV9.

In some embodiments, an AAV VP capsid polypeptide preferentially targets a CNS tissue at an RNA enrichment of at least 500-fold greater than a wild type AAV9. For example, an AAV VP capsid polypeptide comprising a 581-589 region of SEQ ID NO: 18, SEQ ID NO: 3472, SEQ ID NO: 262, and SEQ ID NO: 2028 may preferentially target a CNS tissue at an RNA enrichment of at least 500-fold greater than a wild type AAV9.

In some embodiments, an AAV VP capsid polypeptide preferentially targets a CNS tissue at a DNA enrichment of at least 5-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3846, SEQ ID NO: 3283, SEQ ID NO: 1956, SEQ ID NO: 3297, SEQ ID NO: 4545, SEQ ID NO: 2661, or SEQ ID NO: 1576 may preferentially target a CNS tissue at a DNA enrichment of at least 5-fold greater than a wild type AAV5.

In some embodiments, an AAV VP capsid polypeptide preferentially targets a CNS tissue at a DNA enrichment of at least 10-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, or SEQ ID NO: 424 may preferentially target a CNS tissue at a DNA enrichment of at least 10-fold greater than a wild type AAV5.

In some embodiments, an AAV VP capsid polypeptide preferentially targets a CNS tissue at an RNA enrichment of at least 5-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 3297, SEQ ID NO: 4545, SEQ ID NO: 2661, SEQ ID NO: 1576, SEQ ID NO: 425, SEQ ID NO: 709, SEQ ID NO: 54, SEQ ID NO: 429, SEQ ID NO: 708, SEQ ID NO: 428, SEQ ID NO: 426, SEQ ID NO: 307, SEQ ID NO: 4317, SEQ ID NO: 430, or SEQ ID NO: 885 may preferentially target a CNS tissue at an RNA enrichment of at least 5-fold greater than a wild type AAV5.

In some embodiments, an AAV VP capsid polypeptide preferentially targets a CNS tissue at an RNA enrichment of at least 20-fold greater than a wild type AAV9. For example, an AAV VP capsid polypeptide comprising a 581-589 region of SEQ ID NO: 18, SEQ ID NO: 3472, SEQ ID NO: 262, SEQ ID NO: 3306, SEQ ID NO: 2028, SEQ ID NO: 2791, SEQ ID NO: 424, SEQ ID NO: 2536, SEQ ID NO: 1971, SEQ ID NO: 415, SEQ ID NO: 3283, SEQ ID NO: 1956, SEQ ID NO: 2661, SEQ ID NO: 1576, SEQ ID NO: 425, SEQ ID NO: 709, SEQ ID NO: 54, SEQ ID NO: 430, SEQ ID NO: 569, and SEQ ID NO: 719 may preferentially target a CNS tissue at an RNA enrichment of at least 20-fold greater than a wild type AAV5.

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 2 (SEQ ID NO: 12-SEQ ID NO: 3937) that is expected to confer CNS tissue tropism or preference on a recombinant AAV virion (rAAV), based on a primary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 2 (SEQ ID NO: 12-SEQ ID NO: 3937) at the region corresponding to amino acid residues 445 to 453 in AAV5 VP2 or the region corresponding to amino acid residues 389 to 397 in AAV5 VP3.

TABLE2
Sequences of 581-589 Regions

| SEQ ID | |
|---|---|
| NO | Sequence |
| 12 | AAANMYWWS |
| 13 | AAAQSPEGV |
| 14 | AAAVQWQMM |
| 15 | AACHIMSQT |
| 16 | AAEPQHCYY |
| 17 | AAGQFEWLH |
| 18 | AAGRFNYFG |
| 19 | AAGTHHVMR |
| 20 | AAGVVQRGE |
| 21 | AALNTQLGH |
| 22 | AALSSRQVL |
| 23 | AALSWRSHF |
| 24 | AALYSQRAQ |
| 25 | AAMPINMCC |
| 26 | AAMTQHCPV |
| 27 | AANDCQIWF |
| 28 | AANYYATSW |
| 29 | AAQFDGFHV |
| 30 | AAQLEGFHV |
| 31 | AAQSMVCDS |
| 32 | AARDTMEGK |
| 33 | AARIYLMYV |
| 34 | AASYWGNMY |
| 35 | AATMTNHAW |
| 36 | AATQEYWQG |
| 37 | AAVYGKFSW |
| 38 | AAYLDDQYS |
| 39 | AAYPCQLER |
| 40 | AAYPYICAD |
| 41 | ACGYFTYNV |
| 42 | ACHTMVIEN |
| 43 | ACIYKIAQE |
| 44 | ACKFGQESD |
| 45 | ACYQYKCRR |
| 46 | ADAIRQPVL |
| 47 | ADAMRQPVL |
| 48 | ADAPVNASC |
| 49 | ADSIKLTEY |
| 50 | ADYTMDWHL |
| 51 | AEAMMSAVF |
| 52 | AEAQFQYWS |
| 53 | AEDLSPVPV |
| 54 | AEDYWDLGA |
| 55 | AEIILPGMQ |
| 56 | AEIYVHCQT |
| 57 | AEIYVHWQT |
| 58 | AEKLYGLST |
| 59 | AEREHWFYC |
| 60 | AFAQKCRYH |
| 61 | AFAYELNIN |
| 62 | AFCPISSGI |
| 63 | AFEQWAHNE |
| 64 | AFHAHEQAG |
| 65 | AFHQVHSMG |
| 66 | AFNDKPSCC |
| 67 | AFQNACKSA |
| 68 | AFTYGNHQF |
| 69 | AGASTWMQS |
| 70 | AGCSIQAWC |
| 71 | AGIVQGIWG |
| 72 | AGKCTPVTS |
| 73 | AGKYYQDYQ |
| 74 | AGMPWDGHC |
| 75 | AGQREVPSD |
| 76 | AHAMLDRNF |
| 77 | AHATYNKPT |
| 78 | AHDSASRCA |
| 79 | AHDWTHICA |
| 80 | AHGNSPTAY |
| 81 | AHHLGTMGH |
| 82 | AHKSPDKDV |
| 83 | AHLHNQFMP |
| 84 | AHLTPNLHE |
| 85 | AHMDEFRSG |
| 86 | AHMSTYWGF |
| 87 | AHNPCMHHQ |
| 88 | AHQTSWAWI |
| 89 | AHSDLTGAI |
| 90 | AHYQFQALM |
| 91 | AICQQHSYA |
| 92 | AIDQLSWHG |
| 93 | AIEIFGACV |
| 94 | AIEMTKYPW |
| 95 | AILSIYPSG |
| 96 | AILYQSAGE |
| 97 | AIMHYHRFK |
| 98 | AIQQCHAYA |
| 99 | AIQWFQAPG |
| 100 | AISYMQLEA |
| 101 | AITPRMGMK |
| 102 | AITSCICEK |
| 103 | AIWGYGMNY |
| 104 | AKAYCTSVD |
| 105 | AKCEHDHCW |
| 106 | AKDCVQSQA |
| 107 | AKFVGVAAD |
| 108 | AKIHGAWKA |
| 109 | AKQHALMIE |
| 110 | AKSTMPGYQ |
| 111 | AKTFVMSFG |
| 112 | AKTSLQIDP |
| 113 | ALEASYGYF |
| 114 | ALECGHLPY |
| 115 | ALFYMNADF |
| 116 | ALIQFGQSW |
| 117 | ALKSHGCLT |
| 118 | ALLNRTACW |
| 119 | ALPHIQSEP |
| 120 | ALPHIRSEP |
| 121 | ALTNWGMSG |
| 122 | ALWMFDVFG |
| 123 | AMAQSMVQF |
| 124 | AMDKIPVFQ |
| 125 | AMDPPIREP |
| 126 | AMDRIPIFQ |
| 127 | AMEQVSATF |
| 128 | AMFNGTSMS |
| 129 | AMGIGMRID |
| 130 | AMGPCNGPM |
| 131 | AMGYNGTYP |
| 132 | AMKWVEACP |
| 133 | AMKWVEAWQ |
| 134 | AMKYIGCPM |
| 135 | AMLAYFATE |
| 136 | AMLKNHCYH |
| 137 | AMMLCIWVD |
| 138 | AMMNMNVQF |
| 139 | AMQCFPAQQ |
| 140 | AMQCNQMQD |
| 141 | AMQPTRIVG |
| 142 | AMQPVRIVV |
| 143 | AMRQIIAPC |
| 144 | ANAVTEEWM |
| 145 | ANGDKVAMD |
| 146 | ANITIGPQA |
| 147 | ANIYICPQA |
| 148 | ANLDSHQFE |
| 149 | ANMVAHMMA |
| 150 | ANQHFHAES |
| 151 | ANTRDNAEL |
| 152 | ANTWLAHFE |
| 153 | APCHDKDRE |
| 154 | APIQGQMMM |
| 155 | APKLVGLST |
| 156 | AQCQDMWGH |
| 157 | AQEPFKTIQ |
| 158 | AQFRAIIGN |
| 159 | AQGTMDSWY |
| 160 | AQIQPAMFP |
| 161 | AQMTVCARE |
| 162 | AQMYWSSKD |
| 163 | AQNNKFDGS |
| 164 | AQSFLWKGC |
| 165 | AQVLMEMCT |
| 166 | AQYCTIMAH |
| 167 | ARKFCEGTP |
| 168 | ARQVYEMNG |
| 169 | ARQWYEMTG |
| 170 | ASEMTKGPW |
| 171 | ASFMSWEGA |
| 172 | ASGWAPCSW |
| 173 | ASMQEHHYT |
| 174 | ASQHFHAPQ |
| 175 | ASQHNHEKQ |
| 176 | ASQLIECNA |
| 177 | ASQPRGAEQ |
| 178 | ASQTTHGPL |
| 179 | ASTERAKFG |
| 180 | ASTQQGFWC |
| 181 | ASVCEHTLE |
| 182 | ASVECWQWT |
| 183 | ASVPISHSQ |
| 184 | ASWLCGRNV |
| 185 | ASYNGCMIC |
| 186 | ASYPSSQVF |
| 187 | ASYSICDAG |
| 188 | ATAGIPTAG |
| 189 | ATAQFQMEG |
| 190 | ATCTFNPGI |
| 191 | ATCWVQMGQ |
| 192 | ATEGYNMHC |
| 193 | ATENYNRRG |
| 194 | ATFMPNQSF |
| 195 | ATGMRIAVC |
| 196 | ATGMRIDVC |
| 197 | ATGTYNIQE |
| 198 | ATISKLECQ |
| 199 | ATKTCAEGG |
| 200 | ATLNSMHTQ |
| 201 | ATMCHCVVA |
| 202 | ATMWFTANG |
| 203 | ATMWMQWWD |
| 204 | ATMWVQAYG |
| 205 | ATNQLNESF |
| 206 | ATQGYNMHC |
| 207 | ATQLYMHHK |
| 208 | ATQMYMHCK |
| 209 | ATTFSSASF |
| 210 | ATTTVNRQE |
| 211 | ATVDVKGAH |
| 212 | ATVWSINAD |
| 213 | ATWSADVTA |
| 214 | AVDLTSKHS |
| 215 | AVDPTKDGQ |
| 216 | AVEQLNQPI |
| 217 | AVGCSQHWK |
| 218 | AVKCPESLI |
| 219 | AVMPSHQIT |
| 220 | AVNACSNSH |
| 221 | AVNDCKMWE |
| 222 | AVRIMFMQE |
| 223 | AVSGCRGSA |
| 224 | AVSNAMIAG |
| 225 | AVSNWLETS |
| 226 | AVSPSNFLA |
| 227 | AWCCSSVGE |
| 228 | AWFKTMPSD |
| 229 | AWFSHHQFC |
| 230 | AWHQTQDDM |
| 231 | AWREQEWAH |
| 232 | AWSEFQWFD |
| 233 | AWTPLMSYN |
| 234 | AWVPEGHFP |
| 235 | AWYHTQDDM |
| 236 | AYADMFQCK |
| 237 | AYCLTNGPH |
| 238 | AYCSKMRGE |
| 239 | AYDFVGVQE |
| 240 | AYFLLHASG |
| 241 | AYGPFAMLY |
| 242 | AYNAMHTAW |
| 243 | AYNFKVGSD |
| 244 | AYNIDYQPV |
| 245 | AYNWQQTSA |
| 246 | AYRCYACET |
| 247 | AYRCYAVEQ |
| 248 | CAARCYKAR |
| 249 | CADFKNECA |
| 250 | CADTAWQYT |
| 251 | CAEPKRMEY |
| 252 | CAEWRIMWS |
| 253 | CAGFMQTCQ |
| 254 | CAGGIVCHN |
| 255 | CAGGTVHHN |
| 256 | CAHSPHSHL |
| 257 | CAHYAQWGK |
| 258 | CAILAYAHH |
| 259 | CAIYSGLHI |
| 260 | CAMQCEHAE |
| 261 | CANYWQDMG |
| 262 | CATRFNIGG |
| 263 | CCCTVEREC |
| 264 | CCEEYACAE |
| 265 | CCTPTECQM |
| 266 | CCYPTHCQM |
| 267 | CCYQLGLMP |
| 268 | CDGSKSVGD |
| 269 | CDLCIAACE |
| 270 | CECRYFKFA |
| 271 | CECSYFKHA |
| 272 | CEKVQAHWM |
| 273 | CETYTIHIP |
| 274 | CEVDICNDR |
| 275 | CFLLTNRIS |
| 276 | CFTITQGEW |
| 277 | CFWVRGMWN |
| 278 | CGANYQRFG |
| 279 | CGGTYFALP |
| 280 | CGVNYQWFG |
| 281 | CHAGFPAWP |
| 282 | CHCKTDMWA |
| 283 | CHHHYHTHL |
| 284 | CHNVENCME |
| 285 | CHVNLKAWW |
| 286 | CIENMFVNS |
| 287 | CIGNKSASG |
| 288 | CIHECCDSG |
| 289 | CIHSVHFAA |
| 290 | CIKWYAAST |
| 291 | CILPRGIGQ |
| 292 | CILYLEEAQ |
| 293 | CIMFRSATS |
| 294 | CIMTRAATS |
| 295 | CIRSKPDYN |
| 296 | CISWGGNYG |
| 297 | CIVQKGNMV |
| 298 | CIYSKTGAG |
| 299 | CKAHKQTMS |
| 300 | CKELYPQAV |
| 301 | CKKTTGVCY |
| 302 | CKNPTGVCY |
| 303 | CKQEQLKMP |
| 304 | CLDHINSLS |
| 305 | CLWIYSVMG |
| 306 | CMCRVEQVA |
| 307 | CMKVKQSQP |
| 308 | CMMLMMTGQ |
| 309 | CMWVMIHFE |
| 310 | CMYNVSHWN |
| 311 | CMYTGDGPM |
| 312 | CNFSALPKH |
| 313 | CNGHYTVIS |
| 314 | CNGPNVSEI |
| 315 | CNQTIRPII |
| 316 | CNQTIRPIK |
| 317 | CNSWYAAST |
| 318 | CNVPRLVRH |
| 319 | CPAKHAQAR |
| 320 | CPESSSHQT |
| 321 | CPKQANRSG |
| 322 | CPTFWGHSE |
| 323 | CPWFIKWGI |
| 324 | CQAIVNRLN |
| 325 | CQASHEHAH |
| 326 | CQDAVGRSY |
| 327 | CQDAVGRTF |
| 328 | CQDCYHCDR |
| 329 | CQEDMSSTY |
| 330 | CQKWYESFG |
| 331 | CQSCISDAG |
| 332 | CQYLSFRTV |
| 333 | CRGKSHDHT |
| 334 | CSAAFQLQP |
| 335 | CSALWGGCC |
| 336 | CSAQIKAAC |
| 337 | CSDVSTIYI |
| 338 | CSFQEKAAC |
| 339 | CSHETGAMC |
| 340 | CSMDEYAMA |
| 341 | CSMHMHYVE |
| 342 | CSMTGVMFE |
| 343 | CSRHDLNGD |
| 344 | CSSGFHLFH |
| 345 | CSTGFHCEQ |
| 346 | CSTNCTANP |
| 347 | CSVPGEQAF |
| 348 | CTASWMSWD |
| 349 | CTCQGTGFS |
| 350 | CTCQGTGFT |
| 351 | CTERTKCAS |
| 352 | CTERTMNAS |
| 353 | CTGIQWTGG |
| 354 | CTHECNDSG |
| 355 | CTHPGHQNQ |
| 356 | CTHWFTSLT |
| 357 | CTIAYASMA |
| 358 | CTILATVHH |
| 359 | CTNAYFMSE |
| 360 | CTNTYFMSE |
| 361 | CTQLKECWL |
| 362 | CTTETQCRS |
| 363 | CTTPHDCHF |
| 364 | CTTPNLQVC |
| 365 | CTTSHLIGC |
| 366 | CTVKEMANH |
| 367 | CTWWGHQSM |
| 368 | CVDAKDHCE |
| 369 | CVEWRIMWS |
| 370 | CVHSKMAFH |
| 371 | CVIDVKEAG |
| 372 | CVMEYMDWG |
| 373 | CVNPDDMAQ |
| 374 | CVQQEMGGG |
| 375 | CVSFDACFA |
| 376 | CVSRAWDEK |
| 377 | CVTPKMDLP |
| 378 | CVTTKGDLP |
| 379 | CVVPLTNYY |
| 380 | CWDLVRDCK |
| 381 | CWQFMTAHP |
| 382 | CWQSEGAMM |
| 383 | CWWNFGKDC |
| 384 | CYMPFKMQH |
| 385 | CYNSGDCDN |
| 386 | CYQFWSQYS |
| 387 | CYTEPSNKH |
| 388 | CYWPMTNCT |
| 389 | DACTNKSHM |
| 390 | DADDSNIAW |
| 391 | DADEYKLDC |
| 392 | DAEVTMDAM |
| 393 | DAFHMKQHC |
| 394 | DAFQYPTIG |
| 395 | DAGFTWINC |
| 396 | DAGKFGVDI |
| 397 | DAGWIMNNS |
| 398 | DAGYSMTYH |
| 399 | DAHHGSRCG |
| 400 | DAHMYMVHQ |
| 401 | DAHVAFTDC |
| 402 | DAIHFNMKD |
| 403 | DAITVCAQE |
| 404 | DAIWYGRLG |
| 405 | DALHIGRSG |
| 406 | DAMGYLKGG |
| 407 | DAMQKHSAE |
| 408 | DANLYHPME |
| 409 | DANMQPVTF |
| 410 | DAPLIPQWG |
| 411 | DAQCRNTDG |
| 412 | DAQGIELFT |
| 413 | DAQLLMQAD |
| 414 | DAQLSTSYW |
| 415 | DARVYRALD |
| 416 | DASCATLWS |
| 417 | DASCATPWS |
| 418 | DASHVKSIE |
| 419 | DASIGAGPF |
| 420 | DASIMMEMG |
| 421 | DASIVWTID |
| 422 | DASTQCYMS |
| 423 | DATWEHIRL |
| 424 | DAWCFISSY |
| 425 | DAWLQLKDN |
| 426 | DAWMMMWGS |
| 427 | DAWQMKEAL |
| 428 | DAWQMLSGN |
| 429 | DAWSVLQDK |
| 430 | DAWSYQCYH |
| 431 | DAWYSKSQQ |
| 432 | DAYACSSSF |
| 433 | DAYPSNIAW |
| 434 | DCDAWHANV |
| 435 | DCDIGWGCF |
| 436 | DCHQSPVFY |
| 437 | DCMRNTVFE |
| 438 | DCMWMQNGL |
| 439 | DCNFDCPGA |
| 440 | DCQGQHRVC |
| 441 | DCQQILWDA |
| 442 | DCRTLEEFI |
| 443 | DCSWQTMAF |
| 444 | DCVVPMPWP |
| 445 | DEMKFNRDD |
| 446 | DFAACNSGF |
| 447 | DFACHNNFT |
| 448 | DFAHHSTGV |
| 449 | DFASCDICS |
| 450 | DFASFNDCS |
| 451 | DFAYHSTGV |
| 452 | DFDPWFITT |
| 453 | DFELQDTSF |
| 454 | DFEYSGQLG |
| 455 | DFEYSHDWN |
| 456 | DFGGVMGTA |
| 457 | DFHTMHQFE |
| 458 | DFIHNCHCD |
| 459 | DFINMPYWS |
| 460 | DFMQNSVHY |
| 461 | DFRHVRRSL |
| 462 | DFSQHKMHQ |
| 463 | DFSVTSHMG |
| 464 | DFVTNPNYE |
| 465 | DFYTFSFEN |
| 466 | DGAVYVKQD |
| 467 | DGDAKLVDG |
| 468 | DGGWFMQGE |
| 469 | DGIVQGVWG |
| 470 | DGKLQSTCI |
| 471 | DGKLQSTCM |
| 472 | DGQPYMTYS |
| 473 | DGSFVQMTQ |
| 474 | DGYMCMSGG |
| 475 | DHCSMCCGN |
| 476 | DHKHNVIYP |
| 477 | DHMQWGKFD |
| 478 | DHRHSFMGF |
| 479 | DHVMLRMIN |
| 480 | DHWYFLYHG |
| 481 | DICEGPKQY |
| 482 | DICQVIDNE |
| 483 | DICSDSWKV |
| 484 | DIDSFMHCL |
| 485 | DIGFMDTRA |
| 486 | DIICFNWQV |
| 487 | DIKCERDAD |
| 488 | DIMRLSWPI |
| 489 | DIQLYWMNQ |
| 490 | DIREMMCKN |
| 491 | DIRRAFAHC |
| 492 | DISFGEYNP |
| 493 | DISSMYWWH |
| 494 | DISWGGLME |
| 495 | DISYFLYHG |
| 496 | DIWIQMVNT |
| 497 | DKCCDTGLS |
| 498 | DKCYMTSSF |
| 499 | DKFPTHFSD |
| 500 | DKHEMVNVG |
| 501 | DKIFTIACP |
| 502 | DKIFYGACP |
| 503 | DKLPKVCDW |
| 504 | DKLTQSAYQ |
| 505 | DKLVYTQDR |
| 506 | DKNATAIWE |
| 507 | DKQFANHFT |
| 508 | DKQWAHHSM |
| 509 | DKRICHLNY |
| 510 | DKTVQSLAL |
| 511 | DKVAVDPYR |
| 512 | DKYMFQRDG |
| 513 | DKYMFWRDE |
| 514 | DLEPITTQA |
| 515 | DLFAIKQMW |
| 516 | DLGPISSWM |
| 517 | DLHRFDADY |
| 518 | DLIVKKSTG |
| 519 | DLIWNHLRT |
| 520 | DLLGIINGG |
| 521 | DLQYRLCQQ |
| 522 | DLYGQPNYC |
| 523 | DLYIMSDAN |
| 524 | DMCEQNNMQ |
| 525 | DMCVQNNGQ |
| 526 | DMEGCCGCA |
| 527 | DMHMKDMDS |
| 528 | DMHRFDADY |
| 529 | DMLPNNEWP |
| 530 | DMTEWNFLF |
| 531 | DMVFNNEWP |
| 532 | DMVMPEMYG |
| 533 | DNADLHHEC |
| 534 | DNCSDGWKV |
| 535 | DNKCYHMLM |
| 536 | DNKCYSMAM |
| 537 | DNMYPECMQ |
| 538 | DNRMWYMCG |
| 539 | DNRMWYMYG |
| 540 | DNVHPDSQA |
| 541 | DNVHRDCQA |
| 542 | DNVQISDAE |
| 543 | DPEFMAHCE |
| 544 | DPEFMTHCE |
| 545 | DPHQLPKKS |
| 546 | DQGSFNMCR |
| 547 | DQLICNMNE |
| 548 | DQLNPGVQA |
| 549 | DQLPVQYSL |
| 550 | DQMFHQFVT |
| 551 | DQMPYETNA |
| 552 | DQNINIHTS |
| 553 | DQQKLECYH |
| 554 | DQQSIGDHE |
| 555 | DQSLVDSQY |
| 556 | DQTMESGKT |
| 557 | DQTMFCAWC |
| 558 | DRAQESMGP |
| 559 | DRCWMPYRN |
| 560 | DREIYAHWD |
| 561 | DRENAMFFG |
| 562 | DRMQKNSAE |
| 563 | DRMWIDIAS |
| 564 | DRNVCNVCH |
| 565 | DRSGNWLWA |
| 566 | DRSQSWLWA |
| 567 | DRYYMDALM |
| 568 | DSAFYDRDN |
| 569 | DSASPIMGM |
| 570 | DSDCYTLDC |
| 571 | DSDIQFGIG |
| 572 | DSDYFRQMD |
| 573 | DSEETDYYQ |
| 574 | DSFFYNFGA |
| 575 | DSFILEAMC |
| 576 | DSGQNQQAG |
| 577 | DSHINWFCD |
| 578 | DSHLSFECR |
| 579 | DSHTWVADR |
| 580 | DSKHQMCID |
| 581 | DSKPMYHSD |
| 582 | DSKPSVCQE |
| 583 | DSKPWYHVD |
| 584 | DSMCEHHYY |
| 585 | DSNLDMHGC |
| 586 | DSPEQMIRH |
| 587 | DSQCVNHEE |
| 588 | DSQFIGQGE |
| 589 | DSQHVCGVN |
| 590 | DSQPASEVG |
| 591 | DSRFKSEWN |
| 592 | DSRPESTVG |
| 593 | DSRPFYGSV |
| 594 | DSRPKSTNG |
| 595 | DSSPFFWHS |
| 596 | DSSYFHCCD |
| 597 | DSVNSMQCT |
| 598 | DSYCCVSTH |
| 599 | DSYTTWLDD |
| 600 | DTCCFVANP |
| 601 | DTCNSMQCT |
| 602 | DTCVMYTQD |
| 603 | DTDYNHNSA |
| 604 | DTFTGIMHQ |
| 605 | DTGKPITTA |
| 606 | DTHHTHWQY |
| 607 | DTHIFIRHA |
| 608 | DTHKYTENR |
| 609 | DTHLQMQVI |
| 610 | DTITFKQQH |
| 611 | DTMNHRMTP |
| 612 | DTMPTFSTM |
| 613 | DTMQQVKFN |
| 614 | DTPLCNILC |
| 615 | DTQQYWMQD |
| 616 | DTRCWNHNA |
| 617 | DTRCWNVNC |
| 618 | DTRDCNFFN |
| 619 | DTTDCKKAS |
| 620 | DVAELKSLF |
| 621 | DVAHMQWRG |
| 622 | DVAKFMQML |
| 623 | DVALKNMFS |
| 624 | DVAVKKEYE |
| 625 | DVCAFRLMN |
| 626 | DVCCWTLAP |
| 627 | DVCDEHILY |
| 628 | DVCERMGFT |
| 629 | DVCNFMYSG |
| 630 | DVCRKNVSD |
| 631 | DVDFNELCE |
| 632 | DVDHPCMMF |
| 633 | DVDLNELCP |
| 634 | DVFSWHKDQ |
| 635 | DVHDSQLCY |
| 636 | DVIHTTHSG |
| 637 | DVKMKLSEQ |
| 638 | DVLIATMTC |
| 639 | DVLISTMAC |
| 640 | DVNKHSFNG |
| 641 | DVVKPTLMQ |
| 642 | DVVTPKLMQ |
| 643 | DVWLMRMCA |
| 644 | DVYFIRNAE |
| 645 | DWAEKVWGF |
| 646 | DWCEMKVGD |
| 647 | DWFMAFCGH |
| 648 | DWFQAIIHA |
| 649 | DWGFWMKHE |
| 650 | DWKFNLKDE |
| 651 | DWKPSVCQC |
| 652 | DWMLHADLL |
| 653 | DWRTFQAIA |
| 654 | DWRTPQAIA |
| 655 | DWVPEGHFP |
| 656 | DYAIENSAF |
| 657 | DYDHLDTWT |
| 658 | DYDSIQRQE |
| 659 | DYHPWWVTS |
| 660 | DYILPVIAI |
| 661 | DYILPVTAM |
| 662 | DYLSQFFGI |
| 663 | DYLWMNQEK |
| 664 | DYNRNNRYL |
| 665 | DYQPLSCYP |
| 666 | DYQTMTEHE |
| 667 | DYRDSHVAG |
| 668 | DYSPIDCEQ |
| 669 | DYSYRHIGE |
| 670 | DYTGMHQNM |
| 671 | DYTMMHQNC |
| 672 | DYTTNCMVE |
| 673 | DYVSLINEI |
| 674 | DYVYTNAEA |
| 675 | DYWLMHKID |
| 676 | EACFKDFWF |
| 677 | EACGMMTYC |
| 678 | EACGMTTMC |
| 679 | EACHPDLTP |
| 680 | EACMVNAAF |
| 681 | EACYWGHCP |
| 682 | EADEWWTIL |
| 683 | EADSRANAQ |
| 684 | EAEHMNWLM |
| 685 | EAEPFKTIQ |
| 686 | EAFNAFAGE |
| 687 | EAFNAFATR |
| 688 | EAFVHYCIN |
| 689 | EAGSCAMRV |
| 690 | EAHDDEYQC |
| 691 | EAHTNSICK |
| 692 | EAIPHIMDM |
| 693 | EAITDMYQP |
| 694 | EAITQFAVI |
| 695 | EAKWKECPS |
| 696 | EAMEQPYMF |
| 697 | EAMGYQAVE |
| 698 | EAMPVQKGW |
| 699 | EANVREHFC |
| 700 | EARTTRDCG |
| 701 | EARYSEEVW |
| 702 | EASMPYASM |
| 703 | EASWKMCPS |
| 704 | EATWLQRIM |
| 705 | EAVMWTCNS |
| 706 | EAWFYHADD |
| 707 | EAWMPSASG |
| 708 | EAWMYHQFH |
| 709 | EAWSKLEQP |
| 710 | EAWSMLDAL |
| 711 | EAWWYNFYF |
| 712 | EAYLYNIPM |
| 713 | ECDCGHVHL |
| 714 | ECHPYKTVA |
| 715 | ECLARQRHV |
| 716 | ECTQYVMCN |
| 717 | ECTWNMDAW |
| 718 | ECVSHIPGK |
| 719 | ECWSYQMVD |
| 720 | EDEQHLWND |
| 721 | EEDPLNGWQ |
| 722 | EEEPIWWQV |
| 723 | EEEWIWWQC |
| 724 | EEEWLNGWQ |
| 725 | EELETWIPY |
| 726 | EEMADGDAQ |
| 727 | EEMADGDAR |
| 728 | EEMEGLASE |
| 729 | EEPWQCMTA |
| 730 | EFDTTPCLF |
| 731 | EFEHGNAAS |
| 732 | EFFMVANSP |
| 733 | EFHMMGHFF |
| 734 | EFHYWFRPS |
| 735 | EFISLKSYE |
| 736 | EFLCINHGE |
| 737 | EFLVSGDSG |
| 738 | EFNALSFNV |
| 739 | EFNKLSFNH |
| 740 | EFNNTMMGQ |
| 741 | EFRHLTSMP |
| 742 | EFRHNQMNT |
| 743 | EFSPCVKMR |
| 744 | EFSYNVVHF |
| 745 | EFTKYSNAV |
| 746 | EFTMNQHMW |
| 747 | EFTTNYCHG |
| 748 | EFWVTECDQ |
| 749 | EGCQIPWTA |
| 750 | EGDPFKLQK |
| 751 | EGKQTNKAV |
| 752 | EGLQGNNYY |
| 753 | EGVQLNTDE |
| 754 | EGYMCMSGH |
| 755 | EGYSTHALG |
| 756 | EHAWLWMQS |
| 757 | EHCWYQFGA |
| 758 | EHFKAGCLH |
| 759 | EHGYMRCTK |
| 760 | EHHVTTGCT |
| 761 | EHKHMGCMN |
| 762 | EHKPCHASD |
| 763 | EHMQAKSAW |
| 764 | EHNHMGCMN |
| 765 | EHNWVQRAE |
| 766 | EHQLVGDWF |
| 767 | EHQLVGDWL |
| 768 | EHSWFISYN |
| 769 | EHSWINNMN |
| 770 | EHTFGPVYR |
| 771 | EHTQRGAAP |
| 772 | EHTQRGCAG |
| 773 | EHVQPMQWN |
| 774 | EHWHQMSPS |
| 775 | EHYSAHFFC |
| 776 | EIASKMNQL |
| 777 | EIDSKMHQL |
| 778 | EIGPQPNQD |
| 779 | EIGSVKHWE |
| 780 | EIHKRIAMT |
| 781 | EIKFSAGGI |
| 782 | EILKIDYQV |
| 783 | EINSLMYVD |
| 784 | EISCMHMSM |
| 785 | EISFEYCHE |
| 786 | EISPQENQD |
| 787 | EITEEALCD |
| 788 | EITHYYSTD |
| 789 | EIWSALAAN |
| 790 | EIWVREVNS |
| 791 | EKAHVQYAY |
| 792 | EKDTPTCKM |
| 793 | EKEMISTLE |
| 794 | EKIMVNRAF |
| 795 | EKLNAVQFC |
| 796 | EKMNCLVRW |
| 797 | EKNLYWTWE |
| 798 | EKRINTSPG |
| 799 | EKYRFSNGY |
| 800 | ELCTNTMAS |
| 801 | ELGTMTHSE |
| 802 | ELMSMIIVE |
| 803 | ELQEATHYP |
| 804 | ELRDYHIGM |
| 805 | ELRHAEQSQ |
| 806 | ELRISEQSQ |
| 807 | ELWDYHIGM |
| 808 | ELYMSNWMM |
| 809 | ELYSLAEQI |
| 810 | EMAKYMWDP |
| 811 | EMAMNLKDT |
| 812 | EMAVANSTH |
| 813 | EMCKGLCTT |
| 814 | EMDWTHVSH |
| 815 | EMESLNLLQ |
| 816 | EMESRNLLQ |
| 817 | EMFFYFGSQ |
| 818 | EMHKVPSGM |
| 819 | EMIHIGHRE |
| 820 | EMKTQCHGE |
| 821 | EMKTQTHFE |
| 822 | EMKTTQDAE |
| 823 | EMLPTQLNC |
| 824 | EMLVAHFGN |
| 825 | EMMCRCWLM |
| 826 | EMMQQAFAQ |
| 827 | EMNRYWTWE |
| 828 | EMSEEFSHA |
| 829 | EMYAFWQRG |
| 830 | EMYMMQTDQ |
| 831 | EMYPLFDAI |
| 832 | EMYVTKADD |
| 833 | ENAQMECLM |
| 834 | ENCAVTFCA |
| 835 | ENDQANLYA |
| 836 | ENDRCHTCA |
| 837 | ENDYVHGCM |
| 838 | ENFIQNCPG |
| 839 | ENGPLLCAR |
| 840 | ENKPVDQDK |
| 841 | ENLLEWKKA |
| 842 | ENNTWMQWG |
| 843 | ENQPANGWY |
| 844 | ENQQGNQCV |
| 845 | ENRLVQEVQ |
| 846 | ENRMFQDSE |
| 847 | ENSFYAGAG |
| 848 | ENTTTVAPC |
| 849 | ENVHINQAM |
| 850 | EPVSLQYMD |
| 851 | EPWKTHQQN |
| 852 | EQAEYDSCD |
| 853 | EQAGVASMW |
| 854 | EQCAVWDCM |
| 855 | EQFVSMNTG |
| 856 | EQGFVMFNG |
| 857 | EQGPLGSWF |
| 858 | EQGQLWSWF |
| 859 | EQNEETPFG |
| 860 | EQRNMGERA |
| 861 | EQRYCNSYV |
| 862 | EQTGVNVCL |
| 863 | ERELASATH |
| 864 | ERHPINQAL |
| 865 | ERQAHNNYA |
| 866 | ERTVNLRDC |
| 867 | ESCKTIRLE |
| 868 | ESCLSQNRA |
| 869 | ESEPFECCA |
| 870 | ESESGHMEG |
| 871 | ESGMIAVPH |
| 872 | ESHLWLAME |
| 873 | ESHSTQTYV |
| 874 | ESHYRCMCN |
| 875 | ESISSGVEG |
| 876 | ESLSFQTLV |
| 877 | ESMPFLDEA |
| 878 | ESNPVSMDV |
| 879 | ESTHMLYEM |
| 880 | ESTPQFTSG |
| 881 | ESTQRFDKM |
| 882 | ESTSGWSMA |
| 883 | ESTSNWAMA |
| 884 | ESVLKNQFE |
| 885 | ESWMKLEWQ |
| 886 | ETAIRDLTQ |
| 887 | ETCLSMHYL |
| 888 | ETDVSWCES |
| 889 | ETEFRQLDG |
| 890 | ETEVKSHWD |
| 891 | ETFIFPGAG |
| 892 | ETFVRHADY |
| 893 | ETGKTIYHQ |
| 894 | ETGPRQDLF |
| 895 | ETHFVQGRM |
| 896 | ETHILDQFC |
| 897 | ETHIVFGQW |
| 898 | ETHNKHTDY |
| 899 | ETISSGIEG |
| 900 | ETLAKLHCA |
| 901 | ETLSRSDIC |
| 902 | ETNEVMVQQ |
| 903 | ETRVSWCEW |
| 904 | ETSLSGNQY |
| 905 | ETSTSCAWL |
| 906 | ETTHYYSTF |
| 907 | ETTLHDLWL |
| 908 | ETTLQDLWE |
| 909 | ETVICDGVT |
| 910 | EVCSIHWMG |
| 911 | EVEVLEMGH |
| 912 | EVEVMVYSW |
| 913 | EVEWYYGQS |
| 914 | EVFEEYWSL |
| 915 | EVHGRNQDN |
| 916 | EVHGWNQDN |
| 917 | EVHKGAVMD |
| 918 | EVHKYCCNS |
| 919 | EVHSTQTAV |
| 920 | EVIRSPIEG |
| 921 | EVKPIGHSQ |
| 922 | EVLLSTDEA |
| 923 | EVLVAEWQG |
| 924 | EVLVAFATN |
| 925 | EVLVAVWQG |
| 926 | EVMPYHRTH |
| 927 | EVNQQMKAK |
| 928 | EVRQWQDED |
| 929 | EVSFYAGAI |
| 930 | EVSRQMYSQ |
| 931 | EVTDPCECA |
| 932 | EVTTIAQDH |
| 933 | EVVRRCYEC |
| 934 | EWCLLHVGH |
| 935 | EWEQTWRAP |
| 936 | EWFQAGICD |
| 937 | EWKHDRNDI |
| 938 | EWKHYRNDI |
| 939 | EWQGLQQFT |
| 940 | EWSGLQSCF |
| 941 | EWVFQHLSS |
| 942 | EWYCFLDSG |
| 943 | EYACHNRMP |
| 944 | EYCEFRQMD |
| 945 | EYCKYSLGL |
| 946 | EYEHMMWSQ |
| 947 | EYKSYGNCQ |
| 948 | EYLWEAIEH |
| 949 | EYMQQAFAW |
| 950 | EYNIYIATD |
| 951 | EYQISKTMC |
| 952 | EYQISNTMC |
| 953 | EYSTLPMTT |
| 954 | EYTKYQLGL |
| 955 | EYVSLINEI |
| 956 | EYWLNSSQA |
| 957 | FACWILYIG |
| 958 | FAEIHMQYH |
| 959 | FAEPLENWM |
| 960 | FAGFALHDS |
| 961 | FAGMYRRNS |
| 962 | FAILSGQTL |
| 963 | FAIWNYESK |
| 964 | FALSRNGNG |
| 965 | FALTCEEAP |
| 966 | FALTCNEAG |
| 967 | FAMKVLDIQ |
| 968 | FAMLGYWVH |
| 969 | FANCSTHQD |
| 970 | FAQLNQASM |
| 971 | FASRQGDNH |
| 972 | FASTTAECM |
| 973 | FATAHQKCW |
| 974 | FATQNQSPS |
| 975 | FAVMTSTEN |
| 976 | FCAGCDMAN |
| 977 | FCCHLAAHK |
| 978 | FCCSKANDC |
| 979 | FCDVVKHHL |
| 980 | FCDYHNTIE |
| 981 | FCITGNACA |
| 982 | FCNSYTMLP |
| 983 | FCPNSQMDG |
| 984 | FCQNPTQAM |
| 985 | FCVNQQTCQ |
| 986 | FDALMCWKI |
| 987 | FDANDCRPS |
| 988 | FDKCHMADW |
| 989 | FEACVTECL |
| 990 | FEKTMVAMH |
| 991 | FFGNVMKEQ |
| 992 | FFGWFHMNN |
| 993 | FFHVAWLSI |
| 994 | FFLKLNDVP |
| 995 | FFNHDNIGN |
| 996 | FFNNISTYF |
| 997 | FFNPTTQQN |
| 998 | FFPWEWECW |
| 999 | FFSHIQAAD |
| 1000 | FFSMVENCG |
| 1001 | FFTNRNHEE |
| 1002 | FFVKMMCSH |
| 1003 | FFVTWICRL |
| 1004 | FFYFLDILK |
| 1005 | FFYSTNFGM |
| 1006 | FGAEMHCPM |
| 1007 | FGDFFFNHN |
| 1008 | FGKWLTRLH |
| 1009 | FGNSMMLQS |
| 1010 | FHAMKHVLE |
| 1011 | FHLHTHRED |
| 1012 | FHNSKSEKH |
| 1013 | FHSFIHQHE |
| 1014 | FHTNYICQE |
| 1015 | FHVKVSCDS |
| 1016 | FICHVKEGA |
| 1017 | FICRHIHGI |
| 1018 | FICSSQCGA |
| 1019 | FIEFTIGEF |
| 1020 | FIGLKNAHL |
| 1021 | FIPNKWLGN |
| 1022 | FIQMTMSSD |
| 1023 | FITQYPNAE |
| 1024 | FKFMVDTSS |
| 1025 | FKIECGLFQ |
| 1026 | FKNDSVDHE |
| 1027 | FLEYAVQNQ |
| 1028 | FLQWTESGD |
| 1029 | FLVGQHVHP |
| 1030 | FLVVNHFGH |
| 1031 | FMECLKDTK |
| 1032 | FMEDKQVFS |
| 1033 | FMEYYLWYC |
| 1034 | FMSLVNAGE |
| 1035 | FMTMLGCSM |
| 1036 | FNARRDTWF |
| 1037 | FNGYYETQG |
| 1038 | FNQFEFVSA |
| 1039 | FNRCMEDRG |
| 1040 | FNSNLGAAT |
| 1041 | FNSSYGTNQ |
| 1042 | FNTQLLMSG |
| 1043 | FPEKAIVGQ |
| 1044 | FPEPFPMWG |
| 1045 | FPMPFPMWK |
| 1046 | FQAMIMTNY |
| 1047 | FQAVCIWIS |
| 1048 | FQCSVHKFV |
| 1049 | FQECEAPQH |
| 1050 | FQEVFSFFN |
| 1051 | FQFWRQIME |
| 1052 | FQIPLKAVA |
| 1053 | FQSMQHDNN |
| 1054 | FSADSMCSF |
| 1055 | FSAPITAHF |
| 1056 | FSAPLFHCA |
| 1057 | FSEPLFHLF |
| 1058 | FSEVKGMSG |
| 1059 | FSFRTARQQ |
| 1060 | FSFRTARYL |
| 1061 | FSGTVYCWC |
| 1062 | FSLPNNCDD |
| 1063 | FSNTVYKWC |
| 1064 | FSTHLTDAC |
| 1065 | FSTSYCHED |
| 1066 | FTDNPNWYA |
| 1067 | FTERTKCAA |
| 1068 | FTEYEHCAS |
| 1069 | FTHQRNALT |
| 1070 | FTHTFEACG |
| 1071 | FTIIKNNCL |
| 1072 | FTMDINLND |
| 1073 | FTMQSGDYH |
| 1074 | FTNFKMNMY |
| 1075 | FTNQLPEAN |
| 1076 | FTNYKYSFE |
| 1077 | FTQQEFHGQ |
| 1078 | FTTDYQMIA |
| 1079 | FTVDQLNWQ |
| 1080 | FTWHIESKF |
| 1081 | FTYGFQTIN |
| 1082 | FTYNFMNSW |
| 1083 | FVDEAFAWQ |
| 1084 | FVGFALHDS |
| 1085 | FVHASMINA |
| 1086 | FVIGHHLFP |
| 1087 | FVRGPWQCE |
| 1088 | FVRWSPYPI |
| 1089 | FVSAMSGPC |
| 1090 | FVSKSEEYH |
| 1091 | FVSLMSGPN |
| 1092 | FVTVMQFQD |
| 1093 | FVTVTACDG |
| 1094 | FVWMTGLNM |
| 1095 | FWELNGPKN |
| 1096 | FWGQYHVQG |
| 1097 | FWHEYNFTN |
| 1098 | FWKMLGHNH |
| 1099 | FWMQTEHGK |
| 1100 | FWMQTEHGT |
| 1101 | FWNFFHSHL |
| 1102 | FWNMVDTGG |
| 1103 | FWNQQSQCF |
| 1104 | FWQQYNEAN |
| 1105 | FWRSLDMQY |
| 1106 | FWSNIGAAP |
| 1107 | FWSQYDLMY |
| 1108 | FWTSYDSSH |
| 1109 | FYCSKMRGL |
| 1110 | FYYNYMHHS |
| 1111 | GAAQFGAFY |
| 1112 | GAFIIQKTP |
| 1113 | GAFMGLMTQ |
| 1114 | GAFVGLMTQ |
| 1115 | GAHMHDTHL |
| 1116 | GAKTYGEME |
| 1117 | GASFYEGME |
| 1118 | GATECFNPC |
| 1119 | GATQCFNPF |
| 1120 | GCDKWLGLC |
| 1121 | GCELISVGQ |
| 1122 | GCTPRQEMP |
| 1123 | GDMGCNVKL |
| 1124 | GEEVGCIWG |
| 1125 | GEKTYGEMY |
| 1126 | GEQQMPFIC |
| 1127 | GEYNERMHI |
| 1128 | GFANFMMNF |
| 1129 | GFANFNMTD |
| 1130 | GFDNVSFSY |
| 1131 | GFEEMDAQM |
| 1132 | GFEEQLTSK |
| 1133 | GFEMSTSHS |
| 1134 | GFFLTKQDG |
| 1135 | GFFTQMSQS |
| 1136 | GFGTYLSMH |
| 1137 | GFIDYQMQD |
| 1138 | GFMHYCSHV |
| 1139 | GFMLPIYWC |
| 1140 | GFTFMEASH |
| 1141 | GGAGVHHHT |
| 1142 | GGQTIKEHF |
| 1143 | GGWSDSNDA |
| 1144 | GGYNERMHI |
| 1145 | GHCYTQQQH |
| 1146 | GHDFTQFQE |
| 1147 | GHEDLLAMD |
| 1148 | GHESCGLWT |
| 1149 | GHLECCWSI |
| 1150 | GHSFRYACD |
| 1151 | GHVQAECMY |
| 1152 | GIADYQQQG |
| 1153 | GIFFCQEEK |
| 1154 | GIGWLKHFE |
| 1155 | GIICVTRDD |
| 1156 | GIKCERDAD |
| 1157 | GIKLTGTDH |
| 1158 | GILELHFGV |
| 1159 | GILHLHQCI |
| 1160 | GILNITMDQ |
| 1161 | GIMCREQCD |
| 1162 | GIMKHQETS |
| 1163 | GIMMQQMDP |
| 1164 | GIQQHAIMA |
| 1165 | GIREQEYCP |
| 1166 | GIRWADHWN |
| 1167 | GIVMMQAMD |
| 1168 | GIVNCTRLG |
| 1169 | GIVPLGTNY |
| 1170 | GIYMNQENA |
| 1171 | GIYPWMTYA |
| 1172 | GKQFDEQLE |
| 1173 | GKQFDEQQM |
| 1174 | GKSPMGEMP |
| 1175 | GLFWMWVDN |
| 1176 | GLIFHQSLS |
| 1177 | GLMSASAHS |
| 1178 | GLMTQQNAD |
| 1179 | GLMTQQNSD |
| 1180 | GLNICKNQQ |
| 1181 | GLNKFMQGP |
| 1182 | GLSCKSAWL |
| 1183 | GLYWQCDGM |
| 1184 | GMEHLWNVD |
| 1185 | GMKFTAQGN |
| 1186 | GMKILRTRK |
| 1187 | GMNFCLASH |
| 1188 | GMRDVFDCY |
| 1189 | GMRYHDDLF |
| 1190 | GMSFYNFFE |
| 1191 | GMSSYTCED |
| 1192 | GMTPRMMGM |
| 1193 | GMTSWGCWE |
| 1194 | GNEGADAEE |
| 1195 | GNEKCGTAV |
| 1196 | GNKSFQYAS |
| 1197 | GNMEQEGCP |
| 1198 | GNYLYMHQG |
| 1199 | GPFSFLRGA |
| 1200 | GPFSFLRGG |
| 1201 | GPNGDAARY |
| 1202 | GQAVSHAFS |
| 1203 | GQFWTNDAA |
| 1204 | GQGVAQQCA |
| 1205 | GQHFVMLSS |
| 1206 | GQHLQIGAE |
| 1207 | GQNTTDRSN |
| 1208 | GQPVCCVCM |
| 1209 | GQQHYGWQQ |
| 1210 | GQSFYTCED |
| 1211 | GQVHTSYYG |
| 1212 | GQVQHECMY |
| 1213 | GRCFMPPGR |
| 1214 | GRVLANCAE |
| 1215 | GSDAIMWDC |
| 1216 | GSDAQMWDH |
| 1217 | GSEHKGIGE |
| 1218 | GSEWRQDMG |
| 1219 | GSFMSCEKN |
| 1220 | GSFQQMASL |
| 1221 | GSFWHNDAA |
| 1222 | GSHNTGAWP |
| 1223 | GSMHNSICY |
| 1224 | GSNDRLDSC |
| 1225 | GSTSMITHA |
| 1226 | GTADKPTGI |
| 1227 | GTASLDVLS |
| 1228 | GTCSMTLDN |
| 1229 | GTESLAVLS |
| 1230 | GTGFWWGCY |
| 1231 | GTIHMYCGW |
| 1232 | GTLEHTSST |
| 1233 | GTMPFWAYD |
| 1234 | GTMTCHYHH |
| 1235 | GTPQGKQRQ |
| 1236 | GTPWKEQFK |
| 1237 | GTQFAQGDN |
| 1238 | GTQVRHHDY |
| 1239 | GTQWQVSHW |
| 1240 | GTQWTQWMK |
| 1241 | GTSWELWNA |
| 1242 | GTVLKMNDI |
| 1243 | GTVQAIVCT |
| 1244 | GTWINEQMG |
| 1245 | GVADKPTGY |
| 1246 | GVEGHGQFD |
| 1247 | GVFYVGHWQ |
| 1248 | GVINMLASD |
| 1249 | GVMSCKVIG |
| 1250 | GVMSMSFPV |
| 1251 | GVQHYQLGM |
| 1252 | GVQHYWLGT |
| 1253 | GVQYGSQHF |
| 1254 | GVTTSNYCP |
| 1255 | GVTVCWSNQ |
| 1256 | GVTYHHRQC |
| 1257 | GVVATQRSG |
| 1258 | GVYDTLSSF |
| 1259 | GVYQQFHWF |
| 1260 | GWDWIMCNK |
| 1261 | GWFTYDKQQ |
| 1262 | GWHILPVME |
| 1263 | GWIFITGAD |
| 1264 | GWKHICVHG |
| 1265 | GWNNRDSGP |
| 1266 | GWNQHHSSE |
| 1267 | GWVQSMCMS |
| 1268 | GWYSLAVAN |
| 1269 | GYCMRLFGI |
| 1270 | GYCPCFHAG |
| 1271 | GYERADCWM |
| 1272 | GYESFLRDQ |
| 1273 | GYIPTQSMY |
| 1274 | GYISYGTQE |
| 1275 | GYKMMDCSM |
| 1276 | GYLSFMHNN |
| 1277 | GYMQTLEGL |
| 1278 | GYQNFLDYF |
| 1279 | GYTSMITHV |
| 1280 | GYVQQSKGE |
| 1281 | GYVWLGAMP |
| 1282 | HAALYTNQN |
| 1283 | HAAPGMIHS |
| 1284 | HACFNHFPC |
| 1285 | HACLMYWYD |
| 1286 | HAEELGDCY |
| 1287 | HAEIQGHQE |
| 1288 | HAELLGAFE |
| 1289 | HAHVFIWYA |
| 1290 | HAHVFIWYT |
| 1291 | HAILLDEMR |
| 1292 | HAISIWQTN |
| 1293 | HAKLFPTMI |
| 1294 | HASAMECIS |
| 1295 | HASGFMMQS |
| 1296 | HASTAIMCP |
| 1297 | HATPVCKEY |
| 1298 | HATTCDFDF |
| 1299 | HAYPILQMD |
| 1300 | HCAMTQRFD |
| 1301 | HCAVCQWQA |
| 1302 | HCETFMMQF |
| 1303 | HCNFDIPGA |
| 1304 | HCQPFTGHV |
| 1305 | HCSLYAQER |
| 1306 | HCSNVEFYQ |
| 1307 | HCSPNNCLE |
| 1308 | HCSPNNCPE |
| 1309 | HCVEFMQSN |
| 1310 | HDDAQLDNS |
| 1311 | HDEAQLDNS |
| 1312 | HDFDNPSMY |
| 1313 | HDGPSAKPN |
| 1314 | HDHTPLQSP |
| 1315 | HDNSAMWMV |
| 1316 | HDTMEWGCH |
| 1317 | HDTMEWGFH |
| 1318 | HECWQMTND |
| 1319 | HESMAWTLL |
| 1320 | HFCQYQSST |
| 1321 | HFDNYQRFC |
| 1322 | HFFHNMDYH |
| 1323 | HFGPTNCSM |
| 1324 | HFHQNFHFF |
| 1325 | HFMEMLING |
| 1326 | HFWMMECSG |
| 1327 | HFWVINGYE |
| 1328 | HGCPHFASG |
| 1329 | HGLTIGAAT |
| 1330 | HGSLLSTCH |
| 1331 | HHDSCYVVP |
| 1332 | HHESHLCGY |
| 1333 | HHQHVDNHG |
| 1334 | HHQWLRVME |
| 1335 | HHWPQTPLL |
| 1336 | HIANNFDWE |
| 1337 | HIAQCDVYL |
| 1338 | HIDDWAHGR |
| 1339 | HIDLMQMEY |
| 1340 | HIENKYAHI |
| 1341 | HIEVCTLGD |
| 1342 | HIFMDEMAY |
| 1343 | HIGYKDSLS |
| 1344 | HILIMQGVS |
| 1345 | HIQEELEEQ |
| 1346 | HISEFNRCT |
| 1347 | HISFCSEYW |
| 1348 | HIVQCDVIL |
| 1349 | HIYFQEAQS |
| 1350 | HKGTHAFVM |
| 1351 | HKYCRLSEE |
| 1352 | HLAPYCQQE |
| 1353 | HLDDIISFN |
| 1354 | HLGMWLAPV |
| 1355 | HLQVCNIYH |
| 1356 | HLSFNNWCR |
| 1357 | HMATLAHEA |
| 1358 | HMVIHAIPD |
| 1359 | HMVTLAHGA |
| 1360 | HNASNHECT |
| 1361 | HNEDLGDCY |
| 1362 | HNENTHWFN |
| 1363 | HNGCLMDVA |
| 1364 | HPHDTMKLT |
| 1365 | HPVCCLEQA |
| 1366 | HPYTAKWDV |
| 1367 | HQEVNPTPA |
| 1368 | HQEYFDANQ |
| 1369 | HQISVNNTL |
| 1370 | HQMEYSDFL |
| 1371 | HQMEYSDLL |
| 1372 | HQVQKYRMC |
| 1373 | HRDYFFTYD |
| 1374 | HSASGICCN |
| 1375 | HSCGTYYWA |
| 1376 | HSEMSNILM |
| 1377 | HSFEKKIPL |
| 1378 | HSVVVYEHA |
| 1379 | HSWINNQEH |
| 1380 | HTAQTYSTN |
| 1381 | HTEAHLSAC |
| 1382 | HTECDIQQK |
| 1383 | HTECKTIMH |
| 1384 | HTGCLLDVA |
| 1385 | HTIEGKHMN |
| 1386 | HTIHYGEAS |
| 1387 | HTINMPRWV |
| 1388 | HTMDYNTCR |
| 1389 | HTNQMWGMF |
| 1390 | HTNSYMEIE |
| 1391 | HTNYMQKEY |
| 1392 | HTQLKNCTG |
| 1393 | HTSFTNCEC |
| 1394 | HTSHQGHAE |
| 1395 | HTSLDSWFN |
| 1396 | HTTLCDPCG |
| 1397 | HTTNYGEAS |
| 1398 | HVAVNGFYD |
| 1399 | HVDGIWRDQ |
| 1400 | HVDQSLKYG |
| 1401 | HVELNMKGV |
| 1402 | HVEVPNTDA |
| 1403 | HVFQEGCCI |
| 1404 | HVGTDCMAA |
| 1405 | HVHQALTIS |
| 1406 | HVKVPNTDA |
| 1407 | HVLNGTGGS |
| 1408 | HVMPYMWVG |
| 1409 | HVMQYMWPG |
| 1410 | HVMRYYEPY |
| 1411 | HVNTYQLRG |
| 1412 | HVSAMECIS |
| 1413 | HVSPIQDNM |
| 1414 | HWLSCEKRW |
| 1415 | HWTLCDPEG |
| 1416 | HWVSTLADY |
| 1417 | HYAEITYRQ |
| 1418 | HYCPCHNKY |
| 1419 | HYDLSTCAL |
| 1420 | HYEWADHQW |
| 1421 | HYMQRKEWR |
| 1422 | HYTEVQKTP |
| 1423 | HYVALDAGS |
| 1424 | HYVMFQMSN |
| 1425 | HYVSMMLDE |
| 1426 | HYVVRHEDP |
| 1427 | IAAQPITNE |
| 1428 | IACYEPVGS |
| 1429 | IAEFLQCDY |
| 1430 | IAHNNFEPT |
| 1431 | IAHVVEQMG |
| 1432 | IALSATKDK |
| 1433 | IANVNNGTH |
| 1434 | IATKSGLYH |
| 1435 | IATLHMLSN |
| 1436 | IATWMWKYS |
| 1437 | IAYHTKSTI |
| 1438 | ICACFNKAY |
| 1439 | ICGFFCQQQ |
| 1440 | ICIQSNGRE |
| 1441 | ICLLEAGEK |
| 1442 | ICMQNAMSW |
| 1443 | ICMWKQYCS |
| 1444 | ICQHNGVAY |
| 1445 | ICREQIKWM |
| 1446 | ICRYALLHA |
| 1447 | ICSHSWFQA |
| 1448 | IDAWGHFSE |
| 1449 | IDDFRIVMH |
| 1450 | IDEQCKFGH |
| 1451 | IECSPREYI |
| 1452 | IEDVWECPY |
| 1453 | IEQQGSPLL |
| 1454 | IEYCQGMNF |
| 1455 | IEYYPTAEQ |
| 1456 | IFAHSNCCE |
| 1457 | IFALFDRYN |
| 1458 | IFENHDAAM |
| 1459 | IFILWQCKE |
| 1460 | IFLSKIPCQ |
| 1461 | IFNTRFLRE |
| 1462 | IFSSGGADA |
| 1463 | IFSSGGAYE |
| 1464 | IFTAKNEEM |
| 1465 | IFVIKQQCI |
| 1466 | IFVIYHQSG |
| 1467 | IFYAGDCTF |
| 1468 | IGAEQKVGI |
| 1469 | IGCLAVNHI |
| 1470 | IGGIIGNWG |
| 1471 | IGLSRNSNG |
| 1472 | IGQRIAGYA |
| 1473 | IGVERDYYA |
| 1474 | IGYQIMDME |
| 1475 | IHCDIQCQF |
| 1476 | IHEFHIAAM |
| 1477 | IHNFYYAST |
| 1478 | IHNHYTACQ |
| 1479 | IHQSFQQWE |
| 1480 | IHSQCQQGQ |
| 1481 | IHSYKDAHL |
| 1482 | IHTQTLISE |
| 1483 | IIAQQMRHH |
| 1484 | IIGSCQSFY |
| 1485 | IIHLPDHCR |
| 1486 | IILQNMKSW |
| 1487 | IIMQNMMSW |
| 1488 | IIPQKCSNP |
| 1489 | IISECQFGG |
| 1490 | IITCMDGYF |
| 1491 | IITMDLCTD |
| 1492 | IIWLGSIRN |
| 1493 | IIYHRTNAS |
| 1494 | IIYQLDCAT |
| 1495 | IKDHYIMQE |
| 1496 | IKQVFNQIA |
| 1497 | IKTNDQALL |
| 1498 | IKVQIDQIE |
| 1499 | ILAKWEVCE |
| 1500 | ILEQFYKMG |
| 1501 | ILERHLCGF |
| 1502 | ILHLEHPSD |
| 1503 | ILPFFCCGL |
| 1504 | ILRCVCDYP |
| 1505 | ILSFYWGDY |
| 1506 | ILTKWEVCE |
| 1507 | IMASVDICA |
| 1508 | IMCYKMCEF |
| 1509 | IMDEIHYMM |
| 1510 | IMDLITYAQ |
| 1511 | IMDMIYDCG |
| 1512 | IMDSRLTVA |
| 1513 | IMETADCHM |
| 1514 | IMGLGMRAD |
| 1515 | IMGLRMRAD |
| 1516 | IMGPAMNYD |
| 1517 | IMMANQLSC |
| 1518 | IMTPLAGGH |
| 1519 | IMTPLASGH |
| 1520 | IMTTENAAP |
| 1521 | IMVERWQHA |
| 1522 | INCQTQKEM |
| 1523 | INDHAQIVK |
| 1524 | INEDAQEKM |
| 1525 | INGSRACSY |
| 1526 | INMVSMYAY |
| 1527 | IPALKEGQI |
| 1528 | IPAPIHVNA |
| 1529 | IPDLHDHMG |
| 1530 | IPGTMSHTA |
| 1531 | IPLAMALWT |
| 1532 | IPMGWEFPE |
| 1533 | IPTMQWWNE |
| 1534 | IPWMERSAS |
| 1535 | IQEHLNACE |
| 1536 | IQLGCSAFH |
| 1537 | IQQQMSMCG |
| 1538 | IRDMFEQYT |
| 1539 | IREPTEPYC |
| 1540 | IRLMTPDEN |
| 1541 | IRLPMNSAH |
| 1542 | IRSMSNIME |
| 1543 | IRTLFNITA |
| 1544 | IRTNPWFHE |
| 1545 | ISDCVVWTY |
| 1546 | ISDLGIMWI |
| 1547 | ISEEIVSFE |
| 1548 | ISEGQAESR |
| 1549 | ISELFGAAF |
| 1550 | ISELFGEAE |
| 1551 | ISFGMEAMS |
| 1552 | ISGEGHQAV |
| 1553 | ISGHAQVQP |
| 1554 | ISGNAWGSL |
| 1555 | ISGWRMPCD |
| 1556 | ISNLLCWCP |
| 1557 | ISNPLYQVS |
| 1558 | ISQEYVSLQ |
| 1559 | ISQHNTFCS |
| 1560 | ISQVDWKCH |
| 1561 | ISRSKDAGM |
| 1562 | ISSGCWQFD |
| 1563 | ISVGQWEFI |
| 1564 | ISVIQPQFA |
| 1565 | ISYFTMAAQ |
| 1566 | ISYSSFDCH |
| 1567 | ITCSNHAMM |
| 1568 | ITDLLNVWG |
| 1569 | ITDQLCHSE |
| 1570 | ITEHVNFVT |
| 1571 | ITEVRQECA |
| 1572 | ITHKSETMM |
| 1573 | ITHSMGDNM |
| 1574 | ITHVSGSQL |
| 1575 | ITIYELDSG |
| 1576 | ITLASKSMR |
| 1577 | ITMGPEKWG |
| 1578 | ITMQSHFQH |
| 1579 | ITNKTFNDN |
| 1580 | ITNPFFLDN |
| 1581 | ITQDANLPL |
| 1582 | ITTQQAGQT |
| 1583 | IVAVNGFWD |
| 1584 | IVCESIQVH |
| 1585 | IVCTIAGTQ |
| 1586 | IVDYYMEGG |
| 1587 | IVHFGSSWK |
| 1588 | IVHWLSWMP |
| 1589 | IVIWQGEWI |
| 1590 | IVKSEGQKA |
| 1591 | IVLPENHLG |
| 1592 | IVMKGQEIT |
| 1593 | IVMNSKQMS |
| 1594 | IVMSCFDAD |
| 1595 | IVNHVPGRG |
| 1596 | IVQAHMMMS |
| 1597 | IVQMDGASF |
| 1598 | IVQRDGASC |
| 1599 | IVSKEVEAD |
| 1600 | IVTGHLQEG |
| 1601 | IVYCQGSSF |
| 1602 | IWRDSLHAN |
| 1603 | IYEWMQCSH |
| 1604 | IYKWDNKFH |
| 1605 | IYLHKDAFG |
| 1606 | IYLKAQQDP |
| 1607 | IYMSSVCHC |
| 1608 | IYPLQSFCN |
| 1609 | IYSHFHQGQ |
| 1610 | IYTRMDGYF |
| 1611 | IYVDLVDSL |
| 1612 | IYWFHPKGG |
| 1613 | IYYQIAEHF |
| 1614 | KAANDHHVA |
| 1615 | KACPYYDEW |
| 1616 | KADTEHNTN |
| 1617 | KAIHNMYGP |
| 1618 | KAREDLWRE |
| 1619 | KATVEFEWP |
| 1620 | KAYSLESDG |
| 1621 | KCALYEYEV |
| 1622 | KCDVLYTCQ |
| 1623 | KCEAGMFCY |
| 1624 | KCEELKTME |
| 1625 | KCEGHHYVE |
| 1626 | KCESGIFCY |
| 1627 | KCLEGVNFT |
| 1628 | KCWITEDAN |
| 1629 | KDHDPGNWQ |
| 1630 | KEMDSITCF |
| 1631 | KENAFENAA |
| 1632 | KENAFENAS |
| 1633 | KFELSSMIQ |
| 1634 | KFGVTDTSE |
| 1635 | KFMTCDLHN |
| 1636 | KFMTSDLPN |
| 1637 | KFNEETPFG |
| 1638 | KGISNWSFY |
| 1639 | KGIWQGMDA |
| 1640 | KGKSNWSFY |
| 1641 | KGPCIMQGN |
| 1642 | KGPRIIQGN |
| 1643 | KHKWMVYGA |
| 1644 | KHKWMVYHG |
| 1645 | KHNEMIHWY |
| 1646 | KIDVYDCIA |
| 1647 | KIEDMMAQE |
| 1648 | KIHDCDTQY |
| 1649 | KIHDCQTFY |
| 1650 | KIHESQTFY |
| 1651 | KIIWRAVAC |
| 1652 | KKEAKYRGY |
| 1653 | KKIMVKRAF |
| 1654 | KLAPWQQNC |
| 1655 | KLEHNMLNP |
| 1656 | KLEHNMLYP |
| 1657 | KLELIMQPR |
| 1658 | KLHWMCSFL |
| 1659 | KMAFGSAGV |
| 1660 | KMECFMDAH |
| 1661 | KMEMYAKEW |
| 1662 | KMESYDYSD |
| 1663 | KMQLSEGVP |
| 1664 | KMVDLQHSL |
| 1665 | KMWIDKYFH |
| 1666 | KMYYFYCNS |
| 1667 | KNGNTDTSE |
| 1668 | KNLTGVAHH |
| 1669 | KNWNPWDSP |
| 1670 | KPDEVHNLN |
| 1671 | KPMEWQQHH |
| 1672 | KPTLEWAHE |
| 1673 | KQDAKERIW |
| 1674 | KQDTEVLLG |
| 1675 | KQDVSKWFK |
| 1676 | KQDVSKWQC |
| 1677 | KQLICDVHH |
| 1678 | KQNDHTHMA |
| 1679 | KQNIEQVLH |
| 1680 | KRQAHNHYA |
| 1681 | KSCCIDHQM |
| 1682 | KSEPGFKNH |
| 1683 | KSGIMENDE |
| 1684 | KSGYGELSE |
| 1685 | KSSSSYIWG |
| 1686 | KSTGCPRGG |
| 1687 | KSYPIFNEG |
| 1688 | KTDQCNEAT |
| 1689 | KTEQKLTPT |
| 1690 | KTFLDVQSV |
| 1691 | KTKYSNWRM |
| 1692 | KTLPWYRPH |
| 1693 | KTSHCEAMG |
| 1694 | KTTHHWMTE |
| 1695 | KTTPPWMTE |
| 1696 | KVCSIVADH |
| 1697 | KVDMLDIVD |
| 1698 | KVEDVVLNE |
| 1699 | KVEIKQMDQ |
| 1700 | KVGMDQTAV |
| 1701 | KVPKRAYFE |
| 1702 | KVSQQEGIV |
| 1703 | KVTTETGVP |
| 1704 | KWDMICGYD |
| 1705 | KWDNIMLMN |
| 1706 | KWDYQMQMN |
| 1707 | KWEWSKCYL |
| 1708 | KWMPFDNTF |
| 1709 | KWPVMANLH |
| 1710 | KWTELCNLY |
| 1711 | KWYSFSFRA |
| 1712 | KYDTVAHWE |
| 1713 | KYGESNQCQ |
| 1714 | KYWISQDCA |
| 1715 | LAATSAQCQ |
| 1716 | LACKGTNPT |
| 1717 | LACYKDGGV |
| 1718 | LADRCQCCY |
| 1719 | LAEFEDGGF |
| 1720 | LAGLDSNTA |
| 1721 | LAGPNHDHQ |
| 1722 | LAHTYGRDT |
| 1723 | LAHVSTQFN |
| 1724 | LAIGAEIAL |
| 1725 | LAIHEATYP |
| 1726 | LAIREATYH |
| 1727 | LAISLVDSY |
| 1728 | LALPYDDFS |
| 1729 | LAMYSQYRG |
| 1730 | LAPPYTRSN |
| 1731 | LASFWLNPQ |
| 1732 | LASPSNESH |
| 1733 | LATEHMSWD |
| 1734 | LATHCYGGR |
| 1735 | LATNTYAYS |
| 1736 | LATVQTQAP |
| 1737 | LAVSHANFF |
| 1738 | LAWIETRDG |
| 1739 | LAYLLGHKE |
| 1740 | LAYYSLAFG |
| 1741 | LCAPCGMSV |
| 1742 | LCAQELYMC |
| 1743 | LCAVCQLQA |
| 1744 | LCAVWDKMD |
| 1745 | LCELNSAWN |
| 1746 | LCERFHEDT |
| 1747 | LCFLMIMCD |
| 1748 | LCFLMSNCD |
| 1749 | LCGWAPCSW |
| 1750 | LCMYSQYRS |
| 1751 | LCTLVGMES |
| 1752 | LCTMEGPYR |
| 1753 | LCTNMMDMS |
| 1754 | LCVGLSNCP |
| 1755 | LDLAHSIPA |
| 1756 | LDTKTLANS |
| 1757 | LECQYHNFQ |
| 1758 | LEDRISATF |
| 1759 | LEGSSQFQD |
| 1760 | LEHSSGFMN |
| 1761 | LEVTNCNAA |
| 1762 | LEWFCWIGS |
| 1763 | LEWVCWSGS |
| 1764 | LFAKPDALL |
| 1765 | LFAVHMGYY |
| 1766 | LFCNIGKTF |
| 1767 | LFELTSVQN |
| 1768 | LFEMTYRNW |
| 1769 | LFEYYRRMA |
| 1770 | LFGIAIWYV |
| 1771 | LFHNAQDDR |
| 1772 | LFHYIQRTS |
| 1773 | LFIKVKDTS |
| 1774 | LFKHCAHHN |
| 1775 | LFKHMYENA |
| 1776 | LFKPTQNEF |
| 1777 | LFLGKDAMM |
| 1778 | LFLHDNRFL |
| 1779 | LFLQLYRHC |
| 1780 | LFMPISSAV |
| 1781 | LFMPVQRIC |
| 1782 | LFMQNNNWM |
| 1783 | LFMQSVQQT |
| 1784 | LFNSYSTRE |
| 1785 | LFQKMTYEP |
| 1786 | LFQQTLRWQ |
| 1787 | LFQQYNSPF |
| 1788 | LFRVMYCQE |
| 1789 | LFRYKTSLW |
| 1790 | LFSFYNNTS |
| 1791 | LFSHELRYY |
| 1792 | LFSIMHAME |
| 1793 | LFSQNEACS |
| 1794 | LFTDTKRPE |
| 1795 | LFTLVQSNS |
| 1796 | LFVGMSCEL |
| 1797 | LFVQRYQES |
| 1798 | LFVYDLLFH |
| 1799 | LGELGNSFG |
| 1800 | LGERALRDL |
| 1801 | LGMDCWQWE |
| 1802 | LGNHFNNWM |
| 1803 | LGNPCNLGD |
| 1804 | LGSLCNCWF |
| 1805 | LGSPFYECG |
| 1806 | LGTLMSQHC |
| 1807 | LHFQIDAED |
| 1808 | LHHRVYQDA |
| 1809 | LHPTFWQEK |
| 1810 | LHQDIMMAG |
| 1811 | LHQLHIMQA |
| 1812 | LIALFMDYR |
| 1813 | LIATWVNAN |
| 1814 | LICLAQAWK |
| 1815 | LIDERFVQG |
| 1816 | LIKPFIDKA |
| 1817 | LIMMSNWQD |
| 1818 | LINHGQTSC |
| 1819 | LINQFIACT |
| 1820 | LINSTMVSG |
| 1821 | LISLAAYIE |
| 1822 | LISWKQTNS |
| 1823 | LIYSNIKAQ |
| 1824 | LKAPCQMDR |
| 1825 | LKDELVMLG |
| 1826 | LKGYKPSKF |
| 1827 | LKNVIHDGC |
| 1828 | LKSWFGQSP |
| 1829 | LKVEKVMLG |
| 1830 | LKWPVVNCD |
| 1831 | LLGYAQCST |
| 1832 | LLKDYMNST |
| 1833 | LLKTCDIFR |
| 1834 | LLMGFWMQD |
| 1835 | LLNKYENMG |
| 1836 | LLRFYGCCM |
| 1837 | LMEAASRSY |
| 1838 | LMLTYSDHY |
| 1839 | LMNRGEAND |
| 1840 | LMNYSGSSM |
| 1841 | LMQTFPQKC |
| 1842 | LMRQGVLFH |
| 1843 | LMSSTSAHY |
| 1844 | LMTGFMQMG |
| 1845 | LMTPQVKQA |
| 1846 | LMTTQVKQA |
| 1847 | LMVAKTWDC |
| 1848 | LMVFKQLAN |
| 1849 | LMVHGLVYS |
| 1850 | LMWMTPWDN |
| 1851 | LMYMIGTGP |
| 1852 | LMYMIITGP |
| 1853 | LMYQKDCAQ |
| 1854 | LNCHLTNST |
| 1855 | LNESHPPTD |
| 1856 | LNGHYTVQS |
| 1857 | LNKCCSLFY |
| 1858 | LNMDYNKCQ |
| 1859 | LNNACLSDE |
| 1860 | LNNAGYRPC |
| 1861 | LNVMSIHCC |
| 1862 | LPANGQLLA |
| 1863 | LPHAMQKMY |
| 1864 | LPHDKIKDM |
| 1865 | LPHFWGHSE |
| 1866 | LPHKMQNMY |
| 1867 | LPVAMQISH |
| 1868 | LPVDKIKDC |
| 1869 | LQAFYFHDQ |
| 1870 | LQARVGKVF |
| 1871 | LQDRCVCCY |
| 1872 | LQDTMMANE |
| 1873 | LQHLKKYMV |
| 1874 | LQITHNGWQ |
| 1875 | LQMTHNGCQ |
| 1876 | LQPWWLGMV |
| 1877 | LQSTYKMFT |
| 1878 | LQYSLGQQT |
| 1879 | LRAVWAKMD |
| 1880 | LRDKVDWYA |
| 1881 | LRELYTGMF |
| 1882 | LRIGVLSQF |
| 1883 | LRIPFGAAL |
| 1884 | LRMPFSNGV |
| 1885 | LRRECSTCL |
| 1886 | LRTLQIDSM |
| 1887 | LRTPHNHGL |
| 1888 | LRVDTNQQC |
| 1889 | LRVTHDVCS |
| 1890 | LRVTHNVCS |
| 1891 | LSACTVQNM |
| 1892 | LSAFVSHMK |
| 1893 | LSAKEELSG |
| 1894 | LSCKMTNPT |
| 1895 | LSEESGYCS |
| 1896 | LSFQMVDQQ |
| 1897 | LSFYYCGPH |
| 1898 | LSFYYPLHY |
| 1899 | LSGHTYQDN |
| 1900 | LSHETHMGN |
| 1901 | LSIPYQGLN |
| 1902 | LSKDCVAFR |
| 1903 | LSKDCVTFR |
| 1904 | LSLHYLHHH |
| 1905 | LSLNQKWVH |
| 1906 | LSNMVLAAD |
| 1907 | LSPLLDCGN |
| 1908 | LSPTFNHCE |
| 1909 | LSQAMSPGC |
| 1910 | LSQPINHQM |
| 1911 | LSQPYGAGD |
| 1912 | LSSIWYAAN |
| 1913 | LSSYDAKYH |
| 1914 | LSTMTQGSG |
| 1915 | LSVHTCQEQ |
| 1916 | LSVLAGLGG |
| 1917 | LSVQFNNQS |
| 1918 | LSWETKGCG |
| 1919 | LSWHCGEEN |
| 1920 | LTADPQERF |
| 1921 | LTAVKQEGY |
| 1922 | LTCNCNYWQ |
| 1923 | LTCSKHLQP |
| 1924 | LTEFIQAAQ |
| 1925 | LTEKGMLDC |
| 1926 | LTEKGMLDW |
| 1927 | LTEWLYQEM |
| 1928 | LTGWFNTRD |
| 1929 | LTHILDEFC |
| 1930 | LTISHNVSG |
| 1931 | LTLGMSSHE |
| 1932 | LTLGMSSHK |
| 1933 | LTLPFKPEY |
| 1934 | LTLTWGQTY |
| 1935 | LTMCYESNN |
| 1936 | LTMVGEYVN |
| 1937 | LTRFLNCQR |
| 1938 | LTRWNEEKE |
| 1939 | LTSFVDCVQ |
| 1940 | LTSRYNVTH |
| 1941 | LTSSKEFEH |
| 1942 | LTSYENWGW |
| 1943 | LTTHFVEED |
| 1944 | LTTMVKKAA |
| 1945 | LTTSLNGFD |
| 1946 | LTVGHYVYD |
| 1947 | LTVPNESAF |
| 1948 | LTVSKEFSH |
| 1949 | LTVWQQKME |
| 1950 | LTYLLGHKE |
| 1951 | LVADMYERS |
| 1952 | LVCMQGRSN |
| 1953 | LVDQSGDGV |
| 1954 | LVECSNLVA |
| 1955 | LVGERKNAY |
| 1956 | LVGWTLQHV |
| 1957 | LVITKMLQP |
| 1958 | LVKLATYTH |
| 1959 | LVLPVEFYM |
| 1960 | LVNLATYTH |
| 1961 | LVTTEMGTN |
| 1962 | LVTWAANEQ |
| 1963 | LVVEDWIRC |
| 1964 | LWAAVQIDH |
| 1965 | LWCPKEIER |
| 1966 | LWDTSMLKE |
| 1967 | LWEGLATCL |
| 1968 | LWEKCEFDQ |
| 1969 | LWFAITKER |
| 1970 | LWGSFGASS |
| 1971 | LWGWTLQHQ |
| 1972 | LWIKQHVKG |
| 1973 | LWKGYLKSN |
| 1974 | LWMNTGQYH |
| 1975 | LWNIYGTME |
| 1976 | LWNQQDWCR |
| 1977 | LWNTRGIYN |
| 1978 | LWQYSHAWQ |
| 1979 | LWTPCNHLM |
| 1980 | LWYQMLHGL |
| 1981 | LYCVWPAGT |
| 1982 | LYERYWVKQ |
| 1983 | LYFFMGTQA |
| 1984 | LYFGFPMSG |
| 1985 | LYFNMVQGL |
| 1986 | LYFRMGTVA |
| 1987 | LYGWVDLEG |
| 1988 | LYLQHQIDT |
| 1989 | LYRKIMSIV |
| 1990 | LYSYMLHDR |
| 1991 | LYVMKGIQV |
| 1992 | LYVQSHCAV |
| 1993 | LYWFYLEIH |
| 1994 | MAAEYESCS |
| 1995 | MAFFKSNLH |
| 1996 | MAGHYFNSR |
| 1997 | MAGQWCCPE |
| 1998 | MAHPDQQHY |
| 1999 | MAIEMPLHW |
| 2000 | MAKQEINWI |
| 2001 | MALPVTSIR |
| 2002 | MAMNQPEWS |
| 2003 | MAMPIHDRI |
| 2004 | MAQHAITTH |
| 2005 | MAQKREVGV |
| 2006 | MASPYTHVW |
| 2007 | MATCYMYAD |
| 2008 | MATHGNSKT |
| 2009 | MATVNLRDC |
| 2010 | MAVEFCQSD |
| 2011 | MAYCNHFSD |
| 2012 | MAYHSAQCI |
| 2013 | MAYPTQLIF |
| 2014 | MAYTPEFGN |
| 2015 | MCDDPRAWQ |
| 2016 | MCDEKFSYG |
| 2017 | MCDEKNSMG |
| 2018 | MCEPNQGMQ |
| 2019 | MCGPSLVYA |
| 2020 | MCHNYAGVD |
| 2021 | MCHPYFSDG |
| 2022 | MCLDSIMFG |
| 2023 | MCNGFCGIW |
| 2024 | MCNIHNIWD |
| 2025 | MCNYWITDC |
| 2026 | MCSVYQACM |
| 2027 | MCTADQKSI |
| 2028 | MDDICEFYA |
| 2029 | MDHPCQQHY |
| 2030 | MDNCPPMWN |
| 2031 | MEDLWTVGP |
| 2032 | MEFLIMADE |
| 2033 | MENEANIRG |
| 2034 | MESCKLWGV |
| 2035 | MEVPDFMSY |
| 2036 | MFDLYHDFK |
| 2037 | MFEKRNHWG |
| 2038 | MFEMQYCSP |
| 2039 | MFERMYVVN |
| 2040 | MFHAQIHCM |
| 2041 | MFNMFTEWR |
| 2042 | MFNMLTDSA |
| 2043 | MFQFMECLA |
| 2044 | MFTISGLHA |
| 2045 | MGAEHMCVQ |
| 2046 | MGARHMNQE |
| 2047 | MGATKSDDF |
| 2048 | MGDWLGAPT |
| 2049 | MGEGLSFYA |
| 2050 | MGGVANGSS |
| 2051 | MGHWGAEMP |
| 2052 | MGNHLCHYW |
| 2053 | MGNICNVMF |
| 2054 | MGPLLFLFD |
| 2055 | MGSMRIQTD |
| 2056 | MGSPAAAVG |
| 2057 | MGSQQIQTD |
| 2058 | MGTVEKICH |
| 2059 | MGVQLPIVE |
| 2060 | MGWHYGGWS |
| 2061 | MHDSCYVMP |
| 2062 | MHGPSLAYA |
| 2063 | MHHSVVAQF |
| 2064 | MHLEKNEMM |
| 2065 | MHMEVLWGH |
| 2066 | MHMLYKFNC |
| 2067 | MHMSGQSWH |
| 2068 | MHMSKQSWL |
| 2069 | MHMTATIFG |
| 2070 | MHMVKGGWT |
| 2071 | MHMVPGGWM |
| 2072 | MHNAHGIIE |
| 2073 | MHNYYEMCS |
| 2074 | MHQTLQCIG |
| 2075 | MHSFYGLSG |
| 2076 | MHTVYMESI |
| 2077 | MHVNFNEQR |
| 2078 | MHWFYNTGE |
| 2079 | MIAINGGEQ |
| 2080 | MIDLGVIDN |
| 2081 | MIFHSTGAA |
| 2082 | MIHHRDHSI |
| 2083 | MIHPLQSHY |
| 2084 | MIHQMQTLD |
| 2085 | MILLHNQGT |
| 2086 | MIMGYNFEF |
| 2087 | MIMQGVMQA |
| 2088 | MIMQGVVQA |
| 2089 | MINCTAELA |
| 2090 | MINCTAEVA |
| 2091 | MINFNSGTC |
| 2092 | MINFPSMIN |
| 2093 | MINWKAPRA |
| 2094 | MINWKAQCA |
| 2095 | MIQQMMFYT |
| 2096 | MIRGQDNWL |
| 2097 | MIRIMTGQY |
| 2098 | MISAPPDTC |
| 2099 | MITMFDGMC |
| 2100 | MITMYDGMC |
| 2101 | MIYSRGNQP |
| 2102 | MKDIKQVDC |
| 2103 | MKFPNHAEW |
| 2104 | MKGEMEQNA |
| 2105 | MKHPMVNNE |
| 2106 | MKMNYGLVC |
| 2107 | MKSEQQILA |
| 2108 | MKTTMVMAP |
| 2109 | MKWFVFDSN |
| 2110 | MLCKCNADT |
| 2111 | MLLLIPFAP |
| 2112 | MLSTYMPIS |
| 2113 | MLYVNMGDR |
| 2114 | MMAVIRQQS |
| 2115 | MMEQSNWHY |
| 2116 | MMGFKDISC |
| 2117 | MMHWIDMCT |
| 2118 | MMIPWIGIA |
| 2119 | MMLKQGPIY |
| 2120 | MMNPNTEPH |
| 2121 | MMNTWGFMP |
| 2122 | MMNYNDHLH |
| 2123 | MMPGETVRG |
| 2124 | MMQTIQADV |
| 2125 | MMSSNVLAE |
| 2126 | MMVKRDLND |
| 2127 | MMYISCSCT |
| 2128 | MNADVCLTG |
| 2129 | MNEMAQHDR |
| 2130 | MNEQYRHGV |
| 2131 | MNEYKGENY |
| 2132 | MNEYKHGNY |
| 2133 | MNGFTMTYH |
| 2134 | MNGRPEQMA |
| 2135 | MNGRPNQML |
| 2136 | MNINDETQC |
| 2137 | MNMMLMSNS |
| 2138 | MNMTMQYIT |
| 2139 | MNPLGPLLW |
| 2140 | MNRFTHNDQ |
| 2141 | MNSMKPNSV |
| 2142 | MNSWRNTSD |
| 2143 | MPHGWCNTI |
| 2144 | MPHHTSDPF |
| 2145 | MPNQYHSMD |
| 2146 | MQDFMFCIT |
| 2147 | MQDYWTLGA |
| 2148 | MQEKIHMCT |
| 2149 | MQELITFDE |
| 2150 | MQELITFGE |
| 2151 | MQHQNQSVC |
| 2152 | MQKTLCGSY |
| 2153 | MQNYLQHCE |
| 2154 | MQSLYETFG |
| 2155 | MQVMYEASE |
| 2156 | MRDLGVIWN |
| 2157 | MRELMGAYC |
| 2158 | MRIPSGVGQ |
| 2159 | MRNTFNYLC |
| 2160 | MSAARVYDL |
| 2161 | MSAECLHSD |
| 2162 | MSAIHDWGQ |
| 2163 | MSAIPTNGM |
| 2164 | MSAQVDCQF |
| 2165 | MSATLEAAF |
| 2166 | MSDDSTFDF |
| 2167 | MSDDSTVDS |
| 2168 | MSDLVPKWA |
| 2169 | MSDPCVDNH |
| 2170 | MSDPCVDNQ |
| 2171 | MSDQKEHMS |
| 2172 | MSDTCVANQ |
| 2173 | MSEPFNYCG |
| 2174 | MSERCYAGG |
| 2175 | MSEVCPNDI |
| 2176 | MSEVCSNDI |
| 2177 | MSFQKPKWS |
| 2178 | MSGMGDDFP |
| 2179 | MSHEYLFYG |
| 2180 | MSINMPLHW |
| 2181 | MSIPSFMQK |
| 2182 | MSLLIPLAP |
| 2183 | MSNNSVGSY |
| 2184 | MSQFKAYLF |
| 2185 | MSQLVHRCE |
| 2186 | MSQSTYHAG |
| 2187 | MSRHDLNWD |
| 2188 | MSVHFIYMY |
| 2189 | MSVHTWDHY |
| 2190 | MTAILQDNW |
| 2191 | MTAQIGHPI |
| 2192 | MTDPYAGAK |
| 2193 | MTDVHKHLG |
| 2194 | MTELKTFGE |
| 2195 | MTGEMIQNA |
| 2196 | MTGQASDLI |
| 2197 | MTGYGQHDA |
| 2198 | MTITHHVNY |
| 2199 | MTKHNMIVP |
| 2200 | MTLPWGQQY |
| 2201 | MTLTNMAYL |
| 2202 | MTMDENTCR |
| 2203 | MTMPVMINQ |
| 2204 | MTMTATTFG |
| 2205 | MTQRVVHWG |
| 2206 | MTQYLQDSV |
| 2207 | MTQYNKKVN |
| 2208 | MTRCEGLFT |
| 2209 | MTSHVQHRF |
| 2210 | MTVLICDYE |
| 2211 | MVALVDGHR |
| 2212 | MVAVYNRQR |
| 2213 | MVCPSNCAN |
| 2214 | MVCTSLRFK |
| 2215 | MVDFGNMQP |
| 2216 | MVEYCYTQE |
| 2217 | MVILSVETE |
| 2218 | MVKQGGDWR |
| 2219 | MVLKICTAK |
| 2220 | MVMPVNIVD |
| 2221 | MVMQDNASM |
| 2222 | MVMSKQADL |
| 2223 | MVNHHYMTG |
| 2224 | MVQMDVHWE |
| 2225 | MVSLITSRT |
| 2226 | MVTEAHDYY |
| 2227 | MVVQSPIVE |
| 2228 | MWDVAHQSC |
| 2229 | MWISRDQDH |
| 2230 | MWNPMYIWN |
| 2231 | MWREFTMQQ |
| 2232 | MWTEFNEYV |
| 2233 | MWWRESAMR |
| 2234 | MYADFQSSY |
| 2235 | MYCMKPNRQ |
| 2236 | MYDSTHAVQ |
| 2237 | MYDSVHAVQ |
| 2238 | MYGQQSLWN |
| 2239 | MYHEANSGF |
| 2240 | MYHSKMTEA |
| 2241 | MYHSYQNSD |
| 2242 | MYKLLNDFS |
| 2243 | MYKQMGMAD |
| 2244 | MYLRDHNGL |
| 2245 | MYRERRAGY |
| 2246 | MYVSYMTHC |
| 2247 | MYYHCITAQ |
| 2248 | NAAMQDINH |
| 2249 | NACFFQNDP |
| 2250 | NACMIERAK |
| 2251 | NACMIEWAK |
| 2252 | NACNAYGRG |
| 2253 | NACQNDLYW |
| 2254 | NADHRFDAV |
| 2255 | NADKIGMLD |
| 2256 | NADMLMNGS |
| 2257 | NADPQQYQE |
| 2258 | NAEANWEYQ |
| 2259 | NAELQQADI |
| 2260 | NAENYQRLY |
| 2261 | NAERYQKLY |
| 2262 | NAESEDMLE |
| 2263 | NAESRFAQM |
| 2264 | NAFSEYVYD |
| 2265 | NAGSHAIQS |
| 2266 | NALPCAFNE |
| 2267 | NAMLFHHAM |
| 2268 | NAMYSHFLY |
| 2269 | NANSHAIHS |
| 2270 | NANWLHTQI |
| 2271 | NAPPCAFNE |
| 2272 | NASLERKWN |
| 2273 | NASQYQLSA |
| 2274 | NATDMGISF |
| 2275 | NATTIHMWF |
| 2276 | NAVFVQNLR |
| 2277 | NAVLQQYQE |
| 2278 | NAYSPESDG |
| 2279 | NCAERMKFC |
| 2280 | NCAEYFDHC |
| 2281 | NCAIQLDQQ |
| 2282 | NCEPIPFFP |
| 2283 | NCIINHGRD |
| 2284 | NCLEVVRAC |
| 2285 | NCLPMPSLH |
| 2286 | NCLQIGMQF |
| 2287 | NCNIKEAMD |
| 2288 | NCPREVATT |
| 2289 | NCQNTTHYA |
| 2290 | NCQPFMDYH |
| 2291 | NCQPIMDTH |
| 2292 | NCQRVNWTE |
| 2293 | NCTLFQDSH |
| 2294 | NCVQSFAVD |
| 2295 | NCVSRKKFA |
| 2296 | NDFMGAPEP |
| 2297 | NDVIYIYQG |
| 2298 | NEAHLWGHV |
| 2299 | NEAILFAYH |
| 2300 | NEAPYSTWQ |
| 2301 | NESNPQNGW |
| 2302 | NFAQFTKFQ |
| 2303 | NFCKQGNTC |
| 2304 | NFDQIGGCR |
| 2305 | NFDQMKCAQ |
| 2306 | NFEQINPLG |
| 2307 | NFLSSCSSA |
| 2308 | NFNQSSSTM |
| 2309 | NFRHTDQCG |
| 2310 | NFTFQCGIH |
| 2311 | NFVHIQSGE |
| 2312 | NFVTSGQMG |
| 2313 | NFYMIQSSN |
| 2314 | NFYMSGQAS |
| 2315 | NGEQWHKGG |
| 2316 | NGGSYIPCM |
| 2317 | NGILMHTTF |
| 2318 | NGPSYISWV |
| 2319 | NGQHFDMVG |
| 2320 | NGTGVVECG |
| 2321 | NHAPLLAFW |
| 2322 | NHASTGAGT |
| 2323 | NHCPVQKAT |
| 2324 | NHCSMCCGN |
| 2325 | NHDEKLWAP |
| 2326 | NHDYVKQTS |
| 2327 | NHDYYCQTS |
| 2328 | NHELYAQVA |
| 2329 | NHGQLKFPV |
| 2330 | NHGSREIDI |
| 2331 | NHILKMEFW |
| 2332 | NHNCVEMHG |
| 2333 | NHPYDIGQY |
| 2334 | NHRVNYEMY |
| 2335 | NHSWVEMFG |
| 2336 | NHVAYYMAK |
| 2337 | NHYPLMVEW |
| 2338 | NIAANWMFD |
| 2339 | NIAGYWMFD |
| 2340 | NICNLNQVE |
| 2341 | NICTWNEAC |
| 2342 | NICVKNKLH |
| 2343 | NIELKTYFF |
| 2344 | NIERKTYAF |
| 2345 | NIFDWQFYA |
| 2346 | NIHCKLHWY |
| 2347 | NIICFNWQV |
| 2348 | NIIHLMDYA |
| 2349 | NIIQLMDYA |
| 2350 | NINCIQIGK |
| 2351 | NINHYNEGE |
| 2352 | NIRCGNDAS |
| 2353 | NISEPSQDY |
| 2354 | NISFGTPPN |
| 2355 | NISFGTPSN |
| 2356 | NITQMYKHI |
| 2357 | NIVSQTHQE |
| 2358 | NIYHESHCG |
| 2359 | NKFNGMHCG |
| 2360 | NKFWSIDTS |
| 2361 | NKNTHAAVW |
| 2362 | NLFLTNGMG |
| 2363 | NLFLWLMMD |
| 2364 | NLHPRCHWI |
| 2365 | NLKCIMIQC |
| 2366 | NLVPFQTCE |
| 2367 | NMMDLLTHC |
| 2368 | NMMDLRTHC |
| 2369 | NMMPRVCWQ |
| 2370 | NMNTFGKMN |
| 2371 | NMTMAHQAS |
| 2372 | NMYHVQRFS |
| 2373 | NNAIMWASP |
| 2374 | NNGPGNIGM |
| 2375 | NNGVHIFCE |
| 2376 | NNLPLLTSV |
| 2377 | NPDTMSHTA |
| 2378 | NPEMDLRCA |
| 2379 | NPEMDLWCA |
| 2380 | NPIPGYFDQ |
| 2381 | NPIVMNCLS |
| 2382 | NPQPWLGMW |
| 2383 | NPTVLMAHL |
| 2384 | NQEEGHCLY |
| 2385 | NQGPQMHGF |
| 2386 | NQHVIYDVT |
| 2387 | NQKAMCWMY |
| 2388 | NQNFLGNWG |
| 2389 | NQVLLDDGM |
| 2390 | NRCDFSLFE |
| 2391 | NRCFTTIEP |
| 2392 | NRCFTTTEP |
| 2393 | NREFQMIHC |
| 2394 | NREMIYAAF |
| 2395 | NRLVVNGPH |
| 2396 | NRVAKIWRW |
| 2397 | NSAKKEQGV |
| 2398 | NSCTDNSSS |
| 2399 | NSEHEGMWD |
| 2400 | NSEQKQHLQ |
| 2401 | NSFAFDMTE |
| 2402 | NSGIPNNLA |
| 2403 | NSGYECHAL |
| 2404 | NSGYEWHAL |
| 2405 | NSIPYQFAV |
| 2406 | NSNSQARVY |
| 2407 | NSQWVQMFG |
| 2408 | NSTYATWAC |
| 2409 | NSYQIGGTR |
| 2410 | NTDHKMWDS |
| 2411 | NTEHSPATN |
| 2412 | NTGLENNIP |
| 2413 | NTGYYNQQC |
| 2414 | NTHHVTQWH |
| 2415 | NTHQMAVVY |
| 2416 | NTIHRESPA |
| 2417 | NTMLKEMGP |
| 2418 | NTMPLQPME |
| 2419 | NTMYWWPRE |
| 2420 | NTNFYQQMS |
| 2421 | NTQLHDNFG |
| 2422 | NTSHGCTYY |
| 2423 | NTSMLNNGS |
| 2424 | NTTIYQQQP |
| 2425 | NVDGQCTVA |
| 2426 | NVDGRHPMT |
| 2427 | NVDHRFDAV |
| 2428 | NVDQKCQAN |
| 2429 | NVGYCKWQS |
| 2430 | NVISLLNCR |
| 2431 | NVNAPKKQM |
| 2432 | NVQFHMMMS |
| 2433 | NVQQQCNFQ |
| 2434 | NVSFTEIGW |
| 2435 | NVSGYAAIQ |
| 2436 | NVSWYAAIQ |
| 2437 | NVTHISVGT |
| 2438 | NVVHHPVAP |
| 2439 | NVVHVENIG |
| 2440 | NVWTCNYQP |
| 2441 | NVYMRTCNM |
| 2442 | NWAGVQRIM |
| 2443 | NWEMAQHDR |
| 2444 | NWFLTHPIL |
| 2445 | NWLVQHAFQ |
| 2446 | NWTQTQAEA |
| 2447 | NWYSTNTCF |
| 2448 | NYDFLHHVE |
| 2449 | NYELYKQVA |
| 2450 | NYGPKMLTP |
| 2451 | NYMQTTNDD |
| 2452 | NYNHFFAAD |
| 2453 | NYQSEMINW |
| 2454 | NYQSFGLFA |
| 2455 | NYYANGNWP |
| 2456 | PACYSDVLN |
| 2457 | PADLCDTCQ |
| 2458 | PCFFRHNYW |
| 2459 | PCPTAINQH |
| 2460 | PDDDFGSCE |
| 2461 | PDDYFGTCE |
| 2462 | PDECGFGLT |
| 2463 | PDMVNDWWY |
| 2464 | PDNQFSRVV |
| 2465 | PDQYLVIGD |
| 2466 | PDSPGVCSC |
| 2467 | PEFYVKKFD |
| 2468 | PEFYVQKFD |
| 2469 | PFAEKAPDV |
| 2470 | PGHSDKSSN |
| 2471 | PGLLIPNGK |
| 2472 | PHATYNKPT |
| 2473 | PHPDSAVQL |
| 2474 | PIIKGEPAD |
| 2475 | PIIKRVSWT |
| 2476 | PILNVMCAE |
| 2477 | PKHSDKSHN |
| 2478 | PKMIKQPVS |
| 2479 | PKQYWQHWF |
| 2480 | PKRIRYRTK |
| 2481 | PKRYQAWTW |
| 2482 | PKSCFQCTA |
| 2483 | PKWWPIQYD |
| 2484 | PLCCTHWGT |
| 2485 | PLQNWFSAQ |
| 2486 | PLRAECQWA |
| 2487 | PLRPECPWA |
| 2488 | PMSVTNSAY |
| 2489 | PMVHGLVYS |
| 2490 | PPDMQSAKW |
| 2491 | PPFKAAKYS |
| 2492 | PPMISHCEP |
| 2493 | PPPLKAPMY |
| 2494 | PPPPPPPPP |
| 2495 | PQTLWDKSG |
| 2496 | PRHTMLGPS |
| 2497 | PRNPLQVQN |
| 2498 | PRPLKAPMY |
| 2499 | PSYTMHACP |
| 2500 | PTAAFMQFM |
| 2501 | PTDGINISG |
| 2502 | PTEFHINVQ |
| 2503 | PTEKTHGPL |
| 2504 | PTHNLCMRV |
| 2505 | PTQFYGPAF |
| 2506 | PTRLTVYRC |
| 2507 | PTYPYEWFQ |
| 2508 | PVEVGQNQC |
| 2509 | PVHFWTQAK |
| 2510 | PVTHPQTSK |
| 2511 | PWRTRVGAR |
| 2512 | PWTEMPNKI |
| 2513 | PWTEMSNKI |
| 2514 | PYCWLSTAF |
| 2515 | PYKTQEGDG |
| 2516 | PYKTQEWYG |
| 2517 | PYQPLSCPP |
| 2518 | QACNITNSG |
| 2519 | QADNARSVH |
| 2520 | QADTLQHWD |
| 2521 | QAEGHFTNC |
| 2522 | QAEHRLNAD |
| 2523 | QAGFIQNNQ |
| 2524 | QAGGSGFNE |
| 2525 | QAGSSFHQA |
| 2526 | QAHISPSMC |
| 2527 | QAHTNMQVH |
| 2528 | QAHWWHAHH |
| 2529 | QAIANNAMQ |
| 2530 | QAIHMPLFT |
| 2531 | QAILRQNLP |
| 2532 | QAIPRENPM |
| 2533 | QAIPYSFCE |
| 2534 | QAIQVGMCH |
| 2535 | QAITGDRSN |
| 2536 | QALMLTRQQ |
| 2537 | QAMHKADHS |
| 2538 | QAMPNNMCC |
| 2539 | QAMTNGIAC |
| 2540 | QANFGMTQL |
| 2541 | QANPCSFGN |
| 2542 | QANPRNRTA |
| 2543 | QANSNIHQP |
| 2544 | QANWTECID |
| 2545 | QAPDQSYMP |
| 2546 | QASWHSVDQ |
| 2547 | QATLEWAHE |
| 2548 | QATLRSSCS |
| 2549 | QATMIHHDD |
| 2550 | QATNEHIFW |
| 2551 | QATVCHFHA |
| 2552 | QATWEGIRD |
| 2553 | QATWQCRYF |
| 2554 | QAVCSSNAM |
| 2555 | QAVFEDADN |
| 2556 | QAWNFTIFH |
| 2557 | QAWSYDNER |
| 2558 | QAYAYLNSS |
| 2559 | QAYQYRSCK |
| 2560 | QCANGYQSQ |
| 2561 | QCAPCDICC |
| 2562 | QCDFMIIEY |
| 2563 | QCDFRRKDY |
| 2564 | QCEPIPFEP |
| 2565 | QCEQDAQQE |
| 2566 | QCGEIHRAY |
| 2567 | QCGQQMKMC |
| 2568 | QCHHGYIYE |
| 2569 | QCHQNYGLI |
| 2570 | QCITGNAGA |
| 2571 | QCLQTNPYN |
| 2572 | QCMFRIDWD |
| 2573 | QCMFRIDWY |
| 2574 | QCNWIHNTM |
| 2575 | QCQHDMHCE |
| 2576 | QCQYPSHFM |
| 2577 | QCSGYHWMK |
| 2578 | QCSWHGVDQ |
| 2579 | QCTQMVCCG |
| 2580 | QCVEPPQNC |
| 2581 | QCVEPPQNW |
| 2582 | QCVKFMQYN |
| 2583 | QCVMDGCMA |
| 2584 | QDDLIYQTG |
| 2585 | QDFYIMAMG |
| 2586 | QDMSAWPDG |
| 2587 | QDVVNIEAA |
| 2588 | QEIPAMQQQ |
| 2589 | QETMIGQSF |
| 2590 | QEVFELDNM |
| 2591 | QEVRQFCSS |
| 2592 | QFAFRGSHI |
| 2593 | QFAIQEGAI |
| 2594 | QFCTSCASN |
| 2595 | QFEGQSKHE |
| 2596 | QFEGWVHNE |
| 2597 | QFGLINEHV |
| 2598 | QFHQQVIWS |
| 2599 | QFILTPRDF |
| 2600 | QFKNNWQIE |
| 2601 | QFLDNQQAF |
| 2602 | QFLEAPMMH |
| 2603 | QFLSMMNCF |
| 2604 | QFMQHPQMQ |
| 2605 | QFNPLQDQC |
| 2606 | QFNYVMIGD |
| 2607 | QFQWSMEHK |
| 2608 | QFRYMGHQI |
| 2609 | QFSEFVRWN |
| 2610 | QFSHIQAAP |
| 2611 | QFSPCVKMF |
| 2612 | QFTIHERID |
| 2613 | QFTYFNCWS |
| 2614 | QFVNWQRLF |
| 2615 | QGAMKQTCK |
| 2616 | QGDFDQTVL |
| 2617 | QGDPDQTTL |
| 2618 | QGEFLIGQS |
| 2619 | QGESRFMFQ |
| 2620 | QGFVVHSPS |
| 2621 | QGGFNYAAS |
| 2622 | QGGYASFHS |
| 2623 | QGHKTMHVF |
| 2624 | QGQHVTLAW |
| 2625 | QGTHTHWLG |
| 2626 | QGTTDMPMY |
| 2627 | QGVSTRCHC |
| 2628 | QHCQGLFWS |
| 2629 | QHCQGLSWS |
| 2630 | QHDFTSCYW |
| 2631 | QHFNMVQGL |
| 2632 | QHIWAPMGM |
| 2633 | QHLHSEWGS |
| 2634 | QHLHSMWGY |
| 2635 | QHMHTTMQY |
| 2636 | QHMPSKRLD |
| 2637 | QHQTIDEQF |
| 2638 | QHRMGFSWM |
| 2639 | QHSTYWTGV |
| 2640 | QHVATQNLG |
| 2641 | QHVSVGDCV |
| 2642 | QHWFVKCCV |
| 2643 | QHYHTCMQY |
| 2644 | QIAHCQYNE |
| 2645 | QIANEDTYA |
| 2646 | QIDIQMAWP |
| 2647 | QIELFMDMD |
| 2648 | QIFGIQFAG |
| 2649 | QIIPGGFKH |
| 2650 | QIKLSPMDP |
| 2651 | QIMQHPQMQ |
| 2652 | QIQCTMISQ |
| 2653 | QIQDFFTLP |
| 2654 | QISNWGFGA |
| 2655 | QITDNLFEY |
| 2656 | QITDWYQFF |
| 2657 | QIVYHLLNG |
| 2658 | QIYSLASTW |
| 2659 | QKCPINQPR |
| 2660 | QKGMMLDCA |
| 2661 | QKGPHNMSE |
| 2662 | QKIPFDHDA |
| 2663 | QKIYSWGPI |
| 2664 | QKQQGTCGN |
| 2665 | QLDTMMYTN |
| 2666 | QLELFNSAY |
| 2667 | QLEPQVNSD |
| 2668 | QLFCWVTSK |
| 2669 | QLFVWVTAK |
| 2670 | QLGGVEFYT |
| 2671 | QLHCVQMQS |
| 2672 | QLKEVMLQY |
| 2673 | QLMVAFLCL |
| 2674 | QLNQWMFAG |
| 2675 | QLSQSSTAY |
| 2676 | QLTAFCWQE |
| 2677 | QLTAFGVQE |
| 2678 | QLVWNQKQQ |
| 2679 | QMAEFEAEA |
| 2680 | QMDHRHTFE |
| 2681 | QMDHRHTVC |
| 2682 | QMEDTFQNS |
| 2683 | QMEQFGIPG |
| 2684 | QMFCKNTDM |
| 2685 | QMFIFQANN |
| 2686 | QMFLWYGHN |
| 2687 | QMGYREERM |
| 2688 | QMHICMYEG |
| 2689 | QMHIVHCTG |
| 2690 | QMHTGATWG |
| 2691 | QMHTQATNG |
| 2692 | QMKPLINDE |
| 2693 | QMMPAYHTE |
| 2694 | QMMYRDTSE |
| 2695 | QMMYWSVIE |
| 2696 | QMNQFGVGL |
| 2697 | QMNQLFCSS |
| 2698 | QMRDIRAMG |
| 2699 | QMRIQTQDP |
| 2700 | QMVQYQWWN |
| 2701 | QMVRFNNSA |
| 2702 | QMVSGDMED |
| 2703 | QMVSYEAYR |
| 2704 | QNAIVTQFA |
| 2705 | QNAPYMENA |
| 2706 | QNCFTYEDQ |
| 2707 | QNENFNTGC |
| 2708 | QNGHTGVAI |
| 2709 | QNICNQQQD |
| 2710 | QNICNQQQY |
| 2711 | QNILAGDCI |
| 2712 | QNKHLNGLM |
| 2713 | QNMLAGDCM |
| 2714 | QNNTTRGFT |
| 2715 | QNQSADVDA |
| 2716 | QNTPQHNFM |
| 2717 | QNVCINQAM |
| 2718 | QNYYYPPMH |
| 2719 | QPAMENTTP |
| 2720 | QPKALELVP |
| 2721 | QPYQQSQMD |
| 2722 | QPYQQSQMG |
| 2723 | QQAMWLHSS |
| 2724 | QQCIPLTWE |
| 2725 | QQECKHKMQ |
| 2726 | QQEQLDFSD |
| 2727 | QQFTIQDHK |
| 2728 | QQLGPKFEK |
| 2729 | QQQQTNFQN |
| 2730 | QQRFCCPWD |
| 2731 | QQSFSVDVN |
| 2732 | QQSFYCANV |
| 2733 | QQSNLNLGK |
| 2734 | QQSVCQVQN |
| 2735 | QQTMMEAFD |
| 2736 | QQVIANLAG |
| 2737 | QRCTWDQAY |
| 2738 | QREEANSGC |
| 2739 | QRGHGMDSG |
| 2740 | QRTTALKDQ |
| 2741 | QRVEVSKCR |
| 2742 | QRYVQEACP |
| 2743 | QRYVQEACS |
| 2744 | QSADEAACS |
| 2745 | QSADFMFMG |
| 2746 | QSAEWYLND |
| 2747 | QSAIFNTSH |
| 2748 | QSASQVLFP |
| 2749 | QSAVWNGCE |
| 2750 | QSCWFSHNP |
| 2751 | QSDNARSVH |
| 2752 | QSFPCMFKD |
| 2753 | QSFPHQQAQ |
| 2754 | QSFPMQSMQ |
| 2755 | QSGAFEKHQ |
| 2756 | QSGCREIDW |
| 2757 | QSGCTLQFP |
| 2758 | QSGNAWGSL |
| 2759 | QSHDFANCA |
| 2760 | QSHHFLNSD |
| 2761 | QSHPSVSNM |
| 2762 | QSHSKHDSK |
| 2763 | QSHSWVSNM |
| 2764 | QSICNQWCQ |
| 2765 | QSLNFNQHC |
| 2766 | QSLPRYGFD |
| 2767 | QSMGKAMNT |
| 2768 | QSMQKMMDF |
| 2769 | QSMWSAFKH |
| 2770 | QSNHFGAGV |
| 2771 | QSNLTEAGQ |
| 2772 | QSNQVIDQF |
| 2773 | QSPYGRKQA |
| 2774 | QSQGVVDTS |
| 2775 | QSQGVVINS |
| 2776 | QSRCLFACI |
| 2777 | QSSSSYLWG |
| 2778 | QSTDDNICH |
| 2779 | QSTHRVGSY |
| 2780 | QSTNMDFCG |
| 2781 | QSTPILGPK |
| 2782 | QSTPTILPK |
| 2783 | QSTTYWNAM |
| 2784 | QSVHRNTRN |
| 2785 | QSVTDNYFY |
| 2786 | QSWNVGFTS |
| 2787 | QSWSQVAFP |
| 2788 | QSWSQVTFP |
| 2789 | QSYIMGSFH |
| 2790 | QTADIETNQ |
| 2791 | QTAEFKRHQ |
| 2792 | QTAQIELYH |
| 2793 | QTAQINCSM |
| 2794 | QTDKPVYSA |
| 2795 | QTEEYKAVE |
| 2796 | QTELARENT |
| 2797 | QTERKFSAL |
| 2798 | QTFSTMGTD |
| 2799 | QTGMQWTGG |
| 2800 | QTGSAQSAF |
| 2801 | QTGTFGINS |
| 2802 | QTILDVQSV |
| 2803 | QTKDWGFVH |
| 2804 | QTKVLFERD |
| 2805 | QTLSRDDWC |
| 2806 | QTLSRLDIC |
| 2807 | QTMTQQYPE |
| 2808 | QTNMVDRIF |
| 2809 | QTNPLGCMC |
| 2810 | QTNTREGWG |
| 2811 | QTQMGQYAS |
| 2812 | QTRLWNNYC |
| 2813 | QTSEEQSDW |
| 2814 | QTSLTDSIL |
| 2815 | QTSPKFCMC |
| 2816 | QTSYSYADP |
| 2817 | QTTDMFQSF |
| 2818 | QTTPYVWVF |
| 2819 | QTTQETAQC |
| 2820 | QTTVEKICH |
| 2821 | QTVEWTTQQ |
| 2822 | QTVIQWHDW |
| 2823 | QTVKNVYSA |
| 2824 | QTWFKGFAC |
| 2825 | QTYQYRSCK |
| 2826 | QVEGSNMHW |
| 2827 | QVESRCMFQ |
| 2828 | QVEVGEKYH |
| 2829 | QVEYGNHDM |
| 2830 | QVFCKHEYT |
| 2831 | QVHAINDGH |
| 2832 | QVHHNMQVH |
| 2833 | QVHLPVEQG |
| 2834 | QVIPAMMKQ |
| 2835 | QVMHTLRTY |
| 2836 | QVNQGFKAS |
| 2837 | QVQSGQFIF |
| 2838 | QVSCVIMTP |
| 2839 | QVSQQEGIV |
| 2840 | QVTFVHCEN |
| 2841 | QVTHKWFAM |
| 2842 | QVTNQNKTR |
| 2843 | QVTPNFSCD |
| 2844 | QVVDMQDWC |
| 2845 | QVVPRYYSD |
| 2846 | QWAKMFADN |
| 2847 | QWAPIMPLS |
| 2848 | QWARWSVQD |
| 2849 | QWCESNSHL |
| 2850 | QWDCCVQDR |
| 2851 | QWDKVDGMM |
| 2852 | QWDLVGAVG |
| 2853 | QWDNKNQFP |
| 2854 | QWDTAQDCQ |
| 2855 | QWDYARQNF |
| 2856 | QWEAFEFIL |
| 2857 | QWEAFGHID |
| 2858 | QWEIYGKMN |
| 2859 | QWEKQNAWG |
| 2860 | QWENQPAWG |
| 2861 | QWHPKHSFA |
| 2862 | QWKHLHWTI |
| 2863 | QWMLKDSLK |
| 2864 | QWMQNQRYM |
| 2865 | QWMSKISDL |
| 2866 | QWNKNQNAP |
| 2867 | QWNLVGKVG |
| 2868 | QWPEYTTEA |
| 2869 | QWPHYTTEC |
| 2870 | QWPPTHDMG |
| 2871 | QWQDDNSQF |
| 2872 | QWQLTGHLN |
| 2873 | QWQRFNHGV |
| 2874 | QWQSFLTED |
| 2875 | QWRIMTQDP |
| 2876 | QWSQARSQY |
| 2877 | QWSQMDRMQ |
| 2878 | QWVMKHSYH |
| 2879 | QYAITYQEG |
| 2880 | QYAMFEKMS |
| 2881 | QYAMSFTQM |
| 2882 | QYDPYIQGD |
| 2883 | QYDQASDML |
| 2884 | QYFLSMDTE |
| 2885 | QYFQSNNQC |
| 2886 | QYFQTSNSR |
| 2887 | QYGLYKDTQ |
| 2888 | QYRSLDSDH |
| 2889 | QYRTPDEAI |
| 2890 | QYRTQDEAL |
| 2891 | QYVGMYTQQ |
| 2892 | QYVHMLGEC |
| 2893 | RAAGNSIDV |
| 2894 | RACMEGWPK |
| 2895 | RADNRCGQD |
| 2896 | RADSNKQFE |
| 2897 | RAEHVLNAD |
| 2898 | RAEPINHYN |
| 2899 | RAGMCENFG |
| 2900 | RAGMEPNFG |
| 2901 | RAIDCHRLN |
| 2902 | RANAESDQP |
| 2903 | RATLGASLH |
| 2904 | RATQWDSYP |
| 2905 | RAVQLEYCQ |
| 2906 | RCDSNDGQM |
| 2907 | RCDSVEVQY |
| 2908 | RCFFKMEAV |
| 2909 | RCIGKEDMT |
| 2910 | RCVAEPNEP |
| 2911 | RCVDLSMAS |
| 2912 | RCYEQHMWP |
| 2913 | RDDELLEPG |
| 2914 | REAYLNYVA |
| 2915 | REEMFVYMP |
| 2916 | REFELSIGH |
| 2917 | REGKVFDCY |
| 2918 | REMCSTGHF |
| 2919 | REQMVFRDQ |
| 2920 | RFADSKLHD |
| 2921 | RFDFLQSWF |
| 2922 | RFDLMLKDL |
| 2923 | RFDLQMGGG |
| 2924 | RFDMQHCFY |
| 2925 | RFDMSHQGR |
| 2926 | RFDNTTQYG |
| 2927 | RFDQFLMHM |
| 2928 | RFDQYTFLE |
| 2929 | RFEEMDFDM |
| 2930 | RFELLMEDH |
| 2931 | RFELTQNSL |
| 2932 | RFEMSHEQW |
| 2933 | RFEPSMKHC |
| 2934 | RFEPYMKIC |
| 2935 | RFEVSMREA |
| 2936 | RFIQMEITQ |
| 2937 | RFKMQFVTS |
| 2938 | RFSVRDYAP |
| 2939 | RFVPNCMSI |
| 2940 | RFYFTAEMN |
| 2941 | RGDKIHACF |
| 2942 | RGGLQFVEA |
| 2943 | RGHETYGPI |
| 2944 | RGKPTWTEK |
| 2945 | RGLQRVRTC |
| 2946 | RHDEQPFCK |
| 2947 | RHGFKDSGE |
| 2948 | RHGNFEAMA |
| 2949 | RIDDWAHGR |
| 2950 | RIDHAHFQW |
| 2951 | RIETPCHGS |
| 2952 | RIQQQEDLG |
| 2953 | RKKVEVVVE |
| 2954 | RKMWQSHCE |
| 2955 | RLNYRHYSK |
| 2956 | RMAEPVENE |
| 2957 | RMDTSSRAE |
| 2958 | RMDTSVRCE |
| 2959 | RMEGCWGCA |
| 2960 | RMKWYLPCV |
| 2961 | RMMWQSHCD |
| 2962 | RNAKEVPLP |
| 2963 | RNDVWEHVF |
| 2964 | RNLNLQTTI |
| 2965 | RNWLPENYT |
| 2966 | RPEAHPHYA |
| 2967 | RPEANKHYA |
| 2968 | RQDIFPHQD |
| 2969 | RRDCIIPMM |
| 2970 | RSAWMDYTC |
| 2971 | RSDFIAHMH |
| 2972 | RSEHFEHIG |
| 2973 | RSFMCGLIG |
| 2974 | RSGQGEGHM |
| 2975 | RSMLVERNT |
| 2976 | RSTPEAQQY |
| 2977 | RSWTACEID |
| 2978 | RTESWSSLA |
| 2979 | RTEWMKGVD |
| 2980 | RTEYKEGYF |
| 2981 | RTFMNDMCE |
| 2982 | RTFYVMGTE |
| 2983 | RTKYCSAFG |
| 2984 | RTSVEMCTM |
| 2985 | RVDAFNRGT |
| 2986 | RVDIGMGWK |
| 2987 | RVDPFPIAH |
| 2988 | RVDQRHLPT |
| 2989 | RVDVFSYCG |
| 2990 | RVGFMEGGM |
| 2991 | RVLDNPAEY |
| 2992 | RVMEYAAEW |
| 2993 | RVQMSCYTE |
| 2994 | RVQSGHQRG |
| 2995 | RVTMTSETC |
| 2996 | RVVQRYLPT |
| 2997 | RVWECVCSQ |
| 2998 | RWAEMHVQC |
| 2999 | RWDFVWYIQ |
| 3000 | RWDVDHQSC |
| 3001 | RWDVEQMVG |
| 3002 | RWDVMLEDR |
| 3003 | RWEAYGSQN |
| 3004 | RWEIRQMDM |
| 3005 | RWEPVQIFK |
| 3006 | RWHQGPCHP |
| 3007 | RWNTVFKID |
| 3008 | RYDEQPFCK |
| 3009 | RYDSQHQFK |
| 3010 | RYESFTKHP |
| 3011 | RYPCTIVEQ |
| 3012 | RYQPDPMLM |
| 3013 | RYTEMSCDD |
| 3014 | SAAAWWANG |
| 3015 | SAAEKAMDQ |
| 3016 | SADFLFKSF |
| 3017 | SADRLDLVL |
| 3018 | SAHHSHCNP |
| 3019 | SAINMHYIE |
| 3020 | SAKDPLQAN |
| 3021 | SALAHILSL |
| 3022 | SAMCSGSLL |
| 3023 | SAMDSLGVH |
| 3024 | SAMSVMDNM |
| 3025 | SAMTAIRMS |
| 3026 | SAMWFTANG |
| 3027 | SANNDAFLC |
| 3028 | SASHGGLAY |
| 3029 | SATCIPHSR |
| 3030 | SATDMDGAF |
| 3031 | SATFCGINV |
| 3032 | SATLHPLSL |
| 3033 | SATPHLYCH |
| 3034 | SATPHWYCH |
| 3035 | SAVASILNH |
| 3036 | SAVGGCWGA |
| 3037 | SAVGSHSSS |
| 3038 | SAWQSSVCM |
| 3039 | SCCFQIADR |
| 3040 | SCEQDGMDR |
| 3041 | SCFCMQHPI |
| 3042 | SCFLDHASG |
| 3043 | SCFRFTQAW |
| 3044 | SCGCMHEDF |
| 3045 | SCGCSLYQV |
| 3046 | SCGHSLGCF |
| 3047 | SCHTDGQWP |
| 3048 | SCKAHAAAP |
| 3049 | SCLAMDHVY |
| 3050 | SCLQMFSWH |
| 3051 | SCNFEFLWM |
| 3052 | SCNIGQYWP |
| 3053 | SCQHAIANQ |
| 3054 | SCQHPHDWE |
| 3055 | SCQLAIGHE |
| 3056 | SCQPVNEHE |
| 3057 | SCVQNMINC |
| 3058 | SCYSPIADR |
| 3059 | SDGMVHNCW |
| 3060 | SDLFKMFIE |
| 3061 | SDQPMHCWR |
| 3062 | SDQPMSCWG |
| 3063 | SDQPTMCNV |
| 3064 | SELYKCPYF |
| 3065 | SETICEWAP |
| 3066 | SFCKMCSFG |
| 3067 | SFCLFENIT |
| 3068 | SFDRGMWTV |
| 3069 | SFDRMMWTQ |
| 3070 | SFDTCNYFV |
| 3071 | SFEKYAGSD |
| 3072 | SFFYGTGSE |
| 3073 | SFGPLQNCG |
| 3074 | SFGTGCKCL |
| 3075 | SFHPLMHIA |
| 3076 | SFHPTPNDA |
| 307 | SFIGTNRDY |
| 3078 | SFIHMSHND |
| 3079 | SFLLCHQET |
| 3080 | SFLPIGIQE |
| 3081 | SFNTNQASV |
| 3082 | SFQSTEKCG |
| 3083 | SFSFYKCMQ |
| 3084 | SFSGIGPQH |
| 3085 | SFSKSFAAR |
| 3086 | SFSVFNCSH |
| 3087 | SFSVVQEQS |
| 3088 | SFSWIQSGD |
| 3089 | SFSYCDEPV |
| 3090 | SFWQIDDFN |
| 3091 | SGAGNNYHQ |
| 3092 | SGDSKHCHL |
| 3093 | SGEPMASCQ |
| 3094 | SGEPMTSCQ |
| 3095 | SGERWFVYP |
| 3096 | SGHMYQEGL |
| 3097 | SGISDHCHL |
| 3098 | SGPMSDMIS |
| 3099 | SGTGYVHGG |
| 3100 | SHASISHML |
| 3101 | SHASISHVL |
| 3102 | SHAVKHEQA |
| 3103 | SHDTWCVCT |
| 3104 | SHGFIDLQL |
| 3105 | SHLDFPVGR |
| 3106 | SHLWPNLHE |
| 3107 | SHMMQVAQF |
| 3108 | SHNNNPSGE |
| 3109 | SHQQYPAFW |
| 3110 | SHSCDGCGQ |
| 3111 | SHSSCCMTF |
| 3112 | SHTFINRHT |
| 3113 | SHTGVFKHY |
| 3114 | SHTQMELHN |
| 3115 | SIDSFMPIY |
| 3116 | SIEHWCCCQ |
| 3117 | SIFQIGQSP |
| 3118 | SIGQAWQSP |
| 3119 | SIHNAKEYR |
| 3120 | SIHPKQSRY |
| 3121 | SIIFLVSSG |
| 3122 | SIKYEDHGQ |
| 3123 | SILFNPNGR |
| 3124 | SIMPMNCGS |
| 3125 | SIMWRSDWT |
| 3126 | SINNALTIN |
| 3127 | SIQHCRDYY |
| 3128 | SIQLTNQCE |
| 3129 | SIQPCRDYC |
| 3130 | SISHMQARA |
| 3131 | SISSIEGYP |
| 3132 | SIYQLSWHG |
| 3133 | SKNTVGIGH |
| 3134 | SLADQVHVA |
| 3135 | SLDSIFACG |
| 3136 | SLGMDGMGV |
| 3137 | SLGNKLHIT |
| 3138 | SLRSYVDSF |
| 3139 | SLSEMRWAA |
| 3140 | SLSQYFESF |
| 3141 | SLTSSFAVL |
| 3142 | SLYNRYIHF |
| 3143 | SMAICMHDN |
| 3144 | SMDVTHFFE |
| 3145 | SMEPYIFGS |
| 3146 | SMFTHQLAQ |
| 3147 | SMFYVRLVC |
| 3148 | SMGYSEVFG |
| 3149 | SMHCSGYAL |
| 3150 | SMHHWAKGP |
| 3151 | SMHYYDTFE |
| 3152 | SMLQTTEQC |
| 3153 | SMLQTTSQM |
| 3154 | SMMCRCWLM |
| 3155 | SMNPNLTGD |
| 3156 | SMSFYQACH |
| 3157 | SMTNVPNLA |
| 3158 | SMWVPPNAA |
| 3159 | SMYMRVCYT |
| 3160 | SMYTMCTNH |
| 3161 | SMYTTEDIQ |
| 3162 | SNAMMFFCS |
| 3163 | SNDRVKKMD |
| 3164 | SNEANISVE |
| 3165 | SNMHYMRGS |
| 3166 | SNMPLMNMA |
| 3167 | SNSFYQTLG |
| 3168 | SNSHPFQSH |
| 3169 | SNSLYKKER |
| 3170 | SNWAIVKCD |
| 3171 | SNWQIVKTD |
| 3172 | SPISVNNTL |
| 3173 | SPLPMYNQE |
| 3174 | SPMFYGSAH |
| 3175 | SPNMFCEMQ |
| 3176 | SPYQYQLAE |
| 3177 | SQCVMQFPL |
| 3178 | SQGAGHCGN |
| 3179 | SQGPKHCGD |
| 3180 | SQIDLCVDM |
| 3181 | SQILEDLMG |
| 3182 | SQKQFSMQH |
| 3183 | SQKTICGSY |
| 3184 | SQMNCYGGC |
| 3185 | SQNMSVTQY |
| 3186 | SQNYHSMYA |
| 3187 | SQQHCQHDQ |
| 3188 | SQSFYFNHD |
| 3189 | SQTYVDMYA |
| 3190 | SRCMSSMCY |
| 3191 | SREYFATTF |
| 3192 | SREYFETTF |
| 3193 | SRGYRESDS |
| 3194 | SRKFCEGTP |
| 3195 | SRSPMMDMQ |
| 3196 | SSAAIYADF |
| 3197 | SSACYYAKG |
| 3198 | SSASSSEIC |
| 3199 | SSDCNRRCH |
| 3200 | SSEWLESAE |
| 3201 | SSFPEQQCH |
| 3202 | SSFYVQQCP |
| 3203 | SSGDSYQDY |
| 3204 | SSGHYNQLI |
| 3205 | SSGIMCADN |
| 3206 | SSGITAQCT |
| 3207 | SSHEICHDG |
| 3208 | SSHQNIGQP |
| 3209 | SSILMQMNC |
| 3210 | SSIQMQMQC |
| 3211 | SSISFQALG |
| 3212 | SSLYKFNHH |
| 3213 | SSMQQQLTW |
| 3214 | SSQHVNGVW |
| 3215 | SSQRITCEW |
| 3216 | SSQWSSGMY |
| 3217 | SSQYNTCEW |
| 3218 | SSTGEIQYG |
| 3219 | SSTGFDCEQ |
| 3220 | SSTGYPRGG |
| 3221 | SSTRDVCFC |
| 3222 | SSTSSSEIC |
| 3223 | SSVEFPWQT |
| 3224 | SSVPSMQCG |
| 3225 | SSYPESGWQ |
| 3226 | STAACTTWC |
| 3227 | STAHIIPDN |
| 3228 | STCHQWDAG |
| 3229 | STDDRMDAA |
| 3230 | STEQCGEWY |
| 3231 | STEVRWTGA |
| 3232 | STGWSAIAN |
| 3233 | STHQPCAQN |
| 3234 | STLQNIQTH |
| 3235 | STLTPVKLE |
| 3236 | STMQVIDIS |
| 3237 | STNEVWASM |
| 3238 | STNHVDDPL |
| 3239 | STQNNDMYC |
| 3240 | STRLIEQFG |
| 3241 | STRVMEQFG |
| 3242 | STSPYHRQN |
| 3243 | STTHRDNYE |
| 3244 | STTPQMHVS |
| 3245 | STTRFNFWN |
| 3246 | STWMSWEAI |
| 3247 | SVADQVQVA |
| 3248 | SVAQRPEMI |
| 3249 | SVAQRSEMF |
| 3250 | SVCQTTQHQ |
| 3251 | SVDLGTMEQ |
| 3252 | SVEQMRKIP |
| 3253 | SVHMLDDFM |
| 3254 | SVIPKHKNS |
| 3255 | SVIQTEHFD |
| 3256 | SVLCLSMGG |
| 3257 | SVNTMWYPD |
| 3258 | SVRMFDAQY |
| 3259 | SVRVQTWIG |
| 3260 | SVSPCFCNS |
| 3261 | SVSYESTIF |
| 3262 | SVTHDLHSF |
| 3263 | SVVFSAAEV |
| 3264 | SVVVVQTGK |
| 3265 | SVYMMTEGW |
| 3266 | SWAMISTCS |
| 3267 | SWDHLFTDQ |
| 3268 | SWISKGHCE |
| 3269 | SWLFSQDLK |
| 3270 | SWMWITNPN |
| 3271 | SWQSFILCE |
| 3272 | SWSERCSFE |
| 3273 | SWVKQDTMA |
| 3274 | SWVMLLPIH |
| 3275 | SYCHIMGVQ |
| 3276 | SYDAEQRNG |
| 3277 | SYESFRSMH |
| 3278 | SYGVIMTEE |
| 3279 | SYNPWNQHA |
| 3280 | SYQFAQYWL |
| 3281 | SYQRWDSCD |
| 3282 | SYSPCFCRS |
| 3283 | TAAFAYKYE |
| 3284 | TAALFEGSK |
| 3285 | TAAQKFMAN |
| 3286 | TAAYGDRYD |
| 3287 | TACDFISLT |
| 3288 | TADEFSCCQ |
| 3289 | TADTRVNHY |
| 3290 | TAEAFQKFI |
| 3291 | TAENIQTHN |
| 3292 | TAEVRWPCP |
| 3293 | TAFECMICG |
| 3294 | TAFECMIDG |
| 3295 | TAFNAQCGQ |
| 3296 | TAGGSDAEK |
| 3297 | TAGRFNYFD |
| 3298 | TAHLETDQN |
| 3299 | TAHLHMGNI |
| 3300 | TAIPMNELV |
| 3301 | TAITGFNIH |
| 3302 | TAKAQNAFE |
| 3303 | TAMASMWLS |
| 3304 | TAMHSAYHS |
| 3305 | TANKCDCSD |
| 3306 | TANVYRSGQ |
| 3307 | TASHCSFNQ |
| 3308 | TASMLMMTP |
| 3309 | TASNGCINS |
| 3310 | TASPMVEHW |
| 3311 | TATFSDFGF |
| 3312 | TATLSMWLS |
| 3313 | TAVEKNTLW |
| 3314 | TAVKLEQGS |
| 3315 | TAVNYWNTS |
| 3316 | TAYAFDCNC |
| 3317 | TCAPSSSMD |
| 3318 | TCCMWTFIW |
| 3319 | TCCSFSFYS |
| 3320 | TCCTVERES |
| 3321 | TCDHASDIE |
| 3322 | TCDYNVDTT |
| 3323 | TCEAAQAQH |
| 3324 | TCGKAMEQY |
| 3325 | TCGLWKNLV |
| 3326 | TCGTTVRCG |
| 3327 | TCIHVAGVP |
| 3328 | TCISNHHTC |
| 3329 | TCLPNPMQL |
| 3330 | TCQQMGDIG |
| 3331 | TCQYEMVCD |
| 3332 | TCQYVMLCD |
| 3333 | TCTSGGSWL |
| 3334 | TCVEFMQWN |
| 3335 | TCVFTDHQC |
| 3336 | TCYFFGEQE |
| 3337 | TDAGMFAHD |
| 3338 | TDDPIHNFE |
| 3339 | TDEQCKFGH |
| 3340 | TEGPMHRAW |
| 3341 | TELSYDAFL |
| 3342 | TEQFCGIML |
| 3343 | TESMVMYER |
| 3344 | TFCCMDMIN |
| 3345 | TFDLMQAIR |
| 3346 | TFDPFTTSQ |
| 3347 | TFEPCQSGV |
| 3348 | TFHQSIVGE |
| 3349 | TFISLQQSS |
| 3350 | TFLMLVDDQ |
| 3351 | TFNIAYVQS |
| 3352 | TFSQEGVSI |
| 3353 | TFVIVDDQN |
| 3354 | TFWSSSNYE |
| 3355 | TGGMYYVQN |
| 3356 | TGKWLVYQA |
| 3357 | TGLFIRTMK |
| 3358 | TGLLIPNGK |
| 3359 | TGNDHHEVI |
| 3360 | TGNYVHSNQ |
| 3361 | TGRIKDAGP |
| 3362 | THAQIGTFD |
| 3363 | THAVTGILY |
| 3364 | THECYQMWH |
| 3365 | THEMVNENG |
| 3366 | THGYMQSHN |
| 3367 | THMNVVRSF |
| 3368 | THNTVPVER |
| 3369 | THQNTWLFY |
| 3370 | THRHSDMGF |
| 3371 | THTQYWAMQ |
| 3372 | TIAVIGSAA |
| 3373 | TICKTEIGH |
| 3374 | TICNCMFGA |
| 3375 | TICQTEFGH |
| 3376 | TIDGMMGDH |
| 3377 | TIEAKWECM |
| 3378 | TIEANWEYQ |
| 3379 | TIECEQRET |
| 3380 | TIEPGCQCT |
| 3381 | TIEWKLHMG |
| 3382 | TIFYYGGNI |
| 3383 | TIGEMVNPS |
| 3384 | TIHQSQFIC |
| 3385 | TIHSSVSAS |
| 3386 | TILCDQQQA |
| 3387 | TIMNWNDAK |
| 3388 | TIMSDGHTH |
| 3389 | TINLTTQSV |
| 3390 | TIRLTEQSV |
| 3391 | TISELQSEF |
| 3392 | TISIFTNCR |
| 3393 | TISLEYCTF |
| 3394 | TISWLGGCH |
| 3395 | TIVNMQMGN |
| 3396 | TIVWMCVNW |
| 3397 | TKEPCTVGV |
| 3398 | TKEPTTVGF |
| 3399 | TKFHMMIQT |
| 3400 | TKNCVASTF |
| 3401 | TKQFELKDE |
| 3402 | TKQFETKSE |
| 3403 | TKQHYDSPA |
| 3404 | TLCGLDSEY |
| 3405 | TLCGNTQCP |
| 3406 | TLEGSDSEY |
| 3407 | TLNNLHRVA |
| 3408 | TLPLFWRLK |
| 3409 | TLQMVMNKG |
| 3410 | TLRDSECYS |
| 3411 | TLTYYEEGE |
| 3412 | TLVPCQTCE |
| 3413 | TLVRGMHQI |
| 3414 | TLVRSDYDP |
| 3415 | TLVRSDYYP |
| 3416 | TLYMHEKLG |
| 3417 | TMANLCWRT |
| 3418 | TMCPHGIRC |
| 3419 | TMCPKMWQH |
| 3420 | TMDQGHCMI |
| 3421 | TMIHHQNAD |
| 3422 | TMLHSQNME |
| 3423 | TMMCDQQQA |
| 3424 | TMPPFPCHM |
| 3425 | TMPYSQWAM |
| 3426 | TMSDRQQDT |
| 3427 | TMSGYCCSF |
| 3428 | TMSVHYGQG |
| 3429 | TMTSTLQST |
| 3430 | TMVAGKMRC |
| 3431 | TMWEDNQNM |
| 3432 | TMYFNANTQ |
| 3433 | TMYTKTSMF |
| 3434 | TNEGVLHHT |
| 3435 | TNHELAEKP |
| 3436 | TNHELAMYP |
| 3437 | TNHPKCKKD |
| 3438 | TNIQKAMGA |
| 3439 | TNKTLPATF |
| 3440 | TNLVYQFLE |
| 3441 | TNQSQNCMF |
| 3442 | TPAMEISMF |
| 3443 | TPCISHCEP |
| 3444 | TPEEFTMSP |
| 3445 | TPGTLQGHH |
| 3446 | TPLMMCQAS |
| 3447 | TPNSFNCAS |
| 3448 | TPTLESNCR |
| 3449 | TQAECITVP |
| 3450 | TQCHDMWGH |
| 3451 | TQDFCSVCD |
| 3452 | TQECYYDQF |
| 3453 | TQEPIHHQN |
| 3454 | TQFPVEYMS |
| 3455 | TQGFYNVGV |
| 3456 | TQQNCMTGN |
| 3457 | TQSGYHFWC |
| 3458 | TQTILNCGY |
| 3459 | TQVLMEMCT |
| 3460 | TQWTEKRQA |
| 3461 | TRCFIDQGI |
| 3462 | TRIEQEWNH |
| 3463 | TSDASVQQS |
| 3464 | TSDMTMENQ |
| 3465 | TSDWWCQKC |
| 3466 | TSEGGIIYN |
| 3467 | TSFGISWLH |
| 3468 | TSFNMICNS |
| 3469 | TSGQPGISW |
| 3470 | TSHINIWGQ |
| 3471 | TSHWSAVQM |
| 3472 | TSHYITFTP |
| 3473 | TSLGQWTHF |
| 3474 | TSLHSSMGH |
| 3475 | TSLHYQFFA |
| 3476 | TSMSFDSTH |
| 3477 | TSNFEPFSP |
| 3478 | TSQCAHQWS |
| 3479 | TSQRNIEAG |
| 3480 | TSQRNIEVA |
| 3481 | TSQWLMNMQ |
| 3482 | TSSFMDCVD |
| 3483 | TSTIEELAQ |
| 3484 | TSTMAGPCI |
| 3485 | TSTQRQDVF |
| 3486 | TSTRLYPVD |
| 3487 | TSVMCNQTR |
| 3488 | TSVTIQFWE |
| 3489 | TSVWWCKKE |
| 3490 | TSVWWCQKE |
| 3491 | TSWIQTWGS |
| 3492 | TTAIRDLTM |
| 3493 | TTEKYWEHR |
| 3494 | TTFIIPIPS |
| 3495 | TTFRNINPA |
| 3496 | TTGPNPNCY |
| 3497 | TTGWRYQTS |
| 3498 | TTHQLAVVY |
| 3499 | TTKTCAEGG |
| 3500 | TTLAHILSL |
| 3501 | TTNGYMSMD |
| 3502 | TTNLPQPEM |
| 3503 | TTNQWMNYY |
| 3504 | TTQFCGSML |
| 3505 | TTSTSIIYH |
| 3506 | TTTGINYHH |
| 3507 | TTTIYQQQP |
| 3508 | TTVDNGFHS |
| 3509 | TTYQSGWHG |
| 3510 | TTYYCPTSQ |
| 3511 | TVAPHWIDH |
| 3512 | TVAPSNCAN |
| 3513 | TVDMLHMAS |
| 3514 | TVDQFDKYN |
| 3515 | TVDQYCVRA |
| 3516 | TVDSEVNAD |
| 3517 | TVENMPTYP |
| 3518 | TVENNYGGY |
| 3519 | TVKMIHVDW |
| 3520 | TVMHDHCDN |
| 3521 | TVMLESNCR |
| 3522 | TVNAESKGC |
| 3523 | TVNHWQQIC |
| 3524 | TVNLCDIYK |
| 3525 | TVNQSVADM |
| 3526 | TVQQDGAQM |
| 3527 | TVQQDGHQM |
| 3528 | TVTHMSQHC |
| 3529 | TVYKNEQVA |
| 3530 | TWAPSMYWM |
| 3531 | TWCQIQGMG |
| 3532 | TWDSCQDFY |
| 3533 | TWDSYIQRD |
| 3534 | TWEHCQYFE |
| 3535 | TWHETDDFI |
| 3536 | TWIEQEWNH |
| 3537 | TWIQEGHYG |
| 3538 | TWIQHMGTF |
| 3539 | TWISSQHCH |
| 3540 | TWMHMMSYR |
| 3541 | TWNCAGLLP |
| 3542 | TWNMIGQLP |
| 3543 | TWQFYQHGC |
| 3544 | TWSFMGCVD |
| 3545 | TWSHCSYFE |
| 3546 | TWSNYQWYQ |
| 3547 | TWTEQEWNH |
| 3548 | TWVTTMAMQ |
| 3549 | TWYQIEGAA |
| 3550 | TWYQKTGAA |
| 3551 | TWYTKRTEG |
| 3552 | TYDNEMSET |
| 3553 | TYDQMQTEF |
| 3554 | TYEQSQVLR |
| 3555 | TYEWYMGDW |
| 3556 | TYFLLHASG |
| 3557 | TYGKAMEQY |
| 3558 | TYGQWMEKA |
| 3559 | TYISNQQSS |
| 3560 | TYKTHTIGG |
| 3561 | TYLFSNQSR |
| 3562 | TYLYRKTVY |
| 3563 | TYPLAWLKQ |
| 3564 | TYSPMSGIE |
| 3565 | TYSPMSGIV |
| 3566 | TYSPMSGKA |
| 3567 | TYSPMSGKD |
| 3568 | TYSQMQTFF |
| 3569 | TYSSKYQGD |
| 3570 | TYSSWYQGE |
| 3571 | TYVKFLVHP |
| 3572 | TYVYNHKAF |
| 3573 | VAANTYCYG |
| 3574 | VADSSWNQP |
| 3575 | VADYTACPM |
| 3576 | VAGNATPSL |
| 3577 | VAGPMWVQT |
| 3578 | VAHCHVATE |
| 3579 | VAHPDMKYI |
| 3580 | VAILGLNPT |
| 3581 | VAIPVDYHT |
| 3582 | VAIYKTECI |
| 3583 | VAIYPGIIE |
| 3584 | VALIIDEWT |
| 3585 | VALSRNSIG |
| 3586 | VANLDYDPS |
| 3587 | VANPINRTA |
| 3588 | VANQNYAIC |
| 3589 | VAQSIQEVA |
| 3590 | VARPTMEEM |
| 3591 | VATALMMSL |
| 3592 | VATHIQVMH |
| 3593 | VAVVAEHCA |
| 3594 | VAYAFQCNC |
| 3595 | VAYLIQDAL |
| 3596 | VCEQWNEHL |
| 3597 | VCETIPIKE |
| 3598 | VCMLHVNCE |
| 3599 | VCMLHVNFE |
| 3600 | VCQRILELR |
| 3601 | VCRQMNDER |
| 3602 | VCTLIQDAH |
| 3603 | VCTPIIDCS |
| 3604 | VCVPIIHET |
| 3605 | VCYNADVPG |
| 3606 | VDAWDNTAC |
| 3607 | VDTHMNQKQ |
| 3608 | VDTLNQDAH |
| 3609 | VDTVWMLPG |
| 3610 | VEMQANKGL |
| 3611 | VESPVLTMG |
| 3612 | VESPVYNMG |
| 3613 | VFEGGFPYI |
| 3614 | VFFSVYQDK |
| 3615 | VFGIMPCGF |
| 3616 | VFGIMPCWQ |
| 3617 | VFHSMYNSM |
| 3618 | VFNKAIEWK |
| 3619 | VFNLAQNWH |
| 3620 | VFNPYIDVG |
| 3621 | VFNTFDESM |
| 3622 | VFNTFDQQM |
| 3623 | VFRSASNKH |
| 3624 | VFRSVDVVD |
| 3625 | VFRVKCAAD |
| 3626 | VFWMQLRDH |
| 3627 | VFYLSSQSC |
| 3628 | VGDHKNCMN |
| 3629 | VGEKTHGPL |
| 3630 | VGEPHFHSD |
| 3631 | VGGYSNQWV |
| 3632 | VGHWKVQSC |
| 3633 | VGHWKVQSY |
| 3634 | VGIMRNDTG |
| 3635 | VGIQTQSCF |
| 3636 | VGKGIEKVQ |
| 3637 | VGLALNCES |
| 3638 | VGNPYHHGR |
| 3639 | VGQGIEKVQ |
| 3640 | VGQHIWAQD |
| 3641 | VGQREVPSD |
| 3642 | VGRWHYCTE |
| 3643 | VGTSFACSN |
| 3644 | VGVQCSNGG |
| 3645 | VHAPNNLVA |
| 3646 | VHDKWNAHT |
| 3647 | VHDMCQWSD |
| 3648 | VHELFMDMD |
| 3649 | VHKCADYMC |
| 3650 | VHQPTQMHH |
| 3651 | VHQPTQSHH |
| 3652 | VHSFSEVTM |
| 3653 | VHTTYCQRE |
| 3654 | VHYRLCEHW |
| 3655 | VIDRLMACS |
| 3656 | VIEFFKAQC |
| 3657 | VIFSMYTCF |
| 3658 | VIGLCMGIV |
| 3659 | VIGLCTGIV |
| 3660 | VIIEEQDNS |
| 3661 | VIKHALAEI |
| 3662 | VIMYWHHCS |
| 3663 | VIQSYQAVF |
| 3664 | VKESDNWQQ |
| 3665 | VKFAWQKIN |
| 3666 | VKMEYQGGR |
| 3667 | VKQLRQDGF |
| 3668 | VLAMRVRHK |
| 3669 | VLSNWEVAA |
| 3670 | VMAPYMWDP |
| 3671 | VMDCVNAYW |
| 3672 | VMDFYMSGP |
| 3673 | VMDQYKLKV |
| 3674 | VMEFYKAQC |
| 3675 | VMEFYMSPP |
| 3676 | VMEHWGSQY |
| 3677 | VMEWWGSLY |
| 3678 | VMGNSMCLF |
| 3679 | VMGPCMNYD |
| 3680 | VMHINEQAM |
| 3681 | VMMHYGGFY |
| 3682 | VMQGFNYGF |
| 3683 | VMQMINLAH |
| 3684 | VMSDEWQTI |
| 3685 | VMTICQSQE |
| 3686 | VNADITFGA |
| 3687 | VNGREPRWK |
| 3688 | VNGREPRWQ |
| 3689 | VNITYTLQP |
| 3690 | VNMMCGHES |
| 3691 | VNRIRDMSV |
| 3692 | VNVDCNVQQ |
| 3693 | VPEISWSNH |
| 3694 | VPICCNLPM |
| 3695 | VPLGFNFAG |
| 3696 | VQHMSQSDY |
| 3697 | VQHMSQSGT |
| 3698 | VQHQEEFQT |
| 3699 | VQIRTIRQE |
| 3700 | VQKQEHAKY |
| 3701 | VQMWTIRQE |
| 3702 | VQNWKLDKI |
| 3703 | VQQFCFQHQ |
| 3704 | VRIQFSQAL |
| 3705 | VRQMIDHLA |
| 3706 | VRSGQQAIF |
| 3707 | VSAAWGQME |
| 3708 | VSAFGHKAG |
| 3709 | VSASFQQHV |
| 3710 | VSAVVGQME |
| 3711 | VSCEQGKIK |
| 3712 | VSDLWQSQE |
| 3713 | VSEHCQEGN |
| 3714 | VSEHCQEVF |
| 3715 | VSEPFEHCD |
| 3716 | VSEPHFHKD |
| 3717 | VSERDKIYG |
| 3718 | VSHNQIYGM |
| 3719 | VSLGTHAHH |
| 3720 | VSMNTPFCL |
| 3721 | VSMWYDRDD |
| 3722 | VSSAMTWMT |
| 3723 | VSSQYNHLE |
| 3724 | VSTRFPMAQ |
| 3725 | VSTRMYACS |
| 3726 | VSVNEDRAG |
| 3727 | VTAATGAST |
| 3728 | VTAPSNLVA |
| 3729 | VTCRQEGGG |
| 3730 | VTDQIWVHN |
| 3731 | VTDRADQNY |
| 3732 | VTDRIEQYH |
| 3733 | VTEHCVRQP |
| 3734 | VTEIRPSFG |
| 3735 | VTELNMAAW |
| 3736 | VTEPLEHCF |
| 3737 | VTESWNEHL |
| 3738 | VTGFGPATP |
| 3739 | VTHLSASSD |
| 3740 | VTIPVDYFT |
| 3741 | VTNVCQCEA |
| 3742 | VTQFCLSGK |
| 3743 | VTQSIQEVA |
| 3744 | VTSCGQENC |
| 3745 | VTTGMQIEE |
| 3746 | VTVAVQPCS |
| 3747 | VTVNGNAGW |
| 3748 | VVCDMLTSL |
| 3749 | VVCQPVPCV |
| 3750 | VVDNPMMGQ |
| 3751 | VVGLEGACK |
| 3752 | VVHEITINS |
| 3753 | VVHIEWRPT |
| 3754 | VVIPMPCCS |
| 3755 | VVKCKPQFC |
| 3756 | VVPPCMDCG |
| 3757 | VVSARVNVQ |
| 3758 | VVTAEFEFV |
| 3759 | VVVLEGSCK |
| 3760 | VWEIFDAMS |
| 3761 | VWSGQQAIF |
| 3762 | VWVCQHWVQ |
| 3763 | VYDEWTKNV |
| 3764 | VYEQMAYCP |
| 3765 | VYGHMQNWF |
| 3766 | VYLPYYWQF |
| 3767 | VYPDSAVQL |
| 3768 | VYQHWWRVH |
| 3769 | VYQHYFVDQ |
| 3770 | VYSFFYQNE |
| 3771 | VYTMRSALE |
| 3772 | VYVLASAQK |
| 3773 | VYYCEIREH |
| 3774 | WADDKKHYG |
| 3775 | WAECRYQTT |
| 3776 | WAGVEQRGE |
| 3777 | WAGWIPSSI |
| 3778 | WAIPMPHCQ |
| 3779 | WCCRPINAN |
| 3780 | WCDSVEPPG |
| 3781 | WCEHVIMNP |
| 3782 | WDAYYTNGI |
| 3783 | WDGQKMWYC |
| 3784 | WDSPKNFWI |
| 3785 | WDVRDHAFY |
| 3786 | WEVDVCNDR |
| 3787 | WEWNAKHKC |
| 3788 | WFEHASHVF |
| 3789 | WFKGVDHRQ |
| 3790 | WFMYLTNAL |
| 3791 | WFQTMDHVM |
| 3792 | WFYRHNHVE |
| 3793 | WGDARMIGP |
| 3794 | WGDKQATDR |
| 3795 | WHDYQLAAE |
| 3796 | WHFKIHVQP |
| 3797 | WHFLMIFYI |
| 3798 | WHGFGKWSR |
| 3799 | WHSHYLLHH |
| 3800 | WHVSHWPKL |
| 3801 | WHYPWDDWH |
| 3802 | WIGSKNSCE |
| 3803 | WITSKIKSD |
| 3804 | WITSKINSD |
| 3805 | WIYPFSFGL |
| 3806 | WKAIVLEYW |
| 3807 | WKWWADYLA |
| 3808 | WLFMHRWAD |
| 3809 | WLIEIERHG |
| 3810 | WLPLCFPCA |
| 3811 | WLPLCFPWA |
| 3812 | WLPMVMNKG |
| 3813 | WMEYINHEA |
| 3814 | WMMQYIIFP |
| 3815 | WMSEQMFKG |
| 3816 | WNNVELSTP |
| 3817 | WNQHKRDQR |
| 3818 | WNRELQPPG |
| 3819 | WNSCHQTWI |
| 3820 | WNYWLLPDD |
| 3821 | WPDSESEEV |
| 3822 | WPHTMDHVM |
| 3823 | WPKQGNAME |
| 3824 | WPQQGNAME |
| 3825 | WQAVIYANQ |
| 3826 | WQKLQGNRG |
| 3827 | WRFCIKIGM |
| 3828 | WSQSVAKML |
| 3829 | WSTGCMQGM |
| 3830 | WTANYDYYE |
| 3831 | WTCWLLGWF |
| 3832 | WVAHTTGHA |
| 3833 | WVEIKVMDQ |
| 3834 | WVYSLGRAE |
| 3835 | WWNHWLWNG |
| 3836 | WWRASHRDD |
| 3837 | WWWLSTGNQ |
| 3838 | YADMFQCEK |
| 3839 | YADMFQCVK |
| 3840 | YAGPNMHWG |
| 3841 | YAHLLNQQA |
| 3842 | YAILHQTSK |
| 3843 | YAITQSCFT |
| 3844 | YAITQSRST |
| 3845 | YAIWNMHFT |
| 3846 | YALQITRSQ |
| 3847 | YALVMGIHN |
| 3848 | YAQLMYIDP |
| 3849 | YASPQSGAW |
| 3850 | YATERYCVC |
| 3851 | YATSMHAHK |
| 3852 | YCCNQEPFA |
| 3853 | YCEPVQDNF |
| 3854 | YCISLIYHH |
| 3855 | YCQPVDQFY |
| 3856 | YCYLSSASC |
| 3857 | YCYVCQMTT |
| 3858 | YDNSVGHSL |
| 3859 | YDTNQIVCP |
| 3860 | YEDNNVMQI |
| 3861 | YEGRPSDAQ |
| 3862 | YESCNLTRG |
| 3863 | YFDWFYNHM |
| 3864 | YFDYHERKG |
| 3865 | YFGTSLRAQ |
| 3866 | YFHWHDCIG |
| 3867 | YFTTMHVMG |
| 3868 | YFWSNHFFR |
| 3869 | YGNLMGVGP |
| 3870 | YGTLCGVGI |
| 3871 | YHAPMLMCN |
| 3872 | YHIWCVYWA |
| 3873 | YIELRHREA |
| 3874 | YIFACGVKG |
| 3875 | YKPNYEIME |
| 3876 | YKQRISHWL |
| 3877 | YLGMWLAPV |
| 3878 | YLIPWPRLG |
| 3879 | YLMPHWFII |
| 3880 | YLSDMCWRG |
| 3881 | YLSFMCAIQ |
| 3882 | YLTLQGMQE |
| 3883 | YMDPVVAMN |
| 3884 | YMDRGNIGE |
| 3885 | YMERGNIGE |
| 3886 | YMSNIIHHN |
| 3887 | YMTTQTGYP |
| 3888 | YMTYIQSQE |
| 3889 | YNKQMMNCP |
| 3890 | YPAPYYPAE |
| 3891 | YPIQCSVSF |
| 3892 | YPYQGHNWT |
| 3893 | YQAHYMSMT |
| 3894 | YQDIHQCIF |
| 3895 | YQFCKPSDD |
| 3896 | YQFCKPSDE |
| 3897 | YQGEVERAF |
| 3898 | YQGYEPHMH |
| 3899 | YQHHYHLPW |
| 3900 | YQMQMTYVW |
| 3901 | YQQQMFERQ |
| 3902 | YQSQGIWIE |
| 3903 | YQTLCMHQD |
| 3904 | YQTVFTLYF |
| 3905 | YSAHWCKGD |
| 3906 | YSGVRVTGY |
| 3907 | YSKHQMCID |
| 3908 | YSNSTIMNL |
| 3909 | YSQFFSAEE |
| 3910 | YSQLMHGGH |
| 3911 | YSQSWNRLG |
| 3912 | YSQSWNRLW |
| 3913 | YSSELECQW |
| 3914 | YSTICNFSI |
| 3915 | YSTIHAGHP |
| 3916 | YSTTMHVMG |
| 3917 | YSVSFGCQS |
| 3918 | YTDPVLHNP |
| 3919 | YTGMSCTMK |
| 3920 | YTHEESAHF |
| 3921 | YTKTYVDCF |
| 3922 | YTQHLVGQY |
| 3923 | YTTFTEHWQ |
| 3924 | YVAEAHCSY |
| 3925 | YVCPSVNWN |
| 3926 | YVEDRNGWH |
| 3927 | YVENRNGWH |
| 3928 | YVHDHLHQQ |
| 3929 | YVNIHNRHG |
| 3930 | YVNIHNRNG |
| 3931 | YVNKDEFRF |
| 3932 | YVSVTLQNS |
| 3933 | YVTASWHGF |
| 3934 | YVYIRKNAL |
| 3935 | YWQSMLWQG |
| 3936 | YYCLRFVLE |
| 3937 | YYTQFSSDY |

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 3 (SEQ ID NO: 3938-SEQ ID NO: 4237) that is expected to confer CNS tissue tropism or preference on a recombinant AAV virion (rAAV), based on a primary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 3 (SEQ ID NO: 3938-SEQ ID NO: 4237) at the region corresponding to amino acid residues 445 to 453 in AAV5 VP2 or the region corresponding to amino acid residues 389 to 397 in AAV5 VP3.

TABLE 3
Additional Sequences of 581-589 Regions

| SEQ ID | |
|---|---|
| NO | Sequence |
| 3938 | EAKWSGLGR |
| 3939 | QAYFYLMSS |
| 3940 | LFRQNQQFY |
| 3941 | QWRGYLSKA |
| 3942 | SFGWFDAHI |
| 3943 | DANQYHVSY |
| 3944 | QAFTNLDQM |
| 3945 | LGVYNQSFE |
| 3946 | HQSFVQCVH |
| 3947 | QYQSQWMSF |
| 3948 | HYSQYQNFT |
| 3949 | GNQQVNQQM |
| 3950 | LFNSMHQFD |
| 3951 | QASVYFWQQ |
| 3952 | RQSWVFVNH |
| 3953 | DAYKVQCYC |
| 3954 | EFMTSMTMM |
| 3955 | QFALINFDQ |
| 3956 | DASMIIMML |
| 3957 | QFSLVSLEA |
| 3958 | QHLFSNIIP |
| 3959 | TWFDMVMCD |
| 3960 | HAEYKDAGS |
| 3961 | QNSFYSQMG |
| 3962 | ELQQIYCAG |
| 3963 | SFEHLDCLL |
| 3964 | FYAFNMSDN |
| 3965 | LSWMQYEFL |
| 3966 | QSFNYLFYG |
| 3967 | RWQFIHLSK |
| 3968 | TFLMNFALD |
| 3969 | RWDTYGSWM |
| 3970 | GQQQSFSYQ |
| 3971 | LFTHCILNE |
| 3972 | PTDNINFQM |
| 3973 | SFVTFNNSW |
| 3974 | FTSSYKCYF |
| 3975 | QAHHYNILN |
| 3976 | LFDQFYSWS |
| 3977 | QWMGLIRWK |
| 3978 | HYLPFMYMQ |
| 3979 | LFNRSLRYE |
| 3980 | FWQLKHGAC |
| 3981 | LSLNELALL |
| 3982 | INFEMMDTD |
| 3983 | LSHTYIEIA |
| 3984 | DVAKYMYML |
| 3985 | PINSGIYWH |
| 3986 | ECSTSMGQF |
| 3987 | TSVFYQSNG |
| 3988 | TYSSFNFTC |
| 3989 | CSSFYFESK |
| 3990 | EATYTNAAC |
| 3991 | DAWQSNGWQ |
| 3992 | MIILILPYS |
| 3993 | EADTHWHHN |
| 3994 | RHLFWDILE |
| 3995 | EFMNASHHF |
| 3996 | QFQSVMQQY |
| 3997 | MWNQQDCKD |
| 3998 | RSEPCKNYG |
| 3999 | SIIQANSSF |
| 4000 | DATTSYVYC |
| 4001 | RFDINLDQF |
| 4002 | QWTENVMAR |
| 4003 | DSFYSSQQA |
| 4004 | PVTQYMSFM |
| 4005 | HSQVQQMQI |
| 4006 | FVQFWVGYQ |
| 4007 | YSKWIGIDQ |
| 4008 | YFSQYNVQS |
| 4009 | CAAHATAHQ |
| 4010 | VHSQITCED |
| 4011 | VIFNFYSMG |
| 4012 | LFSLRMIDR |
| 4013 | SWYCMQAIE |
| 4014 | LFLTLSTFN |
| 4015 | QAIGYRIWC |
| 4016 | QILLFNACR |
| 4017 | RTMDTNMFD |
| 4018 | LIQNCSSFG |
| 4019 | QWMVQHTNF |
| 4020 | LFSYVQNVH |
| 4021 | SMMFYRHCQ |
| 4022 | DATWIMQDN |
| 4023 | FAFQSNIEA |
| 4024 | SFDLMFRFY |
| 4025 | IAITWTSQC |
| 4026 | HGFYVFFFI |
| 4027 | MWMLKIDCT |
| 4028 | GFDYQGLSE |
| 4029 | RWFLQEQSN |
| 4030 | WFCSFYALQ |
| 4031 | HFMSYNSFF |
| 4032 | RSFLSYGLK |
| 4033 | NPMVTLANR |
| 4034 | TAQSIGSQH |
| 4035 | HFLSSMHQG |
| 4036 | LAMSSMFFH |
| 4037 | RVSNMLNCT |
| 4038 | DAQMIMSIE |
| 4039 | KIFTIHQFE |
| 4040 | DNDFFQAQF |
| 4041 | NIHYYGSGY |
| 4042 | RFDLFHEQY |
| 4043 | QFMLIEARF |
| 4044 | LFLYYHSAT |
| 4045 | SCSSHSQCC |
| 4046 | NFSSIGCHF |
| 4047 | IASWNQYHF |
| 4048 | EASSMQFWA |
| 4049 | QYEKSMTAF |
| 4050 | DAHHIMEGN |
| 4051 | LQIWYTEMF |
| 4052 | QMGNIYSSV |
| 4053 | NQSFYLHMS |
| 4054 | TSESSGVGG |
| 4055 | ELSFYQQFF |
| 4056 | MMVYFMQQA |
| 4057 | QAWFYQFDK |
| 4058 | GSDIHHEQF |
| 4059 | CAYMLGISQ |
| 4060 | EAMWIHFFG |
| 4061 | FFAANGQGC |
| 4062 | RFDLQNLMK |
| 4063 | QLRMWCCIH |
| 4064 | VDSTSSAFM |
| 4065 | RYQFWLNFT |
| 4066 | RFDSVQQYF |
| 4067 | IIHRYARWC |
| 4068 | DATHMITQS |
| 4069 | LHFFYSHSN |
| 4070 | TAAPIHVWM |
| 4071 | QALHIDCLH |
| 4072 | KFSTMLHAT |
| 4073 | NYSSSFVQM |
| 4074 | TFHQTSHWF |
| 4075 | SRLLNFCCI |
| 4076 | QADYSYSFL |
| 4077 | DYNRSDSYR |
| 4078 | LYGHKMQEN |
| 4079 | RFENSLTNE |
| 4080 | VAMPNMEYM |
| 4081 | AMCFYYMLE |
| 4082 | KGEQSIIFI |
| 4083 | FAMYVWMQA |
| 4084 | LFHSWMRYD |
| 4085 | FFTLKWHQF |
| 4086 | YAYAYQFCI |
| 4087 | AFIKHYLFE |
| 4088 | LTSNMLARD |
| 4089 | FSIMLMSSG |
| 4090 | QIQQYLSQF |
| 4091 | MALLKDGIE |
| 4092 | RYENFHQMD |
| 4093 | CAATNYHIH |
| 4094 | LFREISMNA |
| 4095 | QWHLFDAGV |
| 4096 | SANGGSIQM |
| 4097 | ATMSIMWAH |
| 4098 | DVSSTEAQF |
| 4099 | FLTSYTEKM |
| 4100 | SFSSLSGVA |
| 4101 | QHVQFTCLN |
| 4102 | AAQPWEHYY |
| 4103 | QMVSGDMGD |
| 4104 | LFSQYNGFM |
| 4105 | SGHQISDLF |
| 4106 | FMQIMQMQF |
| 4107 | LKLQLFNCG |
| 4108 | RFEQSMSVQ |
| 4109 | GNFFNNAIW |
| 4110 | FLMQYGDSC |
| 4111 | FAGVLLMCC |
| 4112 | FAFTSLINA |
| 4113 | LYSNLKRRR |
| 4114 | TISFCEFFN |
| 4115 | TFSLIFQMS |
| 4116 | NSGVVQSCF |
| 4117 | TGLEKIHFL |
| 4118 | FYYMYNSVV |
| 4119 | HTSHLGSQF |
| 4120 | QSVHVFYSW |
| 4121 | RYEPTSFMP |
| 4122 | LWAQYCKSE |
| 4123 | RFDQQGEYD |
| 4124 | QALQMDFLY |
| 4125 | DFTSTMTNG |
| 4126 | RQACAQNFQ |
| 4127 | AQYQWQMQF |
| 4128 | QWVVYHWQI |
| 4129 | LFQFQHRCR |
| 4130 | YTIMMHHLM |
| 4131 | LYQLFKRCY |
| 4132 | HAYQSITTS |
| 4133 | FFADFNKHC |
| 4134 | HSGFILALG |
| 4135 | TSWMMLQTH |
| 4136 | HIMYIGQYP |
| 4137 | VYNFLLDLG |
| 4138 | SFLPINLYA |
| 4139 | QSYQIQWIG |
| 4140 | DHFTFNFKC |
| 4141 | QDFSTFQSY |
| 4142 | RFDSKMENS |
| 4143 | AAVTAIMGE |
| 4144 | SGTSFGAML |
| 4145 | WSNPFIHYQ |
| 4146 | HVAQYNFAK |
| 4147 | RIIIHQFNE |
| 4148 | QAILAQNCM |
| 4149 | SFCMQMSSW |
| 4150 | GSQSNYHTA |
| 4151 | SMDPFSQNQ |
| 4152 | HGYFCQSFD |
| 4153 | DRHFFFFGI |
| 4154 | QYMSSQQCF |
| 4155 | NFTSNQQSY |
| 4156 | DCSLLNIWG |
| 4157 | MATSSSSSL |
| 4158 | NIMVYAQAR |
| 4159 | NAYTSIHNN |
| 4160 | LMQLFGVLR |
| 4161 | DSGSGITWH |
| 4162 | LFMHFNNMC |
| 4163 | HATSVAHHY |
| 4164 | ISEGHKNVL |
| 4165 | MNIVIEDKI |
| 4166 | AAQHSMLDS |
| 4167 | FWQYVNQQE |
| 4168 | RALDHLFSI |
| 4169 | QWEKYLKIM |
| 4170 | EAYNYQEMS |
| 4171 | LAYHTSWCQ |
| 4172 | NFCNCDFIL |
| 4173 | HHLFLFDSI |
| 4174 | FYQSAHFIF |
| 4175 | QGSYVACAE |
| 4176 | QAYCSHSCE |
| 4177 | QYRTTQDQN |
| 4178 | MNAPVFAQE |
| 4179 | ISSFQWAIA |
| 4180 | IMDLMQGCH |
| 4181 | QYQQNGTQH |
| 4182 | LYMHRGLYR |
| 4183 | CGYLWSSFA |
| 4184 | QSGESLSLM |
| 4185 | QNGQMQACN |
| 4186 | QFTSQSVQY |
| 4187 | LCSQYFDRI |
| 4188 | LIAPMFTDL |
| 4189 | RYCEKNQGK |
| 4190 | YSNSLWDSI |
| 4191 | FYALTMSCQ |
| 4192 | DTCYLQHAE |
| 4193 | LSLLAYNHI |
| 4194 | IWQLFNDCM |
| 4195 | LIGMQPFCN |
| 4196 | ESLSAYDKQ |
| 4197 | SFNQCQSTC |
| 4198 | RWSLIQQFE |
| 4199 | LFSSWMCFY |
| 4200 | GGISMHFFD |
| 4201 | QIGTIQAHS |
| 4202 | EWMYMQSGE |
| 4203 | QFYQNSQMP |
| 4204 | LATCSGQSI |
| 4205 | LSFDSNAHL |
| 4206 | SILFHQLIR |
| 4207 | SWQFSKQYL |
| 4208 | YALFELHND |
| 4209 | IICHIHTMM |
| 4210 | IFDPKIAWF |
| 4211 | LGHTQACML |
| 4212 | VMDSMHYFM |
| 4213 | RWDQLMVGE |
| 4214 | LFQFWKNCH |
| 4215 | LYAFYMSSE |
| 4216 | LFMSWQSTA |
| 4217 | KYENYQMLR |
| 4218 | DAILQSTHI |
| 4219 | LFNFYTVDW |
| 4220 | QAVSVTASI |
| 4221 | NHVSFQQSM |
| 4222 | FWASTGAYS |
| 4223 | KSQSTVYQD |
| 4224 | QTDHHHHCH |
| 4225 | QSFPIQAPA |
| 4226 | QMQQMSVMI |
| 4227 | QIHIQFDHC |
| 4228 | HYRGSKQWE |
| 4229 | CFDFQFEHF |
| 4230 | RASQMSGSM |
| 4231 | QASILQKNH |
| 4232 | QVSQQNQHF |
| 4233 | QYQQCNFGC |
| 4234 | ASFSNPAAT |
| 4235 | EAMYNNSFC |
| 4236 | DAYTSMSDW |
| 4237 | GNANFGNLL |

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 4 (SEQ ID NO: 5234-SEQ ID NO: 5807) that is expected to confer CNS tissue tropism or preference on a recombinant AAV virion (rAAV), based on a primary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 4 (SEQ ID NO: 5234-SEQ ID NO: 5807) at the region corresponding to amino acid residues 445 to 453 in AAV5 VP2 or the region corresponding to amino acid residues 389 to 397 in AAV5 VP3.

TABLE 4
Additional Sequences of 581-589 Regions
for CNS Tropism

| SEQ ID | |
|---|---|
| NO | Sequence |
| 5234 | AADGYVAQD |
| 5235 | AAMSSSHSY |
| 5236 | AARMVGEFY |
| 5237 | ACSQIGQDD |
| 5238 | ADYSMMWHM |
| 5239 | AEHMVVDHE |
| 5240 | AERDHKTIY |
| 5241 | AFARQEYAN |
| 5242 | AGMYKCQED |
| 5243 | AHQTAWAWI |
| 5244 | AHQTPWAWI |
| 5245 | AIGPALCLT |
| 5246 | AIGPDRSNN |
| 5247 | AKCTILNFQ |
| 5248 | AKRECTHIP |
| 5249 | ALQYCQGKD |
| 5250 | ANGTRECQS |
| 5251 | ANLISQGHC |
| 5252 | ANRMIWYPY |
| 5253 | AQCHDMWGH |
| 5254 | ARKNCDHLN |
| 5255 | ARSGINGHE |
| 5256 | ASMHEETCL |
| 5257 | ASRSCMTQE |
| 5258 | ATDMIFVSN |
| 5259 | ATGTYNLQE |
| 5260 | AVAEIRPEP |
| 5261 | AVHWHRKCE |
| 5262 | AVNEAQDNR |
| 5263 | AVTQLSVER |
| 5264 | AVYLDHVFS |
| 5265 | AWSYTGACY |
| 5266 | AYAPSKNAM |
| 5267 | CAGMAWSHS |
| 5268 | CAWHMKQGN |
| 5269 | CCCVPRSIY |
| 5270 | CCLWSYWAF |
| 5271 | CHILDGLHD |
| 5272 | CIMLNGCFQ |
| 5273 | CIQPTWDSY |
| 5274 | CIRFMMREG |
| 5275 | CKQLFCELG |
| 5276 | CNEVCRNYL |
| 5277 | CNYPIIWDK |
| 5278 | CRMITMMNE |
| 5279 | CVDTNRMWQ |
| 5280 | CVFSRNNYQ |
| 5281 | CVKPFLTGW |
| 5282 | CVNLMAVNN |
| 5283 | CVRIGMREP |
| 5284 | CWGPIMHPF |
| 5285 | DARKTYICY |
| 5286 | DATRAQTGE |
| 5287 | DCRCYDCHD |
| 5288 | DEAKKSCWQ |
| 5289 | DEGYIMFAP |
| 5290 | DGEFLHEVL |
| 5291 | DHCHCIAHP |
| 5292 | DHIHIERAN |
| 5293 | DHLSICCQN |
| 5294 | DIATMRNTW |
| 5295 | DICQKQSPN |
| 5296 | DIEFLDCEN |
| 5297 | DKMSLQVQI |
| 5298 | DLVDLRSWM |
| 5299 | DMTLQSVSS |
| 5300 | DNYWIVPFA |
| 5301 | DPGWYGLAP |
| 5302 | DQFCPFDHY |
| 5303 | DQRMLLKEL |
| 5304 | DQSMMTIGQ |
| 5305 | DRKQADQIY |
| 5306 | DSAKQWHHQ |
| 5307 | DTAFMHHHV |
| 5308 | DTHHVHGWQ |
| 5309 | DVCRKPVSH |
| 5310 | DVIRDMIAE |
| 5311 | DVKSWAVCD |
| 5312 | DVMQYQNHM |
| 5313 | DVRACMEDL |
| 5314 | DVTQTRYCY |
| 5315 | DWRYFCVSL |
| 5316 | EAYITYNKA |
| 5317 | ECIFAPRMS |
| 5318 | EDGLIVYGP |
| 5319 | EERPWEWET |
| 5320 | EFDWLLGTI |
| 5321 | EFKCVSDCL |
| 5322 | EGATAWQDR |
| 5323 | EHSSPREDN |
| 5324 | EICMRSGFI |
| 5325 | EIIQAHLIN |
| 5326 | EITQHFVKH |
| 5327 | EKRLEGWVY |
| 5328 | ELPDTEVPW |
| 5329 | ELRMSFTKV |
| 5330 | EPLVCISAG |
| 5331 | EPMPMPVWN |
| 5332 | EQLRDLNIE |
| 5333 | EQPHISIKG |
| 5334 | ERFKTFCEE |
| 5335 | ERKTTADWK |
| 5336 | ERQAHNHYA |
| 5337 | ERTACKMKS |
| 5338 | ERYYPISSF |
| 5339 | ESRQVQDCY |
| 5340 | ETEIRQMDG |
| 5341 | ETHRGTVGL |
| 5342 | EVRSTRKNH |
| 5343 | EWEGVSQFR |
| 5344 | EWHARHHYW |
| 5345 | EYCEFMSEA |
| 5346 | EYSRCCHQE |
| 5347 | FAESMWCDR |
| 5348 | FAPEFDNHC |
| 5349 | FAQQIINAS |
| 5350 | FCVKSPRVD |
| 5351 | FKPFAQQWN |
| 5352 | FLTCDQFEW |
| 5353 | FNTPYAHIG |
| 5354 | FQTSINYYG |
| 5355 | FRAVYYYHS |
| 5356 | FRDVCMHDV |
| 5357 | FSYQIQCQN |
| 5358 | FTEWKPVFF |
| 5359 | FTHSCCCLS |
| 5360 | GAGPVQSEE |
| 5361 | GAKYIQNTD |
| 5362 | GCLTSECAY |
| 5363 | GCMCIRHAY |
| 5364 | GDMCEDMCT |
| 5365 | GDVSYDAEA |
| 5366 | GEMTRCFHN |
| 5367 | GFQRTHVDQ |
| 5368 | GFTPDRCRN |
| 5369 | GGCCGGELN |
| 5370 | GGDGRVHNK |
| 5371 | GGDRMATDR |
| 5372 | GGSYVSAPD |
| 5373 | GHMFETFWG |
| 5374 | GIDLMRQIN |
| 5375 | GIHQLIHHC |
| 5376 | GLFHPQFGQ |
| 5377 | GMSDCEVSF |
| 5378 | GNECFHNVE |
| 5379 | GNKSPDECF |
| 5380 | GPARSFEAC |
| 5381 | GQPVWCVCM |
| 5382 | GREITQVMS |
| 5383 | GRWMCNEAE |
| 5384 | GSCCLNNET |
| 5385 | GSKIIMHTE |
| 5386 | GSQQTTLSC |
| 5387 | GSYIVQNHC |
| 5388 | GTEPIMWRD |
| 5389 | GTIRMPEHR |
| 5390 | GTNAYMAHK |
| 5391 | GVHHRCRMT |
| 5392 | GVITVDAKG |
| 5393 | GWDMLSVAK |
| 5394 | GYKFIGRSR |
| 5395 | GYNYYWQHA |
| 5396 | GYTQQDEHF |
| 5397 | HEGLIMRLE |
| 5398 | HEVRRYGQN |
| 5399 | HFEVRSVQR |
| 5400 | HGFKCQYSF |
| 5401 | HHRVNYEMY |
| 5402 | HIGWMGWLR |
| 5403 | HLMPRNQCF |
| 5404 | HLWMCPPRY |
| 5405 | HMGIAYNDH |
| 5406 | HMSQAGVVS |
| 5407 | HPLPNTEIC |
| 5408 | HQSPIHTNT |
| 5409 | HSLVQQLHP |
| 5410 | HSTNLFEAK |
| 5411 | HTLQWEAVR |
| 5412 | HTRKEGENS |
| 5413 | HVTMNQLYS |
| 5414 | IADDYFMCH |
| 5415 | IAKIGNCVW |
| 5416 | IAMRCWGNE |
| 5417 | IATHNHILN |
| 5418 | IATKSNCHK |
| 5419 | ICNMDFRCA |
| 5420 | IECSPRESI |
| 5421 | IEHCSNRYA |
| 5422 | IGQYFNFHF |
| 5423 | IHFTSQSCL |
| 5424 | IHGIDCYEI |
| 5425 | IHPMMRENG |
| 5426 | IIRQSEQYH |
| 5427 | ILINDREWG |
| 5428 | IMCHARMMA |
| 5429 | IMNPTAQNN |
| 5430 | IPAEWDSWR |
| 5431 | IPRPFFTMH |
| 5432 | IQAVMASGW |
| 5433 | IRPQYAKMD |
| 5434 | IRRSMQQQN |
| 5435 | ISQRGTLWC |
| 5436 | ISRMLRQSS |
| 5437 | ISTKFGKEI |
| 5438 | KAIWQGTEN |
| 5439 | KALHYAMIK |
| 5440 | KARQDDVNT |
| 5441 | KATHCDKYV |
| 5442 | KATVEGSWR |
| 5443 | KAVLFLKDC |
| 5444 | KCADLIIRS |
| 5445 | KCLPYMEMF |
| 5446 | KCNDIVLYH |
| 5447 | KCNVITYHG |
| 5448 | KCTMITPCF |
| 5449 | KDGYREAWM |
| 5450 | KDNQSCEWN |
| 5451 | KEAYTDMSC |
| 5452 | KENKIMQAS |
| 5453 | KFGFMYNEF |
| 5454 | KGEIEGGAK |
| 5455 | KGLITENNY |
| 5456 | KGTPNWWGP |
| 5457 | KHITWNHCL |
| 5458 | KHIVGQDGG |
| 5459 | KHTLAEMYG |
| 5460 | KIDAHSNME |
| 5461 | KIGCMYSSQ |
| 5462 | KIIFELGMD |
| 5463 | KISPWDGFY |
| 5464 | KIWAGVNLG |
| 5465 | KIYMQCDQE |
| 5466 | KKTECEMIV |
| 5467 | KKTLYEMAC |
| 5468 | KMAGIGCWY |
| 5469 | KMCWWPESM |
| 5470 | KMHGNERCF |
| 5471 | KMLHKVWID |
| 5472 | KMMAFEKQC |
| 5473 | KMNTWDQAR |
| 5474 | KNELENVIN |
| 5475 | KPAGKNTCW |
| 5476 | KPDHAMAQQ |
| 5477 | KPLTYTVGS |
| 5478 | KQKTQNRNA |
| 5479 | KQLFMKFWN |
| 5480 | KRTDLHAAD |
| 5481 | KRVQQMETM |
| 5482 | KRWLHMYQG |
| 5483 | KSAPRENFN |
| 5484 | KSGWGAPYE |
| 5485 | KSMTCVYYP |
| 5486 | KSTYYNLKW |
| 5487 | KSVHYETCE |
| 5488 | KSWVWVDTE |
| 5489 | KTCLCIFTK |
| 5490 | KTDPIAEYC |
| 5491 | KTEKWASHE |
| 5492 | KTFTKEVTP |
| 5493 | KTLPFAIYH |
| 5494 | KTMADMYNR |
| 5495 | KTQTHTSEP |
| 5496 | KTSNVMDQM |
| 5497 | KTVETYAAH |
| 5498 | KVAEGQNML |
| 5499 | KVAGGHETT |
| 5500 | KVDTVRNPK |
| 5501 | KVESTTQLC |
| 5502 | KWDDMHTQM |
| 5503 | KYMQENVLS |
| 5504 | LAAIQHCAM |
| 5505 | LACTPRCIF |
| 5506 | LADCMLNHH |
| 5507 | LAESKKRNK |
| 5508 | LATTTSYHC |
| 5509 | LCTSTERQG |
| 5510 | LEQLDRCMY |
| 5511 | LFMTYSARN |
| 5512 | LFYSPMHTH |
| 5513 | LILQRNVKD |
| 5514 | LLWRRVKRT |
| 5515 | LNELPRAEY |
| 5516 | LNHPIADYD |
| 5517 | LPPPEEKWL |
| 5518 | LPTQVYGHN |
| 5519 | LTCMHASTP |
| 5520 | LTPRSEGLP |
| 5521 | LTQYKALAQ |
| 5522 | LVGRTYQHQ |
| 5523 | LWDRFCCPV |
| 5524 | LWSRFISMD |
| 5525 | LYMMALLPT |
| 5526 | MAAITQNVA |
| 5527 | MAGHIGHHS |
| 5528 | MAQPYGRTA |
| 5529 | MARQCHSFH |
| 5530 | MCRPVMMIE |
| 5531 | MCSQTISMR |
| 5532 | MDGHFSVGC |
| 5533 | MDGRDEIIH |
| 5534 | MDPDIPHCR |
| 5535 | MDVQQIISG |
| 5536 | MEHTMDFGV |
| 5537 | MFDMMSVLK |
| 5538 | MFVVNQNWA |
| 5539 | MGQRSAANY |
| 5540 | MGRPHAWWG |
| 5541 | MIKPLLVVG |
| 5542 | MIRPSINGS |
| 5543 | MKNQPTGAS |
| 5544 | MKTIMQDYF |
| 5545 | MLFDFEEHN |
| 5546 | MLGVAPGHL |
| 5547 | MLPLEMKNL |
| 5548 | MMVRELMSS |
| 5549 | MPSWYIAQA |
| 5550 | MQHLPPAMT |
| 5551 | MQLRIHTNS |
| 5552 | MQYRGMSAS |
| 5553 | MRQRTYICQ |
| 5554 | MRRKTPEDD |
| 5555 | MRYHWDEAG |
| 5556 | MSLQHHFCT |
| 5557 | MSNLEYYEA |
| 5558 | MTDNNGPLF |
| 5559 | MTGIGKCWN |
| 5560 | MTRSCQTEG |
| 5561 | MYTGMRCWR |
| 5562 | NAGMKGVDV |
| 5563 | NAGVVKVWP |
| 5564 | NAIPYFVQA |
| 5565 | NAKYSMLYG |
| 5566 | NAQRLAVGM |
| 5567 | NARVMAKRE |
| 5568 | NCPMYDMIS |
| 5569 | NERDQCQWP |
| 5570 | NHSLCWDSK |
| 5571 | NLERCNTMM |
| 5572 | NMWYSFVMI |
| 5573 | NQREWHGLA |
| 5574 | NQRGFCKDE |
| 5575 | NRCGVSKCC |
| 5576 | NRIQMTDFQ |
| 5577 | NSTRNTSYY |
| 5578 | NSTRWYHTG |
| 5579 | NTAKLGWGL |
| 5580 | NTCEGWSHF |
| 5581 | NTGVKFCAC |
| 5582 | NTQYSAKND |
| 5583 | NTRIVCDLS |
| 5584 | NTSELLMAQ |
| 5585 | NVCDNGHEI |
| 5586 | NVFPAETHV |
| 5587 | NVMGTQAKE |
| 5588 | NVTTMFGQK |
| 5589 | NWEVIGRWH |
| 5590 | NWNHCHQWR |
| 5591 | NWTGNMQWA |
| 5592 | NYCKYNKMR |
| 5593 | PAWRRYCWT |
| 5594 | PEKWIHMHE |
| 5595 | PIHTYKRHP |
| 5596 | PIKGVAVQE |
| 5597 | PLNQEQIPQ |
| 5598 | PRNLVAMYW |
| 5599 | PVKPIIHAS |
| 5600 | PWMHTMLNP |
| 5601 | QAAVTKEYW |
| 5602 | QAKMILDGT |
| 5603 | QANPYNHGI |
| 5604 | QAVIAVDGR |
| 5605 | QCAHFYQTR |
| 5606 | QCGCAPMDS |
| 5607 | QCGFIRLNE |
| 5608 | QCQSQEWFH |
| 5609 | QDCERQVFA |
| 5610 | QEAGYTRHN |
| 5611 | QFCICCDGM |
| 5612 | QGENAQHHN |
| 5613 | QGFSTDVKP |
| 5614 | QGQHVMVHS |
| 5615 | QHLMTFSHE |
| 5616 | QHQAFFVFT |
| 5617 | QIKTHSNVF |
| 5618 | QKPTIWDHF |
| 5619 | QKSMLNSIQ |
| 5620 | QLKRNEQTS |
| 5621 | QMKGATEFC |
| 5622 | QMNAIQLCV |
| 5623 | QMVTQLNWE |
| 5624 | QNKHNWWIM |
| 5625 | QPKMWMMAS |
| 5626 | QPRVYEGFY |
| 5627 | QPYWVAVDP |
| 5628 | QQTWEQMQR |
| 5629 | QRGPLQHQQ |
| 5630 | QRNNLFVHD |
| 5631 | QSAHPEPMC |
| 5632 | QSGTCHYHD |
| 5633 | QSRQMMLLH |
| 5634 | QSTPTLGPK |
| 5635 | QSWWHSDTC |
| 5636 | QTDILRDVQ |
| 5637 | QTGTGHWFE |
| 5638 | QTGVMAAFG |
| 5639 | QTLGGHMWT |
| 5640 | QVACHMTNP |
| 5641 | QVHIGSWPP |
| 5642 | QVMQEKVRC |
| 5643 | QVNMFMMPC |
| 5644 | QVRFEQNPA |
| 5645 | QVSFVGCNH |
| 5646 | QVSHISEHW |
| 5647 | QWAPLQTWA |
| 5648 | QWEEVWWEA |
| 5649 | QYLLIPHLP |
| 5650 | QYQSTGVVR |
| 5651 | QYWWPATHD |
| 5652 | RAERYFKDE |
| 5653 | RAIYNDMTE |
| 5654 | RCVQAHFHK |
| 5655 | RDIWERPCV |
| 5656 | REEWKNASN |
| 5657 | REMMIVNCD |
| 5658 | RGKPILNQQ |
| 5659 | RITQGESGM |
| 5660 | RKWMNAKAT |
| 5661 | RLAWIEQSP |
| 5662 | RMMWQSHCG |
| 5663 | RMVLAYGNF |
| 5664 | RNDWQQVFS |
| 5665 | RQTTYDCLD |
| 5666 | RREAMDDSP |
| 5667 | RSDEMYVTP |
| 5668 | RTRISQMFG |
| 5669 | RTVPTQEMS |
| 5670 | RVAGEGIQP |
| 5671 | RVWSLEWHL |
| 5672 | RYNNCPNLW |
| 5673 | RYQSHCWLH |
| 5674 | SADRTAMFH |
| 5675 | SALYRWGHV |
| 5676 | SANQRYHDW |
| 5677 | SAPRNEITR |
| 5678 | SCQLLNDGG |
| 5679 | SFKSVSQYN |
| 5680 | SFPMILVNC |
| 5681 | SIAQDQVWS |
| 5682 | SIRYDFYVP |
| 5683 | SIYQKWVTN |
| 5684 | SKGDLCHQQ |
| 5685 | SKGWADPCP |
| 5686 | SMQYFFKTE |
| 5687 | SMRDLIAEA |
| 5688 | SMSHNGQCL |
| 5689 | SNRYYSRIF |
| 5690 | SPNGRGLCG |
| 5691 | SQIQIRQAD |
| 5692 | SQMKYAHNQ |
| 5693 | SQSRTIIQE |
| 5694 | SRDIAKSST |
| 5695 | SRLYKKRLF |
| 5696 | SRTRMYMDH |
| 5697 | SRVMIHVYV |
| 5698 | SSVVMAKQP |
| 5699 | STLEDEKPY |
| 5700 | STQKVPVLA |
| 5701 | STRPIACHS |
| 5702 | SVETARCSG |
| 5703 | SVGYIYDQS |
| 5704 | SVKDPMDWG |
| 5705 | SVTPDSITT |
| 5706 | TAGQQPTFM |
| 5707 | TARDREHAM |
| 5708 | TARSTSDKC |
| 5709 | TCKWYEESI |
| 5710 | TCTNSEPHP |
| 5711 | TCYCHRAHE |
| 5712 | TEEVQFVVH |
| 5713 | TFMFNSEWS |
| 5714 | TFMRKICGM |
| 5715 | TFVRKPSLM |
| 5716 | THSSCWYQD |
| 5717 | TISQIPWVR |
| 5718 | TKLSMMGCE |
| 5719 | TKQFFTAHC |
| 5720 | TKYHYHVKV |
| 5721 | TLEPAYRHE |
| 5722 | TLLLVGKDR |
| 5723 | TMAQKGLWF |
| 5724 | TMPDRDPTY |
| 5725 | TMRLFWDMG |
| 5726 | TNFPTSMHG |
| 5727 | TPKSEGICC |
| 5728 | TQAKIECCS |
| 5729 | TQEACHHDP |
| 5730 | TQNACKVRT |
| 5731 | TRHPTIAMH |
| 5732 | TRILNEFFC |
| 5733 | TSEPHCSYA |
| 5734 | TSERHSCMV |
| 5735 | TSMEFTQHQ |
| 5736 | TSNHNMALE |
| 5737 | TTAYHRSDH |
| 5738 | TTTITNRLC |
| 5739 | TTTNQFMFQ |
| 5740 | TVCGSFHGC |
| 5741 | TVIRDSRSY |
| 5742 | TVRHQMDAY |
| 5743 | TVTHMSTWG |
| 5744 | TWHKQLVGV |
| 5745 | TYASTRNFH |
| 5746 | TYDTRNHCL |
| 5747 | TYLHQPEDS |
| 5748 | TYWCPLMMN |
| 5749 | VAADSRMMI |
| 5750 | VADTWQVHC |
| 5751 | VADWIGHTP |
| 5752 | VAYPIQTDV |
| 5753 | VCGWWHQYW |
| 5754 | VCHNPDPFT |
| 5755 | VFGSTRSAA |
| 5756 | VFNLMLQDK |
| 5757 | VGIWFIPCR |
| 5758 | VHVHCMIGS |
| 5759 | VICRMKKHS |
| 5760 | VIKVNTVNP |
| 5761 | VKAWWHDHQ |
| 5762 | VKHTAVDVP |
| 5763 | VLKSCDRHG |
| 5764 | VMFYWRYNS |
| 5765 | VPMWAHWQN |
| 5766 | VPTGRRVKL |
| 5767 | VPVATMIHH |
| 5768 | VRADNMPVM |
| 5769 | VSAYIERQW |
| 5770 | VSKFIAASW |
| 5771 | VSRCGIDRS |
| 5772 | VSVQSRAQL |
| 5773 | VTMTTKSVW |
| 5774 | VWWMIIHTS |
| 5775 | WAIFGDIHH |
| 5776 | WAYYIRLQP |
| 5777 | WCEAVGDYA |
| 5778 | WCESWGPQG |
| 5779 | WCRCKRFIE |
| 5780 | WDGRPDLST |
| 5781 | WFSIIMSKL |
| 5782 | WINEANCLM |
| 5783 | WIRSAQRPG |
| 5784 | WKDDLRSTY |
| 5785 | WKWYGLPHH |
| 5786 | WPNIKHQPI |
| 5787 | WPTMLSAHM |
| 5788 | WSCRSICPP |
| 5789 | WVEPEQCFG |
| 5790 | WVKQKDVLC |
| 5791 | WVYSHEKHY |
| 5792 | YDLWGWSTR |
| 5793 | YDYWMQAPT |
| 5794 | YEHPIMWPV |
| 5795 | YEICKHGHF |
| 5796 | YFADAWLKT |
| 5797 | YFPSIQCYE |
| 5798 | YGGSEHWGP |
| 4799 | YGNSVTCTS |
| 5800 | YHCCFAMQS |
| 5801 | YMSWYQIRN |
| 5802 | YPAQYTAFS |
| 5803 | YPQPCSCHS |
| 5804 | YPVHMMPPL |
| 5805 | YRDRCWNRI |
| 5806 | YTELMKKAC |
| 5807 | YTNHYYNAR |

In some embodiments, the engineered AAV VP capsid polypeptide confers CNS tissue tropism or preference, wherein the CNS tissue is selected from the group consisting of hippocampus: (dentate gyrus, CA1 and CA3); cerebellum, hypothalamus, cortex: (occipital, temporal and forebrain); substantia nigra, thalamus, and any combination thereof.

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 10 that is expected to confer cardiac muscle tissue tropism or preference on a recombinant AAV virion (rAAV), based on a secondary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 10 at the region corresponding to amino acid residues 445 to 453 in AAV5 VP2 or the region corresponding to amino acid residues 389 to 397 in AAV5 VP3.

In some embodiments, a 581-589 region of an AAV5 VP capsid polypeptide has a sequence that is at least 75%, at least 77.7%, at least 85%, at least 88.8%, or 100% identical to a sequence provided in TABLE 10. In some embodiments, a 581-589 region of an AAV5 VP capsid polypeptide has a sequence that is at least 75%, at least 77.7%, at least 85%, at least 88.8% or 100% identical to SEQ ID NO: 348. In some embodiments, a 581-589 region of an AAV5 VP capsid polypeptide has a sequence that is at least 75%, at least 77.7%, at least 85%, at least 88.8% or 100% identical to SEQ ID NO: 2536. In some embodiments, a 581-589 region of an AAV5 VP capsid polypeptide has a sequence that is at least 75%, at least 77.7%, at least 85%, at least 88.8% or 100% identical to SEQ ID NO: 5607. In some embodiments, a 581-589 region of an AAV5 VP capsid polypeptide has a sequence that is at least 75%, at least 77.7%, at least 85%, at least 88.8% or 100% identical to SEQ ID NO: 1576. In some embodiments, a 581-589 region of an AAV5 VP capsid polypeptide has a sequence that is at least 75%, at least 77.7%, at least 85%, at least 88.8% or 100% identical to SEQ ID NO: 4955. In some embodiments, a 581-589 region of an AAV5 VP capsid polypeptide has a sequence that is at least 75%, at least 77.7%, at least 85%, at least 88.8% or 100% identical to SEQ ID NO: 5017. In some embodiments, a 581-589 region of an AAV5 VP capsid polypeptide has a sequence that is at least 75%, at least 77.7%, at least 85%, at least 88.8% or 100% identical to SEQ ID NO: 4349. In some embodiments, a 581-589 region of an AAV5 VP capsid polypeptide has a sequence that is at least 75%, at least 77.7%, at least 85%, at least 88.8% or 100% identical to SEQ ID NO: 4964. In some embodiments, a 581-589 region of an AAV5 VP capsid polypeptide has a sequence that is at least 75%, at least 77.7%, at least 85%, at least 88.8% or 100% identical to SEQ ID NO: 293. In some embodiments, a 581-589 region of an AAV5 VP capsid polypeptide has a sequence that is at least 75%, at least 77.7%, at least 85%, at least 88.8% or 100% identical to SEQ ID NO: 4314. In some embodiments, a 581-589 region of an AAV5 VP capsid polypeptide has a sequence that is at least 75%, at least 77.7%, at least 85%, at least 88.8% or 100% identical to SEQ ID NO: 4995. In some embodiments, a 581-589 region of an AAV5 VP capsid polypeptide has a sequence that is at least 75%, at least 77.7%, at least 85%, at least 88.8% or 100% identical to SEQ ID NO: 4366. In some embodiments, a 581-589 region of an AAV5 VP capsid polypeptide has a sequence that is at least 75%, at least 77.7%, at least 85%, at least 88.8% or 100% identical to SEQ ID NO: 4961. In some embodiments, a 581-589 region of an AAV5 VP capsid polypeptide has a sequence that is at least 75%, at least 77.7%, at least 85%, at least 88.8% or 100% identical to SEQ ID NO: 4952. In some embodiments, a 581-589 region of an AAV5 VP capsid polypeptide has a sequence that is at least 75%, at least 77.7%, at least 85%, at least 88.8% or 100% identical to SEQ ID NO: 5075. In some embodiments, a 581-589 region of an AAV5 VP capsid polypeptide has a sequence that is at least 75%, at least 77.7%, at least 85%, at least 88.8% or 100% identical to SEQ ID NO: 4354. In some embodiments, a 581-589 region of an AAV5 VP capsid polypeptide has a sequence that is at least 75%, at least 77.7%, at least 85%, at least 88.8% or 100% identical to SEQ ID NO: 5060. In some embodiments, a 581-589 region of an AAV5 VP capsid polypeptide has a sequence that is at least 75%, at least 77.7%, at least 85%, at least 88.8% or 100% identical to SEQ ID NO: 5138. In some embodiments, a 581-589 region of an AAV5 VP capsid polypeptide has a sequence that is at least 75%, at least 77.7%, at least 85%, at least 88.8% or 100% identical to SEQ ID NO: 5206. In some embodiments, a 581-589 region of an AAV5 VP capsid polypeptide has a sequence that is at least 75%, at least 77.7%, at least 85%, at least 88.8% or 100% identical to SEQ ID NO: 4288. In some embodiments, a 581-589 region of an AAV5 VP capsid polypeptide has a sequence that is at least 75%, at least 77.7%, at least 85%, at least 88.8% or 100% identical to SEQ ID NO: 5092. In some embodiments, a 581-589 region of an AAV5 VP capsid polypeptide has a sequence that is at least 75%, at least 77.7%, at least 85%, at least 88.8% or 100% identical to SEQ ID NO: 4059. In some embodiments, a 581-589 region of an AAV5 VP capsid polypeptide has a sequence that is at least 75%, at least 77.7%, at least 85%, at least 88.8% or 100% identical to SEQ ID NO: 4295. In some embodiments, a 581-589 region of an AAV5 VP capsid polypeptide has a sequence that is at least 75%, at least 77.7%, at least 85%, at least 88.8% or 100% identical to SEQ ID NO: 5030. In some embodiments, a 581-589 region of an AAV5 VP capsid polypeptide has a sequence that is at least 75%, at least 77.7%, at least 85%, at least 88.8% or 100% identical to SEQ ID NO: 4938. In some embodiments, a 581-589 region of an AAV5 VP capsid polypeptide has a sequence that is at least 75%, at least 77.7%, at least 85%, at least 88.8% or 100% identical to SEQ ID NO: 2661. In some embodiments, a 581-589 region of an AAV5 VP capsid polypeptide has a sequence that is at least 75%, at least 77.7%, at least 85%, at least 88.8% or 100% identical to SEQ ID NO: 5215. In some embodiments, a 581-589 region of an AAV5 VP capsid polypeptide has a sequence that is at least 75%, at least 77.7%, at least 85%, at least 88.8% or 100% identical to SEQ ID NO: 4960. In some embodiments, a 581-589 region of an AAV5 VP capsid polypeptide has a sequence that is at least 75%, at least 77.7%, at least 85%, at least 88.8% or 100% identical to SEQ ID NO: 4969. In some embodiments, a 581-589 region of an AAV5 VP capsid polypeptide has a sequence that is at least 75%, at least 77.7%, at least 85%, at least 88.8% or 100% identical to SEQ ID NO: 5023.

In some embodiments, a 581-589 region of an AAV5 VP capsid polypeptide has a sequence of SEQ ID NO: 348. In some embodiments, a 581-589 region of an AAV5 VP capsid polypeptide has a sequence of SEQ ID NO: 2536. In some embodiments, a 581-589 region of an AAV5 VP capsid polypeptide has a sequence of SEQ ID NO: 5607. In some embodiments, a 581-589 region of an AAV5 VP capsid polypeptide has a sequence of SEQ ID NO: 1576. In some embodiments, a 581-589 region of an AAV5 VP capsid polypeptide has a sequence of SEQ ID NO: 4955. In some embodiments, a 581-589 region of an AAV5 VP capsid polypeptide has a sequence of SEQ ID NO: 5017. In some embodiments, a 581-589 region of an AAV5 VP capsid polypeptide has a sequence of SEQ ID NO: 4349. In some embodiments, a 581-589 region of an AAV5 VP capsid polypeptide has a sequence of SEQ ID NO: 4964. In some embodiments, a 581-589 region of an AAV5 VP capsid polypeptide has a sequence of SEQ ID NO: 293. In some embodiments, a 581-589 region of an AAV5 VP capsid polypeptide has a sequence of SEQ ID NO: 4314. In some embodiments, a 581-589 region of an AAV5 VP capsid polypeptide has a sequence of SEQ ID NO: 4995. In some embodiments, a 581-589 region of an AAV5 VP capsid polypeptide has a sequence of SEQ ID NO: 4366. In some embodiments, a 581-589 region of an AAV5 VP capsid polypeptide has a sequence of SEQ ID NO: 4961. In some embodiments, a 581-589 region of an AAV5 VP capsid polypeptide has a sequence of SEQ ID NO: 4952. In some embodiments, a 581-589 region of an AAV5 VP capsid polypeptide has a sequence of SEQ ID NO: 5075. In some embodiments, a 581-589 region of an AAV5 VP capsid polypeptide has a sequence of SEQ ID NO: 4354. In some embodiments, a 581-589 region of an AAV5 VP capsid polypeptide has a sequence of SEQ ID NO: 5060. In some embodiments, a 581-589 region of an AAV5 VP capsid polypeptide has a sequence of SEQ ID NO: 5138. In some embodiments, a 581-589 region of an AAV5 VP capsid polypeptide has a sequence of SEQ ID NO: 5206. In some embodiments, a 581-589 region of an AAV5 VP capsid polypeptide has a sequence of SEQ ID NO: 4288. In some embodiments, a 581-589 region of an AAV5 VP capsid polypeptide has a sequence of SEQ ID NO: 5092. In some embodiments, a 581-589 region of an AAV5 VP capsid polypeptide has a sequence of SEQ ID NO: 4059. In some embodiments, a 581-589 region of an AAV5 VP capsid polypeptide has a sequence of SEQ ID NO: 4295. In some embodiments, a 581-589 region of an AAV5 VP capsid polypeptide has a sequence of SEQ ID NO: 5030. In some embodiments, a 581-589 region of an AAV5 VP capsid polypeptide has a sequence of SEQ ID NO: 4938. In some embodiments, a 581-589 region of an AAV5 VP capsid polypeptide has a sequence of SEQ ID NO: 2661. In some embodiments, a 581-589 region of an AAV5 VP capsid polypeptide has a sequence of SEQ ID NO: 5215. In some embodiments, a 581-589 region of an AAV5 VP capsid polypeptide has a sequence of SEQ ID NO: 4960. In some embodiments, a 581-589 region of an AAV5 VP capsid polypeptide has a sequence of SEQ ID NO: 4969. In some embodiments, a 581-589 region of an AAV5 VP capsid polypeptide has a sequence of SEQ ID NO: 5023. In some embodiments, a 581-589 region of an AAV5 VP capsid polypeptide has a sequence that is at least 75%, at least 77.7%, at least 85%, at least 88.8% or 100% identical to SEQ ID NO: 4934.

In some embodiments, a 581-589 region of an AAV5 VP capsid polypeptide has a sequence of SEQ ID NO: 348 with 1 or 2 conservative substitutions. In some embodiments, a 581-589 region of an AAV5 VP capsid polypeptide has a sequence of SEQ ID NO: 2536 with 1 or 2 conservative substitutions. In some embodiments, a 581-589 region of an AAV5 VP capsid polypeptide has a sequence of SEQ ID NO: 5607 with 1 or 2 conservative substitutions. In some embodiments, a 581-589 region of an AAV5 VP capsid polypeptide has a sequence of SEQ ID NO: 1576 with 1 or 2 conservative substitutions. In some embodiments, a 581-589 region of an AAV5 VP capsid polypeptide has a sequence of SEQ ID NO: 4955 with 1 or 2 conservative substitutions. In some embodiments, a 581-589 region of an AAV5 VP capsid polypeptide has a sequence of SEQ ID NO: 5017 with 1 or 2 conservative substitutions. In some embodiments, a 581-589 region of an AAV5 VP capsid polypeptide has a sequence of SEQ ID NO: 4349 with 1 or 2 conservative substitutions. In some embodiments, a 581-589 region of an AAV5 VP capsid polypeptide has a sequence of SEQ ID NO: 4964 with 1 or 2 conservative substitutions. In some embodiments, a 581-589 region of an AAV5 VP capsid polypeptide has a sequence of SEQ ID NO: 293 with 1 or 2 conservative substitutions. In some embodiments, a 581-589 region of an AAV5 VP capsid polypeptide has a sequence of SEQ ID NO: 4314 with 1 or 2 conservative substitutions. In some embodiments, a 581-589 region of an AAV5 VP capsid polypeptide has a sequence of SEQ ID NO: 4995 with 1 or 2 conservative substitutions. In some embodiments, a 581-589 region of an AAV5 VP capsid polypeptide has a sequence of SEQ ID NO: 4366 with 1 or 2 conservative substitutions. In some embodiments, a 581-589 region of an AAV5 VP capsid polypeptide has a sequence of SEQ ID NO: 4961 with 1 or 2 conservative substitutions. In some embodiments, a 581-589 region of an AAV5 VP capsid polypeptide has a sequence of SEQ ID NO: 4952 with 1 or 2 conservative substitutions. In some embodiments, a 581-589 region of an AAV5 VP capsid polypeptide has a sequence of SEQ ID NO: 5075 with 1 or 2 conservative substitutions. In some embodiments, a 581-589 region of an AAV5 VP capsid polypeptide has a sequence of SEQ ID NO: 4354 with 1 or 2 conservative substitutions. In some embodiments, a 581-589 region of an AAV5 VP capsid polypeptide has a sequence of SEQ ID NO: 5060 with 1 or 2 conservative substitutions. In some embodiments, a 581-589 region of an AAV5 VP capsid polypeptide has a sequence of SEQ ID NO: 5138 with 1 or 2 conservative substitutions. In some embodiments, a 581-589 region of an AAV5 VP capsid polypeptide has a sequence of SEQ ID NO: 5206 with 1 or 2 conservative substitutions. In some embodiments, a 581-589 region of an AAV5 VP capsid polypeptide has a sequence of SEQ ID NO: 4288 with 1 or 2 conservative substitutions. In some embodiments, a 581-589 region of an AAV5 VP capsid polypeptide has a sequence of SEQ ID NO: 5092 with 1 or 2 conservative substitutions. In some embodiments, a 581-589 region of an AAV5 VP capsid polypeptide has a sequence of SEQ ID NO: 4059 with 1 or 2 conservative substitutions. In some embodiments, a 581-589 region of an AAV5 VP capsid polypeptide has a sequence of SEQ ID NO: 4295 with 1 or 2 conservative substitutions. In some embodiments, a 581-589 region of an AAV5 VP capsid polypeptide has a sequence of SEQ ID NO: 5030 with 1 or 2 conservative substitutions. In some embodiments, a 581-589 region of an AAV5 VP capsid polypeptide has a sequence of SEQ ID NO: 4938 with 1 or 2 conservative substitutions. In some embodiments, a 581-589 region of an AAV5 VP capsid polypeptide has a sequence of SEQ ID NO: 2661 with 1 or 2 conservative substitutions. In some embodiments, a 581-589 region of an AAV5 VP capsid polypeptide has a sequence of SEQ ID NO: 5215 with 1 or 2 conservative substitutions. In some embodiments, a 581-589 region of an AAV5 VP capsid polypeptide has a sequence of SEQ ID NO: 4960 with 1 or 2 conservative substitutions. In some embodiments, a 581-589 region of an AAV5 VP capsid polypeptide has a sequence of SEQ ID NO: 4969 with 1 or 2 conservative substitutions. In some embodiments, a 581-589 region of an AAV5 VP capsid polypeptide has a sequence of SEQ ID NO: 5023 with 1 or 2 conservative substitutions. In some embodiments, a 581-589 region of an AAV5 VP capsid polypeptide has a sequence of SEQ ID NO: 4934 with 1 or 2 conservative substitutions. The conservative substitutions may be at any position in the 581-589 region.

In some embodiments, an AAV VP capsid polypeptide preferentially targets a cardiac muscle tissue at a DNA enrichment of at least 25-fold greater than a wild type AAV9. For example, an AAV VP capsid polypeptide comprising a 581-589 region of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5092, SEQ ID NO: 4059, SEQ ID NO: 4295, SEQ ID NO: 5030, SEQ ID NO: 4938, SEQ ID NO: 2661, SEQ ID NO: 5215, SEQ ID NO: 4960, SEQ ID NO: 4969, SEQ ID NO: 5023, SEQ ID NO: 4934, SEQ ID NO: 4545, SEQ ID NO: 4963, SEQ ID NO: 4363, SEQ ID NO: 5159, or SEQ ID NO: 4973 may preferentially target a cardiac muscle tissue at a DNA enrichment of at least 25-fold greater than a wild type AAV9.

In some embodiments, an AAV VP capsid polypeptide preferentially targets a cardiac muscle tissue at a DNA enrichment of at least 30-fold greater than the wild type AAV9. For example, an AAV VP capsid polypeptide comprising a 581-589 region of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, or SEQ ID NO: 4366 may preferentially target a cardiac muscle tissue at a DNA enrichment of at least 30-fold greater than the wild type AAV9.

In some embodiments, an AAV VP capsid polypeptide preferentially targets a cardiac muscle tissue at a DNA enrichment of at least 50-fold greater than the wild type AAV9. For example, an AAV VP capsid polypeptide comprising a 581-589 region of SEQ ID NO: 348 or SEQ ID NO: 2536 may preferentially target a cardiac muscle tissue at a DNA enrichment of at least 50-fold greater than the wild type AAV9.

In some embodiments, an AAV VP capsid polypeptide preferentially targets a cardiac muscle tissue at an RNA enrichment of at least 10-fold greater than a wild type AAV9. For example, an AAV VP capsid polypeptide comprising a 581-589 region of SEQ ID NO: 348, SEQ ID NO: 5052, SEQ ID NO: 5193, SEQ ID NO: 5138, SEQ ID NO: 5163, SEQ ID NO: 4952, SEQ ID NO: 293, SEQ ID NO: 4317, SEQ ID NO: 5075, SEQ ID NO: 386, SEQ ID NO: 4288, SEQ ID NO: 4995, SEQ ID NO: 5030, SEQ ID NO: 4986, SEQ ID NO: 5206, SEQ ID NO: 4338, SEQ ID NO: 5159, SEQ ID NO: 4346, SEQ ID NO: 5060, SEQ ID NO: 4949, SEQ ID NO: 5017, SEQ ID NO: 5215, SEQ ID NO: 4314, SEQ ID NO: 5080, SEQ ID NO: 4964, SEQ ID NO: 4379, SEQ ID NO: 4304, SEQ ID NO: 18, SEQ ID NO: 4363, SEQ ID NO: 5027, SEQ ID NO: 4349, SEQ ID NO: 5208, or SEQ ID NO: 4938 may preferentially target a cardiac muscle tissue at an RNA enrichment of at least 10-fold greater than a wild type AAV9.

In some embodiments, an AAV VP capsid polypeptide preferentially targets a cardiac muscle tissue at an RNA enrichment of at least 20-fold or greater than the wild type AAV9. For example, an AAV VP capsid polypeptide comprising a 581-589 region of SEQ ID NO: 348, SEQ ID NO: 5052, SEQ ID NO: 5193, SEQ ID NO: 5138, or SEQ ID NO: 5163 may preferentially target a cardiac muscle tissue at an RNA enrichment of at least 20-fold or greater than the wild type AAV9.

In some embodiments, an AAV VP capsid polypeptide preferentially targets a cardiac muscle tissue at an RNA enrichment of at least 40-fold or greater than the wild type AAV9. For example, an AAV VP capsid polypeptide comprising a 581-589 region of SEQ ID NO: 348 may preferentially target a cardiac muscle tissue at an RNA enrichment of at least 40-fold or greater than the wild type AAV9.

In some embodiments, an AAV VP capsid polypeptide preferentially targets a cardiac muscle tissue at at a DNA enrichment of at least 5-fold greater than a wild type AAV5. For example, an AAV VP capsid polypeptide comprising a 581-589 region of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5092, SEQ ID NO: 4059, SEQ ID NO: 4295, SEQ ID NO: 5030, SEQ ID NO: 4938, SEQ ID NO: 2661, SEQ ID NO: 5215, SEQ ID NO: 4960, SEQ ID NO: 4969, SEQ ID NO: 5023, SEQ ID NO: 4934, SEQ ID NO: 4545, SEQ ID NO: 4963, SEQ ID NO: 4363, SEQ ID NO: 5159, or SEQ ID NO: 4973 may preferentially target a cardiac muscle tissue at a DNA enrichment of at least 5-fold greater than a wild type AAV5.

In some embodiments, an AAV VP capsid polypeptide preferentially targets a cardiac muscle tissue at a DNA enrichment of at least 10-fold greater than the wild type AAV5. For example, an AAV VP capsid polypeptide comprising a 581-589 region of SEQ ID NO: 348 or SEQ ID NO: 2536 may preferentially target a cardiac muscle tissue at a DNA enrichment of at least 10-fold greater than the wild type AAV5.

In some embodiments, an AAV VP capsid polypeptide preferentially targets a cardiac muscle tissue at an RNA enrichment of at least 5-fold greater than a wild type AAV5. For example, an AAV VP capsid polypeptide comprising a 581-589 region of SEQ ID NO: 348, SEQ ID NO: 5052, SEQ ID NO: 5193, SEQ ID NO: 5138, SEQ ID NO: 5163, SEQ ID NO: 4952, SEQ ID NO: 293, or SEQ ID NO: 4317 may preferentially target a cardiac muscle tissue at an RNA enrichment of at least 5-fold greater than a wild type AAV5.

In some embodiments, an AAV VP capsid polypeptide preferentially targets a cardiac muscle tissue at an RNA enrichment of at least 10-fold greater than the wild type AAV5. For example, an AAV VP capsid polypeptide comprising a 581-589 region of SEQ ID NO: 348 may preferentially target a cardiac muscle tissue at an RNA enrichment of at least 10-fold greater than the wild type AAV5.

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 11 that is expected to confer skeletal muscle tissue tropism or preference on a recombinant AAV virion (rAAV), based on a secondary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 11 at the region corresponding to amino acid residues 445 to 453 in AAV5 VP2 or the region corresponding to amino acid residues 389 to 397 in AAV5 VP3.

In some embodiments, a 581-589 region of an AAV5 VP capsid polypeptide has a sequence that is at least 75%, at least 77.7%, at least 85%, at least 88.8%, or 100% identical to a sequence provided in TABLE 11. In some embodiments, a 581-589 region of an AAV5 VP capsid polypeptide has a sequence that is at least 75%, at least 77.7%, at least 85%, at least 88.8% or 100% identical to SEQ ID NO: 3472. In some embodiments, a 581-589 region of an AAV5 VP capsid polypeptide has a sequence that is at least 75%, at least 77.7%, at least 85%, at least 88.8% or 100% identical to SEQ ID NO: 3297. In some embodiments, a 581-589 region of an AAV5 VP capsid polypeptide has a sequence that is at least 75%, at least 77.7%, at least 85%, at least 88.8% or 100% identical to SEQ ID NO: 2661. In some embodiments, a 581-589 region of an AAV5 VP capsid polypeptide has a sequence that is at least 75%, at least 77.7%, at least 85%, at least 88.8% or 100% identical to SEQ ID NO: 4545. In some embodiments, a 581-589 region of an AAV5 VP capsid polypeptide has a sequence that is at least 75%, at least 77.7%, at least 85%, at least 88.8% or 100% identical to SEQ ID NO: 348. In some embodiments, a 581-589 region of an AAV5 VP capsid polypeptide has a sequence that is at least 75%, at least 77.7%, at least 85%, at least 88.8% or 100% identical to SEQ ID NO: 18. In some embodiments, a 581-589 region of an AAV5 VP capsid polypeptide has a sequence that is at least 75%, at least 77.7%, at least 85%, at least 88.8% or 100% identical to SEQ ID NO: 4349. In some embodiments, a 581-589 region of an AAV5 VP capsid polypeptide has a sequence that is at least 75%, at least 77.7%, at least 85%, at least 88.8% or 100% identical to SEQ ID NO: 293. In some embodiments, a 581-589 region of an AAV5 VP capsid polypeptide has a sequence that is at least 75%, at least 77.7%, at least 85%, at least 88.8% or 100% identical to SEQ ID NO: 5017. In some embodiments, a 581-589 region of an AAV5 VP capsid polypeptide has a sequence that is at least 75%, at least 77.7%, at least 85%, at least 88.8% or 100% identical to SEQ ID NO: 4955. In some embodiments, a 581-589 region of an AAV5 VP capsid polypeptide has a sequence that is at least 75%, at least 77.7%, at least 85%, at least 88.8% or 100% identical to SEQ ID NO: 4366. In some embodiments, a 581-589 region of an AAV5 VP capsid polypeptide has a sequence that is at least 75%, at least 77.7%, at least 85%, at least 88.8% or 100% identical to SEQ ID NO: 5092. In some embodiments, a 581-589 region of an AAV5 VP capsid polypeptide has a sequence that is at least 75%, at least 77.7%, at least 85%, at least 88.8% or 100% identical to SEQ ID NO: 210. In some embodiments, a 581-589 region of an AAV5 VP capsid polypeptide has a sequence that is at least 75%, at least 77.7%, at least 85%, at least 88.8% or 100% identical to SEQ ID NO: 4059. In some embodiments, a 581-589 region of an AAV5 VP capsid polypeptide has a sequence that is at least 75%, at least 77.7%, at least 85%, at least 88.8% or 100% identical to SEQ ID NO: 2536. In some embodiments, a 581-589 region of an AAV5 VP capsid polypeptide has a sequence that is at least 75%, at least 77.7%, at least 85%, at least 88.8% or 100% identical to SEQ ID NO: 5206. In some embodiments, a 581-589 region of an AAV5 VP capsid polypeptide has a sequence that is at least 75%, at least 77.7%, at least 85%, at least 88.8% or 100% identical to SEQ ID NO: 1971. In some embodiments, a 581-589 region of an AAV5 VP capsid polypeptide has a sequence that is at least 75%, at least 77.7%, at least 85%, at least 88.8% or 100% identical to SEQ ID NO: 262. In some embodiments, a 581-589 region of an AAV5 VP capsid polypeptide has a sequence that is at least 75%, at least 77.7%, at least 85%, at least 88.8% or 100% identical to SEQ ID NO: 4343. In some embodiments, a 581-589 region of an AAV5 VP capsid polypeptide has a sequence that is at least 75%, at least 77.7%, at least 85%, at least 88.8% or 100% identical to SEQ ID NO: 4995. In some embodiments, a 581-589 region of an AAV5 VP capsid polypeptide has a sequence that is at least 75%, at least 77.7%, at least 85%, at least 88.8% or 100% identical to SEQ ID NO: 4009. In some embodiments, a 581-589 region of an AAV5 VP capsid polypeptide has a sequence that is at least 75%, at least 77.7%, at least 85%, at least 88.8% or 100% identical to SEQ ID NO: 5190. In some embodiments, a 581-589 region of an AAV5 VP capsid polypeptide has a sequence that is at least 75%, at least 77.7%, at least 85%, at least 88.8% or 100% identical to SEQ ID NO: 724. In some embodiments, a 581-589 region of an AAV5 VP capsid polypeptide has a sequence that is at least 75%, at least 77.7%, at least 85%, at least 88.8% or 100% identical to SEQ ID NO: 4288. In some embodiments, a 581-589 region of an AAV5 VP capsid polypeptide has a sequence that is at least 75%, at least 77.7%, at least 85%, at least 88.8% or 100% identical to SEQ ID NO: 4973. In some embodiments, a 581-589 region of an AAV5 VP capsid polypeptide has a sequence that is at least 75%, at least 77.7%, at least 85%, at least 88.8% or 100% identical to SEQ ID NO: 4314. In some embodiments, a 581-589 region of an AAV5 VP capsid polypeptide has a sequence that is at least 75%, at least 77.7%, at least 85%, at least 88.8% or 100% identical to SEQ ID NO: 1561. In some embodiments, a 581-589 region of an AAV5 VP capsid polypeptide has a sequence that is at least 75%, at least 77.7%, at least 85%, at least 88.8% or 100% identical to SEQ ID NO: 5147. In some embodiments, a 581-589 region of an AAV5 VP capsid polypeptide has a sequence that is at least 75%, at least 77.7%, at least 85%, at least 88.8% or 100% identical to SEQ ID NO: 4238. In some embodiments, a 581-589 region of an AAV5 VP capsid polypeptide has a sequence that is at least 75%, at least 77.7%, at least 85%, at least 88.8% or 100% identical to SEQ ID NO: 4961.

In some embodiments, a 581-589 region of an AAV5 VP capsid polypeptide has a sequence of SEQ ID NO: 3472. In some embodiments, a 581-589 region of an AAV5 VP capsid polypeptide has a sequence of SEQ ID NO: 3297. In some embodiments, a 581-589 region of an AAV5 VP capsid polypeptide has a sequence of SEQ ID NO: 2661. In some embodiments, a 581-589 region of an AAV5 VP capsid polypeptide has a sequence of SEQ ID NO: 4545. In some embodiments, a 581-589 region of an AAV5 VP capsid polypeptide has a sequence of SEQ ID NO: 348. In some embodiments, a 581-589 region of an AAV5 VP capsid polypeptide has a sequence of SEQ ID NO: 18. In some embodiments, a 581-589 region of an AAV5 VP capsid polypeptide has a sequence of SEQ ID NO: 4349. In some embodiments, a 581-589 region of an AAV5 VP capsid polypeptide has a sequence of SEQ ID NO: 293. In some embodiments, a 581-589 region of an AAV5 VP capsid polypeptide has a sequence of SEQ ID NO: 5017. In some embodiments, a 581-589 region of an AAV5 VP capsid polypeptide has a sequence of SEQ ID NO: 4955. In some embodiments, a 581-589 region of an AAV5 VP capsid polypeptide has a sequence of SEQ ID NO: 4366. In some embodiments, a 581-589 region of an AAV5 VP capsid polypeptide has a sequence of SEQ ID NO: 5092. In some embodiments, a 581-589 region of an AAV5 VP capsid polypeptide has a sequence of SEQ ID NO: 210. In some embodiments, a 581-589 region of an AAV5 VP capsid polypeptide has a sequence of SEQ ID NO: 4059. In some embodiments, a 581-589 region of an AAV5 VP capsid polypeptide has a sequence of SEQ ID NO: 2536. In some embodiments, a 581-589 region of an AAV5 VP capsid polypeptide has a sequence of SEQ ID NO: 5206. In some embodiments, a 581-589 region of an AAV5 VP capsid polypeptide has a sequence of SEQ ID NO: 1971. In some embodiments, a 581-589 region of an AAV5 VP capsid polypeptide has a sequence of SEQ ID NO: 262. In some embodiments, a 581-589 region of an AAV5 VP capsid polypeptide has a sequence of SEQ ID NO: 4343. In some embodiments, a 581-589 region of an AAV5 VP capsid polypeptide has a sequence of SEQ ID NO: 4995. In some embodiments, a 581-589 region of an AAV5 VP capsid polypeptide has a sequence of SEQ ID NO: 4009. In some embodiments, a 581-589 region of an AAV5 VP capsid polypeptide has a sequence of SEQ ID NO: 5190. In some embodiments, a 581-589 region of an AAV5 VP capsid polypeptide has a sequence of SEQ ID NO: 724. In some embodiments, a 581-589 region of an AAV5 VP capsid polypeptide has a sequence of SEQ ID NO: 4288. In some embodiments, a 581-589 region of an AAV5 VP capsid polypeptide has a sequence of SEQ ID NO: 4973. In some embodiments, a 581-589 region of an AAV5 VP capsid polypeptide has a sequence of SEQ ID NO: 4314. In some embodiments, a 581-589 region of an AAV5 VP capsid polypeptide has a sequence of SEQ ID NO: 1561. In some embodiments, a 581-589 region of an AAV5 VP capsid polypeptide has a sequence of SEQ ID NO: 5147. In some embodiments, a 581-589 region of an AAV5 VP capsid polypeptide has a sequence of SEQ ID NO: 4238. In some embodiments, a 581-589 region of an AAV5 VP capsid polypeptide has a sequence of SEQ ID NO: 4961.

In some embodiments, a 581-589 region of an AAV5 VP capsid polypeptide has a sequence of SEQ ID NO: 3472 with 1 or 2 conservative substitutions. In some embodiments, a 581-589 region of an AAV5 VP capsid polypeptide has a sequence of SEQ ID NO: 3297 with 1 or 2 conservative substitutions. In some embodiments, a 581-589 region of an AAV5 VP capsid polypeptide has a sequence of SEQ ID NO: 2661 with 1 or 2 conservative substitutions. In some embodiments, a 581-589 region of an AAV5 VP capsid polypeptide has a sequence of SEQ ID NO: 4545 with 1 or 2 conservative substitutions. In some embodiments, a 581-589 region of an AAV5 VP capsid polypeptide has a sequence of SEQ ID NO: 348 with 1 or 2 conservative substitutions. In some embodiments, a 581-589 region of an AAV5 VP capsid polypeptide has a sequence of SEQ ID NO: 18 with 1 or 2 conservative substitutions. In some embodiments, a 581-589 region of an AAV5 VP capsid polypeptide has a sequence of SEQ ID NO: 4349 with 1 or 2 conservative substitutions. In some embodiments, a 581-589 region of an AAV5 VP capsid polypeptide has a sequence of SEQ ID NO: 293 with 1 or 2 conservative substitutions. In some embodiments, a 581-589 region of an AAV5 VP capsid polypeptide has a sequence of SEQ ID NO: 5017 with 1 or 2 conservative substitutions. In some embodiments, a 581-589 region of an AAV5 VP capsid polypeptide has a sequence of SEQ ID NO: 4955 with 1 or 2 conservative substitutions. In some embodiments, a 581-589 region of an AAV5 VP capsid polypeptide has a sequence of SEQ ID NO: 4366 with 1 or 2 conservative substitutions. In some embodiments, a 581-589 region of an AAV5 VP capsid polypeptide has a sequence of SEQ ID NO: 5092 with 1 or 2 conservative substitutions. In some embodiments, a 581-589 region of an AAV5 VP capsid polypeptide has a sequence of SEQ ID NO: 210 with 1 or 2 conservative substitutions. In some embodiments, a 581-589 region of an AAV5 VP capsid polypeptide has a sequence of SEQ ID NO: 4059 with 1 or 2 conservative substitutions. In some embodiments, a 581-589 region of an AAV5 VP capsid polypeptide has a sequence of SEQ ID NO: 2536 with 1 or 2 conservative substitutions. In some embodiments, a 581-589 region of an AAV5 VP capsid polypeptide has a sequence of SEQ ID NO: 5206 with 1 or 2 conservative substitutions. In some embodiments, a 581-589 region of an AAV5 VP capsid polypeptide has a sequence of SEQ ID NO: 1971 with 1 or 2 conservative substitutions. In some embodiments, a 581-589 region of an AAV5 VP capsid polypeptide has a sequence of SEQ ID NO: 262 with 1 or 2 conservative substitutions. In some embodiments, a 581-589 region of an AAV5 VP capsid polypeptide has a sequence of SEQ ID NO: 4343 with 1 or 2 conservative substitutions. In some embodiments, a 581-589 region of an AAV5 VP capsid polypeptide has a sequence of SEQ ID NO: 4995 with 1 or 2 conservative substitutions. In some embodiments, a 581-589 region of an AAV5 VP capsid polypeptide has a sequence of SEQ ID NO: 4009 with 1 or 2 conservative substitutions. In some embodiments, a 581-589 region of an AAV5 VP capsid polypeptide has a sequence of SEQ ID NO: 5190 with 1 or 2 conservative substitutions. In some embodiments, a 581-589 region of an AAV5 VP capsid polypeptide has a sequence of SEQ ID NO: 724 with 1 or 2 conservative substitutions. In some embodiments, a 581-589 region of an AAV5 VP capsid polypeptide has a sequence of SEQ ID NO: 4288 with 1 or 2 conservative substitutions. In some embodiments, a 581-589 region of an AAV5 VP capsid polypeptide has a sequence of SEQ ID NO: 4973 with 1 or 2 conservative substitutions. In some embodiments, a 581-589 region of an AAV5 VP capsid polypeptide has a sequence of SEQ ID NO: 4314 with 1 or 2 conservative substitutions. In some embodiments, a 581-589 region of an AAV5 VP capsid polypeptide has a sequence of SEQ ID NO: 1561 with 1 or 2 conservative substitutions. In some embodiments, a 581-589 region of an AAV5 VP capsid polypeptide has a sequence of SEQ ID NO: 5147 with 1 or 2 conservative substitutions. In some embodiments, a 581-589 region of an AAV5 VP capsid polypeptide has a sequence of SEQ ID NO: 4238 with 1 or 2 conservative substitutions. In some embodiments, a 581-589 region of an AAV5 VP capsid polypeptide has a sequence of SEQ ID NO: 4961 with 1 or 2 conservative substitutions. The conservative substitutions may be at any position in the 581-589 region.

In some embodiments, an AAV VP capsid polypeptide preferentially targets a skeletal muscle tissue at a DNA enrichment of at least 15-fold greater than a wild type AAV9. For example, an AAV VP capsid polypeptide comprising a 581-589 region of SEQ ID NO: 3472, SEQ ID NO: 3297, SEQ ID NO: 2661, SEQ ID NO: 4545, SEQ ID NO: 348, SEQ ID NO: 18, SEQ ID NO: 4349, SEQ ID NO: 293, SEQ ID NO: 5017, SEQ ID NO: 4955, SEQ ID NO: 4366, SEQ ID NO: 5092, SEQ ID NO: 210, SEQ ID NO: 4059, SEQ ID NO: 2536, SEQ ID NO: 5206, or SEQ ID NO: 1971 may preferentially target a skeletal muscle tissue at a DNA enrichment of at least 15-fold greater than a wild type AAV9.

In some embodiments, an AAV VP capsid polypeptide preferentially targets a skeletal muscle tissue at a DNA enrichment of at least 20-fold greater than the wild type AAV9. For example, an AAV VP capsid polypeptide comprising a 581-589 region of SEQ ID NO: 3472, SEQ ID NO: 3297, SEQ ID NO: 2661, SEQ ID NO: 4545, or SEQ ID NO: 348 may preferentially target a skeletal muscle tissue at a DNA enrichment of at least 20-fold greater than the wild type AAV9.

In some embodiments, an AAV VP capsid polypeptide preferentially targets a skeletal muscle tissue at a DNA enrichment of at least 30-fold greater than the wild type AAV9. For example, an AAV VP capsid polypeptide comprising a 581-589 region of SEQ ID NO: 3472 or SEQ ID NO: 3297 may preferentially target a skeletal muscle tissue at a DNA enrichment of at least 30-fold greater than the wild type AAV9.

In some embodiments, an AAV VP capsid polypeptide preferentially targets a skeletal muscle tissue at an RNA enrichment of at least 2-fold greater than a wild type AAV9. For example, an AAV VP capsid polypeptide comprising a 581-589 region of SEQ ID NO: 5138, SEQ ID NO: 3472, SEQ ID NO: 1934, SEQ ID NO: 3283, SEQ ID NO: 5817 (KTGTRDSAR), SEQ ID NO: 278, SEQ ID NO: 1634, SEQ ID NO: 1391, or SEQ ID NO: 1537 may preferentially target a skeletal muscle tissue at an RNA enrichment of at least 2-fold greater than a wild type AAV9.

In some embodiments, an AAV VP capsid polypeptide preferentially targets a skeletal muscle tissue at an RNA enrichment of at least 10-fold or greater than the wild type AAV9. For example, an AAV VP capsid polypeptide comprising a 581-589 region of SEQ ID NO: 5138, SEQ ID NO: 3472, SEQ ID NO: 1934, SEQ ID NO: 3283, SEQ ID NO: 5817, or SEQ ID NO: 278 may preferentially target a skeletal muscle tissue at an RNA enrichment of at least 10-fold or greater than the wild type AAV9.

In some embodiments, an AAV VP capsid polypeptide preferentially targets a skeletal muscle tissue at an RNA enrichment of at least 25-fold or greater than the wild type AAV9. For example, an AAV VP capsid polypeptide comprising a 581-589 region of SEQ ID NO: 5138, SEQ ID NO: 3472, SEQ ID NO: 1934, or SEQ ID NO: 3283 may preferentially target a skeletal muscle tissue at an RNA enrichment of at least 25-fold or greater than the wild type AAV9.

In some embodiments, an AAV VP capsid polypeptide preferentially targets a skeletal muscle tissue at a DNA enrichment of at least 4-fold greater than a wild type AAV5. For example, an AAV VP capsid polypeptide comprising a 581-589 region of SEQ ID NO: 3472, SEQ ID NO: 3297, SEQ ID NO: 2661, SEQ ID NO: 4545, SEQ ID NO: 348, SEQ ID NO: 18, SEQ ID NO: 4349, SEQ ID NO: 293, SEQ ID NO: 5017, SEQ ID NO: 4955, or SEQ ID NO: 4366 may preferentially target a skeletal muscle tissue at a DNA enrichment of at least 4-fold greater than a wild type AAV5.

In some embodiments, an AAV VP capsid polypeptide preferentially targets a skeletal muscle tissue at a DNA enrichment of at least 5-fold greater than the wild type AAV5. For example, an AAV VP capsid polypeptide comprising a 581-589 region of SEQ ID NO: 3472, SEQ ID NO: 3297, SEQ ID NO: 2661, SEQ ID NO: 4545, or SEQ ID NO: 348 may preferentially target a skeletal muscle tissue at a DNA enrichment of at least 5-fold greater than the wild type AAV5.

In some embodiments, an AAV VP capsid polypeptide preferentially targets a skeletal muscle tissue at an RNA enrichment of at least that of a wild type AAV5. For example, an AAV VP capsid polypeptide comprising a 581-589 region of SEQ ID NO: 5138, SEQ ID NO: 3472, SEQ ID NO: 1934, SEQ ID NO: 3283, SEQ ID NO: 5817, or SEQ ID NO: 278 may preferentially target a skeletal muscle tissue at an RNA enrichment of at least that of a wild type AAV5.

In some embodiments, an AAV VP capsid polypeptide preferentially targets a skeletal muscle tissue at an RNA enrichment of at least 3-fold greater than the wild type AAV5. For example, an AAV VP capsid polypeptide comprising a 581-589 region of SEQ ID NO: 5138, SEQ ID NO: 3472, SEQ ID NO: 1934, or SEQ ID NO: 3283 may preferentially target a skeletal muscle tissue at an RNA enrichment of at least 3-fold greater than the wild type AAV5.

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 12 that is expected to confer muscle tissue tropism or preference on a recombinant AAV virion (rAAV), based on a secondary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 12 at the region corresponding to amino acid residues 445 to 453 in AAV5 VP2 or the region corresponding to amino acid residues 389 to 397 in AAV5 VP3.

In some embodiments, a 581-589 region of an AAV5 VP capsid polypeptide has a sequence that is at least 75%, at least 77.7%, at least 85%, at least 88.8%, or 100% identical to a sequence provided in TABLE 12. In some embodiments, a 581-589 region of an AAV5 VP capsid polypeptide has a sequence that is at least 75%, at least 77.7%, at least 85%, at least 88.8% or 100% identical to SEQ ID NO: 348. In some embodiments, a 581-589 region of an AAV5 VP capsid polypeptide has a sequence that is at least 75%, at least 77.7%, at least 85%, at least 88.8% or 100% identical to SEQ ID NO: 2536. In some embodiments, a 581-589 region of an AAV5 VP capsid polypeptide has a sequence that is at least 75%, at least 77.7%, at least 85%, at least 88.8% or 100% identical to SEQ ID NO: 4955. In some embodiments, a 581-589 region of an AAV5 VP capsid polypeptide has a sequence that is at least 75%, at least 77.7%, at least 85%, at least 88.8% or 100% identical to SEQ ID NO: 5017. In some embodiments, a 581-589 region of an AAV5 VP capsid polypeptide has a sequence that is at least 75%, at least 77.7%, at least 85%, at least 88.8% or 100% identical to SEQ ID NO: 4349. In some embodiments, a 581-589 region of an AAV5 VP capsid polypeptide has a sequence that is at least 75%, at least 77.7%, at least 85%, at least 88.8% or 100% identical to SEQ ID NO: 293. In some embodiments, a 581-589 region of an AAV5 VP capsid polypeptide has a sequence that is at least 75%, at least 77.7%, at least 85%, at least 88.8% or 100% identical to SEQ ID NO: 1576. In some embodiments, a 581-589 region of an AAV5 VP capsid polypeptide has a sequence that is at least 75%, at least 77.7%, at least 85%, at least 88.8% or 100% identical to SEQ ID NO: 2661. In some embodiments, a 581-589 region of an AAV5 VP capsid polypeptide has a sequence that is at least 75%, at least 77.7%, at least 85%, at least 88.8% or 100% identical to SEQ ID NO: 4964. In some embodiments, a 581-589 region of an AAV5 VP capsid polypeptide has a sequence that is at least 75%, at least 77.7%, at least 85%, at least 88.8% or 100% identical to SEQ ID NO: 4314. In some embodiments, a 581-589 region of an AAV5 VP capsid polypeptide has a sequence that is at least 75%, at least 77.7%, at least 85%, at least 88.8% or 100% identical to SEQ ID NO: 4995. In some embodiments, a 581-589 region of an AAV5 VP capsid polypeptide has a sequence that is at least 75%, at least 77.7%, at least 85%, at least 88.8% or 100% identical to SEQ ID NO: 4366. In some embodiments, a 581-589 region of an AAV5 VP capsid polypeptide has a sequence that is at least 75%, at least 77.7%, at least 85%, at least 88.8% or 100% identical to SEQ ID NO: 4545. In some embodiments, a 581-589 region of an AAV5 VP capsid polypeptide has a sequence that is at least 75%, at least 77.7%, at least 85%, at least 88.8% or 100% identical to SEQ ID NO: 4961. In some embodiments, a 581-589 region of an AAV5 VP capsid polypeptide has a sequence that is at least 75%, at least 77.7%, at least 85%, at least 88.8% or 100% identical to SEQ ID NO: 5206. In some embodiments, a 581-589 region of an AAV5 VP capsid polypeptide has a sequence that is at least 75%, at least 77.7%, at least 85%, at least 88.8% or 100% identical to SEQ ID NO: 5092. In some embodiments, a 581-589 region of an AAV5 VP capsid polypeptide has a sequence that is at least 75%, at least 77.7%, at least 85%, at least 88.8% or 100% identical to SEQ ID NO: 3472. In some embodiments, a 581-589 region of an AAV5 VP capsid polypeptide has a sequence that is at least 75%, at least 77.7%, at least 85%, at least 88.8% or 100% identical to SEQ ID NO: 5075. In some embodiments, a 581-589 region of an AAV5 VP capsid polypeptide has a sequence that is at least 75%, at least 77.7%, at least 85%, at least 88.8% or 100% identical to SEQ ID NO: 4059. In some embodiments, a 581-589 region of an AAV5 VP capsid polypeptide has a sequence that is at least 75%, at least 77.7%, at least 85%, at least 88.8% or 100% identical to SEQ ID NO: 4354. In some embodiments, a 581-589 region of an AAV5 VP capsid polypeptide has a sequence that is at least 75%, at least 77.7%, at least 85%, at least 88.8% or 100% identical to SEQ ID NO: 5060. In some embodiments, a 581-589 region of an AAV5 VP capsid polypeptide has a sequence that is at least 75%, at least 77.7%, at least 85%, at least 88.8% or 100% identical to SEQ ID NO: 4288. In some embodiments, a 581-589 region of an AAV5 VP capsid polypeptide has a sequence that is at least 75%, at least 77.7%, at least 85%, at least 88.8% or 100% identical to SEQ ID NO: 4952. In some embodiments, a 581-589 region of an AAV5 VP capsid polypeptide has a sequence that is at least 75%, at least 77.7%, at least 85%, at least 88.8% or 100% identical to SEQ ID NO: 5138. In some embodiments, a 581-589 region of an AAV5 VP capsid polypeptide has a sequence that is at least 75%, at least 77.7%, at least 85%, at least 88.8% or 100% identical to SEQ ID NO: 18. In some embodiments, a 581-589 region of an AAV5 VP capsid polypeptide has a sequence that is at least 75%, at least 77.7%, at least 85%, at least 88.8% or 100% identical to SEQ ID NO: 5030. In some embodiments, a 581-589 region of an AAV5 VP capsid polypeptide has a sequence that is at least 75%, at least 77.7%, at least 85%, at least 88.8% or 100% identical to SEQ ID NO: 3297. In some embodiments, a 581-589 region of an AAV5 VP capsid polypeptide has a sequence that is at least 75%, at least 77.7%, at least 85%, at least 88.8% or 100% identical to SEQ ID NO: 4295. In some embodiments, a 581-589 region of an AAV5 VP capsid polypeptide has a sequence that is at least 75%, at least 77.7%, at least 85%, at least 88.8% or 100% identical to SEQ ID NO: 4363. In some embodiments, a 581-589 region of an AAV5 VP capsid polypeptide has a sequence that is at least 75%, at least 77.7%, at least 85%, at least 88.8% or 100% identical to SEQ ID NO: 4963.

In some embodiments, a 581-589 region of an AAV5 VP capsid polypeptide has a sequence of SEQ ID NO: 348. In some embodiments, a 581-589 region of an AAV5 VP capsid polypeptide has a sequence of SEQ ID NO: 2536. In some embodiments, a 581-589 region of an AAV5 VP capsid polypeptide has a sequence of SEQ ID NO: 4955. In some embodiments, a 581-589 region of an AAV5 VP capsid polypeptide has a sequence of SEQ ID NO: 5017. In some embodiments, a 581-589 region of an AAV5 VP capsid polypeptide has a sequence of SEQ ID NO: 4349. In some embodiments, a 581-589 region of an AAV5 VP capsid polypeptide has a sequence of SEQ ID NO: 293. In some embodiments, a 581-589 region of an AAV5 VP capsid polypeptide has a sequence of SEQ ID NO: 1576. In some embodiments, a 581-589 region of an AAV5 VP capsid polypeptide has a sequence of SEQ ID NO: 2661. In some embodiments, a 581-589 region of an AAV5 VP capsid polypeptide has a sequence of SEQ ID NO: 4964. In some embodiments, a 581-589 region of an AAV5 VP capsid polypeptide has a sequence of SEQ ID NO: 4314. In some embodiments, a 581-589 region of an AAV5 VP capsid polypeptide has a sequence of SEQ ID NO: 4995. In some embodiments, a 581-589 region of an AAV5 VP capsid polypeptide has a sequence of SEQ ID NO: 4366. In some embodiments, a 581-589 region of an AAV5 VP capsid polypeptide has a sequence of SEQ ID NO: 4545. In some embodiments, a 581-589 region of an AAV5 VP capsid polypeptide has a sequence of SEQ ID NO: 4961. In some embodiments, a 581-589 region of an AAV5 VP capsid polypeptide has a sequence of SEQ ID NO: 5206. In some embodiments, a 581-589 region of an AAV5 VP capsid polypeptide has a sequence of SEQ ID NO: 5092. In some embodiments, a 581-589 region of an AAV5 VP capsid polypeptide has a sequence of SEQ ID NO: 3472. In some embodiments, a 581-589 region of an AAV5 VP capsid polypeptide has a sequence of SEQ ID NO: 5075. In some embodiments, a 581-589 region of an AAV5 VP capsid polypeptide has a sequence of SEQ ID NO: 4059. In some embodiments, a 581-589 region of an AAV5 VP capsid polypeptide has a sequence of SEQ ID NO: 4354. In some embodiments, a 581-589 region of an AAV5 VP capsid polypeptide has a sequence of SEQ ID NO: 5060. In some embodiments, a 581-589 region of an AAV5 VP capsid polypeptide has a sequence of SEQ ID NO: 4288. In some embodiments, a 581-589 region of an AAV5 VP capsid polypeptide has a sequence of SEQ ID NO: 4952. In some embodiments, a 581-589 region of an AAV5 VP capsid polypeptide has a sequence of SEQ ID NO: 5138. In some embodiments, a 581-589 region of an AAV5 VP capsid polypeptide has a sequence of SEQ ID NO: 18. In some embodiments, a 581-589 region of an AAV5 VP capsid polypeptide has a sequence of SEQ ID NO: 5030. In some embodiments, a 581-589 region of an AAV5 VP capsid polypeptide has a sequence of SEQ ID NO: 3297. In some embodiments, a 581-589 region of an AAV5 VP capsid polypeptide has a sequence of SEQ ID NO: 4295. In some embodiments, a 581-589 region of an AAV5 VP capsid polypeptide has a sequence of SEQ ID NO: 4363. In some embodiments, a 581-589 region of an AAV5 VP capsid polypeptide has a sequence of SEQ ID NO: 4963.

In some embodiments, a 581-589 region of an AAV5 VP capsid polypeptide has a sequence of SEQ ID NO: 348 with 1 or 2 conservative substitutions. In some embodiments, a 581-589 region of an AAV5 VP capsid polypeptide has a sequence of SEQ ID NO: 2536 with 1 or 2 conservative substitutions. In some embodiments, a 581-589 region of an AAV5 VP capsid polypeptide has a sequence of SEQ ID NO: 4955 with 1 or 2 conservative substitutions. In some embodiments, a 581-589 region of an AAV5 VP capsid polypeptide has a sequence of SEQ ID NO: 5017 with 1 or 2 conservative substitutions. In some embodiments, a 581-589 region of an AAV5 VP capsid polypeptide has a sequence of SEQ ID NO: 4349 with 1 or 2 conservative substitutions. In some embodiments, a 581-589 region of an AAV5 VP capsid polypeptide has a sequence of SEQ ID NO: 293 with 1 or 2 conservative substitutions. In some embodiments, a 581-589 region of an AAV5 VP capsid polypeptide has a sequence of SEQ ID NO: 1576 with 1 or 2 conservative substitutions. In some embodiments, a 581-589 region of an AAV5 VP capsid polypeptide has a sequence of SEQ ID NO: 2661 with 1 or 2 conservative substitutions. In some embodiments, a 581-589 region of an AAV5 VP capsid polypeptide has a sequence of SEQ ID NO: 4964 with 1 or 2 conservative substitutions. In some embodiments, a 581-589 region of an AAV5 VP capsid polypeptide has a sequence of SEQ ID NO: 4314 with 1 or 2 conservative substitutions. In some embodiments, a 581-589 region of an AAV5 VP capsid polypeptide has a sequence of SEQ ID NO: 4995 with 1 or 2 conservative substitutions. In some embodiments, a 581-589 region of an AAV5 VP capsid polypeptide has a sequence of SEQ ID NO: 4366 with 1 or 2 conservative substitutions. In some embodiments, a 581-589 region of an AAV5 VP capsid polypeptide has a sequence of SEQ ID NO: 4545 with 1 or 2 conservative substitutions. In some embodiments, a 581-589 region of an AAV5 VP capsid polypeptide has a sequence of SEQ ID NO: 4961 with 1 or 2 conservative substitutions. In some embodiments, a 581-589 region of an AAV5 VP capsid polypeptide has a sequence of SEQ ID NO: 5206 with 1 or 2 conservative substitutions. In some embodiments, a 581-589 region of an AAV5 VP capsid polypeptide has a sequence of SEQ ID NO: 5092 with 1 or 2 conservative substitutions. In some embodiments, a 581-589 region of an AAV5 VP capsid polypeptide has a sequence of SEQ ID NO: 3472 with 1 or 2 conservative substitutions. In some embodiments, a 581-589 region of an AAV5 VP capsid polypeptide has a sequence of SEQ ID NO: 5075 with 1 or 2 conservative substitutions. In some embodiments, a 581-589 region of an AAV5 VP capsid polypeptide has a sequence of SEQ ID NO: 4059 with 1 or 2 conservative substitutions. In some embodiments, a 581-589 region of an AAV5 VP capsid polypeptide has a sequence of SEQ ID NO: 4354 with 1 or 2 conservative substitutions. In some embodiments, a 581-589 region of an AAV5 VP capsid polypeptide has a sequence of SEQ ID NO: 5060 with 1 or 2 conservative substitutions. In some embodiments, a 581-589 region of an AAV5 VP capsid polypeptide has a sequence of SEQ ID NO: 4288 with 1 or 2 conservative substitutions. In some embodiments, a 581-589 region of an AAV5 VP capsid polypeptide has a sequence of SEQ ID NO: 4952 with 1 or 2 conservative substitutions. In some embodiments, a 581-589 region of an AAV5 VP capsid polypeptide has a sequence of SEQ ID NO: 5138 with 1 or 2 conservative substitutions. In some embodiments, a 581-589 region of an AAV5 VP capsid polypeptide has a sequence of SEQ ID NO: 18 with 1 or 2 conservative substitutions. In some embodiments, a 581-589 region of an AAV5 VP capsid polypeptide has a sequence of SEQ ID NO: 5030 with 1 or 2 conservative substitutions. In some embodiments, a 581-589 region of an AAV5 VP capsid polypeptide has a sequence of SEQ ID NO: 3297 with 1 or 2 conservative substitutions. In some embodiments, a 581-589 region of an AAV5 VP capsid polypeptide has a sequence of SEQ ID NO: 4295 with 1 or 2 conservative substitutions. In some embodiments, a 581-589 region of an AAV5 VP capsid polypeptide has a sequence of SEQ ID NO: 4363 with 1 or 2 conservative substitutions. In some embodiments, a 581-589 region of an AAV5 VP capsid polypeptide has a sequence of SEQ ID NO: 4963 with 1 or 2 conservative substitutions. The conservative substitutions may be at any position in the 581-589 region.

In some embodiments, an AAV VP capsid polypeptide preferentially targets a muscle tissue at a DNA enrichment of at least 20-fold greater than a wild type AAV9. For example, an AAV VP capsid polypeptide comprising a 581-589 region of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, SEQ ID NO: 4366, SEQ ID NO: 4545, SEQ ID NO: 4961, SEQ ID NO: 5206, SEQ ID NO: 5092, SEQ ID NO: 3472, SEQ ID NO: 5075, SEQ ID NO: 4059, SEQ ID NO: 4354, SEQ ID NO: 5060, SEQ ID NO: 4288, SEQ ID NO: 4952, SEQ ID NO: 5138, SEQ ID NO: 18, SEQ ID NO: 5030, SEQ ID NO: 3297, SEQ ID NO: 4295, SEQ ID NO: 4363, SEQ ID NO: 4963, SEQ ID NO: 4973, SEQ ID NO: 5159, SEQ ID NO: 4960, SEQ ID NO: 4938, SEQ ID NO: 5157, SEQ ID NO: 5023, or SEQ ID NO: 4969 may preferentially target a muscle tissue at a DNA enrichment of at least 20-fold greater than a wild type AAV9.

In some embodiments, an AAV VP capsid polypeptide preferentially targets a muscle tissue at a DNA enrichment of at least 25-fold greater than the wild type AAV9. For example, an AAV VP capsid polypeptide comprising a 581-589 region of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, or SEQ ID NO: 4366 may preferentially target a muscle tissue at a DNA enrichment of at least 25-fold greater than the wild type AAV9.

In some embodiments, an AAV VP capsid polypeptide preferentially targets a muscle tissue at a DNA enrichment of at least 30-fold greater than the wild type AAV9. For example, an AAV VP capsid polypeptide comprising a 581-589 region of SEQ ID NO: 348 or SEQ ID NO: 2536 may preferentially target a muscle tissue at a DNA enrichment of at least 30-fold greater than the wild type AAV9.

In some embodiments, an AAV VP capsid polypeptide preferentially targets a muscle tissue at an RNA enrichment of at least 5-fold greater than a wild type AAV9. For example, an AAV VP capsid polypeptide comprising a 581-589 region of SEQ ID NO: 5138, SEQ ID NO: 3472, SEQ ID NO: 348, SEQ ID NO: 5052, SEQ ID NO: 5193, SEQ ID NO: 5026, SEQ ID NO: 278, SEQ ID NO: 5163, SEQ ID NO: 293, SEQ ID NO: 4943, SEQ ID NO: 4952, SEQ ID NO: 4317, SEQ ID NO: 5075, SEQ ID NO: 386, SEQ ID NO: 4986, SEQ ID NO: 4995, SEQ ID NO: 4288, SEQ ID NO: 5030, SEQ ID NO: 5206, SEQ ID NO: 5159, SEQ ID NO: 4949, SEQ ID NO: 4338, SEQ ID NO: 5060, SEQ ID NO: 4346, SEQ ID NO: 5215, SEQ ID NO: 5017, SEQ ID NO: 4314, SEQ ID NO: 5080, SEQ ID NO: 4964, SEQ ID NO: 4363, SEQ ID NO: 4379, SEQ ID NO: 5027, SEQ ID NO: 4304, SEQ ID NO: 18, SEQ ID NO: 4349, SEQ ID NO: 4938, or SEQ ID NO: 2536 may preferentially target a muscle tissue at an RNA enrichment of at least 5-fold greater than a wild type AAV9.

In some embodiments, an AAV VP capsid polypeptide preferentially targets a muscle tissue at an RNA enrichment of at least 10-fold or greater than the wild type AAV9. For example, an AAV VP capsid polypeptide comprising a 581-589 region of SEQ ID NO: 5138, SEQ ID NO: 3472, SEQ ID NO: 348, SEQ ID NO: 5052, SEQ ID NO: 5193, SEQ ID NO: 5026, SEQ ID NO: 278, or SEQ ID NO: 5163 may preferentially target a muscle tissue at an RNA enrichment of at least 10-fold or greater than the wild type AAV9.

In some embodiments, an AAV VP capsid polypeptide preferentially targets a muscle tissue at an RNA enrichment of at least 100-fold or greater than the wild type AAV9. For example, an AAV VP capsid polypeptide comprising a 581-589 region of SEQ ID NO: 5138 may preferentially target a muscle tissue at an RNA enrichment of at least 100-fold or greater than the wild type AAV9.

In some embodiments, an AAV VP capsid polypeptide preferentially targets a muscle tissue at a DNA enrichment of at least 5-fold greater than a wild type AAV5. For example, an AAV VP capsid polypeptide comprising a 581-589 region of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, SEQ ID NO: 4366, SEQ ID NO: 4545, SEQ ID NO: 4961, SEQ ID NO: 5206, SEQ ID NO: 5092, SEQ ID NO: 3472, SEQ ID NO: 5075, SEQ ID NO: 4059, SEQ ID NO: 4354, or SEQ ID NO: 5060 may preferentially target a muscle tissue at a DNA enrichment of at least 5-fold greater than a wild type AAV5.

In some embodiments, an AAV VP capsid polypeptide preferentially targets a muscle tissue at a DNA enrichment of at least 10-fold greater than the wild type AAV5. For example, an AAV VP capsid polypeptide comprising a 581-589 region of SEQ ID NO: 348 may preferentially target a muscle tissue at a DNA enrichment of at least 10-fold greater than the wild type AAV5.

In some embodiments, an AAV VP capsid polypeptide preferentially targets a muscle tissue at an RNA enrichment of at least that of a wild type AAV5. For example, an AAV VP capsid polypeptide comprising a 581-589 region of SEQ ID NO: 5138, SEQ ID NO: 3472, SEQ ID NO: 348, SEQ ID NO: 5052, SEQ ID NO: 5193, SEQ ID NO: 5026, SEQ ID NO: 278, SEQ ID NO: 5163, SEQ ID NO: 293, SEQ ID NO: 4943, SEQ ID NO: 4952, SEQ ID NO: 4317, SEQ ID NO: 5075, SEQ ID NO: 386, SEQ ID NO: 4986, or SEQ ID NO: 4995 may preferentially target a muscle tissue at an RNA enrichment of at least that of a wild type AAV5.

In some embodiments, an AAV VP capsid polypeptide preferentially targets a muscle tissue at an RNA enrichment of at least 2-fold greater than the wild type AAV5. For example, an AAV VP capsid polypeptide comprising a 581-589 region of SEQ ID NO: 5138, SEQ ID NO: 3472, SEQ ID NO: 348, and SEQ ID NO: 5052 may preferentially target a muscle tissue at an RNA enrichment of at least 2-fold greater than the wild type AAV5.

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 5 (SEQ ID NO: 4238-SEQ ID NO: 4933) that is expected to confer muscle tissue tropism or preference on a recombinant AAV virion (rAAV), based on a primary screen; and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 5 (SEQ ID NO: 4238-SEQ ID NO: 4933) at the region corresponding to amino acid residues 445 to 453 in AAV5 VP2 or the region corresponding to amino acid residues 389 to 397 in AAV5 VP3.

TABLE 5
Sequences of 581-589 Regions for Muscle
Tropism

| SEQ ID | |
|---|---|
| NO | Sequence |
| 4238 | AACFKQLMQ |
| 4239 | AACQWQSQR |
| 4240 | AACSFAIQR |
| 4241 | AADKPAHNP |
| 4242 | AAIQAMACG |
| 4243 | AALSINDQP |
| 4244 | AAMPHQKRE |
| 4245 | AAQYVMSLA |
| 4246 | AAREGVKLE |
| 4247 | ACATYQMTW |
| 4248 | ADCSIIYPE |
| 4249 | ADPRPAKGS |
| 4250 | AFQRTYQFH |
| 4251 | AGPFMVGFW |
| 4252 | AGTWENMSG |
| 4253 | AHILMGHNP |
| 4254 | AHLWYHQCA |
| 4255 | AIEQGLTMA |
| 4256 | AIFCAHYSY |
| 4257 | AKMYWDGHT |
| 4258 | AMFPQLWAQ |
| 4259 | AMNPKQMCF |
| 4260 | ANIPQTAWN |
| 4261 | APYCPTYIS |
| 4262 | AQGCMHRWQ |
| 4263 | AQIMLPSSK |
| 4264 | AQSTARHIE |
| 4265 | AQTEFGFME |
| 4266 | ARCLSVIEQ |
| 4267 | ARQLESFWT |
| 4268 | ASFYQLLIP |
| 4269 | ASIVLRDSF |
| 4270 | ASNPLNCPN |
| 4271 | ASRFEDLWE |
| 4272 | ASTEWDMHN |
| 4273 | ASVELMKVC |
| 4274 | ASYLWCTMP |
| 4275 | ATDRIRTMM |
| 4276 | ATELGKDSG |
| 4277 | ATGRAAYHE |
| 4278 | ATIWPVESW |
| 4279 | ATLCTQRGS |
| 4280 | ATLFYNAFW |
| 4281 | ATTAVGDAL |
| 4282 | ATWISHNAS |
| 4283 | AVALSFVSS |
| 4284 | AVCEYLRMD |
| 4285 | AVTQWMLYA |
| 4286 | AYPERLYSD |
| 4287 | AYRQMCIER |
| 4288 | CACKVSMSG |
| 4289 | CACSIGHHR |
| 4290 | CAFQMYDSP |
| 4291 | CAKQYDKGI |
| 4292 | CAMLLQAVQ |
| 4293 | CAQPQHNWC |
| 4294 | CATFVNKLS |
| 4295 | CAVKFHAYD |
| 4296 | CDGELGRKC |
| 4297 | CDGRGIPGC |
| 4298 | CDGSCKYYQ |
| 4299 | CDWMMANLG |
| 4300 | CEPEGWIRA |
| 4301 | CEPFNINWW |
| 4302 | CESHEYRGH |
| 4303 | CFMPMMYYE |
| 4304 | CFRLHEMHQ |
| 4305 | CFRNILDDS |
| 4306 | CGRCNWRGY |
| 4307 | CHGYTWERP |
| 4308 | CHWFRCSCA |
| 4309 | CIAFVKQCC |
| 4310 | CIARTDYQP |
| 4311 | CIGKWPISR |
| 4312 | CIHRLIEDN |
| 4313 | CIMSMMWSQ |
| 4314 | CIMYRSEAS |
| 4315 | CIQATKAYC |
| 4316 | CIRHAQDCY |
| 4317 | CITHQQMAV |
| 4318 | CKEWYVRDR |
| 4319 | CKPANWVRY |
| 4320 | CKTHIWGFP |
| 4321 | CKVIVNIGA |
| 4322 | CKWIPWQCD |
| 4323 | CKWPCTWNS |
| 4324 | CKYCCDSHE |
| 4325 | CLMEKISDF |
| 4326 | CLPFFINRW |
| 4327 | CMESYSISE |
| 4328 | CMINLFDAC |
| 4329 | CMNESGQFH |
| 4330 | CMQRPHTAP |
| 4331 | CMSQFHLER |
| 4332 | CNETPWHKG |
| 4333 | CPAILNVAC |
| 4334 | CPKWSNHDQ |
| 4335 | CPMKHIQDR |
| 4336 | CPQQDDWPG |
| 4337 | CPWFGFLGW |
| 4338 | CQATFSRAY |
| 4339 | CQCNWWGRP |
| 4340 | CQQRYGHGK |
| 4341 | CRCPLSIMS |
| 4342 | CSDLWRIPF |
| 4343 | CSHKKEQYD |
| 4344 | CSICHYDHA |
| 4345 | CSIYQQMFQ |
| 4346 | CSMSTQRGA |
| 4347 | CSSLFMFNT |
| 4348 | CSSNWPMGI |
| 4349 | CSSYLVTAN |
| 4350 | CSTARGYDL |
| 4351 | CSTESSNSM |
| 4352 | CSTTYGRKE |
| 4353 | CSWYTHAGA |
| 4354 | CTFMVYSQG |
| 4355 | CTFQSTSAW |
| 4356 | CTKTFDRTE |
| 4357 | CTNIQSVFH |
| 4358 | CTPTQWGSA |
| 4359 | CVATKSRML |
| 4360 | CVAVPLSMS |
| 4361 | CVGFWTQMN |
| 4362 | CVGTVFHTA |
| 4363 | CVHIKVDGT |
| 4364 | CVHVKEQSQ |
| 4365 | CVIYNDMQI |
| 4366 | CVLFRHVDQ |
| 4367 | CVMPLGMAA |
| 4368 | CVNMKYVDS |
| 4369 | CVQSEWGSK |
| 4370 | CVQWQMNDA |
| 4371 | CVSLHSISM |
| 4372 | CVYQFDFTQ |
| 4373 | CWELLTWQW |
| 4374 | CWFDFMITE |
| 4375 | CWGYTPDWN |
| 4376 | CYAPKFIGD |
| 4377 | CYIDYRMSY |
| 4378 | CYLEIPNNF |
| 4379 | CYNLFNSMY |
| 4380 | CYSLRPDGP |
| 4381 | DAKAHIFSF |
| 4382 | DASECKWYN |
| 4383 | DDLIPINPW |
| 4384 | DDVAEKQCC |
| 4385 | DEHTIDVMQ |
| 4386 | DENGTWPHG |
| 4387 | DEPENMYSS |
| 4388 | DFFWPRPKA |
| 4389 | DFPRPENHW |
| 4390 | DHECVHGSG |
| 4391 | DHELKGACS |
| 4392 | DHLWFSSPG |
| 4393 | DHNPIMMWC |
| 4394 | DHVECWPQG |
| 4395 | DIYPWVCYY |
| 4396 | DKCHWASEG |
| 4397 | DLGNDRWRF |
| 4398 | DMHSYNNAK |
| 4399 | DMKGRQDGA |
| 4400 | DQSPLRTQS |
| 4401 | DRMMYARNG |
| 4402 | DRNQLYSSF |
| 4403 | DSACAGWGL |
| 4404 | DSDYVLVDY |
| 4405 | DSIAKGVAH |
| 4406 | DSPFGWFNC |
| 4407 | DSPWSKWQP |
| 4408 | DTKDYPKQS |
| 4409 | DTKPVEEVK |
| 4410 | DTLHKLSDC |
| 4411 | DTRCYGEMF |
| 4412 | DTVQMLCQY |
| 4413 | DVHCICRYD |
| 4414 | DVHKWSCAK |
| 4415 | DVICRHHGM |
| 4416 | DVILSQEYN |
| 4417 | DVLKVRIQE |
| 4418 | DVRIPCCQH |
| 4419 | DVTMYAAWS |
| 4420 | DVVLCNQWR |
| 4421 | DVWNDLNSM |
| 4422 | DWKLTYPQG |
| 4423 | DWMVVRKEG |
| 4424 | DWPHIMFNY |
| 4425 | DYTFKRYVC |
| 4426 | EAEQCMIGQ |
| 4427 | EAGWFNHST |
| 4428 | EAHDLAGNM |
| 4429 | EAHKMISTR |
| 4430 | EAKASAHTC |
| 4431 | EAQLYPDML |
| 4432 | EASCMSIWT |
| 4433 | ECGHIFYPA |
| 4434 | ECIDVMQNR |
| 4435 | ECQLVKTVP |
| 4436 | ECTVICSSS |
| 4437 | ECVGGNIWG |
| 4438 | EERKCNLGC |
| 4439 | EFRNYWSPY |
| 4440 | EFWSVKNHD |
| 4441 | EHHICSKSA |
| 4442 | EHMKLFLSE |
| 4443 | EIATHITFQ |
| 4444 | EIGMVMDMN |
| 4445 | EIMALDSPC |
| 4446 | EKEWGTPMW |
| 4447 | EKFPPHGDV |
| 4448 | ELKWQVAQF |
| 4449 | EMGPYPYPS |
| 4450 | EMKIDEHWP |
| 4451 | EMLLQPMYG |
| 4452 | ENCVISVHH |
| 4453 | ENGFIHKQS |
| 4454 | ENQPYWQVY |
| 4455 | EPEGIYQPM |
| 4456 | EQDDYMIPP |
| 4457 | EQESWEMGP |
| 4458 | ESESANHEG |
| 4459 | ESFAKHDQN |
| 4460 | ESGSQSGQA |
| 4461 | ESSQVNWHR |
| 4462 | ESTGESTLG |
| 4463 | ETQPHADYE |
| 4464 | ETTKWEAQH |
| 4465 | EVGCSWMWG |
| 4466 | EVGNYPRIP |
| 4467 | EVGRQQWMD |
| 4468 | EVKADIAQM |
| 4469 | EVKSANATW |
| 4470 | EVPAQTMQS |
| 4471 | EVQKRLVAE |
| 4472 | EVTCESKIE |
| 4473 | FCDLAHHYE |
| 4474 | FCRDFVKDM |
| 4475 | FDKWPKPQC |
| 4476 | FDPADVQKW |
| 4477 | FFESWQYPY |
| 4478 | FHWREPYVE |
| 4479 | FKRHEIPEY |
| 4480 | FMWITISNY |
| 4481 | FRQPYVWDK |
| 4482 | FSCSLYRHG |
| 4483 | FTTSQTLAE |
| 4484 | FVGSRNCID |
| 4485 | FYFQNIHPW |
| 4486 | GCYQSNVIP |
| 4487 | GDCPYSMQP |
| 4488 | GDGHGMWKM |
| 4489 | GDPKSKHDC |
| 4490 | GEHHCKFPM |
| 4491 | GEKWELYPM |
| 4492 | GEPRPNYVD |
| 4493 | GFEFMLNCP |
| 4494 | GGDRWLGYW |
| 4495 | GGEDEKPGC |
| 4496 | GGHFGHITW |
| 4497 | GHDFTNQDR |
| 4498 | GHFLDRWRD |
| 4499 | GKFCMPRNY |
| 4500 | GKKCGWPND |
| 4501 | GKSDMDCVE |
| 4502 | GKWQQQMVL |
| 4503 | GNPNCCLTG |
| 4504 | GPCCKWCTE |
| 4505 | GQNHVIVGA |
| 4506 | GRADCSAHP |
| 4507 | GSAQNMFGW |
| 4508 | GSCLKIAQE |
| 4509 | GSGLHMGYK |
| 4510 | GSRDSPISF |
| 4511 | GTAPPGTYS |
| 4512 | GTATMMHTH |
| 4513 | GVDLDNDRE |
| 4514 | GVFPMLMHS |
| 4515 | GVGWDSCHN |
| 4516 | GVIYRHHDP |
| 4517 | GVNFEDGAY |
| 4518 | GVTERFCPD |
| 4519 | GVWSKNTPF |
| 4520 | GWMSHEYPF |
| 4521 | GWWPGKPMD |
| 4522 | GYPRDPHET |
| 4523 | HAAMSGEGM |
| 4524 | HAEWACSGR |
| 4525 | HASYMCGLW |
| 4526 | HCEAVLVHG |
| 4527 | HDIRNVRML |
| 4528 | HDRWPKNCM |
| 4529 | HEPFRISGE |
| 4530 | HHWLGDCNS |
| 4531 | HIGTQYRQY |
| 4532 | HMRKTLIWK |
| 4533 | HPQFKSHEY |
| 4534 | HQYWMIPDG |
| 4535 | HSEPKLDIP |
| 4536 | HTAQLMAHE |
| 4537 | HTIAANMVA |
| 4538 | HTPTDAHVG |
| 4539 | HTTPAAFHN |
| 4540 | HVDPGFASL |
| 4541 | HWPFPWMRI |
| 4542 | IAFMKSVEG |
| 4543 | IAMAYKGQI |
| 4544 | IAYYEKMWS |
| 4545 | ICMSRTQMK |
| 4546 | IDPQGWLTH |
| 4547 | IDSFGELRE |
| 4548 | IGVRYAVQE |
| 4549 | IIRVQVDGS |
| 4550 | IMCKCPVWC |
| 4551 | IMEIVGIGN |
| 4552 | INSRTNSMC |
| 4553 | IPWPPVKGD |
| 4554 | IQKMWDHFQ |
| 4555 | IQRTMEANG |
| 4556 | IQVVKMAHG |
| 4557 | ISADQSMQD |
| 4558 | ISAWQDFDA |
| 4559 | ISEIHYTQC |
| 4560 | ITGGFNKDK |
| 4561 | ITNWFQQDK |
| 4562 | ITQVWCSDG |
| 4563 | ITTCYPNQT |
| 4564 | IVCQSHVMN |
| 4565 | IVTQRHYQI |
| 4566 | IYRPVDSFR |
| 4567 | KACDSKESW |
| 4568 | KANEDLKQF |
| 4569 | KCDYEHYFA |
| 4570 | KCKCPKMPQ |
| 4571 | KEHVYGMCP |
| 4572 | KFMSQEFGP |
| 4573 | KGCLLDSDH |
| 4574 | KGMDTTIVP |
| 4575 | KHERDPPQY |
| 4576 | KKFQRALTC |
| 4577 | KMEPVELFT |
| 4578 | KMQFIDACA |
| 4579 | KMRDGTEQK |
| 4580 | KNALYVSYQ |
| 4581 | KPPWINYPR |
| 4582 | KQAQALEYF |
| 4583 | KQDCYFWHT |
| 4584 | KSWDTIQYT |
| 4585 | KSYICEDFF |
| 4586 | KTEWLMWYN |
| 4587 | KTVIMEDFK |
| 4588 | KVTDCNQSG |
| 4589 | KWMHYDSIC |
| 4590 | LACQIDRFH |
| 4591 | LAELYLNMR |
| 4592 | LAGNQMDAD |
| 4593 | LAHGRCFHE |
| 4594 | LALSANLGG |
| 4595 | LAPPITWTD |
| 4596 | LASLAMAKA |
| 4597 | LATHALMAH |
| 4598 | LAWSQLLTP |
| 4599 | LCEPIAIRD |
| 4600 | LCIRSLANG |
| 4601 | LCMRYGSKA |
| 4602 | LDRPRFTDW |
| 4603 | LGQPVDDYS |
| 4604 | LHNLYEMSP |
| 4605 | LIEPDSFRG |
| 4606 | LKAEKNTMS |
| 4607 | LMVDVQRCK |
| 4608 | LMYCRYCPS |
| 4609 | LNAEWDEWT |
| 4610 | LNFLWAMLW |
| 4611 | LPCSLLRGD |
| 4612 | LPIPPVPEY |
| 4613 | LPRLNFESK |
| 4614 | LPSAVVMHE |
| 4615 | LQGLSWLFP |
| 4616 | LQNLFSYQE |
| 4617 | LQQQTHWYG |
| 4618 | LRSFIDTMD |
| 4619 | LSFCLGHND |
| 4620 | LSMIPMAQW |
| 4621 | LSYWSGNAA |
| 4622 | LTAPMEEDN |
| 4623 | LTCLQDGDA |
| 4624 | LTCQYQGGQ |
| 4625 | LTMGHTMQA |
| 4626 | LVHEPGAVT |
| 4627 | LVHQKWECK |
| 4628 | LVHREECQI |
| 4629 | LVHTIMMGS |
| 4630 | LYHAYSSGF |
| 4631 | MAANVSEEK |
| 4632 | MACAFGTNF |
| 4633 | MACWKNATE |
| 4634 | MAQIGPLHH |
| 4635 | MASTCTQQD |
| 4636 | MATRLFDSG |
| 4637 | MDFNIDWHT |
| 4638 | MEASNGFGL |
| 4639 | MEDSCRPGF |
| 4640 | MHMPCRVGK |
| 4641 | MIEENEWFG |
| 4642 | MILPIHHQQ |
| 4643 | MKQWPVFPS |
| 4644 | MLGVSTWEW |
| 4645 | MMRIDGLGP |
| 4646 | MNATQMYGQ |
| 4647 | MNGYYHVMN |
| 4648 | MNLGKNMWN |
| 4649 | MNMEYCQFG |
| 4650 | MPRLTTYTW |
| 4651 | MQGFFTRGF |
| 4652 | MQNKQASTP |
| 4653 | MSDLWPFSY |
| 4654 | MTACHTMGR |
| 4655 | MTCDFSAAE |
| 4656 | MTCHFQHMH |
| 4657 | MTCNWGLNG |
| 4658 | MTKPGIPNF |
| 4659 | MTMDWQFQG |
| 4660 | MTSIVMENA |
| 4661 | MTSRVDRTD |
| 4662 | MVENWAKDM |
| 4663 | MVEQFACLP |
| 4664 | MVLCYNQMV |
| 4665 | MVNMHVFHS |
| 4666 | MVSVVRQGS |
| 4667 | MVTEYYHDV |
| 4668 | MWTMFNGQF |
| 4669 | MYDTACVIG |
| 4670 | MYWPDISRN |
| 4671 | NATPWNFGT |
| 4672 | NAVEKMAFN |
| 4673 | NAYMQQAQG |
| 4674 | NCMQMHWDG |
| 4675 | NCNCDEYHY |
| 4676 | NDGFDFWTC |
| 4677 | NELNNGREF |
| 4678 | NEPWPDHLP |
| 4679 | NFCYKNIYA |
| 4680 | NFMCSAGHT |
| 4681 | NGPWDWWIN |
| 4682 | NHFCGEWLQ |
| 4683 | NHWILSCAE |
| 4684 | NIERTVKHM |
| 4685 | NIKHEEMWN |
| 4686 | NIMTVDMWP |
| 4687 | NIPFKERSW |
| 4688 | NKPCWPCPH |
| 4689 | NKSDMSCYG |
| 4690 | NMSVYMKLG |
| 4691 | NPHARTVNG |
| 4692 | NPMPDWNFT |
| 4693 | NPSMGYFGF |
| 4694 | NQRIMDAGL |
| 4695 | NRHLGNNPW |
| 4696 | NRMNYDAWG |
| 4697 | NRWSVYRAE |
| 4698 | NTGCLKQAT |
| 4699 | NTLRHCVHT |
| 4700 | NTMQAMDVH |
| 4701 | NTQWNQKVP |
| 4702 | NVATQFWDM |
| 4703 | NVGMYGQKA |
| 4704 | NVHCQAASQ |
| 4705 | NVIEYQNRD |
| 4706 | NVPPPKFMA |
| 4707 | NYALLGDWE |
| 4708 | PCDVNHTSG |
| 4709 | PCEFSTCVR |
| 4710 | PCKGPPDIS |
| 4711 | PCNDHPCAG |
| 4712 | PCPCKGDAA |
| 4713 | PDAERKPTW |
| 4714 | PDCEKYENE |
| 4715 | PDCFPWDHN |
| 4716 | PDCGEWYPG |
| 4717 | PDGILNTWW |
| 4718 | PDICMQSGW |
| 4719 | PDLWTWQSD |
| 4720 | PDPWFMMRP |
| 4721 | PEAELPDTS |
| 4722 | PEKMRWMHH |
| 4723 | PELTLVNPG |
| 4724 | PFFPIDELG |
| 4725 | PFNCKWGMF |
| 4726 | PFNDHESEK |
| 4727 | PGDQNSYPG |
| 4728 | PGDRQEKMY |
| 4729 | PGKEFQPKW |
| 4730 | PGLLDNIPS |
| 4731 | PGPRPGEMC |
| 4732 | PHMPPFHCD |
| 4733 | PIAPHNDYW |
| 4734 | PIDQNPKFG |
| 4735 | PIPSPYCGF |
| 4736 | PKGSKYHEW |
| 4737 | PKIEPPFHS |
| 4738 | PKRGDSLPN |
| 4739 | PLKSGWTRT |
| 4740 | PLRTEISPW |
| 4741 | PNECARGDC |
| 4742 | PNFFNYHTS |
| 4743 | PNGLAELKP |
| 4744 | PPHCL WNTI |
| 4745 | PPIMMTIAN |
| 4746 | PPISHYWFD |
| 4747 | PPKCHQWQW |
| 4748 | PPLDTEHYF |
| 4749 | PPLISGFET |
| 4750 | PPNDRRFYD |
| 4751 | PPPCLEAPY |
| 4752 | PPPMQGSRM |
| 4753 | PPPWHQFDE |
| 4754 | PPRCEMLCM |
| 4755 | PPRMPYHPY |
| 4756 | PPVRMLFKQ |
| 4757 | PPWYHKGNS |
| 4758 | PQYEQYWQE |
| 4759 | PREYHPEEP |
| 4760 | PRFCMWCCG |
| 4761 | PRLICHERS |
| 4762 | PSIHGWELG |
| 4763 | PVKSEMWPT |
| 4764 | PWERWEHKG |
| 4765 | PYHPPTSGN |
| 4766 | PYLPDGNPM |
| 4767 | PYSCIPDGC |
| 4768 | QAACKIADL |
| 4769 | QCEAVRRCR |
| 4770 | QCLLIHEVG |
| 4771 | QCTKLHDWH |
| 4772 | QEKAMDNFQ |
| 4773 | QEYSQWWDN |
| 4774 | QFGTHHRHN |
| 4775 | QFTHMNWLQ |
| 4776 | QGCHLMTGQ |
| 4777 | QGDFCQQMN |
| 4778 | QGGWNSTCE |
| 4779 | QGKENNVEE |
| 4780 | QHDSKVAWM |
| 4781 | QHVHKDGIE |
| 4782 | QHYQPGPHN |
| 4783 | QIEYAQMVG |
| 4784 | QIQPVSCPN |
| 4785 | QKQYQDCVT |
| 4786 | QMAHVQYQQ |
| 4787 | QNIQMHFLG |
| 4788 | QNRETIVGG |
| 4789 | QPMTREMPP |
| 4790 | QQHQYTSKQ |
| 4791 | QSCMKIHHA |
| 4792 | QSISYTHND |
| 4793 | QSNLKKEDH |
| 4794 | QSSIENRFN |
| 4795 | QSSYYYAQS |
| 4796 | QSVKYNWGH |
| 4797 | QTDVTQSMY |
| 4798 | QTECLKWMR |
| 4799 | QTGLYNVMV |
| 4800 | QTHTQTYSW |
| 4801 | QVDDGKGPE |
| 4802 | QVIAVQDFR |
| 4803 | QVVECQHIG |
| 4804 | QVWDYERGI |
| 4805 | QYCPNSLEF |
| 4806 | QYQQSTVSN |
| 4807 | QYRNIDEDY |
| 4808 | RDMCQWDQP |
| 4809 | RGVHVEVEE |
| 4810 | RMQPINARI |
| 4811 | RMVGLEKAY |
| 4812 | RNQLKIDDM |
| 4813 | RSTGILEHC |
| 4814 | RSTSDPAML |
| 4815 | RTAWDQPFS |
| 4816 | RTNEAMMWN |
| 4817 | RVDYCYYFQ |
| 4818 | SACHNNTLQ |
| 4819 | SAGYLQKVH |
| 4820 | SASEAIAHQ |
| 4821 | SCCQQFNSG |
| 4822 | SDNGEYYRI |
| 4823 | SDNIQVWPW |
| 4824 | SDRQRFIPW |
| 4825 | SFCGLLTGR |
| 4826 | SGCPYWWPS |
| 4827 | SGKEQDSWN |
| 4828 | SHDVSGSCE |
| 4829 | SHFCYSMQE |
| 4830 | SISWKHSEA |
| 4831 | SITVQHEIS |
| 4832 | SKQCTGKEA |
| 4833 | SLAWNRVIQ |
| 4834 | SLPNEGYYP |
| 4835 | SMAWRNVIP |
| 4836 | SMCYVHGKE |
| 4837 | SMEKPSHYQ |
| 4838 | SPWYDQLCW |
| 4839 | SSADNVCWS |
| 4840 | SSCQFVADL |
| 4841 | SSDQYEEMN |
| 4842 | SSLCCNVGW |
| 4843 | STDQYNQDF |
| 4844 | STISEGSKN |
| 4845 | STLRHALIE |
| 4846 | SVAHSPMQC |
| 4847 | SVAMKQIEE |
| 4848 | SVGDNSFEH |
| 4849 | SVLTPHVTF |
| 4850 | SVTFKHQHQ |
| 4851 | SYKEYLHDE |
| 4852 | SYMWLQKGQ |
| 4853 | SYSIKQFHP |
| 4854 | TAAFMTTRD |
| 4855 | TACKWVHAC |
| 4856 | TANPTQGMQ |
| 4857 | TAVDHGLFH |
| 4858 | TCEPAVFGN |
| 4859 | TCEVYRQEC |
| 4860 | TCMRYNQHH |
| 4861 | TCQIAHQEA |
| 4862 | TECISKYPT |
| 4863 | TGHQYEWGY |
| 4864 | THHSSNQFF |
| 4865 | TKTATDFWQ |
| 4866 | TLQWMNAYR |
| 4867 | TMGMKHACC |
| 4868 | TPEHSVPGH |
| 4869 | TPKCGLWKD |
| 4870 | TQDEQRTMP |
| 4871 | TQHPYMKWH |
| 4872 | TSCEWDMMV |
| 4873 | TSMCYRTEE |
| 4874 | TTAFIGMHF |
| 4875 | TTAVLRENG |
| 4876 | TTLAVAHTG |
| 4877 | TVAGWMSHN |
| 4878 | TVAQSEAEF |
| 4879 | TVDMVNREQ |
| 4880 | TVFRVVQCA |
| 4881 | TVGHPGHAC |
| 4882 | TVHMWDQHA |
| 4883 | TWRMKGDDK |
| 4884 | TYNLCTLQP |
| 4885 | VAGIHDVVP |
| 4886 | VAKQLDCLG |
| 4887 | VATELPADY |
| 4888 | VCDQMQSFN |
| 4889 | VGIEMMGSG |
| 4890 | VHTHIIQDL |
| 4891 | VIKNCYEAQ |
| 4892 | VKAELHKTY |
| 4893 | VKEHSHFCP |
| 4894 | VKNTVHEMQ |
| 4895 | VKQLFTKDN |
| 4896 | VKSQSNGWN |
| 4897 | VNFQYNLGI |
| 4898 | VRAVKDTEG |
| 4899 | VSNHRPLMP |
| 4900 | VSQGIPKCE |
| 4901 | VTQMGHMMV |
| 4902 | VTQPKTDQT |
| 4903 | VTYGNMEDR |
| 4904 | VVCRMSGQD |
| 4905 | VVGNANEQK |
| 4906 | VVSKCEEMG |
| 4907 | WDEKPYFGF |
| 4908 | WDWWEVHRA |
| 4909 | WENQGDLTR |
| 4910 | WFQQSIMFI |
| 4911 | WGMPPKDNM |
| 4912 | WGPQNLFQG |
| 4913 | WLWWEEPLW |
| 4914 | WNIAPWDCS |
| 4915 | WTYMEASDF |
| 4916 | YCPKDDMVY |
| 4917 | YCPSKLPRN |
| 4918 | YEIKPEIVG |
| 4919 | YEQNSMWMC |
| 4920 | YGAPATWCD |
| 4921 | YGKIKIYEW |
| 4922 | YGPDLMRNE |
| 4923 | YIDRLTYSF |
| 4924 | YKESDWKIC |
| 4925 | YMPVLKDQA |
| 4926 | YPDLKEPMS |
| 4927 | YPGLRYTRS |
| 4928 | YPPVDQTPY |
| 4929 | YQPRNECIQ |
| 4930 | YQYPHELHY |
| 4931 | YSNVGSRYA |
| 4932 | YTHKLEAGN |
| 4933 | YWDNQWLKI |

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 6 (SEQ ID NO: 4934-SEQ ID NO: 5233) with increased likelihood of conferring muscle tissue tropism or preference on a recombinant AAV virion (rAAV); and (B) is the polypeptide sequence of SEQ ID NO: 5813 (IVPGSVWMIERDVYLQGPIWA, corresponding to residues 590 to 609 of SEQ ID NO: 2); and wherein the capsid polypeptide is capable of assembling into the rAAV and, the capsid does not have the sequence ofany of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 6 (SEQ ID NO: 4934-SEQ ID NO: 5233) at the region corresponding to amino acid residues 445 to 453 in AAV5 VP2 or the region corresponding to amino acid residues 389 to 397 in AAV5 CP3.

TABLE 6
Additional Sequences of 581-589 Regions
for Muscle Tropism

| SEQ ID | |
|---|---|
| NO | Sequence |
| 4934 | CVMSLLKVP |
| 4935 | CWVSYPFWM |
| 4936 | CVMPYSNHP |
| 4937 | PPIKDMLFK |
| 4938 | CVLNWHVGS |
| 4939 | PFPPPHKTL |
| 4940 | GDPPPLSFK |
| 4941 | CVRSLFWVF |
| 4942 | CIVMYSNKY |
| 4943 | CIVFYMKNQ |
| 4944 | CWKWQMMFG |
| 4945 | CVQQQFSAM |
| 4946 | CIVPNKFMW |
| 4947 | CVNYPNQAF |
| 4948 | CVKYSNQQG |
| 4949 | CYHQVFSQG |
| 4950 | CVMNPMVFK |
| 4951 | CVKQRGQRS |
| 4952 | CVILSTRDK |
| 4953 | CWKPWYLHG |
| 4954 | CVMGSYLSM |
| 4955 | CVWSQILGG |
| 4956 | CIKHWFVKF |
| 4957 | CIQLKRKMH |
| 4958 | CIQQLMWQN |
| 4959 | CVGPYRFGG |
| 4960 | CWREFSFQN |
| 4961 | CVYSVWKTS |
| 4962 | CVKLHQFEE |
| 4963 | CIMRWNSYG |
| 4964 | CVGKYGGHS |
| 4965 | CVQLVKEIG |
| 4966 | CVAMLHHYY |
| 4967 | CVMVPKGAG |
| 4968 | EDWMDMDLK |
| 4969 | CIGMKMQNG |
| 4970 | CINFIMKLG |
| 4971 | RIQEPPFPW |
| 4972 | CVLVFSNSG |
| 4973 | CAYMQQMHG |
| 4974 | CYFLIQQNF |
| 4975 | CAVPQVYQS |
| 4976 | RIKEPPDPK |
| 4977 | CVYLKGSLC |
| 4978 | CVYHKEMTS |
| 4979 | CVNFKTRQN |
| 4980 | CDDFKKKMT |
| 4981 | CISNLRMHG |
| 4982 | CWSVMPMAG |
| 4983 | CVNKVHSYN |
| 4984 | CSVLKHHGW |
| 4985 | CIKFFPIQG |
| 4986 | CVFSTSFGE |
| 4987 | CVSLYMDKL |
| 4988 | CVSHSHHGS |
| 4989 | CQMMPMKMD |
| 4990 | CMVPYNMYQ |
| 4991 | CWSQNQQGH |
| 4992 | CVFTIFEAK |
| 4993 | CVKQYMEMN |
| 4994 | CVMTTPLWF |
| 4995 | CVGYRVHSP |
| 4996 | CVLSIRIIS |
| 4997 | CVEYFLLHP |
| 4998 | CPRPLMKNF |
| 4999 | CVGLLEHSV |
| 5000 | CISPVMMYH |
| 5001 | CVVMSQMWM |
| 5002 | CYNMYKMWS |
| 5003 | QRPHRFPRH |
| 5004 | CVMGKTVFN |
| 5005 | CVHFKQFTW |
| 5006 | CVRWKHFGY |
| 5007 | CGNYIMYYK |
| 5008 | CFICIMPKT |
| 5009 | CVIYTRKHE |
| 5010 | CITRQQNEQ |
| 5011 | CILQMIILG |
| 5012 | MVEEPVGPL |
| 5013 | CVSMLGNHP |
| 5014 | CIMWKKPAD |
| 5015 | CVASYTLKP |
| 5016 | CVFQDMLFK |
| 5017 | CFYKIQHKG |
| 5018 | CTVFPIQSG |
| 5019 | CWVYMQGPS |
| 5020 | CISQNPMVY |
| 5021 | CTSPPRMWD |
| 5022 | CVQMMWPFP |
| 5023 | CVYTWGMQG |
| 5024 | CVHVHQLYG |
| 5025 | CIKLGNIIM |
| 5026 | CTRSLLGMG |
| 5027 | CVIRSSDQE |
| 5028 | WHPGRNEKC |
| 5029 | CSFNIHKHT |
| 5030 | CVSQMGKWP |
| 5031 | PIENPDQRI |
| 5032 | CITFYGMWW |
| 5033 | CVYTNVTSN |
| 5034 | CVKQLSYWQ |
| 5035 | CWGYPQVLM |
| 5036 | CIKFKSPSD |
| 5037 | CVTVSGRDY |
| 5038 | CVVWPVLGQ |
| 5039 | CVSWKNSGQ |
| 5040 | CVLRMGTFC |
| 5041 | CVGEFDSHR |
| 5042 | CIMWYTNNP |
| 5043 | CVIWQQITL |
| 5044 | CVVVYLKMC |
| 5045 | CVFPQVGKQ |
| 5046 | CVTQLFHQL |
| 5047 | HDESKSEPE |
| 5048 | CTRHMFRLG |
| 5049 | CIHKHPVSP |
| 5050 | CHFHQMIFY |
| 5051 | CTIPMQIIQ |
| 5052 | CVMMMNTKF |
| 5053 | CIMLHPVSF |
| 5054 | CYKYWPMHL |
| 5055 | CVVQNLFSG |
| 5056 | CVLNYFQFG |
| 5057 | CWYYRFKKN |
| 5058 | PPLSFRPIP |
| 5059 | CWKSMQFQY |
| 5060 | CVVQWGKWN |
| 5061 | DVEDDMEDN |
| 5062 | CFIPYYKMG |
| 5063 | KPEWWGILK |
| 5064 | CWNLFQISR |
| 5065 | CVKEFMMGC |
| 5066 | ADDDEDMEI |
| 5067 | CFWIKKNEI |
| 5068 | CVGSQNHIT |
| 5069 | CAMPYHFYW |
| 5070 | CVQMKVLFG |
| 5071 | CWMNMFSKH |
| 5072 | CVVNQHKFI |
| 5073 | CIKFEWRHN |
| 5074 | PRTPPREQR |
| 5075 | CINKYSGNT |
| 5076 | FHPKPHPQN |
| 5077 | CVGSQLHAM |
| 5078 | CIFMNGFMG |
| 5079 | REPNPLKSL |
| 5080 | CVGRQLMYS |
| 5081 | CVSIHQKNS |
| 5082 | QNGINNFPD |
| 5083 | CVSHSTFQS |
| 5084 | CWPSMQLNY |
| 5085 | CWILMLQMY |
| 5086 | CVGAGGKVT |
| 5087 | CVQHFMEHI |
| 5088 | CVIHIMMYD |
| 5089 | CWGYIHNFL |
| 5090 | HFRQPWPFK |
| 5091 | PHGEPRKIS |
| 5092 | CISNYLKQG |
| 5093 | CVWLKLFWY |
| 5094 | CTHMNLLHM |
| 5095 | CVMFREGSR |
| 5096 | CTIFMKSSN |
| 5097 | CVMTLVMMC |
| 5098 | CITKTKGQS |
| 5099 | CVIEILLWH |
| 5100 | CIMMRIPCG |
| 5101 | CVRSQLCNE |
| 5102 | CVGLKGIFE |
| 5103 | CVFWLGIRS |
| 5104 | CVGLLLFHG |
| 5105 | CVHPFVSWK |
| 5106 | CVQQTMFNL |
| 5107 | CVKTQGFIH |
| 5108 | CWQFFMSLV |
| 5109 | CIVPNQTRM |
| 5110 | RNDWKWPQE |
| 5111 | CRLYYIQHP |
| 5112 | MPFFDWIFK |
| 5113 | CVTHIQTGY |
| 5114 | PHINKWKMY |
| 5115 | IIMDQDSPK |
| 5116 | CWQKRDILF |
| 5117 | MVIFKNGKP |
| 5118 | CFWIHKMAA |
| 5119 | CAFPLNRQK |
| 5120 | LMMCPIHQT |
| 5121 | CMGRQFLQS |
| 5122 | WKNRDEKNI |
| 5123 | CIILLGSRD |
| 5124 | RTNYRPPAC |
| 5125 | CIMQYIKMY |
| 5126 | NIRQYMLQN |
| 5127 | CVFQKMNSD |
| 5128 | CVQLHKNHG |
| 5129 | CVFKMEPTE |
| 5130 | CSNIWVPKM |
| 5131 | CNVSYMFMG |
| 5132 | CVRRSLGLP |
| 5133 | CWYSTFKRG |
| 5134 | CVAMHRHNS |
| 5135 | CIIVKISKG |
| 5136 | WFVWIYPLK |
| 5137 | CGPYIVSLR |
| 5138 | CVSLFLHSG |
| 5139 | CMHQLRNRF |
| 5140 | HNYLPRNRP |
| 5141 | CVGIIQMGF |
| 5142 | CIKLNHHHK |
| 5143 | CWRHNIISP |
| 5144 | WIFFKDFMM |
| 5145 | LPRWMINPY |
| 5146 | PPTHDCKPK |
| 5147 | PPYCLDMKR |
| 5148 | CVHVYNFQM |
| 5149 | CVDSYIRWM |
| 5150 | CFLRLKKHH |
| 5151 | CKVLYIQIG |
| 5152 | CIVNTKKYH |
| 5153 | CVRGIFSDH |
| 5154 | CVGRILRSC |
| 5155 | CVIWYHKYE |
| 5156 | YICFVSYRN |
| 5157 | CWTKLVQHQ |
| 5158 | CVMNWDFFI |
| 5159 | CVMFAHNTQ |
| 5160 | CVLYFTFGK |
| 5161 | CTHMMGKSP |
| 5162 | WDTQLLPNI |
| 5163 | CFIHMQKFP |
| 5164 | CSMPLYSYY |
| 5165 | CIQSFGSGP |
| 5166 | YWGEFIKKS |
| 5167 | CISKKGRWY |
| 5168 | CIKQIKTYE |
| 5169 | CTQPFNRQD |
| 5170 | CVSWLLGQG |
| 5171 | DPEDDPGNE |
| 5172 | CVGHGSHQK |
| 5173 | CWKMFSLHG |
| 5174 | CIMKPDNYP |
| 5175 | CVQQLVQIE |
| 5176 | EEPEPYLKN |
| 5177 | CIRFQNPPF |
| 5178 | CIKWVDIFP |
| 5179 | NGPFGEWKR |
| 5180 | YNPDDKRVI |
| 5181 | DEGHKKFLK |
| 5182 | CVRSYQIVG |
| 5183 | PPRHQWNFY |
| 5184 | CISSKLRGF |
| 5185 | CVTFQLGWQ |
| 5186 | ATYLKMSSP |
| 5187 | CVSGSNFPH |
| 5188 | CVSFYNHME |
| 5189 | CFHVMMKYE |
| 5190 | CVWRITQQE |
| 5191 | DVDELDIHI |
| 5192 | CWAKIMPLF |
| 5193 | CWSHLYFQT |
| 5194 | EVLDWIETE |
| 5195 | CHYQNLKTH |
| 5196 | CVTQQHRDG |
| 5197 | CVVMMQIWP |
| 5198 | PRKYPWKKK |
| 5199 | CGCRFIYYQ |
| 5200 | QRWKHDPLP |
| 5201 | CVSLLEMSI |
| 5202 | HNIKPWKPY |
| 5203 | QENETFKKL |
| 5204 | DVDEEQDIF |
| 5205 | CVACKMKTG |
| 5206 | CFFYPMSMS |
| 5207 | PDERLFNKK |
| 5208 | CSTPMRMYM |
| 5209 | CFWQYGLVT |
| 5210 | CWLPFQMFH |
| 5211 | CTRQSIIVY |
| 5212 | CISVYFMWP |
| 5213 | DPRKLEPRI |
| 5214 | CTIGTMGPF |
| 5215 | CVRTLQSPD |
| 5216 | QDRFNHKPW |
| 5217 | CVSTVLNMG |
| 5218 | GDFPDEDDW |
| 5219 | CVWIWQNSY |
| 5220 | ETEYYQPFL |
| 5221 | YCPHRDRTH |
| 5222 | FEDEPEPWK |
| 5223 | PFDEGEPEI |
| 5224 | CVLKGKHEG |
| 5225 | CVQVLQLGG |
| 5226 | CTISQKMIW |
| 5227 | PKMSLMKFN |
| 5228 | CWSMYNSRY |
| 5229 | CWVMLYRGW |
| 5230 | CWTRQSNWP |
| 5231 | CVSPYTGLL |
| 5232 | PPPFFKYRG |
| 5233 | CTLYFGFIY |

In some embodiments, the engineered AAV VP capsid polypeptide comprises a polypeptide sequence represented by the formula: (A)-(X)—(B) wherein: (A) is the polypeptide sequence of SEQ ID NO: 5812 (VAYNVGGQMATNNQSSTTAP, corresponding to residues 561 to 580 of SEQ ID NO: 2); (X) is the 581-589 region having a polypeptide sequence selected from the list of polypeptides in TABLE 7 (SEQ ID NO: 5808-SEQ ID NO: 5811) with increased likelihood of conferring CNS tissue tropism or preference on a recombinant AAV virion (rAAV); and (B) is the polypeptide sequence of SEQ ID NO: 5813 (IVPGSVWMERDVYLQGPIWA, corresponding to residues 590 to 609 of SEQ ID NO: 2); and wherein the capsid polypeptide is capable of assembling into the rAAV and, the capsid does not have the sequence of any of SEQ ID NO: 1, SEQ ID NO: 3, SEQ ID NO: 4, SEQ ID NO: 5, SEQ ID NO: 6, SEQ ID NO: 7, or SEQ ID NO: 8. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 7 (SEQ ID NO: 5808-SEQ ID NO: 5811) at the region corresponding to amino acid residues 445 to 453 in AAV5 VP2 or the region corresponding to amino acid residues 389 to 397 in AAV5 VP3.

TABLE 7
Additional Sequences of 581-589 Regions
for Muscle Tropism

| SEQ ID NO | Sequence |
|---|---|
| 5808 | DDFGTLPRM |
| 5809 | GIIQVRMEW |
| 5810 | GQFEDLLVL |
| 5811 | RKEGCTSHG |

In some embodiments, the engineered AAV VP capsid polypeptide confers muscle tissue tropism or preference, wherein the muscle tissue is selected from the group consisting of aorta, esophagus, heart (e.g., atrium, ventricle, valves), skeletal muscle (e.g., biceps, brachii, femoris, diaphragm, gastrocnemius, tibialis anterior, triceps, quadriceps, including vastus lateralis)), muscle fibers including type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, and any combination thereof.

Described below are engineered mutated AAV5 VP1 polypeptide sequences that confer stable or improved virion assembly, tissue tropism, or both. In some embodiments, the present disclosure provides an AAV5 VP1 capsid polypeptide having a sequence homology of no more than 98.7% to SEQ ID NO: 1, wherein the AAV5 capsid polypeptide sequence has at least one mutation in a region from a position corresponding to 581 to a position corresponding to 589 of SEQ ID NO: 1. In some embodiments, an engineered AAV5 polypeptide comprises a variant 581-589 region (e.g., comprising at least one amino acid substitution relative to residues 561 to 580 of SEQ ID NO: 1).

Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 2, TABLE 3, TABLE 4, TABLE 5, TABLE 6, or TABLE 7 (SEQ ID NO: 12-SEQ ID NO: 3937, SEQ ID NO: 3938-SEQ ID NO: 4237, SEQ ID NO: 4238-SEQ ID NO: 4933, SEQ ID NO: 4934-SEQ ID NO: 5233, SEQ ID NO: 5234-SEQ ID NO: 5807, or SEQ ID NO: 5808-SEQ ID NO: 5811) at the region corresponding to amino acid residues 445 to 453 in AAV5 VP2 or the region corresponding to amino acid residues 389 to 397 in AAV5 VP3. Also encompassed herein are rAAVs composed of engineered AAV5 VP2 capsid polypeptides and engineered AAV5 VP3 capsid polypeptides comprising a 581-589 region having a sequence selected from the list of polypeptides in TABLE 9, TABLE 10, TABLE 11, or TABLE 10 at the region corresponding to amino acid residues 445 to 453 in AAV5 VP2 or the region corresponding to amino acid residues 389 to 397 in AAV5 VP3.

Engineered VP Polypeptides Competent for rAAV Assembly

In various preferred embodiments, the mutated (engineered, recombinant) VP capsid polypeptides of the present disclosure (e.g., comprising a variant 581-589 region) are capable of forming an assembled virion, and in some instances that exhibit similar or improved stability when compared to a virion that comprises the AAV5 VP1 capsid polypeptide of SEQ ID NO: 1.

The frequency of a given amino acid residue occurring in assembled, purified viruses at a specified position within the 581-589 region, corresponding to position 581 to position 589 of SEQ ID NO: 1, or corresponding to X¹ to X⁹ as generalized in SEQ ID NO: 2, over the frequency of that given amino acid residue occurring at the specified position in the entire plasmid library was analyzed to identify sequence rules for capsid polypeptide sequences that favor viral capsid assembly. Based on the determined amino acid residue frequency, the contribution of each amino acid at each position of the 581-589 region to capsid assembly was determined, and sequences of the 581-589 region (X¹X²X³X⁴X⁵X⁶X⁷X⁸X⁹ of SEQ ID NO: 2) that favor capsid assembly, and rules for selecting sequences that favor capsid assembly, were identified.

Disclosed herein are engineered AAV5 VP capsid polypeptides capable of forming an assembled viral capsid that may exhibit similar or improved stability as compared to wild type AAV5 VP capsid polypeptide, wherein the engineered variant AAV5 VP capsid polypeptide sequence has one or more mutations, wherein the VP1 polypeptide sequence has said one or more mutations in a 581-589 region (corresponding to position 581 to position 589 in SEQ ID NO: 2), and wherein X¹ is selected from A, D, E, G, L, M, N, Q, S, T, or V, or X¹ is selected from A, D, E, M, or T. In some embodiments, X¹ is E; or X² is selected from A, C, D, E, G, H, I, N, P, Q, S, T, or V, or X² is selected from A, S, T, or V, or X² is A; or wherein X³ is selected from A, D, E, G, H, M, N, Q, S, T, or V, or X³ is selected from D, E, N, Q or T, or X³ is D or T; or wherein X⁴ is selected from A, D, E, G, H, N, P, Q, S, or T, or X⁴ is selected from D, E, P, or Q, or X⁴ is E; or wherein X⁵ is selected from A, C, D, E, G, H, N, Q, S, T, or Y, or X⁵ is selected from D, E, N, Q or T, or X⁵ is N; or wherein X⁶ is selected from A, D, E, G, H, N, P, Q, S, or T, or X⁶ is selected from D, N, or Q, or X⁶ is D; or wherein X⁷ is selected from A, C, D, E, G, H, N, Q, S, or T, or X⁷ is selected from A, D, E or G, or X⁷ is A; or wherein X⁸ is selected from A, C, D, E, G, H, N, Q, S, or T, or X⁸ comprises A, D, G, or S, or X⁸ is G; or wherein X⁹ is selected from A, D, E, G, H, N, P, Q, S, or T, or X⁹ is selected from A, D, G, or P, or X⁹ is G.

In various embodiments, the VP polypeptide is capable of forming an assembled viral capsid, and in some instances exhibits similar or improved stability when compared to a virion that comprises the AAV5 VP1 capsid polypeptide of SEQ ID NO:1.

Examples of amino acid substitutions within a 581-589 region that may favor viral capsid assembly are provided in TABLE 8. In some embodiments, the following amino acids can be independently mutated, in any combination, at any one or more positions X¹ to X⁹, with reference to SEQ ID NO: 2, to provide an AAV VP1 capsid that is capable of assembling. Additionally, one or more mutations outside of the X¹ to X⁹ region can be allowed, as long as the capsid is still capable of assembling.

TABLE 8
581-589 Region Amino Acid Variations for Viral Assembly
Viral Assembly Options

X¹ is A, D, E, G, L, M, N, Q, S, T, or V
X¹ is A, D, E, M, or T
X¹ is E
X² is A, C, D, E, G, H, I, N, P, Q, S, T, or V
X² is A, S, T, or V
X² is A
X³ is A, D, E, G, H, M, N, Q, S, T, or V
X³ is D, E, N, Q or T
X³ is D or T
X⁴ is A, D, E, G, H, N, P, Q, S, or T
X⁴ is D, E, P, or Q
X⁴ is E
X⁵ is A, C, D, E, G, H, N, Q, S, T, or Y
X⁵ is D, E, N, Q or T
X⁵ is N.
X⁶ is A, D, E, G, H, N, P, Q, S, or T;
X⁶ is D, N, or Q; or
X⁶ is D.
X⁷ is A, C, D, E, G, H, N, Q, S, or T;
X⁷ is A, D, E or G; or
X⁷ is A.
X⁸ is A, C, D, E, G, H, N, Q, S, or T;
X⁸ is A, D, G, or S; or
X⁸ is G.
X⁹ is A, D, E, G, H, N, P, Q, S, or T;
X⁹ is A, D, G, or P; or
X⁹ is G.

### Variant 581-589 Regions for CNS Tissue Tropism

The present disclosure provides AAV5 virions with a VP capsid polypeptide having at least one mutation in a region with residues that interact with target cells (e.g., a target CNS cell in a target CNS tissue of interest), where the mutation confers increased CNS tissue tropism or preference as compared to a wild type VP capsid polypeptide (e.g., a VP capsid polypeptide of SEQ ID NO: 1, SEQ ID NO: 10, or SEQ ID NO: 11). In some embodiments, provided herein are AAV5 VP1 capsid polypeptide having a sequence homology of at least 80% to SEQ ID NO: 1, wherein the AAV5 VP1 capsid polypeptide has at least one mutation in a 581-589 region, corresponding to residues 581 to 589 of SEQ ID NO: 1, and wherein said at least one mutation drives increased central nervous system (CNS) tropism or preference as compared to the wild type VP capsid polypeptide (SEQ ID NO: 1). The following sequences rules and sequences also apply to the 581-589 region in AAV5 VP2 (amino acid residues 445 to 453; VP2 sequence shown in SEQ ID NO: 10) and in AAV5 VP3 (amino acid residues 389 to 397; VP3 sequences shown in SEQ ID NO: 11). Thus, the present disclosure encompasses AAV5 VP2 capsid polypeptides and AAV5 VP3 capsid polypeptides having one or more amino acid substitutions in the 581-589 regions of VP2 and VP3, corresponding to the AAV5 VP1 amino acid residues of the 581 to 589, where the one or more mutations comport to the rules or sequences in the following section.

VP capsid polypeptides (e.g., V1, VP2, or VP3 capsid polypeptides, or combinations thereof) comprising a CNS tissue-tropic 581-589 region (e.g., any one of SEQ ID NO: 12-SEQ ID NO: 3937 or SEQ ID NO: 3938-SEQ ID NO: 4237, or any 581-589 region adhering to the rules identified herein) may assemble to form an rAAV with altered surface properties compared to a wild type AAV (e.g., comprising a VP1 polypeptide of SEQ ID NO: 1). The surface may form an interface that forms interactions with a target tissue, and the altered surface properties of the rAAV may promote tissue specific interactions (e.g., CNS tissue-specific interactions) between the rAAV and the target tissue. In some embodiments, the altered surface properties may be an altered charge distribution, increased or decreased hydrophobicity, altered availability or distribution of hydrogen bond donors or acceptors, or formation or reshaping of binding pockets. Such altered surface properties may favor interactions between the rAAV and CNS tissue while disfavoring interactions between the rAAV and non-CNS tissue.

A CNS-tropic rAAV assembled from VP capsid polypeptides (e.g., V1, VP2, or VP3 capsid polypeptides, or combinations thereof) comprising a 581-589 region (e.g., any one of SEQ ID NO: 12-SEQ ID NO: 3937 or SEQ ID NO: 3938-SEQ ID NO: 4237, or any 581-589 region adhering to the rules identified herein) may preferentially infect a CNS tissue (e.g., cortex forebrain, cortex occipital, cortex temporal, thalamus, hypothalamus, substantia nigra, hippocampus DG, hippocampus CA1, hippocampus CA3, cerebellum, or combinations thereof) as compared to non-CNS tissue (e.g., liver, skeletal muscle, heart, lung, spleen, lymph node, bone marrow, mammary gland, skin, adrenal gland, thyroid, colon, sciatic nerve, spinal cord, or combinations thereof) at a higher level as compared to wild type AAV5. The CNS-tropic AAV may infect the CNS tissue at a rate that is at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold an infection rate for the non-CNS tissue, as compared to wild type AAV5. In some embodiments, the non-CNS tissue is a liver tissue.

In some embodiments, the CNS-tropic rAAV assembled from VP capsid polypeptides (e.g., V1, VP2, or VP3 capsid polypeptides, or combinations thereof) comprising a 581-589 region (e.g., any one of SEQ ID NO: 12-SEQ ID NO: 3937 or SEQ ID NO: 3938-SEQ ID NO: 4237, or any 581-589 region adhering to the rules identified herein) may deliver a payload to the tissue infected by the rAAV. The payload (e.g., a payload encoding a therapeutic peptide or a therapeutic polynucleotide) may be expressed in the tissue. In some embodiments, an expression level of the payload in a CNS tissue may be at least about 2.5-fold, at least about 2.8-fold, at least about 3-fold, at least about 3.2-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 50-fold, at least about 75-fold, at least about 100-fold, at least about 200-fold, at least about 500-fold higher, or at least about 1000-fold an expression level in a non-CNS tissue (e.g., a liver tissue). In some embodiments, payload delivery to a CNS tissue using a CNS-tropic AAV may have at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold higher specificity for CNS tissue as compared to a wild type AAV5.

CNS-Tropic Sequences

Recombinant AAV viral capsids with specificity for central nervous system (CNS) tissues (CNS-tropic rAAVs) may be identified by screening rAAV libraries comprising VP capsid polypeptides with 581-589 regions, as described herein. The libraries may be screened in non-human primates (NHPs) by systemically administering the rAAV library and identifying sequences of 581-589 regions in VP capsid polypeptides that conferred tissue-tropic accumulation in or infection of CNS tissues (e.g., cortex forebrain, cortex occipital, cortex temporal, thalamus, hypothalamus, substantia nigra, hippocampus DG, hippocampus CA1, hippocampus CA3, cerebellum, or combinations thereof). The CNS-tropic rAAVs may preferentially accumulate in or infect CNS tissues as compared to non-CNS tissues (e.g., liver, skeletal muscle, heart, lung, spleen, lymph node, bone marrow, mammary gland, skin, adrenal gland, thyroid, colon, sciatic nerve, spinal cord, or combinations thereof). Exemplary 581-589 region sequences identified in a primary screen as conferring CNS tissue tropism to an rAAV are provided in TABLE 2 and TABLE 4. Exemplary 581-589 region sequences identified in a secondary screen as conferring CNS tissue tropism to an rAAV are provided in TABLE 9.

A CNS-tropic rAAV assembled from VP capsid polypeptides (e.g., V1, VP2, or VP3 capsid polypeptides, or combinations thereof) comprising a 581-589 region (e.g., any one of SEQ ID NO: 12-SEQ ID NO: 3937) may preferentially infect a CNS tissue (e.g., cortex forebrain, cortex occipital, cortex temporal, thalamus, hypothalamus, substantia nigra, hippocampus DG, hippocampus CA1, hippocampus CA3, cerebellum, or combinations thereof) as compared to non-CNS tissue (e.g., liver, skeletal muscle, heart, lung, spleen, lymph node, bone marrow, mammary gland, skin, adrenal gland, thyroid, colon, sciatic nerve, spinal cord, or combinations thereof).

Disclosed herein are engineered AAV5 VP capsid polypeptides capable of forming an assembled virion that exhibits increased central nervous system tissue tropism as compared to a wild type AAV5 VP capsid polypeptide (e.g., a VP1 of SEQ ID NO: 1, a VP2 of SEQ ID NO: 10, or a VP3 of SEQ ID NO: 11), wherein the engineered variant AAV5 VP capsid polypeptide sequence has one or more amino acid substitutions in a 581-589 region of the VP capsid polypeptide, corresponding to positions 581 to 589 in SEQ ID NO: 2 (X¹X²X³X⁴X⁵X⁶X⁷X⁸X⁹). In some embodiments, X¹ is selected from A, R, N, D, C, E, Q, G, H, I, L, K, M, F, P, S, T, W, Y, or V. In some embodiments, X² is selected from A, R, N, D, C, E, Q, G, H, I, L, K, M, F, P, S, T, W, Y, or V. In some embodiments, X³ is selected from A, R, N, D, C, E, Q, G, H, I, L, K, M, F, P, S, T, W, Y, or V. In some embodiments, X⁴ is selected from A, R, N, D, C, E, Q, G, H, I, L, K, M, F, P, S, T, W, Y, or V. In some embodiments, X⁵ is selected from A, R, N, D, C, E, Q, G, H, I, L, K, M, F, P, S, T, W, Y, or V. In some embodiments, X⁶ is selected from A, R, N, D, C, E, Q, G, H, I, L, K, M, F, P, S, T, W, Y, or V. In some embodiments, X⁷ is selected from A, R, N, D, C, E, Q, G, H, I, L, K, M, F, P, S, T, W, Y, or V. In some embodiments, X⁸ is selected A, R, N, D, C, E, Q, G, H, I, L, K, M, F, P, S, T, W, Y, or V. In some embodiments, X⁹ is selected from A, R, N, D, C, E, Q, G, H, I, L, K, M, F, P, S, T, W, Y, or V.

In some embodiments, a 581-589 region of a CNS tissue-tropic VP capsid polypeptide may comprise a sequence of any one of SEQ ID NO: 12-SEQ ID NO: 3937.

In some embodiments, provided herein are AAV5 VP capsid polypeptide having at least one mutation in a 581-589 region, corresponding to residues 581 to 589 of AAV5 VP1 (e.g., SEQ ID NO: 1) and having at least 70%, at least 75%, at least 80%, at least 85%, at least 90%, at least 92%, at least 95%, at least 97%, at least 99%, or 100% sequence identity to any sequence selected from SEQ ID NO: 12-SEQ ID NO: 3937, wherein said at least one mutation drives increased central nervous system tissue tropism as compared to a wild type AAV5 capsid polypeptide.

### CNS-Tropic Sequences Generated by Machine Learning

Additional 581-589 regions that confer CNS tissue tropism may be generated using machine learning algorithms trained with sequence identified in CNS tissue in in vivo screens. These patterns may be used to generate new sequences of 581-589 regions that increase the likelihood of conferring CNS tissue tropism to VP capsid polypeptides and rAAVs assembled from the VP capsid polypeptides. In some embodiments, new sequences of 581-589 regions may be generated by randomly sampling amino acid residues at each position of 581-589 regions that favored CNS tissue tropism in an in vivo screen. The generated sequences may be tested using random forests or gradient boosting classifiers to predicted CNS tissue-specificity for each 581-589 region. The 581-589 regions with the highest predicted CNS tissue-specificity may be selected as CNS tissue-tropic sequences. For example, a 581-589 region of any one of SEQ ID NO: 3938-SEQ ID NO: 4237 may be selected. 581-589 region sequences generated using machine learning for increased likelihood of conferring CNS tissue tropism to an rAAV are provided in TABLE 3.

Favored amino acid residues and disfavored amino acid residues at each position in the 581-589 region of an engineered AAV5 capsid polypeptide that increase the likelihood of conferring increased CNS tissue tropism as compared to a wild type AAV5 capsid polypeptide may be determined using in vivo data, machine learning (ML) models, or combinations thereof. Recombinant AAV viral capsid libraries may be screened in non-human primates (NHPs) by systemically administering the rAAV library and identifying sequences of 581-589 regions in VP capsid polypeptides that increased the likelihood of conferring tissue-tropic accumulation in or infection of CNS tissues (e.g., cortex forebrain, cortex occipital, cortex temporal, thalamus, hypothalamus, substantia nigra, hippocampus DG, hippocampus CA1, hippocampus CA3, cerebellum, or combinations thereof).

A CNS-tropic rAAV assembled from VP capsid polypeptides (e.g., V1, VP2, or VP3 capsid polypeptides, or combinations thereof) comprising a 581-589 region may preferentially infect a CNS tissue (e.g., cortex forebrain, cortex occipital, cortex temporal, thalamus, hypothalamus, substantia nigra, hippocampus DG, hippocampus CA1, hippocampus CA3, cerebellum, or combinations thereof) as compared to non-CNS tissue (e.g., liver, skeletal muscle, heart, lung, spleen, lymph node, bone marrow, mammary gland, skin, adrenal gland, thyroid, colon, sciatic nerve, spinal cord, or combinations thereof), as compared to a wild type AAV5 capsid polypeptide.

Disclosed herein are engineered AAV5 VP capsid polypeptides capable of forming an assembled virion that exhibits increased central nervous system tissue tropism or preference as compared to a wild type AAV5 VP capsid polypeptide (e.g., a VP1 of SEQ ID NO: 1, a VP2 of SEQ ID NO: 10, or a VP3 of SEQ ID NO: 11), wherein the engineered variant AAV5 VP capsid polypeptide sequence has one or more amino acid substitutions in a 581-589 region of the VP capsid polypeptide, corresponding to positions 581 to 589 in SEQ ID NO: 2 (X¹X²X³X⁴X⁵X⁶X⁷X⁸X⁹). In some embodiments, X¹ is selected from A, R, N, D, C, E, Q, G, H, I, L, K, M, F, P, S, T, W, Y, or V. In some embodiments, X² is selected from A, R, N, D, C, E, Q, G, H, I, L, K, M, F, P, S, T, W, Y, or V. In some embodiments, X³ is selected from A, R, N, D, C, E, Q, G, H, I, L, K, M, F, P, S, T, W, Y, or V. In some embodiments, X⁴ is selected from A, R, N, D, C, E, Q, G, H, I, L, K, M, F, P, S, T, W, Y, or V. In some embodiments, X⁵ is selected from A, R, N, D, C, E, Q, G, H, I, L, K, M, F, P, S, T, W, Y, or V. In some embodiments, X⁶ is selected from A, R, N, D, C, E, Q, G, H, I, L, K, M, F, P, S, T, W, Y, or V. In some embodiments, X⁷ is selected from A, R, N, D, C, E, Q, G, H, I, L, K, M, F, P, S, T, W, Y, or V. In some embodiments, X⁸ is selected A, R, N, D, C, E, Q, G, H, I, L, K, M, F, P, S, T, W, Y, or V. In some embodiments, X⁹ is selected from A, R, N, D, C, E, Q, G, H, I, L, K, M, F, P, S, T, W, Y, or V.

In some embodiments, a 581-589 region of a CNS tissue-tropic VP capsid polypeptide may comprise a sequence of any one of SEQ ID NO: 3938-SEQ ID NO: 4237.

In some embodiments, provided herein are AAV5 VP capsid polypeptide having at least one mutation in a 581-589 region, corresponding to residues 581 to 589 of AAV5 VP1 (e.g., SEQ ID NO: 1) and having at least 70%, at least 75%, at least 80%, at least 85%, at least 90%, at least 92%, at least 95%, at least 97%, at least 99%, or 100% sequence identity to any sequence selected from SEQ ID NO: 3938-SEQ ID NO: 4237, wherein said at least one mutation drives increased central nervous system tissue tropism as compared to a wild type AAV5 capsid polypeptide.

The present disclosure provides a tissue-tropic rAAV comprising a VP capsid polypeptide with a 581-589 variant region, relative to WT AAV5, of SEQ ID NO: 18. The present disclosure provides a tissue-tropic rAAV comprising a VP capsid polypeptide with a 581-589 variant region, relative to WT AAV5, of SEQ ID NO: 54. The present disclosure provides a tissue-tropic rAAV comprising a VP capsid polypeptide with a 581-589 variant region, relative to WT AAV5, of SEQ ID NO: 2028. The present disclosure provides a tissue-tropic rAAV comprising a VP capsid polypeptide with a 581-589 variant region, relative to WT AAV5, of SEQ ID NO: 424. The present disclosure provides a tissue-tropic rAAV comprising a VP capsid polypeptide with a 581-589 variant region, relative to WT AAV5, of SEQ ID NO: 3306. The present disclosure provides a tissue-tropic rAAV comprising a VP capsid polypeptide with a 581-589 variant region, relative to WT AAV5, of SEQ ID NO: 3472. The present disclosure provides a tissue-tropic rAAV comprising a VP capsid polypeptide with a 581-589 variant region, relative to WT AAV5, of SEQ ID NO: 415. The present disclosure provides a tissue-tropic rAAV comprising a VP capsid polypeptide with a 581-589 variant region, relative to WT AAV5, of SEQ ID NO: 709. The present disclosure provides a tissue-tropic rAAV comprising a VP capsid polypeptide with a 581-589 variant region, relative to WT AAV5, of SEQ ID NO: 1971. The present disclosure provides a tissue-tropic rAAV comprising a VP capsid polypeptide with a 581-589 variant region, relative to WT AAV5, of SEQ ID NO: 2791. The present disclosure provides a tissue-tropic rAAV comprising a VP capsid polypeptide with a 581-589 variant region, relative to WT AAV5, of SEQ ID NO: 1956. The present disclosure provides a tissue-tropic rAAV comprising a VP capsid polypeptide with a 581-589 variant region, relative to WT AAV5, of SEQ ID NO: 262. The present disclosure provides a tissue-tropic rAAV comprising a VP capsid polypeptide with a 581-589 variant region, relative to WT AAV5, of SEQ ID NO: 425. The present disclosure provides a tissue-tropic rAAV comprising a VP capsid polypeptide with a 581-589 variant region, relative to WT AAV5, of SEQ ID NO: 2536. The present disclosure provides a tissue-tropic rAAV comprising a VP capsid polypeptide with a 581-589 variant region, relative to WT AAV5, of SEQ ID NO: 708. The present disclosure provides a tissue-tropic rAAV comprising a VP capsid polypeptide with a 581-589 variant region, relative to WT AAV5, of SEQ ID NO: 3283. The present disclosure provides a tissue-tropic rAAV comprising a VP capsid polypeptide with a 581-589 variant region, relative to WT AAV5, of SEQ ID NO: 430. The present disclosure provides a tissue-tropic rAAV comprising a VP capsid polypeptide with a 581-589 variant region, relative to WT AAV5, of SEQ ID NO: 3297. The present disclosure provides a tissue-tropic rAAV comprising a VP capsid polypeptide with a 581-589 variant region, relative to WT AAV5, of SEQ ID NO: 2661. The present disclosure provides a tissue-tropic rAAV comprising a VP capsid polypeptide with a 581-589 variant region, relative to WT AAV5, of SEQ ID NO: 428. The present disclosure provides a tissue-tropic rAAV comprising a VP capsid polypeptide with a 581-589 variant region, relative to WT AAV5, of SEQ ID NO: 885. The present disclosure provides a tissue-tropic rAAV comprising a VP capsid polypeptide with a 581-589 variant region, relative to WT AAV5, of SEQ ID NO: 429. The present disclosure provides a tissue-tropic rAAV comprising a VP capsid polypeptide with a 581-589 variant region, relative to WT AAV5, of SEQ ID NO: 569. The present disclosure provides a tissue-tropic rAAV comprising a VP capsid polypeptide with a 581-589 variant region, relative to WT AAV5, of SEQ ID NO: 724. The present disclosure provides a tissue-tropic rAAV comprising a VP capsid polypeptide with a 581-589 variant region, relative to WT AAV5, of SEQ ID NO: 1576. The present disclosure provides a tissue-tropic rAAV comprising a VP capsid polypeptide with a 581-589 variant region, relative to WT AAV5, of SEQ ID NO: 4545. The present disclosure provides a tissue-tropic rAAV comprising a VP capsid polypeptide with a 581-589 variant region, relative to WT AAV5, of SEQ ID NO: 426. The present disclosure provides a tissue-tropic rAAV comprising a VP capsid polypeptide with a 581-589 variant region, relative to WT AAV5, of SEQ ID NO: 1887. The present disclosure provides a tissue-tropic rAAV comprising a VP capsid polypeptide with a 581-589 variant region, relative to WT AAV5, of SEQ ID NO: 3906. The present disclosure provides a tissue-tropic rAAV comprising a VP capsid polypeptide with a 581-589 variant region, relative to WT AAV5, of SEQ ID NO: 3935. The present disclosure provides a tissue-tropic rAAV comprising a VP capsid polypeptide with a 581-589 variant region, relative to WT AAV5, of SEQ ID NO: 3846. The present disclosure provides a tissue-tropic rAAV comprising a VP capsid polypeptide with a 581-589 variant region, relative to WT AAV5, of SEQ ID NO: 2168. The present disclosure provides a tissue-tropic rAAV comprising a VP capsid polypeptide with a 581-589 variant region, relative to WT AAV5, of SEQ ID NO: 4119. The present disclosure provides a tissue-tropic rAAV comprising a VP capsid polypeptide with a 581-589 variant region, relative to WT AAV5, of SEQ ID NO: 2456. The present disclosure provides a tissue-tropic rAAV comprising a VP capsid polypeptide with a 581-589 variant region, relative to WT AAV5, of SEQ ID NO: 2278. The present disclosure provides a tissue-tropic rAAV comprising a VP capsid polypeptide with a 581-589 variant region, relative to WT AAV5, of SEQ ID NO: 5006. The present disclosure provides a tissue-tropic rAAV comprising a VP capsid polypeptide with a 581-589 variant region, relative to WT AAV5, of SEQ ID NO: 307. The present disclosure provides a tissue-tropic rAAV comprising a VP capsid polypeptide with a 581-589 variant region, relative to WT AAV5, of SEQ ID NO: 5155. The present disclosure provides a tissue-tropic rAAV comprising a VP capsid polypeptide with a 581-589 variant region, relative to WT AAV5, of SEQ ID NO: 2640. The present disclosure provides a tissue-tropic rAAV comprising a VP capsid polypeptide with a 581-589 variant region, relative to WT AAV5, of SEQ ID NO: 4317. The present disclosure provides a tissue-tropic rAAV comprising a VP capsid polypeptide with a 581-589 variant region, relative to WT AAV5, of SEQ ID NO: 1145. The present disclosure provides a tissue-tropic rAAV comprising a VP capsid polypeptide with a 581-589 variant region, relative to WT AAV5, of SEQ ID NO: 23. The present disclosure provides a tissue-tropic rAAV comprising a VP capsid polypeptide with a 581-589 variant region, relative to WT AAV5, of SEQ ID NO: 103. The present disclosure provides a tissue-tropic rAAV comprising a VP capsid polypeptide with a 581-589 variant region, relative to WT AAV5, of SEQ ID NO: 22. The present disclosure provides a tissue-tropic rAAV comprising a VP capsid polypeptide with a 581-589 variant region, relative to WT AAV5, of SEQ ID NO: 4031. The present disclosure provides a tissue-tropic rAAV comprising a VP capsid polypeptide with a 581-589 variant region, relative to WT AAV5, of SEQ ID NO: 1008. The present disclosure provides a tissue-tropic rAAV comprising a VP capsid polypeptide with a 581-589 variant region, relative to WT AAV5, of SEQ ID NO: 4790. The present disclosure provides a tissue-tropic rAAV comprising a VP capsid polypeptide with a 581-589 variant region, relative to WT AAV5, of SEQ ID NO: 2522. The present disclosure provides a tissue-tropic rAAV comprising a VP capsid polypeptide with a 581-589 variant region, relative to WT AAV5, of SEQ ID NO: 1432. The present disclosure provides a tissue-tropic rAAV comprising a VP capsid polypeptide with a 581-589 variant region, relative to WT AAV5, of SEQ ID NO: 2914. The present disclosure provides a tissue-tropic rAAV comprising a VP capsid polypeptide with a 581-589 variant region, relative to WT AAV5, of SEQ ID NO: 5042. The present disclosure provides a tissue-tropic rAAV comprising a VP capsid polypeptide with a 581-589 variant region, relative to WT AAV5, of SEQ ID NO: 2865. The present disclosure provides a tissue-tropic rAAV comprising a VP capsid polypeptide with a 581-589 variant region, relative to WT AAV5, of SEQ ID NO: 4264. The present disclosure provides a tissue-tropic rAAV comprising a VP capsid polypeptide with a 581-589 variant region, relative to WT AAV5, of SEQ ID NO: 964. The present disclosure provides a tissue-tropic rAAV comprising a VP capsid polypeptide with a 581-589 variant region, relative to WT AAV5, of SEQ ID NO: 1268. The present disclosure provides a tissue-tropic rAAV comprising a VP capsid polypeptide with a 581-589 variant region, relative to WT AAV5, of SEQ ID NO: 5065. The present disclosure provides a tissue-tropic rAAV comprising a VP capsid polypeptide with a 581-589 variant region, relative to WT AAV5, of SEQ ID NO: 706. The present disclosure provides a tissue-tropic rAAV comprising a VP capsid polypeptide with a 581-589 variant region, relative to WT AAV5, of SEQ ID NO: 4704. The present disclosure provides a tissue-tropic rAAV comprising a VP capsid polypeptide with a 581-589 variant region, relative to WT AAV5, of SEQ ID NO: 2994. The present disclosure provides a tissue-tropic rAAV comprising a VP capsid polypeptide with a 581-589 variant region, relative to WT AAV5, of SEQ ID NO: 829. The present disclosure provides a tissue-tropic rAAV comprising a VP capsid polypeptide with a 581-589 variant region, relative to WT AAV5, of SEQ ID NO: 1171. The present disclosure provides a tissue-tropic rAAV comprising a VP capsid polypeptide with a 581-589 variant region, relative to WT AAV5, of SEQ ID NO: 1041. The present disclosure provides a tissue-tropic rAAV comprising a VP capsid polypeptide with a 581-589 variant region, relative to WT AAV5, of SEQ ID NO: 5070. The present disclosure provides a tissue-tropic rAAV comprising a VP capsid polypeptide with a 581-589 variant region, relative to WT AAV5, of SEQ ID NO: 139. The present disclosure provides a tissue-tropic rAAV comprising a VP capsid polypeptide with a 581-589 variant region, relative to WT AAV5, of SEQ ID NO: 3304. The present disclosure provides a tissue-tropic rAAV comprising a VP capsid polypeptide with a 581-589 variant region, relative to WT AAV5, of SEQ ID NO: 2431. The present disclosure provides a tissue-tropic rAAV comprising a VP capsid polypeptide with a 581-589 variant region, relative to WT AAV5, of SEQ ID NO: 4288. The present disclosure provides a tissue-tropic rAAV comprising a VP capsid polypeptide with a 581-589 variant region, relative to WT AAV5, of SEQ ID NO: 5795. The present disclosure provides a tissue-tropic rAAV comprising a VP capsid polypeptide with a 581-589 variant region, relative to WT AAV5, of SEQ ID NO: 1300. The present disclosure provides a tissue-tropic rAAV comprising a VP capsid polypeptide with a 581-589 variant region, relative to WT AAV5, of SEQ ID NO: 1770. The present disclosure provides a tissue-tropic rAAV comprising a VP capsid polypeptide with a 581-589 variant region, relative to WT AAV5, of SEQ ID NO: 2830. The present disclosure provides a tissue-tropic rAAV comprising a VP capsid polypeptide with a 581-589 variant region, relative to WT AAV5, of SEQ ID NO: 1972. The present disclosure provides a tissue-tropic rAAV comprising a VP capsid polypeptide with a 581-589 variant region, relative to WT AAV5, of SEQ ID NO: 1649. The present disclosure provides a tissue-tropic rAAV comprising a VP capsid polypeptide with a 581-589 variant region, relative to WT AAV5, of SEQ ID NO: 2515. The present disclosure provides a tissue-tropic rAAV comprising a VP capsid polypeptide with a 581-589 variant region, relative to WT AAV5, of SEQ ID NO: 2849. The present disclosure provides a tissue-tropic rAAV comprising a VP capsid polypeptide with a 581-589 variant region, relative to WT AAV5, of SEQ ID NO: 3796. The present disclosure provides a tissue-tropic rAAV comprising a VP capsid polypeptide with a 581-589 variant region, relative to WT AAV5, of SEQ ID NO: 5815 (SYKMIHNTA). The present disclosure provides a tissue-tropic rAAV comprising a VP capsid polypeptide with a 581-589 variant region, relative to WT AAV5, of SEQ ID NO: 118. The present disclosure provides a tissue-tropic rAAV comprising a VP capsid polypeptide with a 581-589 variant region, relative to WT AAV5, of SEQ ID NO: 4766. The present disclosure provides a tissue-tropic rAAV comprising a VP capsid polypeptide with a 581-589 variant region, relative to WT AAV5, of SEQ ID NO: 770. The present disclosure provides a tissue-tropic rAAV comprising a VP capsid polypeptide with a 581-589 variant region, relative to WT AAV5, of SEQ ID NO: 1069. The present disclosure provides a tissue-tropic rAAV comprising a VP capsid polypeptide with a 581-589 variant region, relative to WT AAV5, of SEQ ID NO: 3061. The present disclosure provides a tissue-tropic rAAV comprising a VP capsid polypeptide with a 581-589 variant region, relative to WT AAV5, of SEQ ID NO: 4290. The present disclosure provides a tissue-tropic rAAV comprising a VP capsid polypeptide with a 581-589 variant region, relative to WT AAV5, of SEQ ID NO: 4261. The present disclosure provides a tissue-tropic rAAV comprising a VP capsid polypeptide with a 581-589 variant region, relative to WT AAV5, of SEQ ID NO: 4239. The present disclosure provides a tissue-tropic rAAV comprising a VP capsid polypeptide with a 581-589 variant region, relative to WT AAV5, of SEQ ID NO: 3478. The present disclosure provides a tissue-tropic rAAV comprising a VP capsid polypeptide with a 581-589 variant region, relative to WT AAV5, of SEQ ID NO: 2671. The present disclosure provides a tissue-tropic rAAV comprising a VP capsid polypeptide with a 581-589 variant region, relative to WT AAV5, of SEQ ID NO: 256. The present disclosure provides a tissue-tropic rAAV comprising a VP capsid polypeptide with a 581-589 variant region, relative to WT AAV5, of SEQ ID NO: 1256. The present disclosure provides a tissue-tropic rAAV comprising a VP capsid polypeptide with a 581-589 variant region, relative to WT AAV5, of SEQ ID NO: 719. The present disclosure provides a tissue-tropic rAAV comprising a VP capsid polypeptide with a 581-589 variant region, relative to WT AAV5, of SEQ ID NO: 1448. The present disclosure provides a tissue-tropic rAAV comprising a VP capsid polypeptide with a 581-589 variant region, relative to WT AAV5, of SEQ ID NO: 280. The present disclosure provides a tissue-tropic rAAV comprising a VP capsid polypeptide with a 581-589 variant region, relative to WT AAV5, of SEQ ID NO: 4338. The present disclosure provides a tissue-tropic rAAV comprising a VP capsid polypeptide with a 581-589 variant region, relative to WT AAV5, of SEQ ID NO: 5037. The present disclosure provides a tissue-tropic rAAV comprising a VP capsid polypeptide with a 581-589 variant region, relative to WT AAV5, of SEQ ID NO: 1901. The present disclosure provides a tissue-tropic rAAV comprising a VP capsid polypeptide with a 581-589 variant region, relative to WT AAV5, of SEQ ID NO: 438. The present disclosure provides a tissue-tropic rAAV comprising a VP capsid polypeptide with a 581-589 variant region, relative to WT AAV5, of SEQ ID NO: 2834. The present disclosure provides a tissue-tropic rAAV comprising a VP capsid polypeptide with a 581-589 variant region, relative to WT AAV5, of SEQ ID NO: 5491. The present disclosure provides a tissue-tropic rAAV comprising a VP capsid polypeptide with a 581-589 variant region, relative to WT AAV5, of SEQ ID NO: 4591. The present disclosure provides a tissue-tropic rAAV comprising a VP capsid polypeptide with a 581-589 variant region, relative to WT AAV5, of SEQ ID NO: 4936.

### Variant 581-589 Regions for CNS Targeting

In some embodiments, provided herein are AAV5 VP capsid polypeptide having at least one mutation in a 581-589 region, corresponding to residues 581 to 589 of AAV5 VP1, and having at least 70%, at least 75%, at least 80%, at least 85%, at least 90%, at least 92%, at least 95%, at least 97%, at least 99%, or 100% sequence identity to any sequence selected from SEQ ID NO: 12-SEQ ID NO: 3937, SEQ ID NO: 3938-SEQ ID NO: 4237, or SEQ ID NO: 5234-SEQ ID NO: 5807, wherein said at least one mutation drives increased CNS tissue tropism as compared to a wild type AAV5 capsid polypeptide.

### Variant 581-589 Regions for Muscle Tissue Tropism

The present disclosure provides AAV5 virions with a VP capsid polypeptide having at least one mutation in a region with residues that interact with target cells (e.g., a target muscle cell in a target muscle tissue of interest), where the at least one mutation confers increased muscle tissue tropism or preference as compared to a wild type VP capsid polypeptide (e.g., a VP capsid polypeptide of SEQ ID NO: 1, SEQ ID NO: 10, or SEQ ID NO: 11). In some embodiments, provided herein are AAV5 VP1 capsid polypeptide having a sequence homology of at least 80% to SEQ ID NO: 1, wherein the AAV5 VP1 capsid polypeptide has at least one mutation in a 581-589 region, corresponding to residues 581 to 589 of SEQ ID NO: 1, and wherein said at least one mutation drives increased muscle tropism or preference as compared to the wild type VP capsid polypeptide of SEQ ID NO: 1. The following sequences rules and sequences also apply to the 581-589 region in AAV5 VP2 (amino acid residues 445 to 453; VP2 sequence shown in SEQ ID NO: 10) and in AAV5 VP3 (amino acid residues 389 to 397; VP3 sequences shown in SEQ ID NO: 11). Thus, the present disclosure encompasses AAV5 VP2 capsid polypeptides and AAV5 VP3 capsid polypeptides having one or more amino acid substitutions in the 581-589 regions of VP2 and VP3, corresponding to the AAV5 VP1 amino acid residues of the 581 to 589, where the one or more mutations comport to the rules or sequences in the following section.

VP capsid polypeptides (e.g., V1, VP2, or VP3 capsid polypeptides, or combinations thereof) comprising a muscle-tropic 581-589 region (e.g., any one of SEQ ID NO: 4238-SEQ ID NO: 4933 or SEQ ID NO: 4934-SEQ ID NO: 5233, or any 581-589 region adhering to the rules identified herein) may assemble to form an rAAV with altered surface properties compared to a wild type AAV (e.g., comprising a VP1 polypeptide of SEQ ID NO: 1). The surface may form an interface that forms interactions with a target tissue, and the altered surface properties of the rAAV may promote tissue specific interactions (e.g., muscle-specific interactions) between the rAAV and the target tissue.

A muscle-tropic rAAV assembled from VP capsid polypeptides (e.g., V1, VP2, or VP3 capsid polypeptides, or combinations thereof) comprising a 581-589 region (e.g., any one of SEQ ID NO: 4238-SEQ ID NO: 4933 or SEQ ID NO: 4934-SEQ ID NO: 5233, or any 581-589 region adhering to the rules identified herein) may preferentially infect a muscle tissue (e.g., aorta, esophagus, heart (including atrium, ventricle, valves), skeletal muscle (including biceps, brachii, femoris, diaphragm, gastrocnemius, tibialis anterior, triceps, quadriceps, including vastus lateralis)), or muscle fibers (e.g., type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof) as compared to non-muscle tissue (e.g., liver, CNS, heart, lung, spleen, lymph node, bone marrow, mammary gland, skin, adrenal gland, thyroid, colon, sciatic nerve, spinal cord, or combinations thereof) at a higher level as compared to wild type AAV5. The muscle-tropic AAV may infect the muscle tissue at a rate that is at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold an infection rate for the non-muscle tissue, as compared to wild type AAV5. In some embodiments, the non-muscle tissue is a liver tissue.

In some embodiments, the muscle-tropic rAAV assembled from VP capsid polypeptides (e.g., V1, VP2, or VP3 capsid polypeptides, or combinations thereof) comprising a 581-589 region (e.g., any one of SEQ ID NO: 4238-SEQ ID NO: 4933 or SEQ ID NO: 4934-SEQ ID NO: 5233, or any 581-589 region adhering to the rules identified herein) may deliver a payload to the tissue infected by the rAAV. The payload (e.g., a payload encoding a therapeutic peptide or a therapeutic polynucleotide) may be expressed in the tissue. In some embodiments, an expression level of the payload in a muscle tissue may be at least about 2.5-fold, at least about 2.8-fold, at least about 3-fold, at least about 3.2-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 50-fold, at least about 75-fold, at least about 100-fold, at least about 200-fold, at least about 500-fold higher, or at least about 1000-fold an expression level in a non-muscle tissue (e.g., a liver tissue). In some embodiments, payload delivery to a muscle tissue using a CNS-tropic AAV may have at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.2-fold, at least about 2.4-fold, at least about 2.6-fold, at least about 2.8-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 20-fold, at least about 30-fold, at least about 40-fold, at least about 50-fold, at least about 100-fold, at least about 200-fold, at least about 300-fold, at least about 400-fold, or at least about 500-fold higher specificity for muscle tissue as compared to a wild type AAV5.

### Muscle-Tropic Sequences

Recombinant AAV viral capsids with specificity for muscle tissue (muscle-tropic rAAVs) may be identified by screening rAAV libraries comprising VP capsid polypeptides with variant 581-589 regions, as described herein. The libraries may be screened in non-human primates (NHPs) by systemically administering the rAAV library and identifying sequences of 581-589 regions in VP capsid polypeptides that conferred tissue-tropic accumulation in or infection of muscle tissues (e.g., aorta, esophagus, heart (including atrium, ventricle, valves), skeletal muscle (including biceps, brachii, femoris, diaphragm, gastrocnemius, tibialis anterior, triceps, quadriceps, including vastus lateralis)), or muscle fibers (e.g., type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof) to the rAAVs. The muscle-tropic rAAVs may preferentially accumulate in or infect muscle tissues as compared to non-muscle tissues (e.g., liver, CNS, heart, lung, spleen, lymph node, bone marrow, mammary gland, skin, adrenal gland, thyroid, colon, sciatic nerve, spinal cord, or combinations thereof). Exemplary region sequences identified in a primary screen as conferring muscle tropism to an rAAV are provided in TABLE 5 and TABLE 7. Exemplary region sequences identified in a secondary screen as conferring cardiac muscle tropism to an rAAV are provided in TABLE 10. Exemplary region sequences identified in a secondary screen as conferring skeletal muscle tropism to an rAAV are provided in TABLE 11. Exemplary region sequences identified in a secondary screen as conferring muscle tropism to an rAAV are provided in TABLE 12.

A muscle-tropic rAAV assembled from VP capsid polypeptides (e.g., V1, VP2, or VP3 capsid polypeptides, or combinations thereof) comprising a 581-589 region (e.g., any one of SEQ ID NO: 4238-SEQ ID NO: 4933) may preferentially infect a muscle tissue (e.g., aorta, esophagus, heart (including atrium, ventricle, valves), skeletal muscle (including biceps, brachii, femoris, diaphragm, gastrocnemius, tibialis anterior, triceps, quadriceps, including vastus lateralis)), or muscle fibers (e.g., type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof) as compared to non-muscle tissue (e.g., liver, CNS, heart, lung, spleen, lymph node, bone marrow, mammary gland, skin, adrenal gland, thyroid, colon, sciatic nerve, spinal cord, or combinations thereof).

Disclosed herein are engineered AAV5 VP capsid polypeptides capable of forming an assembled virion that exhibits increased muscle tropism as compared to a wild type AAV5 VP capsid polypeptide (e.g., a VP1 of SEQ ID NO: 1, a VP2 of SEQ ID NO: 10, or a VP3 of SEQ ID NO: 11), wherein the engineered variant AAV5 VP capsid polypeptide sequence has one or more amino acid substitutions in a 581-589 region of the VP capsid polypeptide, corresponding to positions 581 to 589 in SEQ ID NO: 2 (X¹X²X³X⁴X⁵X⁶X⁷X⁸X⁹). In some embodiments, X¹ is selected from A, R, N, D, C, E, Q, G, H, I, L, K, M, F, P, S, T, W, Y, or V. In some embodiments, X² is selected from A, R, N, D, C, E, Q, G, H, I, L, K, M, F, P, S, T, W, Y, or V. In some embodiments, X³ is selected from A, R, N, D, C, E, Q, G, H, I, L, K, M, F, P, S, T, W, Y, or V. In some embodiments, X⁴ is selected from A, R, N, D, C, E, Q, G, H, I, L, K, M, F, P, S, T, W, Y, or V. In some embodiments, X⁵ is selected from A, R, N, D, C, E, Q, G, H, I, L, K, M, F, P, S, T, W, Y, or V. In some embodiments, X⁶ is selected from A, R, N, D, C, E, Q, G, H, I, L, K, M, F, P, S, T, W, Y, or V. In some embodiments, X⁷ is selected from A, R, N, D, C, E, Q, G, H, I, L, K, M, F, P, S, T, W, Y, or V. In some embodiments, X⁸ is selected A, R, N, D, C, E, Q, G, H, I, L, K, M, F, P, S, T, W, Y, or V. In some embodiments, X⁹ is selected from A, R, N, D, C, E, Q, G, H, I, L, K, M, F, P, S, T, W, Y, or V.

In some embodiments, a 581-589 region of a muscle-tropic VP capsid polypeptide may comprise a sequence of any one of SEQ ID NO: 4238-SEQ ID NO: 4933.

In some embodiments, provided herein are AAV5 VP capsid polypeptide having at least one mutation in a 581-589 region, corresponding to residues 581 to 589 of AAV5 VP1 (e.g., SEQ ID NO: 1) and having at least 70%, at least 75%, at least 80%, at least 85%, at least 90%, at least 92%, at least 95%, at least 97%, at least 99%, or 100% sequence identity to any sequence selected from SEQ ID NO: 4238-SEQ ID NO: 4933, wherein said at least one mutation drives increased muscle tissue tropism as compared to a wild type AAV5 capsid polypeptide.

### Muscle-Tropic Sequences Generated by Machine Learning

Additional 581-589 regions that confer muscle tissue tropism or preference may be generated using machine learning algorithms trained with sequence identified in muscle tissue in in vivo screens. These patterns may be used to generate new sequences of 581-589 regions that increase the likelihood of conferring muscle tissue tropism to VP capsid polypeptides and rAAVs assembled from the VP capsid polypeptides. In some embodiments, new sequences of 581-589 regions may be generated by randomly sampling amino acid residues at each position of 581-589 regions that favored muscle tissue tropism in an in vivo screen. The generated sequences may be tested using random forests or gradient boosting classifiers to predicted muscle-specificity for each 581-589 region. The 581-589 regions with the highest predicted muscle-specificity may be selected as muscle-tropic sequences. For example, a 581-589 region of any one of SEQ ID NO: 4934-SEQ ID NO: 5233 may be selected. 581-589 region sequences generated using machine learning for increased likelihood of conferring muscle tropism to an rAAV are provided in TABLE 6.

Recombinant AAV viral capsids libraries may be screened in non-human primates (NHPs) by systemically administering the rAAV library and identifying sequences of 581-589 regions in VP capsid polypeptides that increased the likelihood of conferring tissue-tropic accumulation in or infection of muscle tissues (e.g., aorta, esophagus, heart (including atrium, ventricle, valves), skeletal muscle (including biceps, brachii, femoris, diaphragm, gastrocnemius, tibialis anterior, triceps, quadriceps, including vastus lateralis)), or muscle fibers (e.g., type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof.

A muscle-tropic rAAV assembled from VP capsid polypeptides (e.g., V1, VP2, or VP3 capsid polypeptides, or combinations thereof) comprising a 581-589 region may preferentially infect a muscle tissue (e.g., aorta, esophagus, heart (including atrium, ventricle, valves), skeletal muscle (including biceps, brachii, femoris, diaphragm, gastrocnemius, tibialis anterior, triceps, quadriceps, including vastus lateralis)), or muscle fibers (e.g., type I (slow oxidative) fibers, type IC fibers, type II fibers, type IIC fibers, type IIA (fast oxidative/glycolytic) fibers, type IIAX fibers, type IIXA fibers, type IIX (fast glycolytic) fibers, or combinations thereof) as compared to non-muscle tissue (e.g., liver, CNS, heart, lung, spleen, lymph node, bone marrow, mammary gland, skin, adrenal gland, thyroid, colon, sciatic nerve, spinal cord, or combinations thereof), as compared to a wild type AAV5 capsid polypeptide.

Disclosed herein are engineered AAV5 VP capsid polypeptides capable of forming an assembled virion that exhibits increased muscle tissue tropism as compared to a wild type AAV5 VP capsid polypeptide (e.g., a VP1 of SEQ ID NO: 1, a VP2 of SEQ ID NO: 10, or a VP3 of SEQ ID NO: 11), wherein the engineered variant AAV5 VP capsid polypeptide sequence has one or more amino acid substitutions in a 581-589 region of the VP capsid polypeptide, corresponding to positions 581 to 589 in SEQ ID NO: 2 (X¹X²X³X⁴X⁵X⁶X⁷X⁸X⁹). In some embodiments, X¹ is selected from A, R, N, D, C, E, Q, G, H, I, L, K, M, F, P, S, T, W, Y, or V. In some embodiments, X² is selected from A, R, N, D, C, E, Q, G, H, I, L, K, M, F, P, S, T, W, Y, or V. In some embodiments, X³ is selected from A, R, N, D, C, E, Q, G, H, I, L, K, M, F, P, S, T, W, Y, or V. In some embodiments, X⁴ is selected from A, R, N, D, C, E, Q, G, H, I, L, K, M, F, P, S, T, W, Y, or V. In some embodiments, X⁵ is selected from A, R, N, D, C, E, Q, G, H, I, L, K, M, F, P, S, T, W, Y, or V. In some embodiments, X⁶ is selected from A, R, N, D, C, E, Q, G, H, I, L, K, M, F, P, S, T, W, Y, or V. In some embodiments, X⁷ is selected from A, R, N, D, C, E, Q, G, H, I, L, K, M, F, P, S, T, W, Y, or V. In some embodiments, X⁸ is selected A, R, N, D, C, E, Q, G, H, I, L, K, M, F, P, S, T, W, Y, or V. In some embodiments, X⁹ is selected from A, R, N, D, C, E, Q, G, H, I, L, K, M, F, P, S, T, W, Y, or V.

In some embodiments, a 581-589 region of a muscle-tropic VP capsid polypeptide may comprise a sequence of any one of SEQ ID NO: 4934-SEQ ID NO: 5233.

In some embodiments, provided herein are AAV5 VP capsid polypeptide having at least one mutation in a 581-589 region, corresponding to residues 581 to 589 of AAV5 VP1 (e.g., SEQ ID NO: 1) and having at least 70%, at least 75%, at least 80%, at least 85%, at least 90%, at least 92%, at least 95%, at least 97%, at least 99%, or 100% sequence identity to any sequence selected from SEQ ID NO: 4934-SEQ ID NO: 5233, wherein said at least one mutation drives increased muscle tissue tropism as compared to a wild type AAV5 capsid polypeptide.

### Variant 581-589 Regions for Muscle Targeting

In some embodiments, provided herein are AAV5 VP capsid polypeptide having at least one mutation in a 581-589 region, corresponding to residues 581 to 589 of AAV5 VP1, and having at least 70%, at least 75%, at least 80%, at least 85%, at least 90%, at least 92%, at least 95%, at least 97%, at least 99%, or 100% sequence identity to any sequence selected from SEQ ID NO: 4238-SEQ ID NO: 4933, SEQ ID NO: 4934-SEQ ID NO: 5233, or SEQ ID NO: 5808-SEQ ID NO: 5811, wherein said at least one mutation drives increased muscle tissue tropism as compared to a wild type AAV5 capsid polypeptide.

The present disclosure provides a tissue-tropic rAAV comprising a VP capsid polypeptide with a 581-589 variant region, relative to WT AAV5, of SEQ ID NO: 348. The present disclosure provides a tissue-tropic rAAV comprising a VP capsid polypeptide with a 581-589 variant region, relative to WT AAV5, of SEQ ID NO: 2536. The present disclosure provides a tissue-tropic rAAV comprising a VP capsid polypeptide with a 581-589 variant region, relative to WT AAV5, of SEQ ID NO: 5607. The present disclosure provides a tissue-tropic rAAV comprising a VP capsid polypeptide with a 581-589 variant region, relative to WT AAV5, of SEQ ID NO: 1576. The present disclosure provides a tissue-tropic rAAV comprising a VP capsid polypeptide with a 581-589 variant region, relative to WT AAV5, of SEQ ID NO: 4955. The present disclosure provides a tissue-tropic rAAV comprising a VP capsid polypeptide with a 581-589 variant region, relative to WT AAV5, of SEQ ID NO: 5017. The present disclosure provides a tissue-tropic rAAV comprising a VP capsid polypeptide with a 581-589 variant region, relative to WT AAV5, of SEQ ID NO: 4349. The present disclosure provides a tissue-tropic rAAV comprising a VP capsid polypeptide with a 581-589 variant region, relative to WT AAV5, of SEQ ID NO: 4964. The present disclosure provides a tissue-tropic rAAV comprising a VP capsid polypeptide with a 581-589 variant region, relative to WT AAV5, of SEQ ID NO: 293. The present disclosure provides a tissue-tropic rAAV comprising a VP capsid polypeptide with a 581-589 variant region, relative to WT AAV5, of SEQ ID NO: 4314. The present disclosure provides a tissue-tropic rAAV comprising a VP capsid polypeptide with a 581-589 variant region, relative to WT AAV5, of SEQ ID NO: 4995. The present disclosure provides a tissue-tropic rAAV comprising a VP capsid polypeptide with a 581-589 variant region, relative to WT AAV5, of SEQ ID NO: 4366. The present disclosure provides a tissue-tropic rAAV comprising a VP capsid polypeptide with a 581-589 variant region, relative to WT AAV5, of SEQ ID NO: 4961. The present disclosure provides a tissue-tropic rAAV comprising a VP capsid polypeptide with a 581-589 variant region, relative to WT AAV5, of SEQ ID NO: 4952. The present disclosure provides a tissue-tropic rAAV comprising a VP capsid polypeptide with a 581-589 variant region, relative to WT AAV5, of SEQ ID NO: 5075. The present disclosure provides a tissue-tropic rAAV comprising a VP capsid polypeptide with a 581-589 variant region, relative to WT AAV5, of SEQ ID NO: 4354. The present disclosure provides a tissue-tropic rAAV comprising a VP capsid polypeptide with a 581-589 variant region, relative to WT AAV5, of SEQ ID NO: 5060. The present disclosure provides a tissue-tropic rAAV comprising a VP capsid polypeptide with a 581-589 variant region, relative to WT AAV5, of SEQ ID NO: 5138. The present disclosure provides a tissue-tropic rAAV comprising a VP capsid polypeptide with a 581-589 variant region, relative to WT AAV5, of SEQ ID NO: 5206. The present disclosure provides a tissue-tropic rAAV comprising a VP capsid polypeptide with a 581-589 variant region, relative to WT AAV5, of SEQ ID NO: 4288. The present disclosure provides a tissue-tropic rAAV comprising a VP capsid polypeptide with a 581-589 variant region, relative to WT AAV5, of SEQ ID NO: 5283. The present disclosure provides a tissue-tropic rAAV comprising a VP capsid polypeptide with a 581-589 variant region, relative to WT AAV5, of SEQ ID NO: 5092. The present disclosure provides a tissue-tropic rAAV comprising a VP capsid polypeptide with a 581-589 variant region, relative to WT AAV5, of SEQ ID NO: 4059. The present disclosure provides a tissue-tropic rAAV comprising a VP capsid polypeptide with a 581-589 variant region, relative to WT AAV5, of SEQ ID NO: 4295. The present disclosure provides a tissue-tropic rAAV comprising a VP capsid polypeptide with a 581-589 variant region, relative to WT AAV5, of SEQ ID NO: 5030. The present disclosure provides a tissue-tropic rAAV comprising a VP capsid polypeptide with a 581-589 variant region, relative to WT AAV5, of SEQ ID NO: 4938. The present disclosure provides a tissue-tropic rAAV comprising a VP capsid polypeptide with a 581-589 variant region, relative to WT AAV5, of SEQ ID NO: 2661. The present disclosure provides a tissue-tropic rAAV comprising a VP capsid polypeptide with a 581-589 variant region, relative to WT AAV5, of SEQ ID NO: 5215. The present disclosure provides a tissue-tropic rAAV comprising a VP capsid polypeptide with a 581-589 variant region, relative to WT AAV5, of SEQ ID NO: 4960. The present disclosure provides a tissue-tropic rAAV comprising a VP capsid polypeptide with a 581-589 variant region, relative to WT AAV5, of SEQ ID NO: 4969. The present disclosure provides a tissue-tropic rAAV comprising a VP capsid polypeptide with a 581-589 variant region, relative to WT AAV5, of SEQ ID NO: 5023. The present disclosure provides a tissue-tropic rAAV comprising a VP capsid polypeptide with a 581-589 variant region, relative to WT AAV5, of SEQ ID NO: 4934. The present disclosure provides a tissue-tropic rAAV comprising a VP capsid polypeptide with a 581-589 variant region, relative to WT AAV5, of SEQ ID NO: 4545. The present disclosure provides a tissue-tropic rAAV comprising a VP capsid polypeptide with a 581-589 variant region, relative to WT AAV5, of SEQ ID NO: 4963. The present disclosure provides a tissue-tropic rAAV comprising a VP capsid polypeptide with a 581-589 variant region, relative to WT AAV5, of SEQ ID NO: 4363. The present disclosure provides a tissue-tropic rAAV comprising a VP capsid polypeptide with a 581-589 variant region, relative to WT AAV5, of SEQ ID NO: 5159. The present disclosure provides a tissue-tropic rAAV comprising a VP capsid polypeptide with a 581-589 variant region, relative to WT AAV5, of SEQ ID NO: 4973. The present disclosure provides a tissue-tropic rAAV comprising a VP capsid polypeptide with a 581-589 variant region, relative to WT AAV5, of SEQ ID NO: 5157. The present disclosure provides a tissue-tropic rAAV comprising a VP capsid polypeptide with a 581-589 variant region, relative to WT AAV5, of SEQ ID NO: 4972. The present disclosure provides a tissue-tropic rAAV comprising a VP capsid polypeptide with a 581-589 variant region, relative to WT AAV5, of SEQ ID NO: 4345. The present disclosure provides a tissue-tropic rAAV comprising a VP capsid polypeptide with a 581-589 variant region, relative to WT AAV5, of SEQ ID NO: 5039. The present disclosure provides a tissue-tropic rAAV comprising a VP capsid polypeptide with a 581-589 variant region, relative to WT AAV5, of SEQ ID NO: 5163. The present disclosure provides a tissue-tropic rAAV comprising a VP capsid polypeptide with a 581-589 variant region, relative to WT AAV5, of SEQ ID NO: 5040. The present disclosure provides a tissue-tropic rAAV comprising a VP capsid polypeptide with a 581-589 variant region, relative to WT AAV5, of SEQ ID NO: 4304. The present disclosure provides a tissue-tropic rAAV comprising a VP capsid polypeptide with a 581-589 variant region, relative to WT AAV5, of SEQ ID NO: 18. The present disclosure provides a tissue-tropic rAAV comprising a VP capsid polypeptide with a 581-589 variant region, relative to WT AAV5, of SEQ ID NO: 5193. The present disclosure provides a tissue-tropic rAAV comprising a VP capsid polypeptide with a 581-589 variant region, relative to WT AAV5, of SEQ ID NO: 5077. The present disclosure provides a tissue-tropic rAAV comprising a VP capsid polypeptide with a 581-589 variant region, relative to WT AAV5, of SEQ ID NO: 5814. The present disclosure provides a tissue-tropic rAAV comprising a VP capsid polypeptide with a 581-589 variant region, relative to WT AAV5, of SEQ ID NO: 386. The present disclosure provides a tissue-tropic rAAV comprising a VP capsid polypeptide with a 581-589 variant region, relative to WT AAV5, of SEQ ID NO: 5081. The present disclosure provides a tissue-tropic rAAV comprising a VP capsid polypeptide with a 581-589 variant region, relative to WT AAV5, of SEQ ID NO: 308. The present disclosure provides a tissue-tropic rAAV comprising a VP capsid polypeptide with a 581-589 variant region, relative to WT AAV5, of SEQ ID NO: 22. The present disclosure provides a tissue-tropic rAAV comprising a VP capsid polypeptide with a 581-589 variant region, relative to WT AAV5, of SEQ ID NO: 4338. The present disclosure provides a tissue-tropic rAAV comprising a VP capsid polypeptide with a 581-589 variant region, relative to WT AAV5, of SEQ ID NO: 3846. The present disclosure provides a tissue-tropic rAAV comprising a VP capsid polypeptide with a 581-589 variant region, relative to WT AAV5, of SEQ ID NO: 278. The present disclosure provides a tissue-tropic rAAV comprising a VP capsid polypeptide with a 581-589 variant region, relative to WT AAV5, of SEQ ID NO: 5127. The present disclosure provides a tissue-tropic rAAV comprising a VP capsid polypeptide with a 581-589 variant region, relative to WT AAV5, of SEQ ID NO: 5029. The present disclosure provides a tissue-tropic rAAV comprising a VP capsid polypeptide with a 581-589 variant region, relative to WT AAV5, of SEQ ID NO: 5037. The present disclosure provides a tissue-tropic rAAV comprising a VP capsid polypeptide with a 581-589 variant region, relative to WT AAV5, of SEQ ID NO: 5143. The present disclosure provides a tissue-tropic rAAV comprising a VP capsid polypeptide with a 581-589 variant region, relative to WT AAV5, of SEQ ID NO: 4983. The present disclosure provides a tissue-tropic rAAV comprising a VP capsid polypeptide with a 581-589 variant region, relative to WT AAV5, of SEQ ID NO: 4361. The present disclosure provides a tissue-tropic rAAV comprising a VP capsid polypeptide with a 581-589 variant region, relative to WT AAV5, of SEQ ID NO: 4317. The present disclosure provides a tissue-tropic rAAV comprising a VP capsid polypeptide with a 581-589 variant region, relative to WT AAV5, of SEQ ID NO: 5080. The present disclosure provides a tissue-tropic rAAV comprising a VP capsid polypeptide with a 581-589 variant region, relative to WT AAV5, of SEQ ID NO: 23. The present disclosure provides a tissue-tropic rAAV comprising a VP capsid polypeptide with a 581-589 variant region, relative to WT AAV5, of SEQ ID NO: 4343. The present disclosure provides a tissue-tropic rAAV comprising a VP capsid polypeptide with a 581-589 variant region, relative to WT AAV5, of SEQ ID NO: 4986. The present disclosure provides a tissue-tropic rAAV comprising a VP capsid polypeptide with a 581-589 variant region, relative to WT AAV5, of SEQ ID NO: 4311. The present disclosure provides a tissue-tropic rAAV comprising a VP capsid polypeptide with a 581-589 variant region, relative to WT AAV5, of SEQ ID NO: 5102. The present disclosure provides a tissue-tropic rAAV comprising a VP capsid polypeptide with a 581-589 variant region, relative to WT AAV5, of SEQ ID NO: 5280. The present disclosure provides a tissue-tropic rAAV comprising a VP capsid polypeptide with a 581-589 variant region, relative to WT AAV5, of SEQ ID NO: 5185. The present disclosure provides a tissue-tropic rAAV comprising a VP capsid polypeptide with a 581-589 variant region, relative to WT AAV5, of SEQ ID NO: 964. The present disclosure provides a tissue-tropic rAAV comprising a VP capsid polypeptide with a 581-589 variant region, relative to WT AAV5, of SEQ ID NO: 4935. The present disclosure provides a tissue-tropic rAAV comprising a VP capsid polypeptide with a 581-589 variant region, relative to WT AAV5, of SEQ ID NO: 4379. The present disclosure provides a tissue-tropic rAAV comprising a VP capsid polypeptide with a 581-589 variant region, relative to WT AAV5, of SEQ ID NO: 5052. The present disclosure provides a tissue-tropic rAAV comprising a VP capsid polypeptide with a 581-589 variant region, relative to WT AAV5, of SEQ ID NO: 5027. The present disclosure provides a tissue-tropic rAAV comprising a VP capsid polypeptide with a 581-589 variant region, relative to WT AAV5, of SEQ ID NO: 5173. The present disclosure provides a tissue-tropic rAAV comprising a VP capsid polypeptide with a 581-589 variant region, relative to WT AAV5, of SEQ ID NO: 5055. The present disclosure provides a tissue-tropic rAAV comprising a VP capsid polypeptide with a 581-589 variant region, relative to WT AAV5, of SEQ ID NO: 5123. The present disclosure provides a tissue-tropic rAAV comprising a VP capsid polypeptide with a 581-589 variant region, relative to WT AAV5, of SEQ ID NO: 4335. The present disclosure provides a tissue-tropic rAAV comprising a VP capsid polypeptide with a 581-589 variant region, relative to WT AAV5, of SEQ ID NO: 4936. The present disclosure provides a tissue-tropic rAAV comprising a VP capsid polypeptide with a 581-589 variant region, relative to WT AAV5, of SEQ ID NO: 5208. The present disclosure provides a tissue-tropic rAAV comprising a VP capsid polypeptide with a 581-589 variant region, relative to WT AAV5, of SEQ ID NO: 4633. The present disclosure provides a tissue-tropic rAAV comprising a VP capsid polypeptide with a 581-589 variant region, relative to WT AAV5, of SEQ ID NO: 4359. The present disclosure provides a tissue-tropic rAAV comprising a VP capsid polypeptide with a 581-589 variant region, relative to WT AAV5, of SEQ ID NO: 289. The present disclosure provides a tissue-tropic rAAV comprising a VP capsid polypeptide with a 581-589 variant region, relative to WT AAV5, of SEQ ID NO: 4353. The present disclosure provides a tissue-tropic rAAV comprising a VP capsid polypeptide with a 581-589 variant region, relative to WT AAV5, of SEQ ID NO: 4316. The present disclosure provides a tissue-tropic rAAV comprising a VP capsid polypeptide with a 581-589 variant region, relative to WT AAV5, of SEQ ID NO: 5210. The present disclosure provides a tissue-tropic rAAV comprising a VP capsid polypeptide with a 581-589 variant region, relative to WT AAV5, of SEQ ID NO: 4949. The present disclosure provides a tissue-tropic rAAV comprising a VP capsid polypeptide with a 581-589 variant region, relative to WT AAV5, of SEQ ID NO: 4947. The present disclosure provides a tissue-tropic rAAV comprising a VP capsid polypeptide with a 581-589 variant region, relative to WT AAV5, of SEQ ID NO: 2838. The present disclosure provides a tissue-tropic rAAV comprising a VP capsid polypeptide with a 581-589 variant region, relative to WT AAV5, of SEQ ID NO: 378. The present disclosure provides a tissue-tropic rAAV comprising a VP capsid polypeptide with a 581-589 variant region, relative to WT AAV5, of SEQ ID NO: 5013. The present disclosure provides a tissue-tropic rAAV comprising a VP capsid polypeptide with a 581-589 variant region, relative to WT AAV5, of SEQ ID NO: 5278. The present disclosure provides a tissue-tropic rAAV comprising a VP capsid polypeptide with a 581-589 variant region, relative to WT AAV5, of SEQ ID NO: 1471. The present disclosure provides a tissue-tropic rAAV comprising a VP capsid polypeptide with a 581-589 variant region, relative to WT AAV5, of SEQ ID NO: 4346. The present disclosure provides a tissue-tropic rAAV comprising a VP capsid polypeptide with a 581-589 variant region, relative to WT AAV5, of SEQ ID NO: 297. The present disclosure provides a tissue-tropic rAAV comprising a VP capsid polypeptide with a 581-589 variant region, relative to WT AAV5, of SEQ ID NO: 4993. The present disclosure provides a tissue-tropic rAAV comprising a VP capsid polypeptide with a 581-589 variant region, relative to WT AAV5, of SEQ ID NO: 384. The present disclosure provides a tissue-tropic rAAV comprising a VP capsid polypeptide with a 581-589 variant region, relative to WT AAV5, of SEQ ID NO: 5062. The present disclosure provides a tissue-tropic rAAV comprising a VP capsid polypeptide with a 581-589 variant region, relative to WT AAV5, of SEQ ID NO: 4945. The present disclosure provides a tissue-tropic rAAV comprising a VP capsid polypeptide with a 581-589 variant region, relative to WT AAV5, of SEQ ID NO: 3472. The present disclosure provides a tissue-tropic rAAV comprising a VP capsid polypeptide with a 581-589 variant region, relative to WT AAV5, of SEQ ID NO: 3297. The present disclosure provides a tissue-tropic rAAV comprising a VP capsid polypeptide with a 581-589 variant region, relative to WT AAV5, of SEQ ID NO: 210. The present disclosure provides a tissue-tropic rAAV comprising a VP capsid polypeptide with a 581-589 variant region, relative to WT AAV5, of SEQ ID NO: 1971. The present disclosure provides a tissue-tropic rAAV comprising a VP capsid polypeptide with a 581-589 variant region, relative to WT AAV5, of SEQ ID NO: 262. The present disclosure provides a tissue-tropic rAAV comprising a VP capsid polypeptide with a 581-589 variant region, relative to WT AAV5, of SEQ ID NO: 4009. The present disclosure provides a tissue-tropic rAAV comprising a VP capsid polypeptide with a 581-589 variant region, relative to WT AAV5, of SEQ ID NO: 5190. The present disclosure provides a tissue-tropic rAAV comprising a VP capsid polypeptide with a 581-589 variant region, relative to WT AAV5, of SEQ ID NO: 724. The present disclosure provides a tissue-tropic rAAV comprising a VP capsid polypeptide with a 581-589 variant region, relative to WT AAV5, of SEQ ID NO: 1561. The present disclosure provides a tissue-tropic rAAV comprising a VP capsid polypeptide with a 581-589 variant region, relative to WT AAV5, of SEQ ID NO: 5147. The present disclosure provides a tissue-tropic rAAV comprising a VP capsid polypeptide with a 581-589 variant region, relative to WT AAV5, of SEQ ID NO: 4238. The present disclosure provides a tissue-tropic rAAV comprising a VP capsid polypeptide with a 581-589 variant region, relative to WT AAV5, of SEQ ID NO: 5527. The present disclosure provides a tissue-tropic rAAV comprising a VP capsid polypeptide with a 581-589 variant region, relative to WT AAV5, of SEQ ID NO: 3283. The present disclosure provides a tissue-tropic rAAV comprising a VP capsid polypeptide with a 581-589 variant region, relative to WT AAV5, of SEQ ID NO: 4355. The present disclosure provides a tissue-tropic rAAV comprising a VP capsid polypeptide with a 581-589 variant region, relative to WT AAV5, of SEQ ID NO: 5125. The present disclosure provides a tissue-tropic rAAV comprising a VP capsid polypeptide with a 581-589 variant region, relative to WT AAV5, of SEQ ID NO: 4108. The present disclosure provides a tissue-tropic rAAV comprising a VP capsid polypeptide with a 581-589 variant region, relative to WT AAV5, of SEQ ID NO: 2948. The present disclosure provides a tissue-tropic rAAV comprising a VP capsid polypeptide with a 581-589 variant region, relative to WT AAV5, of SEQ ID NO: 3821. The present disclosure provides a tissue-tropic rAAV comprising a VP capsid polypeptide with a 581-589 variant region, relative to WT AAV5, of SEQ ID NO: 4632. The present disclosure provides a tissue-tropic rAAV comprising a VP capsid polypeptide with a 581-589 variant region, relative to WT AAV5, of SEQ ID NO: 294. The present disclosure provides a tissue-tropic rAAV comprising a VP capsid polypeptide with a 581-589 variant region, relative to WT AAV5, of SEQ ID NO: 4943. The present disclosure provides a tissue-tropic rAAV comprising a VP capsid polypeptide with a 581-589 variant region, relative to WT AAV5, of SEQ ID NO: 1391. The present disclosure provides a tissue-tropic rAAV comprising a VP capsid polypeptide with a 581-589 variant region, relative to WT AAV5, of SEQ ID NO: 5078. The present disclosure provides a tissue-tropic rAAV comprising a VP capsid polypeptide with a 581-589 variant region, relative to WT AAV5, of SEQ ID NO: 3299. The present disclosure provides a tissue-tropic rAAV comprising a VP capsid polypeptide with a 581-589 variant region, relative to WT AAV5, of SEQ ID NO: 2897. The present disclosure provides a tissue-tropic rAAV comprising a VP capsid polypeptide with a 581-589 variant region, relative to WT AAV5, of SEQ ID NO: 1537. The present disclosure provides a tissue-tropic rAAV comprising a VP capsid polypeptide with a 581-589 variant region, relative to WT AAV5, of SEQ ID NO: 5255. The present disclosure provides a tissue-tropic rAAV comprising a VP capsid polypeptide with a 581-589 variant region, relative to WT AAV5, of SEQ ID NO: 141. The present disclosure provides a tissue-tropic rAAV comprising a VP capsid polypeptide with a 581-589 variant region, relative to WT AAV5, of SEQ ID NO: 5038. The present disclosure provides a tissue-tropic rAAV comprising a VP capsid polypeptide with a 581-589 variant region, relative to WT AAV5, of SEQ ID NO: 5153. The present disclosure provides a tissue-tropic rAAV comprising a VP capsid polypeptide with a 581-589 variant region, relative to WT AAV5, of SEQ ID NO: 1181. The present disclosure provides a tissue-tropic rAAV comprising a VP capsid polypeptide with a 581-589 variant region, relative to WT AAV5, of SEQ ID NO: 307. The present disclosure provides a tissue-tropic rAAV comprising a VP capsid polypeptide with a 581-589 variant region, relative to WT AAV5, of SEQ ID NO: 1204. The present disclosure provides a tissue-tropic rAAV comprising a VP capsid polypeptide with a 581-589 variant region, relative to WT AAV5, of SEQ ID NO: 4404. The present disclosure provides a tissue-tropic rAAV comprising a VP capsid polypeptide with a 581-589 variant region, relative to WT AAV5, of SEQ ID NO: 5106. The present disclosure provides a tissue-tropic rAAV comprising a VP capsid polypeptide with a 581-589 variant region, relative to WT AAV5, of SEQ ID NO: 2779. The present disclosure provides a tissue-tropic rAAV comprising a VP capsid polypeptide with a 581-589 variant region, relative to WT AAV5, of SEQ ID NO: 1824. The present disclosure provides a tissue-tropic rAAV comprising a VP capsid polypeptide with a 581-589 variant region, relative to WT AAV5, of SEQ ID NO: 5116. The present disclosure provides a tissue-tropic rAAV comprising a VP capsid polypeptide with a 581-589 variant region, relative to WT AAV5, of SEQ ID NO: 3179. The present disclosure provides a tissue-tropic rAAV comprising a VP capsid polypeptide with a 581-589 variant region, relative to WT AAV5, of SEQ ID NO: 3306. The present disclosure provides a tissue-tropic rAAV comprising a VP capsid polypeptide with a 581-589 variant region, relative to WT AAV5, of SEQ ID NO: 98. The present disclosure provides a tissue-tropic rAAV comprising a VP capsid polypeptide with a 581-589 variant region, relative to WT AAV5, of SEQ ID NO: 1872. The present disclosure provides a tissue-tropic rAAV comprising a VP capsid polypeptide with a 581-589 variant region, relative to WT AAV5, of SEQ ID NO: 1268. The present disclosure provides a tissue-tropic rAAV comprising a VP capsid polypeptide with a 581-589 variant region, relative to WT AAV5, of SEQ ID NO: 1634. The present disclosure provides a tissue-tropic rAAV comprising a VP capsid polypeptide with a 581-589 variant region, relative to WT AAV5, of SEQ ID NO: 1060. The present disclosure provides a tissue-tropic rAAV comprising a VP capsid polypeptide with a 581-589 variant region, relative to WT AAV5, of SEQ ID NO: 4941. The present disclosure provides a tissue-tropic rAAV comprising a VP capsid polypeptide with a 581-589 variant region, relative to WT AAV5, of SEQ ID NO: 4981. The present disclosure provides a tissue-tropic rAAV comprising a VP capsid polypeptide with a 581-589 variant region, relative to WT AAV5, of SEQ ID NO: 5274. The present disclosure provides a tissue-tropic rAAV comprising a VP capsid polypeptide with a 581-589 variant region, relative to WT AAV5, of SEQ ID NO: 5816. The present disclosure provides a tissue-tropic rAAV comprising a VP capsid polypeptide with a 581-589 variant region, relative to WT AAV5, of SEQ ID NO: 2842. The present disclosure provides a tissue-tropic rAAV comprising a VP capsid polypeptide with a 581-589 variant region, relative to WT AAV5, of SEQ ID NO: 1934. The present disclosure provides a tissue-tropic rAAV comprising a VP capsid polypeptide with a 581-589 variant region, relative to WT AAV5, of SEQ ID NO: 5817. The present disclosure provides a tissue-tropic rAAV comprising a VP capsid polypeptide with a 581-589 variant region, relative to WT AAV5, of SEQ ID NO: 3998. The present disclosure provides a tissue-tropic rAAV comprising a VP capsid polypeptide with a 581-589 variant region, relative to WT AAV5, of SEQ ID NO: 5026. The present disclosure provides a tissue-tropic rAAV comprising a VP capsid polypeptide with a 581-589 variant region, relative to WT AAV5, of SEQ ID NO: 4290.

### Variant 581-589 Regions for CNS and Cardiac Muscle Dual Tissue Tropism

The present disclosure provides AAV5 virions with a VP capsid polypeptide having at least one mutation in a region with residues that interact with target cells, where the at least one mutation confers tissue tropism for CNS and cardiac muscle as compared to a wild-type VP capsid polypeptide (e.g., a VP capsid polypeptide of SEQ ID NO: 1, SEQ ID NO: 10, or SEQ ID NO: 11). In some embodiments, provided herein are AAV5 VP capsid polypeptide having at least one mutation in a 581-589 region, corresponding to residues 581 to 589 of AAV5 VP1, and having at least 70%, at least 75%, at least 80%, at least 85%, at least 90%, at least 92%, at least 95%, at least 97%, at least 99%, or 100% sequence identity to any sequence selected from SEQ ID NO: 18, SEQ ID NO: 2536, SEQ ID NO: 3846, SEQ ID NO: 4545, SEQ ID NO: 2661, SEQ ID NO: 1576, SEQ ID NO: 4317, SEQ ID NO: 23, SEQ ID NO: 22, SEQ ID NO: 964, SEQ ID NO: 4288, SEQ ID NO: 4338, SEQ ID NO: 5037, or SEQ ID NO: 4936, wherein said at least one mutation drives CNS and cardiac muscle tissue tropism. The present disclosure provides an rAAV having a VP capsid polypeptide with a 581-589 variant region of SEQ ID NO: 18, wherein the rAAV has tissue tropism for CNS tissue and cardiac muscle tissue. The present disclosure provides an rAAV having a VP capsid polypeptide with a 581-589 variant region of SEQ ID NO: 2536, wherein the rAAV has tissue tropism for CNS tissue and cardiac muscle tissue. The present disclosure provides an rAAV having a VP capsid polypeptide with a 581-589 variant region of SEQ ID NO: 3846, wherein the rAAV has tissue tropism for CNS tissue and cardiac muscle tissue. The present disclosure provides an rAAV having a VP capsid polypeptide with a 581-589 variant region of SEQ ID NO: 4545, wherein the rAAV has tissue tropism for CNS tissue and cardiac muscle tissue. The present disclosure provides an rAAV having a VP capsid polypeptide with a 581-589 variant region of SEQ ID NO: 2661, wherein the rAAV has tissue tropism for CNS tissue and cardiac muscle tissue. The present disclosure provides an rAAV having a VP capsid polypeptide with a 581-589 variant region of SEQ ID NO: 1576, wherein the rAAV has tissue tropism for CNS tissue and cardiac muscle tissue. The present disclosure provides an rAAV having a VP capsid polypeptide with a 581-589 variant region of SEQ ID NO: 4317, wherein the rAAV has tissue tropism for CNS tissue and cardiac muscle tissue. The present disclosure provides an rAAV having a VP capsid polypeptide with a 581-589 variant region of SEQ ID NO: 23, wherein the rAAV has tissue tropism for CNS tissue and cardiac muscle tissue. The present disclosure provides an rAAV having a VP capsid polypeptide with a 581-589 variant region of SEQ ID NO: 22, wherein the rAAV has tissue tropism for CNS tissue and cardiac muscle tissue. The present disclosure provides an rAAV having a VP capsid polypeptide with a 581-589 variant region of SEQ ID NO: 964, wherein the rAAV has tissue tropism for CNS tissue and cardiac muscle tissue. The present disclosure provides an rAAV having a VP capsid polypeptide with a 581-589 variant region of SEQ ID NO: 4288, wherein the rAAV has tissue tropism for CNS tissue and cardiac muscle tissue. The present disclosure provides an rAAV having a VP capsid polypeptide with a 581-589 variant region of SEQ ID NO: 4338, wherein the rAAV has tissue tropism for CNS tissue and cardiac muscle tissue. The present disclosure provides an rAAV having a VP capsid polypeptide with a 581-589 variant region of SEQ ID NO: 5037, wherein the rAAV has tissue tropism for CNS tissue and cardiac muscle tissue. The present disclosure provides an rAAV having a VP capsid polypeptide with a 581-589 variant region of SEQ ID NO: 4936, wherein the rAAV has tissue tropism for CNS tissue and cardiac muscle tissue.

### Variant 581-589 Regions for CNS and Skeletal Muscle Dual Tissue Tropism

The present disclosure provides AAV5 virions with a VP capsid polypeptide having at least one mutation in a region with residues that interact with target cells, where the at least one mutation confers tissue tropism for CNS and skeletal muscle as compared to a wild-type VP capsid polypeptide (e.g., a VP capsid polypeptide of SEQ ID NO: 1, SEQ ID NO: 10, or SEQ ID NO: 11). In some embodiments, provided herein are AAV5 VP capsid polypeptide having at least one mutation in a 581-589 region, corresponding to residues 581 to 589 of AAV5 VP1, and having at least 70%, at least 75%, at least 80%, at least 85%, at least 90%, at least 92%, at least 95%, at least 97%, at least 99%, or 100% sequence identity to any sequence selected from SEQ ID NO: 18, SEQ ID NO: 3472, SEQ ID NO: 262, SEQ ID NO: 3306, SEQ ID NO: 2536, SEQ ID NO: 1971, SEQ ID NO: 3283, SEQ ID NO: 3297, SEQ ID NO: 4545, SEQ ID NO: 2661, SEQ ID NO: 307, SEQ ID NO: 4317, SEQ ID NO: 1268, SEQ ID NO: 4288, SEQ ID NO: 724, or SEQ ID NO: 5037, wherein said at least one mutation drives CNS and skeletal muscle tissue tropism. The present disclosure provides an rAAV having a VP capsid polypeptide with a 581-589 variant region of SEQ ID NO: 18, wherein the rAAV has tissue tropism for CNS tissue and skeletal muscle tissue. The present disclosure provides an rAAV having a VP capsid polypeptide with a 581-589 variant region of SEQ ID NO: 3472, wherein the rAAV has tissue tropism for CNS tissue and skeletal muscle tissue. The present disclosure provides an rAAV having a VP capsid polypeptide with a 581-589 variant region of SEQ ID NO: 262, wherein the rAAV has tissue tropism for CNS tissue and skeletal muscle tissue. The present disclosure provides an rAAV having a VP capsid polypeptide with a 581-589 variant region of SEQ ID NO: 3306, wherein the rAAV has tissue tropism for CNS tissue and skeletal muscle tissue. The present disclosure provides an rAAV having a VP capsid polypeptide with a 581-589 variant region of SEQ ID NO: 2536, wherein the rAAV has tissue tropism for CNS tissue and skeletal muscle tissue. The present disclosure provides an rAAV having a VP capsid polypeptide with a 581-589 variant region of SEQ ID NO: 1971, wherein the rAAV has tissue tropism for CNS tissue and skeletal muscle tissue. The present disclosure provides an rAAV having a VP capsid polypeptide with a 581-589 variant region of SEQ ID NO: 3283, wherein the rAAV has tissue tropism for CNS tissue and skeletal muscle tissue. The present disclosure provides an rAAV having a VP capsid polypeptide with a 581-589 variant region of SEQ ID NO: 3297, wherein the rAAV has tissue tropism for CNS tissue and skeletal muscle tissue. The present disclosure provides an rAAV having a VP capsid polypeptide with a 581-589 variant region of SEQ ID NO: 4545, wherein the rAAV has tissue tropism for CNS tissue and skeletal muscle tissue. The present disclosure provides an rAAV having a VP capsid polypeptide with a 581-589 variant region of SEQ ID NO: 2661, wherein the rAAV has tissue tropism for CNS tissue and skeletal muscle tissue. The present disclosure provides an rAAV having a VP capsid polypeptide with a 581-589 variant region of SEQ ID NO: 307, wherein the rAAV has tissue tropism for CNS tissue and skeletal muscle tissue. The present disclosure provides an rAAV having a VP capsid polypeptide with a 581-589 variant region of SEQ ID NO: 4317, wherein the rAAV has tissue tropism for CNS tissue and skeletal muscle tissue. The present disclosure provides an rAAV having a VP capsid polypeptide with a 581-589 variant region of SEQ ID NO: 1268, wherein the rAAV has tissue tropism for CNS tissue and skeletal muscle tissue. The present disclosure provides an rAAV having a VP capsid polypeptide with a 581-589 variant region of SEQ ID NO: 4288, wherein the rAAV has tissue tropism for CNS tissue and skeletal muscle tissue. The present disclosure provides an rAAV having a VP capsid polypeptide with a 581-589 variant region of SEQ ID NO: 724, wherein the rAAV has tissue tropism for CNS tissue and skeletal muscle tissue. The present disclosure provides an rAAV having a VP capsid polypeptide with a 581-589 variant region of SEQ ID NO: 5037, wherein the rAAV has tissue tropism for CNS tissue and skeletal muscle tissue.

### Variant 581-589 Regions for CNS and Muscle Dual Tissue Tropism

The present disclosure provides AAV5 virions with a VP capsid polypeptide having at least one mutation in a region with residues that interact with target cells, where the at least one mutation confers tissue tropism for CNS and muscle as compared to a wild-type VP capsid polypeptide (e.g., a VP capsid polypeptide of SEQ ID NO: 1, SEQ ID NO: 10, or SEQ ID NO: 11). In some embodiments, provided herein are AAV5 VP capsid polypeptide having at least one mutation in a 581-589 region, corresponding to residues 581 to 589 of AAV5 VP1, and having at least 70%, at least 75%, at least 80%, at least 85%, at least 90%, at least 92%, at least 95%, at least 97%, at least 99%, or 100% sequence identity to any sequence selected from SEQ ID NO: 18, SEQ ID NO: 3472, SEQ ID NO: 262, SEQ ID NO: 2536, SEQ ID NO: 3846, SEQ ID NO: 3297, SEQ ID NO: 4545, SEQ ID NO: 2661, SEQ ID NO: 1576, SEQ ID NO: 4317, SEQ ID NO: 23, SEQ ID NO: 22, SEQ ID NO: 964, SEQ ID NO: 4288, SEQ ID NO: 4290, SEQ ID NO: 4338, or SEQ ID NO: 5037, wherein said at least one mutation drives CNS and muscle tissue tropism. The present disclosure provides an rAAV having a VP capsid polypeptide with a 581-589 variant region of SEQ ID NO: 18, wherein the rAAV has tissue tropism for CNS tissue and muscle tissue. The present disclosure provides an rAAV having a VP capsid polypeptide with a 581-589 variant region of SEQ ID NO: 3472, wherein the rAAV has tissue tropism for CNS tissue and muscle tissue. The present disclosure provides an rAAV having a VP capsid polypeptide with a 581-589 variant region of SEQ ID NO: 262, wherein the rAAV has tissue tropism for CNS tissue and muscle tissue. The present disclosure provides an rAAV having a VP capsid polypeptide with a 581-589 variant region of SEQ ID NO: 2536, wherein the rAAV has tissue tropism for CNS tissue and muscle tissue. The present disclosure provides an rAAV having a VP capsid polypeptide with a 581-589 variant region of SEQ ID NO: 3846, wherein the rAAV has tissue tropism for CNS tissue and muscle tissue. The present disclosure provides an rAAV having a VP capsid polypeptide with a 581-589 variant region of SEQ ID NO: 3297, wherein the rAAV has tissue tropism for CNS tissue and muscle tissue. The present disclosure provides an rAAV having a VP capsid polypeptide with a 581-589 variant region of SEQ ID NO: 4545, wherein the rAAV has tissue tropism for CNS tissue and muscle tissue. The present disclosure provides an rAAV having a VP capsid polypeptide with a 581-589 variant region of SEQ ID NO: 2661, wherein the rAAV has tissue tropism for CNS tissue and muscle tissue. The present disclosure provides an rAAV having a VP capsid polypeptide with a 581-589 variant region of SEQ ID NO: 1576, wherein the rAAV has tissue tropism for CNS tissue and muscle tissue. The present disclosure provides an rAAV having a VP capsid polypeptide with a 581-589 variant region of SEQ ID NO: 4317, wherein the rAAV has tissue tropism for CNS tissue and muscle tissue. The present disclosure provides an rAAV having a VP capsid polypeptide with a 581-589 variant region of SEQ ID NO: 23, wherein the rAAV has tissue tropism for CNS tissue and muscle tissue. The present disclosure provides an rAAV having a VP capsid polypeptide with a 581-589 variant region of SEQ ID NO: 22, wherein the rAAV has tissue tropism for CNS tissue and muscle tissue. The present disclosure provides an rAAV having a VP capsid polypeptide with a 581-589 variant region of SEQ ID NO: 964, wherein the rAAV has tissue tropism for CNS tissue and muscle tissue. The present disclosure provides an rAAV having a VP capsid polypeptide with a 581-589 variant region of SEQ ID NO: 4288, wherein the rAAV has tissue tropism for CNS tissue and muscle tissue. The present disclosure provides an rAAV having a VP capsid polypeptide with a 581-589 variant region of SEQ ID NO: 4290, wherein the rAAV has tissue tropism for CNS tissue and muscle tissue. The present disclosure provides an rAAV having a VP capsid polypeptide with a 581-589 variant region of SEQ ID NO: 4338, wherein the rAAV has tissue tropism for CNS tissue and muscle tissue. The present disclosure provides an rAAV having a VP capsid polypeptide with a 581-589 variant region of SEQ ID NO: 5037, wherein the rAAV has tissue tropism for CNS tissue and muscle tissue.

### Variant 581-589 Regions for Cardiac Muscle and Skeletal Muscle Dual Tissue Tropism

The present disclosure provides AAV5 virions with a VP capsid polypeptide having at least one mutation in a region with residues that interact with target cells, where the at least one mutation confers tissue tropism for cardiac muscle and skeletal muscle as compared to a wild-type VP capsid polypeptide (e.g., a VP capsid polypeptide of SEQ ID NO: 1, SEQ ID NO: 10, or SEQ ID NO: 11). In some embodiments, provided herein are AAV5 VP capsid polypeptide having at least one mutation in a 581-589 region, corresponding to residues 581 to 589 of AAV5 VP1, and having at least 70%, at least 75%, at least 80%, at least 85%, at least 90%, at least 92%, at least 95%, at least 97%, at least 99%, or 100% sequence identity to any sequence selected from SEQ ID NO: 348, SEQ ID NO: 2536, SEQ ID NO: 5607, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5283, SEQ ID NO: 5092, SEQ ID NO: 4059, SEQ ID NO: 4295, SEQ ID NO: 5030, SEQ ID NO: 2661, SEQ ID NO: 4545, SEQ ID NO: 4963, SEQ ID NO: 4363, SEQ ID NO: 5159, SEQ ID NO: 4973, SEQ ID NO: 5157, SEQ ID NO: 4345, SEQ ID NO: 5039, SEQ ID NO: 4304, SEQ ID NO: 18, SEQ ID NO: 386, SEQ ID NO: 308, SEQ ID NO: 278, SEQ ID NO: 5037, SEQ ID NO: 4361, SEQ ID NO: 4317, SEQ ID NO: 4343, SEQ ID NO: 4311, SEQ ID NO: 5055, SEQ ID NO: 4359, SEQ ID NO: 4353, or SEQ ID NO: 4346, wherein said at least one mutation drives cardiac muscle and skeletal muscle tissue tropism. The present disclosure provides an rAAV having a VP capsid polypeptide with a 581-589 variant region of SEQ ID NO: 348, wherein the rAAV has tissue tropism for cardiac muscle tissue and skeletal muscle tissue. The present disclosure provides an rAAV having a VP capsid polypeptide with a 581-589 variant region of SEQ ID NO: 2536, wherein the rAAV has tissue tropism for cardiac muscle tissue and skeletal muscle tissue. The present disclosure provides an rAAV having a VP capsid polypeptide with a 581-589 variant region of SEQ ID NO: 5607, wherein the rAAV has tissue tropism for cardiac muscle tissue and skeletal muscle tissue. The present disclosure provides an rAAV having a VP capsid polypeptide with a 581-589 variant region of SEQ ID NO: 4955, wherein the rAAV has tissue tropism for cardiac muscle tissue and skeletal muscle tissue. The present disclosure provides an rAAV having a VP capsid polypeptide with a 581-589 variant region of SEQ ID NO: 5017, wherein the rAAV has tissue tropism for cardiac muscle tissue and skeletal muscle tissue. The present disclosure provides an rAAV having a VP capsid polypeptide with a 581-589 variant region of SEQ ID NO: 4349, wherein the rAAV has tissue tropism for cardiac muscle tissue and skeletal muscle tissue. The present disclosure provides an rAAV having a VP capsid polypeptide with a 581-589 variant region of SEQ ID NO: 4964, wherein the rAAV has tissue tropism for cardiac muscle tissue and skeletal muscle tissue. The present disclosure provides an rAAV having a VP capsid polypeptide with a 581-589 variant region of SEQ ID NO: 293, wherein the rAAV has tissue tropism for cardiac muscle tissue and skeletal muscle tissue. The present disclosure provides an rAAV having a VP capsid polypeptide with a 581-589 variant region of SEQ ID NO: 4314, wherein the rAAV has tissue tropism for cardiac muscle tissue and skeletal muscle tissue. The present disclosure provides an rAAV having a VP capsid polypeptide with a 581-589 variant region of SEQ ID NO: 4995, wherein the rAAV has tissue tropism for cardiac muscle tissue and skeletal muscle tissue. The present disclosure provides an rAAV having a VP capsid polypeptide with a 581-589 variant region of SEQ ID NO: 4366, wherein the rAAV has tissue tropism for cardiac muscle tissue and skeletal muscle tissue. The present disclosure provides an rAAV having a VP capsid polypeptide with a 581-589 variant region of SEQ ID NO: 4961, wherein the rAAV has tissue tropism for cardiac muscle tissue and skeletal muscle tissue. The present disclosure provides an rAAV having a VP capsid polypeptide with a 581-589 variant region of SEQ ID NO: 4952, wherein the rAAV has tissue tropism for cardiac muscle tissue and skeletal muscle tissue. The present disclosure provides an rAAV having a VP capsid polypeptide with a 581-589 variant region of SEQ ID NO: 5075, wherein the rAAV has tissue tropism for cardiac muscle tissue and skeletal muscle tissue. The present disclosure provides an rAAV having a VP capsid polypeptide with a 581-589 variant region of SEQ ID NO: 4354, wherein the rAAV has tissue tropism for cardiac muscle tissue and skeletal muscle tissue. The present disclosure provides an rAAV having a VP capsid polypeptide with a 581-589 variant region of SEQ ID NO: 5060, wherein the rAAV has tissue tropism for cardiac muscle tissue and skeletal muscle tissue. The present disclosure provides an rAAV having a VP capsid polypeptide with a 581-589 variant region of SEQ ID NO: 5138, wherein the rAAV has tissue tropism for cardiac muscle tissue and skeletal muscle tissue. The present disclosure provides an rAAV having a VP capsid polypeptide with a 581-589 variant region of SEQ ID NO: 5206, wherein the rAAV has tissue tropism for cardiac muscle tissue and skeletal muscle tissue. The present disclosure provides an rAAV having a VP capsid polypeptide with a 581-589 variant region of SEQ ID NO: 4288, wherein the rAAV has tissue tropism for cardiac muscle tissue and skeletal muscle tissue. The present disclosure provides an rAAV having a VP capsid polypeptide with a 581-589 variant region of SEQ ID NO: 5283, wherein the rAAV has tissue tropism for cardiac muscle tissue and skeletal muscle tissue. The present disclosure provides an rAAV having a VP capsid polypeptide with a 581-589 variant region of SEQ ID NO: 5092, wherein the rAAV has tissue tropism for cardiac muscle tissue and skeletal muscle tissue. The present disclosure provides an rAAV having a VP capsid polypeptide with a 581-589 variant region of SEQ ID NO: 4059, wherein the rAAV has tissue tropism for cardiac muscle tissue and skeletal muscle tissue. The present disclosure provides an rAAV having a VP capsid polypeptide with a 581-589 variant region of SEQ ID NO: 4295, wherein the rAAV has tissue tropism for cardiac muscle tissue and skeletal muscle tissue. The present disclosure provides an rAAV having a VP capsid polypeptide with a 581-589 variant region of SEQ ID NO: 5030, wherein the rAAV has tissue tropism for cardiac muscle tissue and skeletal muscle tissue. The present disclosure provides an rAAV having a VP capsid polypeptide with a 581-589 variant region of SEQ ID NO: 2661, wherein the rAAV has tissue tropism for cardiac muscle tissue and skeletal muscle tissue. The present disclosure provides an rAAV having a VP capsid polypeptide with a 581-589 variant region of SEQ ID NO: 4545, wherein the rAAV has tissue tropism for cardiac muscle tissue and skeletal muscle tissue. The present disclosure provides an rAAV having a VP capsid polypeptide with a 581-589 variant region of SEQ ID NO: 4963, wherein the rAAV has tissue tropism for cardiac muscle tissue and skeletal muscle tissue. The present disclosure provides an rAAV having a VP capsid polypeptide with a 581-589 variant region of SEQ ID NO: 4363, wherein the rAAV has tissue tropism for cardiac muscle tissue and skeletal muscle tissue. The present disclosure provides an rAAV having a VP capsid polypeptide with a 581-589 variant region of SEQ ID NO: 5159, wherein the rAAV has tissue tropism for cardiac muscle tissue and skeletal muscle tissue. The present disclosure provides an rAAV having a VP capsid polypeptide with a 581-589 variant region of SEQ ID NO: 4973, wherein the rAAV has tissue tropism for cardiac muscle tissue and skeletal muscle tissue. The present disclosure provides an rAAV having a VP capsid polypeptide with a 581-589 variant region of SEQ ID NO: 5157, wherein the rAAV has tissue tropism for cardiac muscle tissue and skeletal muscle tissue. The present disclosure provides an rAAV having a VP capsid polypeptide with a 581-589 variant region of SEQ ID NO: 4345, wherein the rAAV has tissue tropism for cardiac muscle tissue and skeletal muscle tissue. The present disclosure provides an rAAV having a VP capsid polypeptide with a 581-589 variant region of SEQ ID NO: 5039, wherein the rAAV has tissue tropism for cardiac muscle tissue and skeletal muscle tissue. The present disclosure provides an rAAV having a VP capsid polypeptide with a 581-589 variant region of SEQ ID NO: 4304, wherein the rAAV has tissue tropism for cardiac muscle tissue and skeletal muscle tissue. The present disclosure provides an rAAV having a VP capsid polypeptide with a 581-589 variant region of SEQ ID NO: 18, wherein the rAAV has tissue tropism for cardiac muscle tissue and skeletal muscle tissue. The present disclosure provides an rAAV having a VP capsid polypeptide with a 581-589 variant region of SEQ ID NO: 386, wherein the rAAV has tissue tropism for cardiac muscle tissue and skeletal muscle tissue. The present disclosure provides an rAAV having a VP capsid polypeptide with a 581-589 variant region of SEQ ID NO: 308, wherein the rAAV has tissue tropism for cardiac muscle tissue and skeletal muscle tissue. The present disclosure provides an rAAV having a VP capsid polypeptide with a 581-589 variant region of SEQ ID NO: 278, wherein the rAAV has tissue tropism for cardiac muscle tissue and skeletal muscle tissue. The present disclosure provides an rAAV having a VP capsid polypeptide with a 581-589 variant region of SEQ ID NO: 5037, wherein the rAAV has tissue tropism for cardiac muscle tissue and skeletal muscle tissue. The present disclosure provides an rAAV having a VP capsid polypeptide with a 581-589 variant region of SEQ ID NO: 4361, wherein the rAAV has tissue tropism for cardiac muscle tissue and skeletal muscle tissue. The present disclosure provides an rAAV having a VP capsid polypeptide with a 581-589 variant region of SEQ ID NO: 4317, wherein the rAAV has tissue tropism for cardiac muscle tissue and skeletal muscle tissue. The present disclosure provides an rAAV having a VP capsid polypeptide with a 581-589 variant region of SEQ ID NO: 4343, wherein the rAAV has tissue tropism for cardiac muscle tissue and skeletal muscle tissue. The present disclosure provides an rAAV having a VP capsid polypeptide with a 581-589 variant region of SEQ ID NO: 4311, wherein the rAAV has tissue tropism for cardiac muscle tissue and skeletal muscle tissue. The present disclosure provides an rAAV having a VP capsid polypeptide with a 581-589 variant region of SEQ ID NO: 5055, wherein the rAAV has tissue tropism for cardiac muscle tissue and skeletal muscle tissue. The present disclosure provides an rAAV having a VP capsid polypeptide with a 581-589 variant region of SEQ ID NO: 4359, wherein the rAAV has tissue tropism for cardiac muscle tissue and skeletal muscle tissue. The present disclosure provides an rAAV having a VP capsid polypeptide with a 581-589 variant region of SEQ ID NO: 4353, wherein the rAAV has tissue tropism for cardiac muscle tissue and skeletal muscle tissue. The present disclosure provides an rAAV having a VP capsid polypeptide with a 581-589 variant region of SEQ ID NO: 4346, wherein the rAAV has tissue tropism for cardiac muscle tissue and skeletal muscle tissue.

### Research Tools

An engineered VP capsid polypeptide of the present disclosure, or a recombinant AAV capsid comprising an engineered VP capsid polypeptide, may be used as a research tool in mice. In some embodiments, a recombinant capsid may exhibit tissue tropism in both humans and in a model organism (e.g., non-human primates or mice). In some embodiments, a recombinant capsid may exhibit tissue tropism in both non-human primates and in a second model organism (e.g., mice). A recombinant AAV capsid exhibiting tissue tropism a first organism (e.g., humans or non-human primates) and a second organism (e.g., non-human primates or mice) may be used as a research tool in the second organism to represent behavior of the recombinant AAV capsid, or a recombinant virus comprising the recombinant AAV capsid, in the second organism. For example, a recombinant AAV capsid having 581-589 region sequences of SEQ ID NO: 5027, SEQ ID NO: 4633, SEQ ID NO: 4943, SEQ ID NO: 386, SEQ ID NO: 334, SEQ ID NO: 4309, SEQ ID NO: 4288, SEQ ID NO: 4964, SEQ ID NO: 5017, SEQ ID NO: 5814 (MACKVHLQP), SEQ ID NO: 4335, SEQ ID NO: 348, SEQ ID NO: 4936, SEQ ID NO: 5206, SEQ ID NO: 4238, SEQ ID NO: 5077, or SEQ ID NO: 5603 may be used as a research tool in mice to represent behavior of the recombinant AAV capsid, or a recombinant virus comprising the recombinant AAV capsid in humans or non-human primates. In some embodiments, a recombinant AAV capsid having 581-589 region sequences of SEQ ID NO: 5027, SEQ ID NO: 4633, SEQ ID NO: 4943, SEQ ID NO: 386, SEQ ID NO: 334, SEQ ID NO: 4309, SEQ ID NO: 4288, SEQ ID NO: 4964, SEQ ID NO: 5017, SEQ ID NO: 5814, SEQ ID NO: 4335, SEQ ID NO: 348, SEQ ID NO: 4936, SEQ ID NO: 5206, SEQ ID NO: 4238, SEQ ID NO: 5077, or SEQ ID NO: 5603 exhibits cardiac muscle tissue tropism in both non-human primates and mice.

In some embodiments, a 581-589 region of an AAV5 VP capsid polypeptide has a sequence that is at least 75%, at least 77.7%, at least 85%, at least 88.8% or 100% identical to a sequence provided in TABLE 13.

In some embodiments, a 581-589 region of an AAV5 VP capsid polypeptide has a sequence that is at least 75%, at least 77.7%, at least 85%, at least 88.8% or 100% identical to SEQ ID NO: 5027. In some embodiments, a 581-589 region of an AAV5 VP capsid polypeptide has a sequence that is at least 75%, at least 77.7%, at least 85%, at least 88.8% or 100% identical to SEQ ID NO: 4633. In some embodiments, a 581-589 region of an AAV5 VP capsid polypeptide has a sequence that is at least 75%, at least 77.7%, at least 85%, at least 88.8% or 100% identical to SEQ ID NO: 4943. In some embodiments, a 581-589 region of an AAV5 VP capsid polypeptide has a sequence that is at least 75%, at least 77.7%, at least 85%, at least 88.8% or 100% identical to SEQ ID NO: 386. In some embodiments, a 581-589 region of an AAV5 VP capsid polypeptide has a sequence that is at least 75%, at least 77.7%, at least 85%, at least 88.8% or 100% identical to SEQ ID NO: 334. In some embodiments, a 581-589 region of an AAV5 VP capsid polypeptide has a sequence that is at least 75%, at least 77.7%, at least 85%, at least 88.8% or 100% identical to SEQ ID NO: 4309. In some embodiments, a 581-589 region of an AAV5 VP capsid polypeptide has a sequence that is at least 75%, at least 77.7%, at least 85%, at least 88.8% or 100% identical to SEQ ID NO: 4288. In some embodiments, a 581-589 region of an AAV5 VP capsid polypeptide has a sequence that is at least 75%, at least 77.7%, at least 85%, at least 88.8% or 100% identical to SEQ ID NO: 4964. In some embodiments, a 581-589 region of an AAV5 VP capsid polypeptide has a sequence that is at least 75%, at least 77.7%, at least 85%, at least 88.8% or 100% identical to SEQ ID NO: 5017. In some embodiments, a 581-589 region of an AAV5 VP capsid polypeptide has a sequence that is at least 75%, at least 77.7%, at least 85%, at least 88.8% or 100% identical to SEQ ID NO: 5814. In some embodiments, a 581-589 region of an AAV5 VP capsid polypeptide has a sequence that is at least 75%, at least 77.7%, at least 85%, at least 88.8% or 100% identical to SEQ ID NO: 4335. In some embodiments, a 581-589 region of an AAV5 VP capsid polypeptide has a sequence that is at least 75%, at least 77.7%, at least 85%, at least 88.8% or 100% identical to SEQ ID NO: 348. In some embodiments, a 581-589 region of an AAV5 VP capsid polypeptide has a sequence that is at least 75%, at least 77.7%, at least 85%, at least 88.8% or 100% identical to SEQ ID NO: 4936. In some embodiments, a 581-589 region of an AAV5 VP capsid polypeptide has a sequence that is at least 75%, at least 77.7%, at least 85%, at least 88.8% or 100% identical to SEQ ID NO: 5206. In some embodiments, a 581-589 region of an AAV5 VP capsid polypeptide has a sequence that is at least 75%, at least 77.7%, at least 85%, at least 88.8% or 100% identical to SEQ ID NO: 4238. In some embodiments, a 581-589 region of an AAV5 VP capsid polypeptide has a sequence that is at least 75%, at least 77.7%, at least 85%, at least 88.8% or 100% identical to SEQ ID NO: 5077. In some embodiments, a 581-589 region of an AAV5 VP capsid polypeptide has a sequence that is at least 75%, at least 77.7%, at least 85%, at least 88.8% or 100% identical to SEQ ID NO: 5603.

In some embodiments, a 581-589 region of an AAV5 VP capsid polypeptide has a sequence of SEQ ID NO: 5027. In some embodiments, a 581-589 region of an AAV5 VP capsid polypeptide has a sequence of SEQ ID NO: 4633. In some embodiments, a 581-589 region of an AAV5 VP capsid polypeptide has a sequence of SEQ ID NO: 4943. In some embodiments, a 581-589 region of an AAV5 VP capsid polypeptide has a sequence of SEQ ID NO: 386. In some embodiments, a 581-589 region of an AAV5 VP capsid polypeptide has a sequence of SEQ ID NO: 334. In some embodiments, a 581-589 region of an AAV5 VP capsid polypeptide has a sequence of SEQ ID NO: 4309. In some embodiments, a 581-589 region of an AAV5 VP capsid polypeptide has a sequence of SEQ ID NO: 4288. In some embodiments, a 581-589 region of an AAV5 VP capsid polypeptide has a sequence of SEQ ID NO: 4964. In some embodiments, a 581-589 region of an AAV5 VP capsid polypeptide has a sequence of SEQ ID NO: 5017. In some embodiments, a 581-589 region of an AAV5 VP capsid polypeptide has a sequence of SEQ ID NO: 5814. In some embodiments, a 581-589 region of an AAV5 VP capsid polypeptide has a sequence of SEQ ID NO: 4335. In some embodiments, a 581-589 region of an AAV5 VP capsid polypeptide has a sequence of SEQ ID NO: 348. In some embodiments, a 581-589 region of an AAV5 VP capsid polypeptide has a sequence of SEQ ID NO: 4936. In some embodiments, a 581-589 region of an AAV5 VP capsid polypeptide has a sequence of SEQ ID NO: 5206. In some embodiments, a 581-589 region of an AAV5 VP capsid polypeptide has a sequence of SEQ ID NO: 4238. In some embodiments, a 581-589 region of an AAV5 VP capsid polypeptide has a sequence of SEQ ID NO: 5077. In some embodiments, a 581-589 region of an AAV5 VP capsid polypeptide has a sequence of SEQ ID NO: 5603.

In some embodiments, a 581-589 region of an AAV5 VP capsid polypeptide has a sequence of SEQ ID NO: 5027 with 1 or 2 conservative substitutions. In some embodiments, a 581-589 region of an AAV5 VP capsid polypeptide has a sequence of SEQ ID NO: 4633 with 1 or 2 conservative substitutions. In some embodiments, a 581-589 region of an AAV5 VP capsid polypeptide has a sequence of SEQ ID NO: 4943 with 1 or 2 conservative substitutions. In some embodiments, a 581-589 region of an AAV5 VP capsid polypeptide has a sequence of SEQ ID NO: 386 with 1 or 2 conservative substitutions. In some embodiments, a 581-589 region of an AAV5 VP capsid polypeptide has a sequence of SEQ ID NO: 334 with 1 or 2 conservative substitutions. In some embodiments, a 581-589 region of an AAV5 VP capsid polypeptide has a sequence of SEQ ID NO: 4309 with 1 or 2 conservative substitutions. In some embodiments, a 581-589 region of an AAV5 VP capsid polypeptide has a sequence of SEQ ID NO: 4288 with 1 or 2 conservative substitutions. In some embodiments, a 581-589 region of an AAV5 VP capsid polypeptide has a sequence of SEQ ID NO: 4964 with 1 or 2 conservative substitutions. In some embodiments, a 581-589 region of an AAV5 VP capsid polypeptide has a sequence of SEQ ID NO: 5017 with 1 or 2 conservative substitutions. In some embodiments, a 581-589 region of an AAV5 VP capsid polypeptide has a sequence of SEQ ID NO: 5814 with 1 or 2 conservative substitutions. In some embodiments, a 581-589 region of an AAV5 VP capsid polypeptide has a sequence of SEQ ID NO: 4335 with 1 or 2 conservative substitutions. In some embodiments, a 581-589 region of an AAV5 VP capsid polypeptide has a sequence of SEQ ID NO: 348 with 1 or 2 conservative substitutions. In some embodiments, a 581-589 region of an AAV5 VP capsid polypeptide has a sequence of SEQ ID NO: 4936 with 1 or 2 conservative substitutions. In some embodiments, a 581-589 region of an AAV5 VP capsid polypeptide has a sequence of SEQ ID NO: 5206 with 1 or 2 conservative substitutions. In some embodiments, a 581-589 region of an AAV5 VP capsid polypeptide has a sequence of SEQ ID NO: 4238 with 1 or 2 conservative substitutions. In some embodiments, a 581-589 region of an AAV5 VP capsid polypeptide has a sequence of SEQ ID NO: 5077 with 1 or 2 conservative substitutions. In some embodiments, a 581-589 region of an AAV5 VP capsid polypeptide has a sequence of SEQ ID NO: 5603 with 1 or 2 conservative substitutions. The conservative substitutions may be at any position in the 581-589 region.

### NUMBERED EMBODIMENTS

The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed. 1. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a cardiac muscle tissue at a DNA enrichment of at least 25-fold greater than a wild type AAV9. 2. The VP capsid polypeptide of embodiment 1, wherein the 581-589 region has a sequence selected from the group consisting of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5092, SEQ ID NO: 4059, SEQ ID NO: 4295, SEQ ID NO: 5030, SEQ ID NO: 4938, SEQ ID NO: 2661, SEQ ID NO: 5215, SEQ ID NO: 4960, SEQ ID NO: 4969, SEQ ID NO: 5023, SEQ ID NO: 4934, SEQ ID NO: 4545, SEQ ID NO: 4963, SEQ ID NO: 4363, SEQ ID NO: 5159, and SEQ ID NO: 4973. 3. The VP capsid polypeptide of any one of embodiments 1-2, wherein the recombinant viral capsid is capable of preferentially targeting the cardiac muscle tissue at a DNA enrichment of at least 30-fold greater than the wild type AAV9. 4. The VP capsid polypeptide of embodiment 3, wherein the 581-589 region has a sequence selected from the group consisting of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, and SEQ ID NO: 4366. 5. The VP capsid polypeptide of any one of embodiments 1-4, wherein the recombinant viral capsid is capable of preferentially targeting the cardiac muscle tissue at a DNA enrichment of at least 50-fold greater than the wild type AAV9. 6. The VP capsid polypeptide of embodiment 5, wherein the 581-589 region has a sequence of SEQ ID NO: 348 or SEQ ID NO: 2536. 7. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a cardiac muscle tissue at an RNA enrichment of at least 10-fold greater than a wild type AAV9. 8. The VP capsid polypeptide of embodiment 7, wherein the 581-589 region has a sequence selected from the group consisting of SEQ ID NO: 348, SEQ ID NO: 5052, SEQ ID NO: 5193, SEQ ID NO: 5138, SEQ ID NO: 5163, SEQ ID NO: 4952, SEQ ID NO: 293, SEQ ID NO: 4317, SEQ ID NO: 5075, SEQ ID NO: 386, SEQ ID NO: 4288, SEQ ID NO: 4995, SEQ ID NO: 5030, SEQ ID NO: 4986, SEQ ID NO: 5206, SEQ ID NO: 4338, SEQ ID NO: 5159, SEQ ID NO: 4346, SEQ ID NO: 5060, SEQ ID NO: 4949, SEQ ID NO: 5017, SEQ ID NO: 5215, SEQ ID NO: 4314, SEQ ID NO: 5080, SEQ ID NO: 4964, SEQ ID NO: 4379, SEQ ID NO: 4304, SEQ ID NO: 18, SEQ ID NO: 4363, SEQ ID NO: 5027, SEQ ID NO: 4349, SEQ ID NO: 5208, and SEQ ID NO: 4938. 9. The VP capsid polypeptide of any one of embodiments 7-8, wherein the recombinant viral capsid is capable of preferentially targeting the cardiac muscle tissue at an RNA enrichment of at least 20-fold or greater than the wild type AAV9. 10. The VP capsid polypeptide of embodiment 9, wherein the 581-589 region has a sequence selected from the group consisting of SEQ ID NO: 348, SEQ ID NO: 5052, SEQ ID NO: 5193, SEQ ID NO: 5138, and SEQ ID NO: 5163. 11. The VP capsid polypeptide of any one of embodiments 7-8, wherein the recombinant viral capsid is capable of preferentially targeting the cardiac muscle tissue at an RNA enrichment of at least 40-fold or greater than the wild type AAV9. 12. The VP capsid polypeptide of embodiment 11, wherein the 581-589 region has a sequence of SEQ ID NO: 348. 13. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a cardiac muscle tissue at a DNA enrichment of at least 5-fold greater than a wild type AAV5. 14. The VP capsid polypeptide of embodiment 13, wherein the 581-589 region has a sequence selected from the group consisting of SEQ ID NO: 348, SEQ ID NO: 2536, SEQ ID NO: 1576, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5092, SEQ ID NO: 4059, SEQ ID NO: 4295, SEQ ID NO: 5030, SEQ ID NO: 4938, SEQ ID NO: 2661, SEQ ID NO: 5215, SEQ ID NO: 4960, SEQ ID NO: 4969, SEQ ID NO: 5023, SEQ ID NO: 4934, SEQ ID NO: 4545, SEQ ID NO: 4963, SEQ ID NO: 4363, SEQ ID NO: 5159, and SEQ ID NO: 4973. 15. The VP capsid polypeptide of any one of embodiments 13-14, wherein the recombinant viral capsid is capable of preferentially targeting the cardiac muscle tissue at a DNA enrichment of at least 10-fold greater than the wild type AAV5. 16. The VP capsid polypeptide of embodiment 15, wherein the 581-589 region has a sequence of SEQ ID NO: 348 or SEQ ID NO: 2536. 17. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a cardiac muscle tissue at an RNA enrichment of at least 5-fold greater than a wild type AAV5. 18. The VP capsid polypeptide of embodiment 17, wherein the 581-589 region has a sequence selected from the group consisting of SEQ ID NO: 348, SEQ ID NO: 5052, SEQ ID NO: 5193, SEQ ID NO: 5138, SEQ ID NO: 5163, SEQ ID NO: 4952, SEQ ID NO: 293, and SEQ ID NO: 4317. 19. The VP capsid polypeptide of any one of embodiments 16-17, wherein the recombinant viral capsid is capable of preferentially targeting the cardiac muscle tissue at an RNA enrichment of at least 10-fold greater than the wild type AAV5. 20. The VP capsid polypeptide of embodiment 19, wherein the 581-589 region has a sequence of SEQ ID NO: 348. 21. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 348, (b) SEQ ID NO: 2536, (c) SEQ ID NO: 1576, (d) SEQ ID NO: 4955, (e) SEQ ID NO: 5017, (f) SEQ ID NO: 4349, (g) SEQ ID NO: 4964, (h) SEQ ID NO: 293, (i) SEQ ID NO: 4314, (j) SEQ ID NO: 4995, (k) SEQ ID NO: 4366, (l) SEQ ID NO: 4961, (m) SEQ ID NO: 4952, (n) SEQ ID NO: 5075, (o) SEQ ID NO: 4354, (p) SEQ ID NO: 5060, (q) SEQ ID NO: 5138, (r) SEQ ID NO: 5206, (s) SEQ ID NO: 4288, (t) SEQ ID NO: 5092, (u) SEQ ID NO: 4059, (v) SEQ ID NO: 4295, (w) SEQ ID NO: 5030, (x) SEQ ID NO: 4938, (y) SEQ ID NO: 2661, (z) SEQ ID NO: 5215, (aa) SEQ ID NO: 4960, (bb) SEQ ID NO: 4969, (cc) SEQ ID NO: 5023, or (dd) SEQ ID NO: 4934. 22. The VP capsid polypeptide of embodiment 21, wherein the 581-589 region has a sequence of: (a) SEQ ID NO: 348, (b) SEQ ID NO: 2536, (c) SEQ ID NO: 1576, (d) SEQ ID NO: 4955, (e) SEQ ID NO: 5017, (f) SEQ ID NO: 4349, (g) SEQ ID NO: 4964, (h) SEQ ID NO: 293, (i) SEQ ID NO: 4314, (j) SEQ ID NO: 4995, (k) SEQ ID NO: 4366, (l) SEQ ID NO: 4961, (m) SEQ ID NO: 4952, (n) SEQ ID NO: 5075, (o) SEQ ID NO: 4354, (p) SEQ ID NO: 5060, (q) SEQ ID NO: 5138, (r) SEQ ID NO: 5206, (s) SEQ ID NO: 4288, (t) SEQ ID NO: 5092, (u) SEQ ID NO: 4059, (v) SEQ ID NO: 4295, (w) SEQ ID NO: 5030, (x) SEQ ID NO: 4938, (y) SEQ ID NO: 2661, (z) SEQ ID NO: 5215, (aa) SEQ ID NO: 4960, (bb) SEQ ID NO: 4969, (cc) SEQ ID NO: 5023, or (dd) SEQ ID NO: 4934. 23. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a skeletal muscle tissue at a DNA enrichment of at least 15-fold greater than a wild type AAV9. 24. The VP capsid polypeptide of embodiment 23, wherein the 581-589 region has a sequence selected from the group consisting of SEQ ID NO: 3472, SEQ ID NO: 3297, SEQ ID NO: 2661, SEQ ID NO: 4545, SEQ ID NO: 348, SEQ ID NO: 18, SEQ ID NO: 4349, SEQ ID NO: 293, SEQ ID NO: 5017, SEQ ID NO: 4955, SEQ ID NO: 4366, SEQ ID NO: 5092, SEQ ID NO: 210, SEQ ID NO: 4059, SEQ ID NO: 2536, SEQ ID NO: 5206, and SEQ ID NO: 1971. 25. The VP capsid polypeptide of any one of embodiments 23-24, wherein the recombinant viral capsid is capable of preferentially targeting the skeletal muscle tissue at a DNA enrichment of at least 20-fold greater than the wild type AAV9. 26. The VP capsid polypeptide of embodiment 25, wherein the 581-589 region has a sequence selected from the group consisting of SEQ ID NO: 3472, SEQ ID NO: 3297, SEQ ID NO: 2661, SEQ ID NO: 4545, and SEQ ID NO: 348. 27. The VP capsid polypeptide of any one of embodiments 23-26, wherein the recombinant viral capsid is capable of preferentially targeting the skeletal muscle tissue at a DNA enrichment of at least 30-fold greater than the wild type AAV9. 28. The VP capsid polypeptide of embodiment 27, wherein the 581-589 region has a sequence of SEQ ID NO: 3472 or SEQ ID NO: 3297. 29. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a skeletal muscle tissue at an RNA enrichment of at least 2-fold greater than a wild type AAV9. 30. The VP capsid polypeptide of embodiment 29, wherein the 581-589 region has a sequence selected from the group consisting of SEQ ID NO: 5138, SEQ ID NO: 3472, SEQ ID NO: 1934, SEQ ID NO: 3283, SEQ ID NO: 5817, SEQ ID NO: 278, SEQ ID NO: 1634, SEQ ID NO: 1391, and SEQ ID NO: 1537. 31. The VP capsid polypeptide of any one of embodiments 29-30, wherein the recombinant viral capsid is capable of preferentially targeting the skeletal muscle tissue at an RNA enrichment of at least 10-fold or greater than the wild type AAV9. 32. The VP capsid polypeptide of embodiment 31, wherein the 581-589 region has a sequence selected from the group consisting of SEQ ID NO: 5138, SEQ ID NO: 3472, SEQ ID NO: 1934, SEQ ID NO: 3283, SEQ ID NO: 5817, and SEQ ID NO: 278. 33. The VP capsid polypeptide of any one of embodiments 29-30, wherein the recombinant viral capsid is capable of preferentially targeting the skeletal muscle tissue at an RNA enrichment of at least 25-fold or greater than the wild type AAV9. 34. The VP capsid polypeptide of embodiment 33, wherein the 581-589 region has a sequence selected from the group consisting of SEQ ID NO: 5138, SEQ ID NO: 3472, SEQ ID NO: 1934, or SEQ ID NO: 3283. 35. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a skeletal muscle tissue at a DNA enrichment of at least 4-fold greater than a wild type AAV5. 36. The VP capsid polypeptide of embodiment 35, wherein the 581-589 region has a sequence selected from the group consisting of SEQ ID NO: 3472, SEQ ID NO: 3297, SEQ ID NO: 2661, SEQ ID NO: 4545, SEQ ID NO: 348, SEQ ID NO: 18, SEQ ID NO: 4349, SEQ ID NO: 293, SEQ ID NO: 5017, SEQ ID NO: 4955, and SEQ ID NO: 4366. 37. The VP capsid polypeptide of any one of embodiments 35-36, wherein the recombinant viral capsid is capable of preferentially targeting the skeletal muscle tissue at a DNA enrichment of at least 5-fold greater than the wild type AAV5. 38. The VP capsid polypeptide of embodiment 37, wherein the 581-589 region has a sequence selected from the group consisting of SEQ ID NO: 3472, SEQ ID NO: 3297, SEQ ID NO: 2661, SEQ ID NO: 4545, and SEQ ID NO: 348. 39. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a skeletal muscle tissue at an RNA enrichment of at least that of a wild type AAV5. 40. The VP capsid polypeptide of embodiment 39, wherein the 581-589 region has a sequence selected from the group consisting of SEQ ID NO: 5138, SEQ ID NO: 3472, SEQ ID NO: 1934, SEQ ID NO: 3283, SEQ ID NO: 5817, and SEQ ID NO: 278. 41. The VP capsid polypeptide of any one of embodiments 39-40, wherein the recombinant viral capsid is capable of preferentially targeting the skeletal muscle tissue at an RNA enrichment of at least 3-fold greater than the wild type AAV5. 42. The VP capsid polypeptide of embodiment 41, wherein the 581-589 region has a sequence selected from the group consisting of SEQ ID NO: 5138, SEQ ID NO: 3472, SEQ ID NO: 1934, and SEQ ID NO: 3283. 43. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 3472, (b) SEQ ID NO: 3297, (c) SEQ ID NO: 2661, (d) SEQ ID NO: 4545, (e) SEQ ID NO: 348, (f) SEQ ID NO: 18, (g) SEQ ID NO: 4349, (h) SEQ ID NO: 293, (i) SEQ ID NO: 5017, (j) SEQ ID NO: 4955, (k) SEQ ID NO: 4366, (l) SEQ ID NO: 5092, (m) SEQ ID NO: 210, (n) SEQ ID NO: 4059, (o) SEQ ID NO: 2536, (p) SEQ ID NO: 5206, (q) SEQ ID NO: 1971, (r) SEQ ID NO: 262, (s) SEQ ID NO: 4343, (t) SEQ ID NO: 4995, (u) SEQ ID NO: 4009, (v) SEQ ID NO: 5190, (w) SEQ ID NO: 724, (x) SEQ ID NO: 4288, (y) SEQ ID NO: 4973, (z) SEQ ID NO: 4314, (aa) SEQ ID NO: 1561, (bb) SEQ ID NO: 5147, (cc) SEQ ID NO: 4238, or (dd) SEQ ID NO: 4961. 44. The VP capsid polypeptide of embodiment 43, wherein the 581-589 region has a sequence of: (a) SEQ ID NO: 3472, (b) SEQ ID NO: 3297, (c) SEQ ID NO: 2661, (d) SEQ ID NO: 4545, (e) SEQ ID NO: 348, (f) SEQ ID NO: 18, (g) SEQ ID NO: 4349, (h) SEQ ID NO: 293, (i) SEQ ID NO: 5017, (j) SEQ ID NO: 4955, (k) SEQ ID NO: 4366, (l) SEQ ID NO: 5092, (m) SEQ ID NO: 210, (n) SEQ ID NO: 4059, (o) SEQ ID NO: 2536, (p) SEQ ID NO: 5206, (q) SEQ ID NO: 1971, (r) SEQ ID NO: 262, (s) SEQ ID NO: 4343, (t) SEQ ID NO: 4995, (u) SEQ ID NO: 4009, (v) SEQ ID NO: 5190, (w) SEQ ID NO: 724, (x) SEQ ID NO: 4288, (y) SEQ ID NO: 4973, (z) SEQ ID NO: 4314, (aa) SEQ ID NO: 1561, (bb) SEQ ID NO: 5147, (cc) SEQ ID NO: 4238, or (dd) SEQ ID NO: 4961. 45. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a muscle tissue at a DNA enrichment of at least 20-fold greater than a wild type AAV9. 46. The VP capsid polypeptide of embodiment 45, wherein the 581-589 region has a sequence selected from the group consisting of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, SEQ ID NO: 4366, SEQ ID NO: 4545, SEQ ID NO: 4961, SEQ ID NO: 5206, SEQ ID NO: 5092, SEQ ID NO: 3472, SEQ ID NO: 5075, SEQ ID NO: 4059, SEQ ID NO: 4354, SEQ ID NO: 5060, SEQ ID NO: 4288, SEQ ID NO: 4952, SEQ ID NO: 5138, SEQ ID NO: 18, SEQ ID NO: 5030, SEQ ID NO: 3297, SEQ ID NO: 4295, SEQ ID NO: 4363, SEQ ID NO: 4963, SEQ ID NO: 4973, SEQ ID NO: 5159, SEQ ID NO: 4960, SEQ ID NO: 4938, SEQ ID NO: 5157, SEQ ID NO: 5023, and SEQ ID NO: 4969. 47. The VP capsid polypeptide of any one of embodiments 45-46, wherein the recombinant viral capsid is capable of preferentially targeting the muscle tissue at a DNA enrichment of at least 25-fold greater than the wild type AAV9. 48. The VP capsid polypeptide of embodiment 47, wherein the 581-589 region has a sequence selected from the group consisting of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, and SEQ ID NO: 4366. 49. The VP capsid polypeptide of any one of embodiments 46-48, wherein the recombinant viral capsid is capable of preferentially targeting the muscle tissue at a DNA enrichment of at least 30-fold greater than the wild type AAV9. 50. The VP capsid polypeptide of embodiment 49, wherein the 581-589 region has a sequence of SEQ ID NO: 348 or SEQ ID NO: 2536. 51. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a muscle tissue at an RNA enrichment of at least 5-fold greater than a wild type AAV9. 52. The VP capsid polypeptide of embodiment 51, wherein the 581-589 region has a sequence selected from the group consisting of SEQ ID NO: 5138, SEQ ID NO: 3472, SEQ ID NO: 348, SEQ ID NO: 5052, SEQ ID NO: 5193, SEQ ID NO: 5026, SEQ ID NO: 278, SEQ ID NO: 5163, SEQ ID NO: 293, SEQ ID NO: 4943, SEQ ID NO: 4952, SEQ ID NO: 4317, SEQ ID NO: 5075, SEQ ID NO: 386, SEQ ID NO: 4986, SEQ ID NO: 4995, SEQ ID NO: 4288, SEQ ID NO: 5030, SEQ ID NO: 5206, SEQ ID NO: 5159, SEQ ID NO: 4949, SEQ ID NO: 4338, SEQ ID NO: 5060, SEQ ID NO: 4346, SEQ ID NO: 5215, SEQ ID NO: 5017, SEQ ID NO: 4314, SEQ ID NO: 5080, SEQ ID NO: 4964, SEQ ID NO: 4363, SEQ ID NO: 4379, SEQ ID NO: 5027, SEQ ID NO: 4304, SEQ ID NO: 18, SEQ ID NO: 4349, SEQ ID NO: 4938, and SEQ ID NO: 2536. 53. The VP capsid polypeptide of any one of embodiments 51-52, wherein the recombinant viral capsid is capable of preferentially targeting the muscle tissue at an RNA enrichment of at least 10-fold or greater than the wild type AAV9. 54. The VP capsid polypeptide of embodiment 53, wherein the 581-589 region has a sequence selected from the group consisting of SEQ ID NO: 5138, SEQ ID NO: 3472, SEQ ID NO: 348, SEQ ID NO: 5052, SEQ ID NO: 5193, SEQ ID NO: 5026, SEQ ID NO: 278, and SEQ ID NO: 5163. 55. The VP capsid polypeptide of any one of embodiments 51-52, wherein the recombinant viral capsid is capable of preferentially targeting the muscle tissue at an RNA enrichment of at least 100-fold or greater than the wild type AAV9. 56. The VP capsid polypeptide of embodiment 55, wherein the 581-589 region has a sequence of SEQ ID NO: 5138. 57. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a muscle tissue at a DNA enrichment of at least 5-fold greater than a wild type AAV5. 58. The VP capsid polypeptide of embodiment 57, wherein the 581-589 region has a sequence selected from the group consisting of SEQ ID NO: 348, SEQ ID NO: 2536, SEQ ID NO: 4955, SEQ ID NO: 5017, SEQ ID NO: 4349, SEQ ID NO: 293, SEQ ID NO: 1576, SEQ ID NO: 2661, SEQ ID NO: 4964, SEQ ID NO: 4314, SEQ ID NO: 4995, SEQ ID NO: 4366, SEQ ID NO: 4545, SEQ ID NO: 4961, SEQ ID NO: 5206, SEQ ID NO: 5092, SEQ ID NO: 3472, SEQ ID NO: 5075, SEQ ID NO: 4059, SEQ ID NO: 4354, and SEQ ID NO: 5060. 59. The VP capsid polypeptide of any one of embodiments 57-58, wherein the recombinant viral capsid is capable of preferentially targeting the muscle tissue at a DNA enrichment of at least 10-fold greater than the wild type AAV5. 60. The VP capsid polypeptide of embodiment 59, wherein the 581-589 region has a sequence of SEQ ID NO: 348. 61. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting a muscle tissue at an RNA enrichment of at least that of a wild type AAV5. 62. The VP capsid polypeptide of embodiment 61, wherein the 581-589 region has a sequence selected from the group consisting of SEQ ID NO: 5138, SEQ ID NO: 3472, SEQ ID NO: 348, SEQ ID NO: 5052, SEQ ID NO: 5193, SEQ ID NO: 5026, SEQ ID NO: 278, SEQ ID NO: 5163, SEQ ID NO: 293, SEQ ID NO: 4943, SEQ ID NO: 4952, SEQ ID NO: 4317, SEQ ID NO: 5075, SEQ ID NO: 386, SEQ ID NO: 4986, and SEQ ID NO: 4995. 63. The VP capsid polypeptide of any one of embodiments 61-62, wherein the recombinant viral capsid is capable of preferentially targeting the muscle tissue at an RNA enrichment of at least 2-fold greater than the wild type AAV5. 64. The VP capsid polypeptide of embodiment 63, wherein the 581-589 region has a sequence selected from the group consisting of SEQ ID NO: 5138, SEQ ID NO: 3472, SEQ ID NO: 348, and SEQ ID NO: 5052. 65. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 348, (b) SEQ ID NO: 2536, (c) SEQ ID NO: 4955, (d) SEQ ID NO: 5017, (e) SEQ ID NO: 4349, (f) SEQ ID NO: 293, (g) SEQ ID NO: 1576, (h) SEQ ID NO: 2661, (i) SEQ ID NO: 4964, (j) SEQ ID NO: 4314, (k) SEQ ID NO: 4995, (l) SEQ ID NO: 4366, (m) SEQ ID NO: 4545, (n) SEQ ID NO: 4961, (o) SEQ ID NO: 5206, (p) SEQ ID NO: 5092, (q) SEQ ID NO: 3472, (r) SEQ ID NO: 5075, (s) SEQ ID NO: 4059, (t) SEQ ID NO: 4354, (u) SEQ ID NO: 5060, (v) SEQ ID NO: 4288, (w) SEQ ID NO: 4952, (x) SEQ ID NO: 5138, (y) SEQ ID NO: 18, (z) SEQ ID NO: 5030, (aa) SEQ ID NO: 3297, (bb) SEQ ID NO: 4295, (cc) SEQ ID NO: 4363, or (dd) SEQ ID NO: 4963. 66. The VP capsid polypeptide of embodiment 65, wherein the 581-589 region has a sequence of: (a) SEQ ID NO: 348, (b) SEQ ID NO: 2536, (c) SEQ ID NO: 4955, (d) SEQ ID NO: 5017, (e) SEQ ID NO: 4349, (f) SEQ ID NO: 293, (g) SEQ ID NO: 1576, (h) SEQ ID NO: 2661, (i) SEQ ID NO: 4964, (j) SEQ ID NO: 4314, (k) SEQ ID NO: 4995, (l) SEQ ID NO: 4366, (m) SEQ ID NO: 4545, (n) SEQ ID NO: 4961, (o) SEQ ID NO: 5206, (p) SEQ ID NO: 5092, (q) SEQ ID NO: 3472, (r) SEQ ID NO: 5075, (s) SEQ ID NO: 4059, (t) SEQ ID NO: 4354, (u) SEQ ID NO: 5060, (v) SEQ ID NO: 4288, (w) SEQ ID NO: 4952, (x) SEQ ID NO: 5138, (y) SEQ ID NO: 18, (z) SEQ ID NO: 5030, (aa) SEQ ID NO: 3297, (bb) SEQ ID NO: 4295, (cc) SEQ ID NO: 4363, or (dd) SEQ ID NO: 4963. 67. A viral protein (VP) capsid polypeptide, wherein the VP capsid polypeptide comprises a 581-589 region corresponding to residues 581 to 589 of a VP1 polypeptide, wherein the 581-589 region comprises a sequence having at least 85% identity to any one of any one of SEQ ID NO: 4238-SEQ ID NO: 4933 or SEQ ID NO: 4934-SEQ ID NO: 5233. 68. The VP capsid polypeptide of embodiment 67, wherein the 581-589 region has a sequence of any one of any one of SEQ ID NO: 4238-SEQ ID NO: 4933 or SEQ ID NO: 4934-SEQ ID NO: 5233. 69. The VP capsid polypeptide of any one of embodiments 1-68, wherein the VP capsid polypeptide is capable of assembling into a recombinant viral capsid. 70. A viral protein (VP) capsid polypeptide, wherein the VP capsid polypeptide is capable of assembling into a recombinant viral capsid, and wherein the VP capsid polypeptide comprises a 581-589 region corresponding to residues 581 to 589 of a VP1 polypeptide; wherein the 581-589 region: comprises at least one amino acid substitution as compared to residues 581 to 589 of SEQ ID NO: 1, and confers on the recombinant viral capsid an infection rate for muscle tissue with at least 3-fold higher muscle tissue tropism than a wild type VP capsid polypeptide of SEQ ID NO: 1; wherein the VP capsid polypeptide comprises at least one amino acid substitution as compared to each of SEQ ID NO: 3, SEQ ID NO: 4, SEQ ID NO: 5, SEQ ID NO: 6, SEQ ID NO: 7, and SEQ ID NO: 8. 71. The VP capsid polypeptide of embodiment 70, wherein the 581-589 region comprises a sequence of X¹X²X³X⁴X⁵X⁶X⁷X⁸X⁹, wherein X¹, X², X³, X⁴, X⁵, X⁶, X⁷, X⁸, and X⁹ are each individually selected from A, R, N, D, C, E, Q, G, H, I, L, K, M, F, P, S, T, W, Y, and V. 72. The VP capsid polypeptide of embodiment 70 or embodiment 83, wherein the VP capsid polypeptide has a sequence of SEQ ID NO: 2, wherein residues 581 to 589 of SEQ ID NO: 2 (X¹X²X³X⁴X⁵X⁶X⁷X⁸X⁹) correspond to the 581-589 region. 73. The VP capsid polypeptide of any one of embodiments 70-72, wherein the 581-589 region has a sequence that is at least 70%, at least 77%, at least 80%, at least 88%, at least 90%, or at least 95% identical to any one of SEQ ID NO: 4238-SEQ ID NO: 4933. 74. The VP capsid polypeptide of any one of embodiments 70-72, wherein the 581-589 region has a sequence of any one of SEQ ID NO: 4238-SEQ ID NO: 4933. 75. The VP capsid polypeptide of any one of embodiments 82-84, wherein the 581-589 region has a sequence that is at least 70%, at least 77%, at least 80%, at least 88%, at least 90%, or at least 95% identical to any one of SEQ ID NO: 4934-SEQ ID NO: 5233. 76. The VP capsid polypeptide of any one of embodiments 70-72, wherein the 581-589 region has a sequence of any one of SEQ ID NO: 4934-SEQ ID NO: 5233. 77. The VP capsid polypeptide of any one of embodiments 1-76, wherein the muscle tissue comprises cardiac muscle tissue. 78. The VP capsid polypeptide of any one of embodiments 1-77, wherein the muscle tissue comprises skeletal muscle tissue. 79. The VP capsid polypeptide of any one of embodiments 1-78, wherein the muscle tissue comprises skeletal muscle tissue and cardiac muscle tissue. 80. The VP capsid polypeptide of any one of embodiments 1-79, wherein the VP capsid polypeptide comprises an amino acid sequence at least 70%, at least 80%, at least 85%, at least 90%, at least 95%, at least 97%, at least 98%, at least 98.5%, at least 99%, or at least 99.5% identical to SEQ ID NO: 1. 81. The VP capsid polypeptide of any one of embodiments 1-80, wherein the infection rate for muscle tissue is at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold, or at least 1000-fold higher tissue tropism than an infection rate of the wild type VP capsid polypeptide for muscle tissue. 82. The VP capsid polypeptide of any one of embodiments 1-81, wherein the 581-589 region confers on a recombinant viral capsid assembled from the VP capsid polypeptide improved stability compared to a wild type AAV5 capsid comprising a peptide of SEQ ID NO: 1. 83. The VP capsid polypeptide of any one of embodiments 1-82, wherein the 581-589 region confers lower toxicity upon administration to a subject compared to a wild type VP capsid polypeptide of SEQ ID NO: 1. 84. The VP capsid polypeptide of any one of embodiments 1-83, wherein the muscle tissue is selected from aorta, esophagus, heart, skeletal muscle, and combinations thereof. 85. A pharmaceutical composition comprising the VP capsid polypeptide of any one of embodiments 1-84. 86. The pharmaceutical composition of embodiment 85, wherein the VP capsid polypeptide is assembled into a recombinant viral capsid. 87. The pharmaceutical composition of embodiment 85 or embodiment 86, further comprising a payload encapsidated by the recombinant viral capsid. 88. The pharmaceutical composition of embodiment 87, wherein the payload encodes a therapeutic polynucleotide or a therapeutic peptide. 89. The pharmaceutical composition of embodiment 87, wherein the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof 90. The pharmaceutical composition of embodiment 87, wherein the payload encodes a component of a CRISPR/Cas system, an adenosine deaminase acting on RNA (ADAR) enzyme, a transcriptional activator, or a transcriptional repressor. 91. A recombinant adeno-associated virus (rAAV), comprising the VP capsid polypeptide of any one of embodiments 1-84 assembled into a recombinant viral capsid and a payload encapsidated by the recombinant viral capsid. 92. The rAAV of embodiment 91, further comprising a VP2 polypeptide comprising the 581-589 region and a VP3 polypeptide comprising the 581-589 region. 93. The rAAV of embodiment 91 or embodiment 92, wherein the payload encodes a therapeutic polynucleotide or a therapeutic peptide. 94. The rAAV of embodiment 93, wherein the payload encodes a guide RNA, a tRNA, a suppressor tRNA, a siRNA, a miRNA, an mRNA, a shRNA, a circular RNA, or an antisense oligonucleotide (ASO), a ribozyme, a DNAzyme, an aptamer, or any combination thereof 95. The rAAV of embodiment 93, wherein the payload encodes a component of a CRISPR/Cas system, an adenosine deaminase acting on RNA (ADAR) enzyme, a transcriptional activator, or a transcriptional repressor. 96. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a cardiac muscle tissue; and preferentially delivering the payload to the muscle tissue infected by the rAAV at a DNA enrichment of at least 25-fold greater than a wild type AAV9. 97. The method of embodiment 96, wherein the DNA enrichment is at least 30-fold greater than the wild type AAV9. 98. The method of embodiment 96 or embodiment 97, wherein the DNA enrichment is at least 50-fold greater than the wild type AAV9. 99. A method of transcribing a payload in a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a cardiac muscle tissue; and preferentially transcribing the payload to the muscle tissue infected by the rAAV at an RNA enrichment of at least 10-fold greater than a wild type AAV9. 100. The method of embodiment 99, wherein the RNA enrichment is at least 20-fold greater than the wild type AAV9. 101. The method of embodiment 99 or embodiment 100, wherein the RNA enrichment is at least 40-fold greater than the wild type AAV9. 102. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a cardiac muscle tissue; and preferentially delivering the payload to the muscle tissue infected by the rAAV at a DNA enrichment of at least 5-fold greater than a wild type AAV5. 103. The method of embodiment 102, wherein the DNA enrichment is at least 10-fold greater than the wild type AAV5. 104. A method of transcribing a payload in a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a cardiac muscle tissue; and preferentially transcribing the payload to the muscle tissue infected by the rAAV at an RNA enrichment of at least 5-fold greater than a wild type AAV5. 105. The method of embodiment 104, wherein the RNA enrichment is at least 10-fold greater than the wild type AAV5. 106. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 348, (b) SEQ ID NO: 2536, (c) SEQ ID NO: 1576, (d) SEQ ID NO: 4955, (e) SEQ ID NO: 5017, (f) SEQ ID NO: 4349, (g) SEQ ID NO: 4964, (h) SEQ ID NO: 293, (i) SEQ ID NO: 4314, (j) SEQ ID NO: 4995, (k) SEQ ID NO: 4366, (l) SEQ ID NO: 4961, (m) SEQ ID NO: 4952, (n) SEQ ID NO: 5075, (o) SEQ ID NO: 4354, (p) SEQ ID NO: 5060, (q) SEQ ID NO: 5138, (r) SEQ ID NO: 5206, (s) SEQ ID NO: 4288, (t) SEQ ID NO: 5092, (u) SEQ ID NO: 4059, (v) SEQ ID NO: 4295, (w) SEQ ID NO: 5030, (x) SEQ ID NO: 4938, (y) SEQ ID NO: 2661, (z) SEQ ID NO: 5215, (aa) SEQ ID NO: 4960, (bb) SEQ ID NO: 4969, (cc) SEQ ID NO: 5023, or (dd) SEQ ID NO: 4934; infecting the muscle tissue with the rAAV, wherein the muscle tissue comprises cardiac muscle tissue; and delivering the payload to the muscle tissue infected by the rAAV. 107. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; and infecting the muscle tissue with the rAAV, wherein the muscle tissue comprises a skeletal muscle tissue; preferentially delivering the payload to the muscle tissue infected by the rAAV at a DNA enrichment of at least 15-fold greater than a wild type AAV9. 108. The method of embodiment 107, wherein the DNA enrichment is at least 20-fold greater than the wild type AAV9. 109. The method of embodiment 107 or embodiment 108, wherein the DNA enrichment is at least 30-fold greater than the wild type AAV9. 110. A method of transcribing a payload in a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a skeletal muscle tissue; and preferentially transcribing the payload to the muscle tissue infected by the rAAV at an RNA enrichment of at least 2-fold greater than a wild type AAV9. 111. The method of embodiment 110, wherein the RNA enrichment is at least 10-fold greater than the wild type AAV9. 112. The method of embodiment 110 or embodiment 111, wherein the RNA enrichment is at least 25-fold greater than the wild type AAV9. 113. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a skeletal muscle tissue; and preferentially delivering the payload to the muscle tissue infected by the rAAV at a DNA enrichment of at least 4-fold greater than a wild type AAV5. 114. The method of embodiment 113, wherein the DNA enrichment is at least 5-fold greater than the wild type AAV5. 115. A method of transcribing a payload in a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV, wherein the muscle tissue comprises a skeletal muscle tissue; and preferentially transcribing the payload to the muscle tissue infected by the rAAV at an RNA enrichment of at that of a wild type AAV5. 116. The method of embodiment 115, wherein the RNA enrichment is at least 3-fold greater than the wild type AAV5. 117. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 3472, (b) SEQ ID NO: 3297, (c) SEQ ID NO: 2661, (d) SEQ ID NO: 4545, (e) SEQ ID NO: 348, (f) SEQ ID NO: 18, (g) SEQ ID NO: 4349, (h) SEQ ID NO: 293, (i) SEQ ID NO: 5017, (j) SEQ ID NO: 4955, (k) SEQ ID NO: 4366, (l) SEQ ID NO: 5092, (m) SEQ ID NO: 210, (n) SEQ ID NO: 4059, (o) SEQ ID NO: 2536, (p) SEQ ID NO: 5206, (q) SEQ ID NO: 1971, (r) SEQ ID NO: 262, (s) SEQ ID NO: 4343, or (t) SEQ ID NO: 4995, (u) SEQ ID NO: 4009, (v) SEQ ID NO: 5190, (w) SEQ ID NO: 724, (x) SEQ ID NO: 4288, (y) SEQ ID NO: 4973, (z) SEQ ID NO: 4314, (aa) SEQ ID NO: 1561, (bb) SEQ ID NO: 5147, (cc) SEQ ID NO: 4238, or (dd) SEQ ID NO: 4961; infecting the muscle tissue with the rAAV, wherein the muscle tissue comprises skeletal muscle tissue; and delivering the payload to the muscle tissue infected by the rAAV. 118. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV; and preferentially delivering the payload to the muscle tissue infected by the rAAV at a DNA enrichment of at least 20-fold greater than a wild type AAV9. 119. The method of embodiment 118, wherein the DNA enrichment is at least 25-fold greater than the wild type AAV9. 120. The method of embodiment 118 or embodiment 119, wherein the DNA enrichment is at least 30-fold greater than the wild type AAV9. 121. A method of transcribing a payload in a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV; and preferentially transcribing the payload to the muscle tissue infected by the rAAV at an RNA enrichment of at least 5-fold greater than a wild type AAV9. 122. The method of embodiment 121, wherein the RNA enrichment is at least 10-fold greater than the wild type AAV9. 123. The method of embodiment 121 or embodiment 122, wherein the RNA enrichment is at least 100-fold greater than the wild type AAV9. 124. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV; and preferentially delivering the payload to the muscle tissue infected by the rAAV at a DNA enrichment of at least 5-fold greater than a wild type AAV5. 125. The method of embodiment 124, wherein the DNA enrichment is at least 10-fold greater than the wild type AAV5. 126. A method of transcribing a payload in a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV; and preferentially transcribing the payload to the muscle tissue infected by the rAAV at an RNA enrichment of at least that of a wild type AAV5. 127. The method of embodiment 126, wherein the RNA enrichment is at least 2-fold greater than the wild type AAV5. 128. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 348, (b) SEQ ID NO: 2536, (c) SEQ ID NO: 4955, (d) SEQ ID NO: 5017, (e) SEQ ID NO: 4349, (f) SEQ ID NO: 293, (g) SEQ ID NO: 1576, (h) SEQ ID NO: 2661, (i) SEQ ID NO: 4964, (j) SEQ ID NO: 4314, (k) SEQ ID NO: 4995, (l) SEQ ID NO: 4366, (m) SEQ ID NO: 4545, (n) SEQ ID NO: 4961, (o) SEQ ID NO: 5206, (p) SEQ ID NO: 5092, (q) SEQ ID NO: 3472, (r) SEQ ID NO: 5075, (s) SEQ ID NO: 4059, (t) SEQ ID NO: 4354, (u) SEQ ID NO: 5060, (v) SEQ ID NO: 4288, (w) SEQ ID NO: 4952, (x) SEQ ID NO: 5138, (y) SEQ ID NO: 18, (z) SEQ ID NO: 5030, (aa) SEQ ID NO: 3297, (bb) SEQ ID NO: 4295, (cc) SEQ ID NO: 4363, or (dd) SEQ ID NO: 4963; infecting the muscle tissue with the rAAV; and delivering the payload to the muscle tissue infected by the rAAV. 129. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to residues 581 to 589 of SEQ ID NO: 1, and wherein the VP capsid polypeptide comprises at least one amino acid substitution as compared to each of SEQ ID NO: 3, SEQ ID NO: 4, SEQ ID NO: 5, SEQ ID NO: 6, SEQ ID NO: 7, and SEQ ID NO: 8; infecting the muscle tissue with the rAAV with at least 3-fold higher muscle tissue tropism as compared to a wild type AAV5 capsid comprising a peptide of SEQ ID NO: 1; and delivering the payload to the muscle tissue infected by the rAAV. 130. A method of delivering a payload to a muscle tissue of a subject, the method comprising: administering a recombinant adeno-associated virus (rAAV) to the subject, wherein the rAAV encapsidates the payload, and wherein the rAAV comprises a VP capsid polypeptide of any one of embodiments 1-84; infecting the muscle tissue with the rAAV; and delivering the payload to the muscle tissue infected by the rAAV. 131. The method of any one of embodiments 128-130, wherein the muscle tissue comprises aorta, esophagus, heart, skeletal muscle, or a combination thereof 132. The method of any one of embodiments 96-131, further comprising expressing a therapeutic polypeptide or a therapeutic polynucleotide encoded by the payload in the muscle tissue infected by the rAAV. 133. The method of any one of embodiments 96-132, further comprising producing a therapeutic effect in the muscle tissue of the subject. 134. The method of embodiment 133, wherein the therapeutic effect is produced upon administration of from 1×10⁵ to 5×10¹⁴ rAAVs per kg subject weight. 135. The method of embodiment 133 or embodiment 134, comprising administering an amount of the rAAV sufficient to produce the therapeutic effect without producing a toxicity in the subject. 136. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a cardiac muscle tissue; preferentially delivering the payload to the muscle tissue infected by the rAAV at a DNA enrichment of at least 25-fold greater than a wild type AAV9; and producing a therapeutic effect in the muscle tissue, thereby treating the condition. 137. The method of embodiment 136, wherein the DNA enrichment is at least 30-fold greater than the wild type AAV9. 138. The method of embodiment 136 or embodiment 137, wherein the DNA enrichment is at least 50-fold greater than the wild type AAV9. 139. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a cardiac muscle tissue; preferentially transcribing the payload to the muscle tissue infected by the rAAV at an RNA enrichment of at least 10-fold greater than a wild type AAV9; and producing a therapeutic effect in the muscle tissue, thereby treating the condition. 140. The method of embodiment 139, wherein the RNA enrichment is at least 20-fold greater than the wild type AAV9. 141. The method of embodiment 139 or embodiment 140, wherein the RNA enrichment is at least 40-fold greater than the wild type AAV9. 142. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a cardiac muscle tissue; preferentially delivering the payload to the muscle tissue infected by the rAAV at a DNA enrichment of at least 5-fold greater than a wild type AAV5; and producing a therapeutic effect in the muscle tissue, thereby treating the condition. 143. The method of embodiment 142, wherein the DNA enrichment is at least 10-fold greater than the wild type AAV5. 144. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a cardiac muscle tissue; preferentially transcribing the payload to the muscle tissue infected by the rAAV at an RNA enrichment of at least 5-fold greater than a wild type AAV5; and producing a therapeutic effect in the muscle tissue, thereby treating the condition. 145. The method of embodiment 144, wherein the RNA enrichment is at least 10-fold greater than the wild type AAV5. 146. A method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 348, (b) SEQ ID NO: 2536, (c) SEQ ID NO: 1576, (d) SEQ ID NO: 4955, (e) SEQ ID NO: 5017, (f) SEQ ID NO: 4349, (g) SEQ ID NO: 4964, (h) SEQ ID NO: 293, (i) SEQ ID NO: 4314, (j) SEQ ID NO: 4995, (k) SEQ ID NO: 4366, (l) SEQ ID NO: 4961, (m) SEQ ID NO: 4952, (n) SEQ ID NO: 5075, (o) SEQ ID NO: 4354, (p) SEQ ID NO: 5060, (q) SEQ ID NO: 5138, (r) SEQ ID NO: 5206, (s) SEQ ID NO: 4288, (t) SEQ ID NO: 5092, (u) SEQ ID NO: 4059, (v) SEQ ID NO: 4295, (w) SEQ ID NO: 5030, (x) SEQ ID NO: 4938, (y) SEQ ID NO: 2661, (z) SEQ ID NO: 5215, (aa) SEQ ID NO: 4960, (bb) SEQ ID NO: 4969, (cc) SEQ ID NO: 5023, or (dd) SEQ ID NO: 4934; infecting a muscle tissue of the subject with the rAAV, wherein the muscle tissue comprises cardiac muscle tissue; delivering the payload to the muscle tissue infected by the rAAV; and producing a therapeutic effect in the muscle tissue, thereby treating the condition. 147. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a skeletal muscle tissue; preferentially delivering the payload to the muscle tissue infected by the rAAV at a DNA enrichment of at least 15-fold greater than a wild type AAV9; and producing a therapeutic effect in the muscle tissue, thereby treating the condition. 148. The method of embodiment 147, wherein the DNA enrichment is at least 20-fold greater than the wild type AAV9. 149. The method of embodiment 147 or embodiment 148, wherein the DNA enrichment is at least 30-fold greater than the wild type AAV9. 150. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a skeletal muscle tissue; preferentially transcribing the payload to the muscle tissue infected by the rAAV at an RNA enrichment of at least 2-fold greater than a wild type AAV9. 151. The method of embodiment 150, wherein the RNA enrichment is at least 10-fold greater than the wild type AAV9. 152. The method of embodiment 150 or embodiment 151, wherein the RNA enrichment is at least 25-fold greater than the wild type AAV9. 153. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a skeletal muscle tissue; preferentially delivering the payload to the muscle tissue infected by the rAAV at a DNA enrichment of at least 4-fold greater than a wild type AAV5; and producing a therapeutic effect in the muscle tissue, thereby treating the condition. 154. The method of embodiment 153, wherein the DNA enrichment is at least 5-fold greater than the wild type AAV5. 155. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV, wherein the muscle tissue comprises a skeletal muscle tissue; preferentially transcribing the payload to the muscle tissue infected by the rAAV at an RNA enrichment of at that of a wild type AAV5; and producing a therapeutic effect in the muscle tissue, thereby treating the condition. 156. The method of embodiment 155, wherein the RNA enrichment is at least 3-fold greater than the wild type AAV5. 157. A method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 3472, (b) SEQ ID NO: 3297, (c) SEQ ID NO: 2661, (d) SEQ ID NO: 4545, (e) SEQ ID NO: 348, (f) SEQ ID NO: 18, (g) SEQ ID NO: 4349, (h) SEQ ID NO: 293, (i) SEQ ID NO: 5017, (j) SEQ ID NO: 4955, (k) SEQ ID NO: 4366, (l) SEQ ID NO: 5092, (m) SEQ ID NO: 210, (n) SEQ ID NO: 4059, (o) SEQ ID NO: 2536, (p) SEQ ID NO: 5206, (q) SEQ ID NO: 1971, (r) SEQ ID NO: 262, (s) SEQ ID NO: 4343, (t) SEQ ID NO: 4995, (u) SEQ ID NO: 4009, (v) SEQ ID NO: 5190, (w) SEQ ID NO: 724, (x) SEQ ID NO: 4288, (y) SEQ ID NO: 4973, (z) SEQ ID NO: 4314, (aa) SEQ ID NO: 1561, (bb) SEQ ID NO: 5147, (cc) SEQ ID NO: 4238, or (dd) SEQ ID NO: 4961; infecting a muscle tissue of the subject with the rAAV, wherein the muscle tissue comprises skeletal muscle tissue; delivering the payload to the muscle tissue infected by the rAAV; and producing a therapeutic effect in the muscle tissue, thereby treating the condition. 158. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV; preferentially delivering the payload to the muscle tissue infected by the rAAV at a DNA enrichment of at least 20-fold greater than a wild type AAV9; and producing a therapeutic effect in the muscle tissue, thereby treating the condition. 159. The method of embodiment 158, wherein the DNA enrichment is at least 25-fold greater than the wild type AAV9. 160. The method of embodiment 158 or embodiment 159, wherein the DNA enrichment is at least 30-fold greater than the wild type AAV9. 161. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV; preferentially transcribing the payload to the muscle tissue infected by the rAAV at an RNA enrichment of at least 5-fold greater than a wild type AAV9; and producing a therapeutic effect in the muscle tissue, thereby treating the condition. 162. The method of embodiment 161, wherein the RNA enrichment is at least 10-fold greater than the wild type AAV9. 163. The method of embodiment 161 or embodiment 162, wherein the RNA enrichment is at least 100-fold greater than the wild type AAV9. 164. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting a muscle tissue of the subject with the rAAV; preferentially delivering the payload to the muscle tissue infected by the rAAV at a DNA enrichment of at least 5-fold greater than a wild type AAV5; and producing a therapeutic effect in the muscle tissue, thereby treating the condition. 165. The method of embodiment 164, wherein the DNA enrichment is at least 10-fold greater than the wild type AAV5. 166. A method of treating a condition in a subject, the method comprising: administering a recombinant adeno-associated virus (rAAV) to the subject, wherein the rAAV encapsidates the payload, and wherein the rAAV comprises a VP capsid polypeptide comprising a 581-589 region corresponding to residues 581 to 589 of a VP1 polypeptide, wherein the 581-589 region comprises at least one amino acid substitution as compared to SEQ ID NO: 9; infecting the muscle tissue with the rAAV; preferentially transcribing the payload to the muscle tissue infected by the rAAV at an RNA enrichment of at least that of a wild type AAV5; and producing a therapeutic effect in the muscle tissue, thereby treating the condition. 167. The method of embodiment 166, wherein the RNA enrichment is at least 2-fold greater than the wild type AAV5. 168. A method of treating a condition in a subject, the method comprising: administering a recombinant adeno-associated virus (rAAV) to the subject, wherein the rAAV encapsidates a payload, and wherein the rAAV comprises a VP capsid polypeptide comprising a 581-589 region corresponding to residues 581 to 589 of a VP1 polypeptide, wherein the 581-589 region has a sequence that is at least 85% identical to: (a) SEQ ID NO: 348, (b) SEQ ID NO: 2536, (c) SEQ ID NO: 4955, (d) SEQ ID NO: 5017, (e) SEQ ID NO: 4349, (f) SEQ ID NO: 293, (g) SEQ ID NO: 1576, (h) SEQ ID NO: 2661, (i) SEQ ID NO: 4964, (j) SEQ ID NO: 4314, (k) SEQ ID NO: 4995, (l) SEQ ID NO: 4366, (m) SEQ ID NO: 4545, (n) SEQ ID NO: 4961, (o) SEQ ID NO: 5206, (p) SEQ ID NO: 5092, (q) SEQ ID NO: 3472, (r) SEQ ID NO: 5075, (s) SEQ ID NO: 4059, (t) SEQ ID NO: 4354, (u) SEQ ID NO: 5060, (v) SEQ ID NO: 4288, (w) SEQ ID NO: 4952, (x) SEQ ID NO: 5138, (y) SEQ ID NO: 18, (z) SEQ ID NO: 5030, (aa) SEQ ID NO: 3297, (bb) SEQ ID NO: 4295, (cc) SEQ ID NO: 4363, or (dd) SEQ ID NO: 4963; infecting a muscle tissue of the subject with the rAAV; delivering the payload to the muscle tissue infected by the rAAV; and producing a therapeutic effect in the muscle tissue, thereby treating the condition. 169. A method of treating a condition in a subject, the method comprising: administering a recombinant adeno-associated virus (rAAV) to the subject, wherein the rAAV encapsidates a payload, and wherein the rAAV comprises a VP capsid polypeptide a 581-589 region corresponding to residues 581 to 589 of a VP1 polypeptide, wherein the 581-589 region comprises at least one amino acid substitution as compared to residues 581 to 589 of SEQ ID NO: 1, and wherein the VP capsid polypeptide does not have the sequence of any of SEQ ID NO: 3, SEQ ID NO: 4, SEQ ID NO: 5, SEQ ID NO: 6, SEQ ID NO: 7, or SEQ ID NO: 8; infecting a muscle tissue of the subject with the rAAV with at least 3-fold higher muscle tissue tropism as compared to a wild type AAV5 capsid comprising a peptide of SEQ ID NO: 1; delivering the payload to the muscle tissue infected by the rAAV; and producing a therapeutic effect in the muscle tissue, thereby treating the condition. 170. A method of treating a condition in a subject, the method comprising: administering a recombinant adeno-associated virus (rAAV) to the subject, wherein the rAAV encapsidates a payload, and wherein the rAAV comprises a VP capsid polypeptide of any one of embodiments 1-84; infecting a muscle tissue of the subject with the rAAV; delivering the payload to the muscle tissue infected by the rAAV; and producing a therapeutic effect in the muscle tissue, thereby treating the condition. 171. The method of any one of embodiments 136-170, wherein the condition is a muscle condition. 172. The method of embodiment 171, wherein the muscle condition is a vascular condition, a cardiac condition, a skeletal muscle condition, Charcot-Marie-Tooth disease, Duchenne muscular dystrophy, facioscapulohumeral muscular dystrophy, dysferlinopathy, Pompe disease, limb-girdle muscular dystrophy, myotonic dystrophy, a glycogen storage disorder, X-linked myotubular myopathy, or euchromatic histone-lysine N-methyltransferase 2. 173. The method of any one of embodiments 136-172, wherein the condition is facioscapulohumeral muscular dystrophy syndrome. 174. The method of embodiment 173, wherein the payload encodes DUX4 or a guide RNA or miRNA that targets an mRNA encoding DUX4. 175. The method of any one of embodiments 136-172, wherein the condition is Duchenne muscular dystrophy. 176. The method of embodiment 175, wherein the payload encodes dystrophin or a guide RNA or miRNA that targets an mRNA encoding dystrophin. 177. The method of any one of embodiments 136-176, comprising administering from 1×10⁵ to 5×10¹⁴ rAAVs per kg subject weight. 178. The method of any one of embodiments 96-177, comprising infecting the muscle tissue with at least 2.5-fold, at least 3-fold, at least 3.5-fold, at least 4-fold, at least 5-fold, at least 10-fold, at least 20-fold, at least 50-fold, at least 75-fold, at least 100-fold, at least 200-fold, at least 500-fold, or at least 1000-fold higher tissue tropism than a wild type AAV5 capsid comprising a peptide of SEQ ID NO: 1. 179. The method of any one of embodiments 96-178, wherein the payload encodes a therapeutic protein, therapeutic polynucleotide, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof 180. The method of embodiment 179, wherein the guide RNA is a CRISPR/Cas guide RNA or an ADAR guide RNA. 181. The method of embodiment 179, wherein the therapeutic protein is selected from the group consisting of neurotrophin-3, micro-dystrophin, mini-dystrophin, double homeobox 4 (DUX4), dysferlin, α-glucosidase, FKRP, dystrophin, myotonin-protein kinase, glycogen synthase, myotubularin, and EHMT2. 182. The method embodiment 179, wherein the therapeutic polynucleotide targets an mRNA encoding a protein selected from the group consisting of neurotrophin-3, micro-dystrophin, mini-dystrophin, double homeobox 4 (DUX4), dysferlin, α-glucosidase, FKRP, dystrophin, myotonin-protein kinase, glycogen synthase, myotubularin, and EHMT2. 183. The method of any one of embodiments 96-182, wherein the payload encodes a component of a CRISPR/Cas system, an adenosine deaminase acting on RNA (ADAR) enzyme, a transcriptional activator, or a transcriptional repressor. 184. The method of embodiment 183, wherein the component of the CRISPR/Cas system comprises a Cas3, a Cas8, a Cas10, a Cas9, a Cas4, a Cas12, a Cas13, a guide RNA, or a combination thereof. 185. The method of embodiment 183, wherein the ADAR enzyme is ADAR1 or ADAR2. 186. The method of embodiment 183, wherein the transcriptional activator is VP64. 187. The method of embodiment 183, wherein the transcriptional repressor is KRAB. 188. The method of any one of embodiments 96-187, comprising systemically administering the rAAV to the subject. 189. The method of any one of embodiments 96-188, comprising administering the rAAV via intravenous administration, intramuscular administration, intraperitoneal administration, or oral administration. 190. The method of any one of embodiments 96-189, further comprising expressing a therapeutic polypeptide or a therapeutic polynucleotide encoded by the payload in the muscle tissue infected by the rAAV. 191. The method of embodiment 190, comprising expressing the therapeutic polypeptide or the therapeutic polynucleotide with higher muscle tissue tropism compared to a wild type AAV5 capsid comprising a peptide of SEQ ID NO: 1. 192. The method of embodiment 191, comprising expressing the therapeutic polypeptide or the therapeutic polynucleotide with at least 3-fold, at least 3.5-fold, at least 4-fold, at least 5-fold, at least 10-fold, at least 20-fold, at least 50-fold, at least 75-fold, at least 100-fold, at least 200-fold, at least 500-fold higher, or at least 1000-fold higher tropism compared to the wild type AAV5 capsid. 193. The method of any one of embodiments 96-192, comprising producing a toxicity in the subject that is lower than a toxicity produced upon systemic administration of a comparable number of wild type AAV5 capsids comprising a VP capsid polypeptide of SEQ ID NO: 1. 194. The method of any one of embodiments 96-193, comprising producing a toxicity in the subject that is lower than a toxicity produced upon systemic administration of a number of wild type AAV5 capsids comprising a VP capsid polypeptide of SEQ ID NO: 1 sufficient to deliver a comparable number of payloads to the muscle tissue. 195. The method of any one of embodiments 96-194, comprising producing a concentration of neutralizing antibodies in the subject that is lower than a concentration of neutralizing antibodies produced upon systemic administration of a comparable number of wild type AAV5 capsids comprising a VP capsid polypeptide of SEQ ID NO: 1. 196. The method of any one of embodiments 96-195, comprising producing a concentration of neutralizing antibodies in the subject that is lower than a concentration of neutralizing antibodies produced upon intravitreal administration of a comparable number of wild type AAV2 capsids. 197. The method of any one of embodiments 96-196, comprising producing a concentration of neutralizing antibodies in the subject that is lower than a concentration of neutralizing antibodies produced upon intravitreal administration of a comparable number of wild type AAV8 capsids. 198. The method of any one of embodiments 96-197, comprising producing a concentration of neutralizing antibodies in the subject that is lower than a concentration of neutralizing antibodies produced upon intravitreal administration of a comparable number of wild type AAV9 capsids. 199. The method of any one of embodiments 96-198, wherein the VP capsid polypeptide further comprises one or more mutations outside of the 581-589 region that contributes to reduced production of neutralizing antibodies relative to a wild type AAV capsid. 200. The method of any one of embodiments 96-199, comprising producing a concentration of neutralizing antibodies in the subject that is lower than a concentration of neutralizing antibodies produced upon systemic administration of a number of wild type AAV5 capsids comprising a VP capsid polypeptide of SEQ ID NO: 1 sufficient to deliver a comparable number of payloads to the muscle tissue. 201. A viral protein (VP) capsid polypeptide, wherein the VP capsid polypeptide comprises a 581-589 region corresponding to residues 581 to 589 a VP1 polypeptide, wherein the 581-589 region comprises at least one amino acid substitution as compared to SEQ ID NO: 9, and wherein a recombinant viral capsid comprising the VP capsid polypeptide is capable of preferentially targeting cardiac muscle tissue and a skeletal muscle tissue. 202. The VP capsid polypeptide of embodiment 201, wherein the 581-589 region has a sequence having at least 85% sequence identity to a sequence selected from the group consisting of SEQ ID NO: 348, SEQ ID NO: 2536, SEQ ID NO: 5607, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5283, SEQ ID NO: 5092, SEQ ID NO: 4059, SEQ ID NO: 4295, SEQ ID NO: 5030, SEQ ID NO: 2661, SEQ ID NO: 4545, SEQ ID NO: 4963, SEQ ID NO: 4363, SEQ ID NO: 5159, SEQ ID NO: 4973, SEQ ID NO: 5157, SEQ ID NO: 4345, SEQ ID NO: 5039, SEQ ID NO: 4304, SEQ ID NO: 18, SEQ ID NO: 386, SEQ ID NO: 308, SEQ ID NO: 278, SEQ ID NO: 5037, SEQ ID NO: 4361, SEQ ID NO: 4317, SEQ ID NO: 4343, SEQ ID NO: 4311, SEQ ID NO: 5055, SEQ ID NO: 4359, SEQ ID NO: 4353, and SEQ ID NO: 4346. 203. The VP capsid polypeptide of embodiment 201 or embodiment 202, wherein the 581-589 region has a sequence selected from the group consisting of SEQ ID NO: 348, SEQ ID NO: 2536, SEQ ID NO: 5607, SEQ ID NO: 4955, SEQ ID NO: 5017, SEQ ID NO: 4349, SEQ ID NO: 4964, SEQ ID NO: 293, SEQ ID NO: 4314, SEQ ID NO: 4995, SEQ ID NO: 4366, SEQ ID NO: 4961, SEQ ID NO: 4952, SEQ ID NO: 5075, SEQ ID NO: 4354, SEQ ID NO: 5060, SEQ ID NO: 5138, SEQ ID NO: 5206, SEQ ID NO: 4288, SEQ ID NO: 5283, SEQ ID NO: 5092, SEQ ID NO: 4059, SEQ ID NO: 4295, SEQ ID NO: 5030, SEQ ID NO: 2661, SEQ ID NO: 4545, SEQ ID NO: 4963, SEQ ID NO: 4363, SEQ ID NO: 5159, SEQ ID NO: 4973, SEQ ID NO: 5157, SEQ ID NO: 4345, SEQ ID NO: 5039, SEQ ID NO: 4304, SEQ ID NO: 18, SEQ ID NO: 386, SEQ ID NO: 308, SEQ ID NO: 278, SEQ ID NO: 5037, SEQ ID NO: 4361, SEQ ID NO: 4317, SEQ ID NO: 4343, SEQ ID NO: 4311, SEQ ID NO: 5055, SEQ ID NO: 4359, SEQ ID NO: 4353, and SEQ ID NO: 4346. 204. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to: (a) SEQ ID NO: 5027, (b) SEQ ID NO: 4633, (c) SEQ ID NO: 4943, (d) SEQ ID NO: 386, (e) SEQ ID NO: 334, (f) SEQ ID NO: 4309, (g) SEQ ID NO: 4288, (h) SEQ ID NO: 4964, (i) SEQ ID NO: 5017, (j) SEQ ID NO: 5814, (k) SEQ ID NO: 4335, (l) SEQ ID NO: 348, (m) SEQ ID NO: 4936, (n) SEQ ID NO: 5206, (o) SEQ ID NO: 4238, (p) SEQ ID NO: 5077, or (q) SEQ ID NO: 5603. 205. The VP capsid polypeptide of embodiment 204, wherein the 581-589 region has a sequence of: (a) SEQ ID NO: 5027, (b) SEQ ID NO: 4633, (c) SEQ ID NO: 4943, (d) SEQ ID NO: 386, (e) SEQ ID NO: 334, (f) SEQ ID NO: 4309, (g) SEQ ID NO: 4288, (h) SEQ ID NO: 4964, (i) SEQ ID NO: 5017, (j) SEQ ID NO: 5814, (k) SEQ ID NO: 4335, (l) SEQ ID NO: 348, (m) SEQ ID NO: 4936, (n) SEQ ID NO: 5206, (o) SEQ ID NO: 4238, (p) SEQ ID NO: 5077, or (q) SEQ ID NO: 5603. 206. The VP capsid polypeptide of embodiment 204 or embodiment 205, wherein the 581-589 region confers cardiac tissue tropism in mice and non-human primates. 207. A research method comprising administering a recombinant adeno-associated virus (rAAV) to a model organism, wherein the rAAV comprises a VP capsid polypeptide of any one of embodiments 204-206. 208. The research method of embodiment 207, further comprising evaluating an effect of the rAAV in the model organism. 209. The research method of embodiment 208, wherein the model organism is a mouse. 210. The research method of embodiment 208 or embodiment 209, further comprising inferring an effect of the rAAV in an organism of interest based on the effect of the rAAV in the model organism. 211. The research method of embodiment 210, wherein the organism of interest is a non-human primate or a human. 212. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 18. 213. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3472. 214. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 262. 215. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3306. 216. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2028. 217. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2791. 218. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 424. 219. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2536. 220. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1971. 221. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 415. 222. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3846. 223. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3283. 224. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1956. 225. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3297. 226. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4545. 227. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2661. 228. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1576. 229. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 425. 230. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 709. 231. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2168. 232. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 54. 233. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 429. 234. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 708. 235. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 428. 236. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4119. 237. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3906. 238. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2456. 239. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2278. 240. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5006. 241. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 426. 242. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 307. 243. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5155. 244. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2640. 245. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4317. 246. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1145. 247. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 430. 248. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 885. 249. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 23. 250. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 103. 251. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 22. 252. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4031. 253. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1008. 254. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4790. 255. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2522. 256. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1432. 257. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2914. 258. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3935. 259. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5042. 260. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2865. 261. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4264. 262. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 964. 263. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1268. 264. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5065. 265. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 706. 266. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4704. 267. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 569. 268. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2994. 269. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 829. 270. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1171. 271. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1041. 272. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5070. 273. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 139. 274. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3304. 275. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2431. 276. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4288. 277. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5795. 278. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1300. 279. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 724. 280. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1770. 281. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2830. 282. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1972. 283. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1649. 284. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2515. 285. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2849. 286. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3796. 287. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5815. 288. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 118. 289. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4766. 290. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 770. 291. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1069. 292. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3061. 293. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4290. 294. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4261. 295. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4239. 296. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3478. 297. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1887. 298. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2671. 299. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 256. 300. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1256. 301. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 719. 302. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1448. 303. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 280. 304. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4338. 305. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5037. 306. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1901. 307. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 438. 308. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2834. 309. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5491. 310. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4591. 311. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4936. 312. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 348. 313. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5607. 314. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4955. 315. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5017. 316. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4349. 317. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4964. 318. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 293. 319. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4314. 320. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4995. 321. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4366. 322. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4961. 323. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4952. 324. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5075. 325. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4354. 326. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5060. 327. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5138. 328. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5206. 329. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5283. 330. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5092. 331. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4059. 332. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4295. 333. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5030. 334. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4938. 335. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5215. 336. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4960. 337. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4969. 338. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5023. 339. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4934. 340. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4963. 341. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4363. 342. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5159. 343. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4973. 344. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5157. 345. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4972. 346. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4345. 347. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5039. 348. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5163. 349. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5040. 350. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4304. 351. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5193. 352. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5077. 353. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5814. 354. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 386. 355. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5081. 356. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 308. 357. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 278. 358. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5127. 359. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5029. 360. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5143. 361. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4983. 362. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4361. 363. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5080. 364. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4343. 365. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4986. 366. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4311. 367. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5102. 368. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5280. 369. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5185. 370. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4935. 371. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4379. 372. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5052. 373. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5027. 374. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5173. 375. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5055. 376. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5123. 377. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4335. 378. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5208. 379. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4633. 380. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4359. 381. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 289. 382. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4353. 383. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4316. 384. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5210. 385. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4949. 386. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4947. 387. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2838. 388. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 378. 389. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5013. 390. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5278. 391. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1471. 392. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4346. 393. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 297. 394. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4993. 395. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 384. 396. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5062. 397. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4945. 398. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 210. 399. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4009. 400. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5190. 401. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1561. 402. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5147. 403. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4238. 404. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5527. 405. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4355. 406. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5125. 407. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4108. 408. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2948. 409. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3821. 410. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4632. 411. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 294. 412. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4943. 413. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1391. 414. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5078. 415. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3299. 416. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2897. 417. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1537. 418. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5255. 419. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 141. 420. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5038. 421. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5153. 422. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1181. 423. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1204. 424. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4404. 425. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5106. 426. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2779. 427. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1824. 428. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5116. 429. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3179. 430. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 98. 431. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1872. 432. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1634. 433. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1060. 434. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4941. 435. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 4981. 436. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5274. 437. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5816 (QKMPNNMYG). 438. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 2842. 439. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 1934. 440. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5817. 441. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 3998. 442. A viral protein (VP) capsid polypeptide, wherein the VP capsid polypeptide comprises a 581-589 region corresponding to residues 581 to 589 of a VP1 polypeptide; wherein the 581-589 region has a sequence that is at least 85% identical to SEQ ID NO: 5026. 443. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 18. 444. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3472. 445. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 262. 446. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3306. 447. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2028. 448. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2791. 449. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 424. 450. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2536. 451. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1971. 452. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 415. 453. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3846. 454. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3283. 455. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1956. 456. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3297. 457. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4545. 458. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2661. 459. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1576. 460. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 425. 461. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 709. 462. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2168. 463. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 54. 464. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 429. 465. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 708. 466. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 428. 467. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4119. 468. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3906. 469. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2456. 470. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2278. 471. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5006. 472. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 426. 473. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 307. 474. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5155. 475. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2640. 476. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4317. 477. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1145. 478. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 430. 479. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 885. 480. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 23. 481. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 103. 482. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 22. 483. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4031. 484. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1008. 485. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4790. 486. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2522. 487. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1432. 488. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2914. 489. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3935. 490. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5042. 491. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2865. 492. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4264. 493. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 964. 494. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1268. 495. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5065. 496. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 706. 497. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4704. 498. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 569. 499. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2994. 500. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 829. 501. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1171. 502. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1041. 503. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5070. 504. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 139. 505. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3304. 506. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2431. 507. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4288. 508. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5795. 509. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1300. 510. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 724. 511. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1770. 512. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2830. 513. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1972. 514. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1649. 515. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2515. 516. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2849. 517. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3796. 518. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5815. 519. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 118. 520. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4766. 521. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 770. 522. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1069. 523. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3061. 524. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4290. 525. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4261. 526. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4239. 527. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3478. 528. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1887. 529. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2671. 530. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 256. 531. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1256. 532. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 719. 533. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1448. 534. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 280. 535. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4338. 536. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5037. 537. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1901. 538. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 438. 539. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2834. 540. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5491. 541. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4591. 542. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4936. 543. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 348. 544. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5607. 545. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4955. 546. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5017. 547. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4349. 548. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4964. 549. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 293. 550. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4314. 551. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4995. 552. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4366. 553. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4961. 554. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4952. 555. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5075. 556. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4354. 557. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5060. 558. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5138. 559. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5206. 560. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5283. 561. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5092. 562. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4059. 563. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4295. 564. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5030. 565. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4938. 566. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5215. 567. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4960. 568. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4969. 569. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5023. 570. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4934. 571. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4963. 572. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4363. 573. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5159. 574. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4973. 575. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5157. 576. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4972. 577. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4345. 578. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5039. 579. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5163. 580. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5040. 581. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4304. 582. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5193. 583. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5077. 584. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5814. 585. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 386. 586. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5081. 587. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 308. 588. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 278. 589. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5127. 590. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5029. 591. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5143. 592. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4983. 593. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4361. 594. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5080. 595. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4343. 596. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4986. 597. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4311. 598. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5102. 599. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5280. 600. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5185. 601. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4935. 602. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4379. 603. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5052. 604. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5027. 605. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5173. 606. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5055. 607. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5123. 608. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4335. 609. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5208. 610. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4633. 611. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4359. 612. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 289. 613. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4353. 614. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4316. 615. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5210. 616. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4949. 617. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4947. 618. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2838. 619. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 378. 620. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5013. 621. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5278. 622. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1471. 623. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4346. 624. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 297. 625. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4993. 626. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 384. 627. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5062. 628. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4945. 629. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 210. 630. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4009. 631. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5190. 632. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1561. 633. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5147. 634. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4238. 635. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5527. 636. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4355. 637. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5125. 638. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4108. 639. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2948. 640. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3821. 641. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4632. 642. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 294. 643. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4943. 644. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1391. 645. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5078. 646. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3299. 647. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2897. 648. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1537. 649. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5255. 650. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 141. 651. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5038. 652. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5153. 653. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1181. 654. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1204. 655. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4404. 656. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5106. 657. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2779. 658. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1824. 659. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5116. 660. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3179. 661. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 98. 662. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1872. 663. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1634. 664. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1060. 665. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4941. 666. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 4981. 667. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5274. 668. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5816. 669. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 2842. 670. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 1934. 671. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5817. 672. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 3998. 673. A viral protein (VP) capsid polypeptide, wherein the VP capsid polypeptide comprises a 581-589 region corresponding to residues 581 to 589 of a VP1 polypeptide; wherein the 581-589 region has a sequence of SEQ ID NO: 5026.

### EXAMPLES

Below are examples of specific embodiments for carrying out the present invention. The examples are offered for illustrative purposes only and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature.

### Example 1

### High Throughput Capsid Engineering Systems

This example describes the high throughput capsid engineering systems and methods disclosed herein to discover engineered tissue tropic AAV variants, including CNS tissue-tropic variants and muscle-tropic variants. The high throughput capsid engineering system is schematized in FIG. 1. “Detargeting” may be referred to herein as “sparing” with respect to a particular tissue. For example, liver detargeting or liver sparing may both be used herein to refer to variants or properties of variants that preferentially exhibit reduced homing to liver tissue.

Tissues were harvested and transducing capsid genes are labeled with unique molecular identifiers and barcodes. These variant sequences/UMIs/barcodes were parameterized, and machine learning algorithms were used to identify deterministic features of specific tissue targeting/detargeting capsids.

FIG. 2A provides a side view (top panel) and top view (bottom panel) of key residues of known AAV capsids that have been shown to interact with target cells. FIG. 2B illustrates salient features of the AAV capsid library, showing the region of introduced diversity, with residue numbering corresponding to the numbering of amino acids in AAV5 VP1. The library plasmid includes the invariant rep and diversified cap coding sequences between AAV ITRs (a cis-packaging approach).

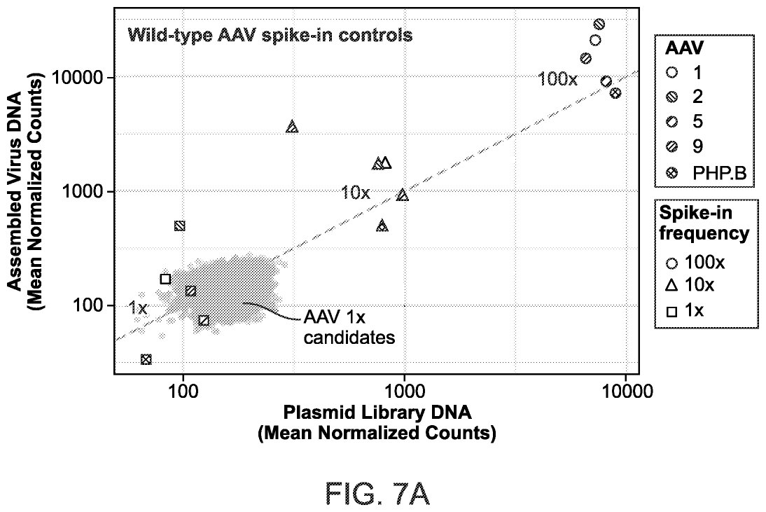

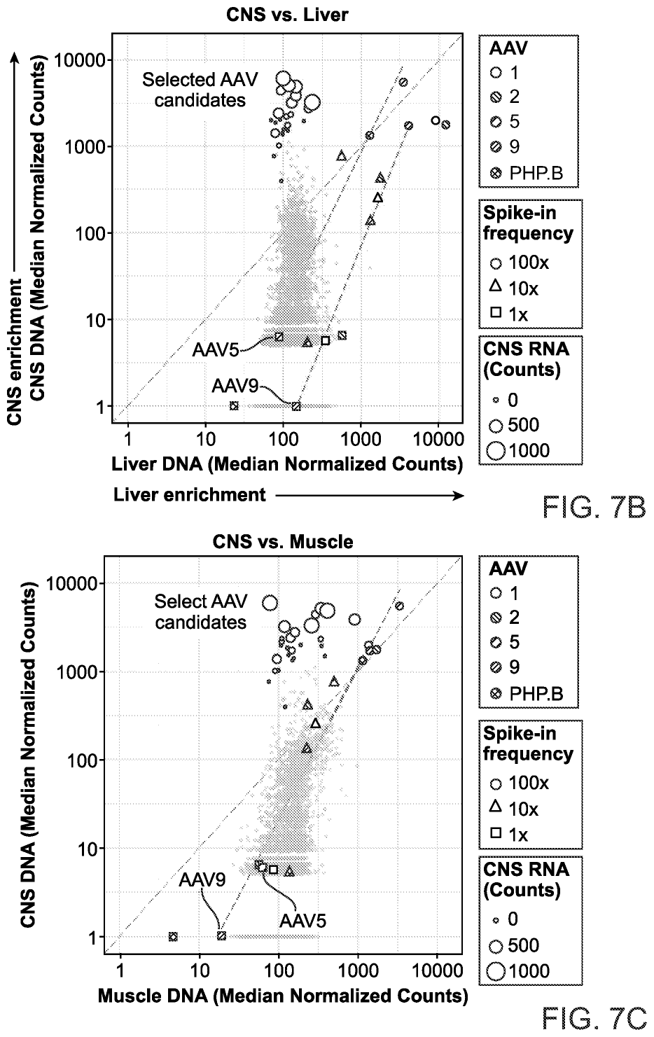

The variants in the AAV capsid library were able to assemble at levels similar to that of wild type AAVs. FIG. 7A illustrates the normalized counts of plasmid library DNA compared to assembled virus DNA for wild type AAV controls (textured points) and for variant AAV candidates (gray circles). Levels of assembled virus for variant AAV candidates were comparable to wild type AAV controls at 1× spike-in frequency.

### Example 2

AAV5 Variants with Tissue Tropism in CNS

This example describes engineered AAV5 variants with tissue tropism in CNS that were discovered using the methods and systems described in EXAMPLE 1.

AAV5 variants found to exhibit CNS tropism included SEQ ID NO: 12-SEQ ID NO: 3937 and SEQ ID NO: 5234-SEQ ID NO: 5807). Variant residues are in the 581-589 region corresponding to positions 581 to 589 of VP1.

TABLE 2 and TABLE 4 provide sequences of 581-589 regions, corresponding to residues 581 to 589 of VP1, for variant AAV VP capsid polypeptides that were found in CNS tissue. The present disclosure, thus, provides for rAAVs composed of engineered AAV5 VP1 capsid polypeptides having a SEQ ID NO: 2, wherein the X¹ to X⁹ region is any one of SEQ ID NO: SEQ ID NO: 12-SEQ ID NO: 3937 or SEQ ID NO: 5234-SEQ ID NO: 5807, as disclosed in TABLE 2 and TABLE 4. Also encompassed herein are rAAVs composed of engineered AAV5 VP2 capsid polypeptides and engineered AAV5 VP3 capsid polypeptides having the sequences disclosed in the below table at the regions in AAV5 VP2 (amino acid residues 445 to 453) and AAV5 VP3 (amino acid residues 389 to 397) corresponding to the amino acids in the AAV5 VP1 581 to 589 region.

Library members selected as CNS-tropic candidates showed greater enrichment in CNS tissue relative to liver (FIG. 7B) or muscle (FIG. 7C) that wild type AAVs or other AAV library members. FIG. 7B shows DNA levels in liver versus CNS for variant AAV candidates (gray circles), wild type controls (textured points), and select AAV candidates with CNS tropism (open circles). CNS-tropic AAV candidates were enriched in CNS tissue relative to liver tissue. FIG. 7C shows DNA levels in muscle versus CNS for variant AAV candidates (gray circles), wild type controls (textured points), and select AAV candidates with CNS tropism (open circles). CNS-tropic AAV candidates were enriched in CNS tissue relative to muscle tissue.

FIG. 4 is a bar graph showing relative accumulation, expressed as log 10 fold change in brain accumulation relative to other tissues, of variant AAV capsids comprising 581-589 regions of SEQ ID NO: 415 (DARVYRALD), SEQ ID NO: 569 (DSASPIMGM), SEQ ID NO: 428 (DAWQMLSGN), SEQ ID NO: 430 (DAWSYQCYH), SEQ ID NO: 708 (EAWMYHQFH), SEQ ID NO: 3906 (YSGVRVTGY), SEQ ID NO: 709 (EAWSKLEQP), SEQ ID NO: 3297 (TAGRFNYFD), SEQ ID NO: 1956 (LVGWTLQHV), SEQ ID NO: 885 (ESWMKLEWQ), SEQ ID NO: 3283 (TAAFAYKYE), SEQ ID NO: 3472 (TSHYITFTP), SEQ ID NO: 426 (DAWMMMWGS), SEQ ID NO: 3306 (TANVYRSGQ), SEQ ID NO: 1971 (LWGWTLQHQ), SEQ ID NO: 425 (DAWLQLKDN), SEQ ID NO: 262 (CATRFNIGG), SEQ ID NO: 424 (DAWCFISSY), SEQ ID NO: 18 (AAGRFNYFG), SEQ ID NO: 54 (AEDYWDLGA), or SEQ ID NO: 2028 (MDDICEFYA). Accumulation was compared to wild type AAV capsids of AAV9, AAV1, AAV PHP.B, AAV2, and AAV5.

### Example 3

### AAV5 Variants with Tissue Tropism in Muscle

This example describes engineered AAV5 variants with tissue tropism in muscle that were discovered using the methods and systems described in EXAMPLE 1.

AAV5 variants found to exhibit muscle tropism included SEQ ID NO: 4238-SEQ ID NO: 4933 and SEQ ID NO: 5808-SEQ ID NO: 5811. Variant residues are in the 581-589 region corresponding to positions 581 to 589 of VP1.

TABLE 5 and TABLE 7 provide sequences of 581-589 regions, corresponding to residues 581 to 589 of VP1, for variant AAV VP capsid polypeptides that were found in muscle tissue. The present disclosure, thus, provides for rAAVs composed of engineered AAV5 VP1 capsid polypeptides having a SEQ ID NO: 2, wherein the X¹ to X⁹ region is any one of SEQ ID NO: 4238-SEQ ID NO: 4933 or SEQ ID NO: 5808-SEQ ID NO: 5811, as disclosed in TABLE 5 and TABLE 7. Also encompassed herein are rAAVs composed of engineered AAV5 VP2 capsid polypeptides and engineered AAV5 VP3 capsid polypeptides having the sequences disclosed in TABLE 5 and TABLE 7 at the regions in AAV5 VP2 (amino acid residues 445 to 453) and AAV5 VP3 (amino acid residues 389 to 397) corresponding to the amino acids in the AAV5 VP1 581 to 589 region.

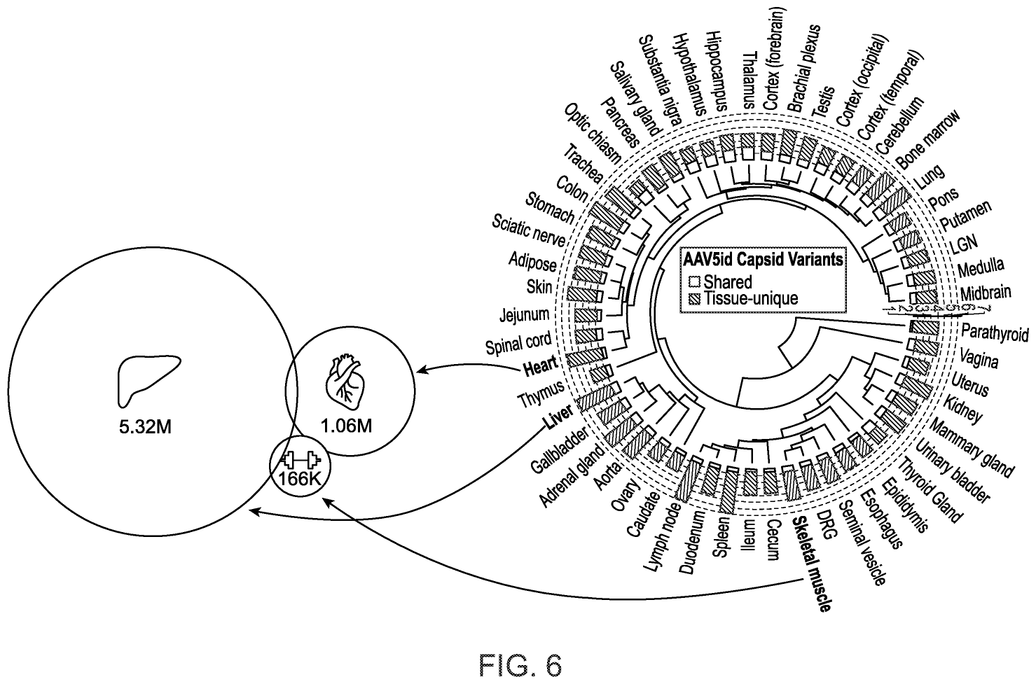

FIG. 6 is a Circos plot depicting variant AAV5 capsids identified in a primary screen performed as illustrated in FIG. 1. Venn diagrams in the figure are not to scale. Over 1 million variant capsids were identified as unique to cardiac tissue, over 100,000 variants were identified as unique to skeletal muscle tissue, and over 1,000 variants were identified as shared between cardiac tissue and skeletal muscle tissue. The variants identified in cardiac tissue, skeletal muscle tissue, or both cardiac and skeletal muscle tissue exhibited low shared identity with liver (about 0.7% overlap) and other tissues (e.g., non-muscle tissues).

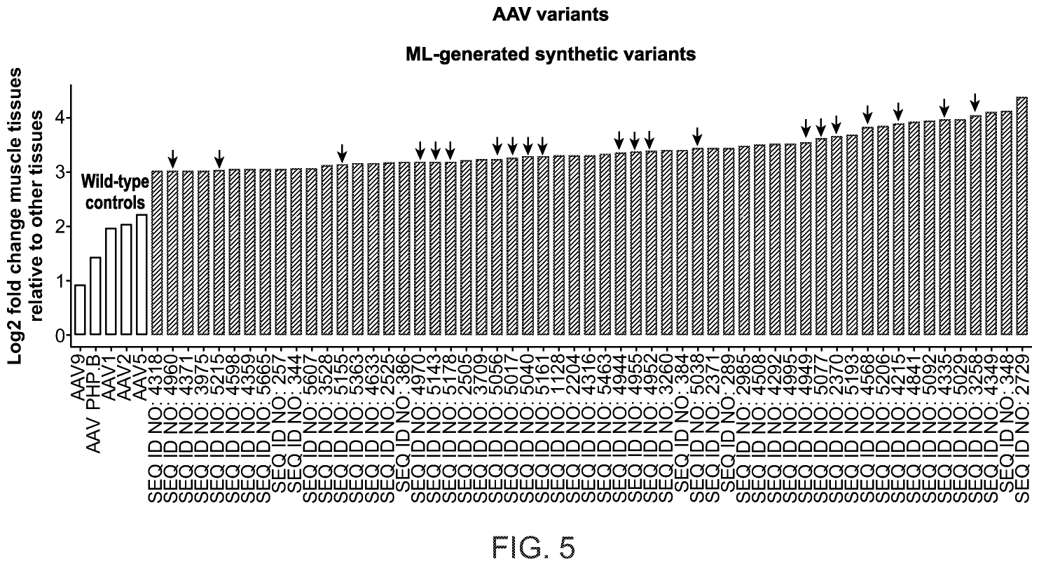

As shown in FIG. 5, muscle-tropic AAV candidates showed 4-fold to 11-fold enrichment in muscle tissue relative to other non-muscle tissues compared to wild type AAV9. FIG. 5 is a bar graph showing relative accumulation, expressed as log 2 fold change in muscle accumulation relative to other tissues, of variant AAV capsids comprising 581-589 regions of SEQ ID NO: 4318 (CKEWYVRDR), SEQ ID NO: 4960 (CWREFSFQN), SEQ ID NO: 4371 (CVSLHSISM), SEQ ID NO: 3975 (QAHHYNILN), SEQ ID NO: 5215 (CVRTLQSPD), SEQ ID NO: 4598 (LAWSQLLTP), SEQ ID NO: 4359 (CVATKSRML), SEQ ID NO: 5665 (RQTTYDCLD), SEQ ID NO: 257 (CAHYAQWGK), SEQ ID NO: 344 (CSSGFHLFH), SEQ ID NO: 5607 (QCGFIRLNE), SEQ ID NO: 3528 (TVTHMSQHC), SEQ ID NO: 5155 (CVIWYHKYE), SEQ ID NO: 5363 (GCMCIRHAY), SEQ ID NO: 4633 (MACWKNATE), SEQ ID NO: 2525 (QAGSSFHQA), SEQ ID NO: 386 (CYQFWSQYS), SEQ ID NO: 4970 (CINFIMKLG), SEQ ID NO: 5143 (CWRHNIISP), SEQ ID NO: 5178 (CIKWVDIFP), SEQ ID NO: 2505 (PTQFYGPAF), SEQ ID NO: 3709 (VSASFQQHV), SEQ ID NO: 5056 (CVLNYFQFG), SEQ ID NO: 5017 (CFYKIQHKG), SEQ ID NO: 5040 (CVLRMGTFC), SEQ ID NO: 5161 (CTHMMGKSP), SEQ ID NO: 1128 (GFANFMMNF), SEQ ID NO: 2204 (MTMTATTFG), SEQ ID NO: 4316 (CIRHAQDCY), SEQ ID NO: 5463 (KISPWDGFY), SEQ ID NO: 4944 (CWKWQMMFG), SEQ ID NO: 4955 (CVWSQILGG), SEQ ID NO: 4952 (CVILSTRDK), SEQ ID NO: 3260 (SVSPCFCNS), SEQ ID NO: 384 (CYMPFKMQH), SEQ ID NO: 5038 (CVVWPVLGQ), SEQ ID NO: 2371 (NMTMAHQAS), SEQ ID NO: 289 (CIHSVHFAA), SEQ ID NO: 2985 (RVDAFNRGT), SEQ ID NO: 4508 (GSCLKIAQE), SEQ ID NO: 4292 (CAMLLQAVQ), SEQ ID NO: 4995 (CVGYRVHSP), SEQ ID NO: 4949 (CYHQVFSQG), SEQ ID NO: 5077 (CVGSQLHAM), SEQ ID NO: 2370 (NMNTFGKMN), SEQ ID NO: 5193 (CWSHLYFQT), SEQ ID NO: 4568 (KANEDLKQF), SEQ ID NO: 5206 (CFFYPMSMS), SEQ ID NO: 4215 (LYAFYMSSE), SEQ ID NO: 4841 (SSDQYEEMN), SEQ ID NO: 5092 (CISNYLKQG), SEQ ID NO: 4335 (CPMKHIQDR), SEQ ID NO: 5029 (CSFNIHKHT), SEQ ID NO: 3258 (SVRMFDAQY), SEQ ID NO: 4349 (CSSYLVTAN), SEQ ID NO: 348 (CTASWMSWD), or SEQ ID NO: 2729 (QQQQTNFQN). AAV variants that were generated using machine learning (ML) are denoted with arrows. Accumulation was compared to wild type AAV capsids of AAV9, AAV PHP.B, AAV1, AAV2, and AAV5.

### Example 4

### Identification of De Novo Variant 581-589 Regions

This example describes identification of de novo variant 581-589 regions using machine learning. Machine learning algorithms were trained using AAV5 variants with CNS tissue tropism identified in EXAMPLE 2 or AAV5 variants with muscle tissue tropism identified in EXAMPLE 3.

Sequences of 581-589 regions that were not identified in the in vivo NHP screen but were predicted by the machine learning analysis to have increased likelihood of either CNS tissue tropism or muscle tropism were generated. After isolating the CNS-specific variants and muscle-specific variants from the in vivo data, novel CNS-specific and muscle-specific sequences were first generated by randomly sampling the amino acid residues at each position of the corresponding population of variants. This created a list of one million variants for each tissue type based on the positional amino acid frequencies observed in the other tissue-specific variants. These one million variants were then tested using random forests classifiers. This calculated a score of predicted tissue-specificity for each variant. The top 300 highest-ranking variants for each tissue type were selected for secondary screening.

TABLE 3 provides sequences of the top 300 581-589 regions, corresponding to residues 581 to 589 of VP1, for variant AAV VP capsid polypeptides that were predicted to be CNS tissue-tropic. The present disclosure, thus, provides for rAAVs composed of engineered AAV5 VP1 capsid polypeptides having a SEQ ID NO: 2, wherein the X¹ to X⁹ region is any one of SEQ ID NO: 3938-SEQ ID NO: 4237, as disclosed in TABLE 3. Also encompassed herein are rAAVs composed of engineered AAV5 VP2 capsid polypeptides and engineered AAV5 VP3 capsid polypeptides having the sequences disclosed in TABLE 3 at the regions in AAV5 VP2 (amino acid residues 445 to 453) and AAV5 VP3 (amino acid residues 389 to 397) corresponding to the amino acids in the AAV5 VP1 581 to 589 region. FIG. 9 shows the CNS prediction score for CNS targeted candidates identified from multiple non-human primates (NHPs), multiple samples, observational enrichment, frequency enrichment, sequence similarity, or machine learning. CNS targeted candidates generated using machine learning had, on average, higher CNS prediction scores than candidates identified from other sources.

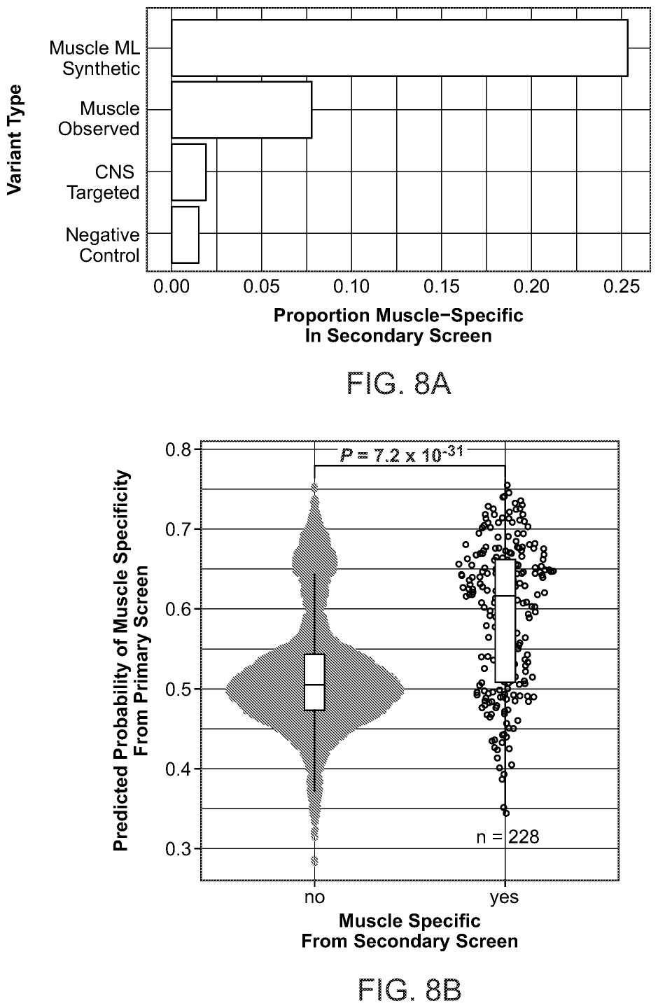

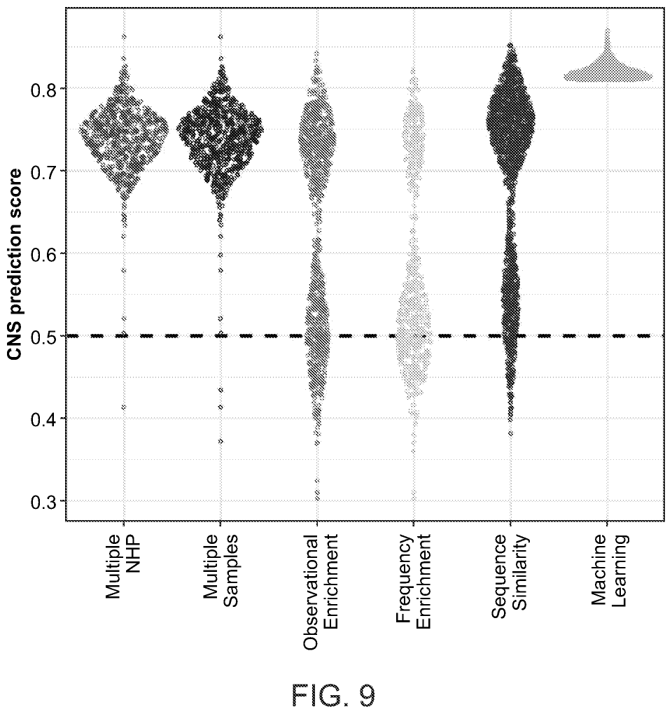

TABLE 6 provides sequences of the top 300 581-589 regions, corresponding to residues 581 to 589 of VP1, for variant AAV VP capsid polypeptides that were predicted to be muscle-tropic. The present disclosure, thus, provides for rAAVs composed of engineered AAV5 VP1 capsid polypeptides having a SEQ ID NO: 2, wherein the X¹ to X⁹ region is any one of SEQ ID NO: 4934-SEQ ID NO: 5233, as disclosed in TABLE 6. Also encompassed herein are rAAVs composed of engineered AAV5 VP2 capsid polypeptides and engineered AAV5 VP3 capsid polypeptides having the sequences disclosed in TABLE 6 at the regions in AAV5 VP2 (amino acid residues 445 to 453) and AAV5 VP3 (amino acid residues 389 to 397) corresponding to the amino acids in the AAV5 VP1 581 to 589 region. FIG. 8A shows a bar graph of the proportion of candidates from different library sub-sets that were identified as muscle-specific in a secondary screen. Muscle candidates generated using machine learning (“Muscle ML Synthetic”) contained a higher proportion of muscle-specific sequences than candidates identified in muscle in a primary screen (“Muscle Observed”), CNS targeted sequences, or negative control sequences. Muscle candidate sequences generated using machine learning were five times more likely to be heart- and/or skeletal muscle-specific than muscle candidates identified in the primary screen and were 20 times more likely to be heart- and/or skeletal muscle-specific than CNS targeted variants. Candidates were classified as muscle-specific if they were >2-fold enriched in heart and/or skeletal muscle relative to all other tissues and viral input (FDR <0.1, permutations with α-RRA to account for consistency across multiple barcodes for each capsid). FIG. 8B shows a box and whisker plot comparing the predicted probability of muscle specificity from primary screen for candidates that were (“yes”) or were not (“no”) identified as muscle-specific in the secondary screen. Candidates were classified as muscle-specific if they were >2-fold enriched in heart and/or skeletal muscle relative to all other tissues and viral input (FDR <0.1, permutations with α-RRA to account for consistency across multiple barcodes for each capsid).

### Example 5

### Secondary Screen of Recombinant AAV Capsid Candidates with Variant 581-589 Regions

This example describes a secondary screen of recombinant AAV capsid candidates with variant 581-589 regions. Six thousand AAV capsid variants identified or generated using the methods described in EXAMPLE 1-EXAMPLE 4 were systemically administered to non-human primate. Fifteen wild type spike-in controls representing wild type AAV1, AAV2, AAV5, AAV9, and AAV PHP.B, each at 1×, 10×, and 100× concentrations, along with 100 AAV capsids with stop codons in the 581-589 region, were also administered. Tissue-specific accumulation and tissue-specific transduction of each capsid were evaluated by quantifying capsid DNA and RNA, respectively, in tissues of interest. Barcode sequences for each capsid were extracted from raw sequencing data after filtering based on sequencing quality (Q30) and exact match to the reference library of 48,090 variants representing the 6,000 variant capsid sequences and 15 wild type spike-in controls.

Enrichment of variant capsids relative to wild type AAVs was quantified for the barcodes associated with each capsid. Enrichment was determined for each capsid for both the RNA and DNA fractions from each of the analyzed tissues, as well as for the input viral library injected into each primate. DNA counts were representative of capsid accumulation, and RNA counts were representative of viral transduction in the tissue. This analysis collectively formed 12 distinct analysis strategies to assess the differential expression of capsids. The different analytical outputs were consolidated into a single z-score to evaluate enrichment of each capsid in a tissue of interest relative to the overall distribution of the capsid differential expression data. The z-score was calculated as follows:

$${Z}_{i} = \frac{{X}_{i} - mean ( X )}{sd ( X )}$$

where Xᵢ is the enrichment of capsid, i, estimated as the median of the log 2(fold change) across capsid barcodes and tissue comparisons. Superior capsid variants were selected based on parameters such as DNA or RNA abundance in a tissue of interest.

### Example 6

### Secondary Screen Results to Identify CNS Tissue-Tropic Recombinant AAV Capsids

This example describes secondary screen results to identify CNS tissue-tropic recombinant AAV capsids. The secondary screen and enrichment analysis were performed as described in EXAMPLE 5. Recombinant AAV capsids containing variant 581-589 regions that were enriched in CNS tissue and exhibited functional transduction in CNS tissue were identified. TABLE 9 provides enrichment data for the 581-589 region sequences of the top 100 CNS tissue-tropic AAV capsids, as identified in the secondary screen. Superior capsids were selected based on parameters including relative DNA and RNA abundance in CNS tissue, as well as additional metrics shown in TABLE 9, including average of z-score, liver DNA fold change over AAV5, liver RNA fold change over AAV5, liver DNA fold change over AAV9, and liver RNA fold change over AAV9.

TABLE 9
Secondary Screen Results for CNS Tissue-Tropic Capsids

| SEQ ID | DNA FC | RNA FC | DNA FC | RNA FC | DNA FC | RNA FC | Z | Liver DNA | Liver RNA | Liver DNA | Liver RNA |
|---|---|---|---|---|---|---|---|---|---|---|---|
| NO | Input | Input | AAV9 | AAV9 | AAV5 | AAV5 | Ave. | FC AAV5 | FC AAV5 | FC AAV9 | FC AAV9 |
| 18 | 191.25 | 274.64 | 1164.65 | 984.41 | 33.20 | 112.33 | 8.61 | 1.34 | 1.22 | 2.78 | 1.25 |
| 3472 | 101.65 | 174.44 | 619.05 | 625.26 | 17.65 | 71.35 | 6.94 | 1.30 | 0.62 | 2.70 | 0.64 |
| 262 | 74.65 | 175.05 | 454.62 | 627.45 | 12.96 | 71.60 | 6.01 | 2.15 | 1.01 | 4.45 | 1.04 |
| 3306 | 71.12 | 123.11 | 433.11 | 441.25 | 12.35 | 50.35 | 7.54 | 1.08 | 0.49 | 2.24 | 0.51 |
| 2028 | 65.57 | 139.59 | 399.28 | 500.34 | 11.38 | 57.09 | 8.53 | 0.87 | 0.35 | 1.79 | 0.36 |
| 2791 | 64.72 | 112.35 | 394.14 | 402.71 | 11.24 | 45.95 | 6.10 | 0.72 | 1.23 | 1.49 | 1.27 |
| 424 | 61.14 | 110.28 | 372.33 | 395.30 | 10.62 | 45.11 | 8.12 | 0.94 | 0.45 | 1.95 | 0.46 |
| 2536 | 57.12 | 117.60 | 347.86 | 421.52 | 9.92 | 48.10 | 5.71 | 1.04 | 0.24 | 2.16 | 0.25 |
| 1971 | 55.09 | 71.39 | 335.48 | 255.89 | 9.56 | 29.20 | 6.10 | 0.89 | 0.62 | 1.84 | 0.64 |
| 415 | 51.53 | 65.45 | 313.81 | 234.59 | 8.95 | 26.77 | 6.68 | 1.64 | 1.52 | 3.40 | 1.56 |
| 3846 | 48.77 | 1.79 | 297.02 | 6.40 | 8.47 | 0.73 | 2.20 | 1.21 | 0.63 | 2.50 | 0.65 |
| 3283 | 45.30 | 119.98 | 275.88 | 430.06 | 7.87 | 49.07 | 5.43 | 0.89 | 0.29 | 1.84 | 0.30 |
| 1956 | 44.62 | 131.11 | 271.74 | 469.93 | 7.75 | 53.62 | 6.06 | 0.98 | 0.29 | 2.04 | 0.30 |
| 3297 | 37.74 | 40.08 | 229.83 | 143.66 | 6.55 | 16.39 | 4.90 | 1.16 | 0.77 | 2.41 | 0.79 |
| 4545 | 32.45 | 39.08 | 197.63 | 140.07 | 5.63 | 15.98 | 3.50 | 0.98 | 0.43 | 2.03 | 0.45 |
| 2661 | 31.72 | 63.55 | 193.14 | 227.79 | 5.51 | 25.99 | 4.80 | 2.18 | 1.24 | 4.51 | 1.27 |
| 1576 | 29.64 | 62.61 | 180.47 | 224.43 | 5.15 | 25.61 | 3.54 | 1.40 | 0.99 | 2.91 | 1.02 |
| 425 | 27.64 | 57.62 | 168.33 | 206.52 | 4.80 | 23.57 | 5.94 | 0.82 | 0.54 | 1.70 | 0.56 |
| 709 | 26.54 | 118.04 | 161.61 | 423.11 | 4.61 | 48.28 | 6.36 | 0.73 | 1.02 | 1.52 | 1.05 |
| 2168 | 26.46 | 1.72 | 161.14 | 6.17 | 4.59 | 0.70 | −0.67 | 1.53 | 0.90 | 3.18 | 0.93 |
| 54 | 24.61 | 81.85 | 149.88 | 293.39 | 4.27 | 33.48 | 5.50 | 0.78 | 0.89 | 1.61 | 0.92 |
| 429 | 24.47 | 29.17 | 149.03 | 104.55 | 4.25 | 11.93 | 4.44 | 0.78 | 0.53 | 1.62 | 0.55 |
| 708 | 23.66 | 25.56 | 144.07 | 91.62 | 4.11 | 10.45 | 5.65 | 0.61 | 0.64 | 1.26 | 0.66 |
| 428 | 23.48 | 38.00 | 143.01 | 136.19 | 4.08 | 15.54 | 4.76 | 0.86 | 0.75 | 1.79 | 0.77 |
| 4119 | 22.63 | 3.10 | 137.79 | 11.11 | 3.93 | 1.27 | −0.09 | 1.43 | 0.60 | 2.96 | 0.62 |
| 3906 | 22.06 | 10.52 | 134.37 | 37.69 | 3.83 | 4.30 | 3.41 | 1.05 | 0.68 | 2.17 | 0.71 |
| 2456 | 21.87 | 4.38 | 133.18 | 15.72 | 3.80 | 1.79 | 0.68 | 0.93 | 0.14 | 1.92 | 0.15 |
| 2278 | 20.11 | 0.18 | 122.45 | 0.65 | 3.49 | 0.07 | 0.93 | 0.84 | 0.28 | 1.74 | 0.29 |
| 5006 | 19.89 | 3.13 | 121.13 | 11.21 | 3.45 | 1.28 | 0.20 | 1.33 | 0.55 | 2.76 | 0.57 |
| 426 | 19.78 | 27.90 | 120.48 | 100.02 | 3.43 | 11.41 | 3.48 | 0.94 | 0.35 | 1.95 | 0.36 |
| 307 | 18.65 | 19.51 | 113.58 | 69.94 | 3.24 | 7.98 | 2.91 | 1.68 | 0.95 | 3.49 | 0.98 |
| 5155 | 17.70 | 0.18 | 107.81 | 0.63 | 3.07 | 0.07 | −0.76 | 1.17 | 0.99 | 2.42 | 1.02 |
| 2640 | 17.65 | 0.18 | 107.51 | 0.63 | 3.07 | 0.07 | −0.40 | 1.54 | 0.98 | 3.18 | 1.02 |
| 4317 | 17.21 | 25.30 | 104.82 | 90.68 | 2.99 | 10.35 | −0.67 | 1.16 | 1.15 | 2.41 | 1.19 |
| 1145 | 17.18 | 4.79 | 104.62 | 17.18 | 2.98 | 1.96 | 0.24 | 1.11 | 0.43 | 2.29 | 0.44 |
| 430 | 17.08 | 112.52 | 104.03 | 403.32 | 2.97 | 46.02 | 5.26 | 0.85 | 0.20 | 1.76 | 0.20 |
| 885 | 16.85 | 26.78 | 102.60 | 95.98 | 2.93 | 10.95 | 4.49 | 0.80 | 0.41 | 1.65 | 0.43 |
| 23 | 15.09 | 5.08 | 91.89 | 18.22 | 2.62 | 2.08 | 1.65 | 1.57 | 0.79 | 3.25 | 0.82 |
| 103 | 14.93 | 0.37 | 90.91 | 1.33 | 2.59 | 0.15 | 2.04 | 1.02 | 0.24 | 2.11 | 0.25 |
| 22 | 14.24 | 7.55 | 86.72 | 27.06 | 2.47 | 3.09 | 1.16 | 1.76 | 0.90 | 3.65 | 0.93 |
| 4031 | 14.08 | 0.08 | 85.76 | 0.29 | 2.44 | 0.03 | −0.83 | 1.19 | 0.53 | 2.46 | 0.55 |
| 1008 | 13.74 | 0.20 | 83.69 | 0.70 | 2.39 | 0.08 | 0.66 | 1.40 | 1.62 | 2.89 | 1.67 |
| 4790 | 13.33 | 0.07 | 81.15 | 0.24 | 2.31 | 0.03 | −0.16 | 1.34 | 1.38 | 2.78 | 1.43 |
| 2522 | 13.08 | 0.14 | 79.63 | 0.50 | 2.27 | 0.06 | −0.05 | 1.28 | 0.97 | 2.65 | 1.00 |
| 1432 | 13.01 | 2.52 | 79.26 | 9.02 | 2.26 | 1.03 | 2.03 | 0.97 | 0.86 | 2.00 | 0.89 |
| 2914 | 12.99 | 3.03 | 79.13 | 10.84 | 2.26 | 1.24 | −0.48 | 1.44 | 0.54 | 2.99 | 0.56 |
| 3935 | 12.91 | 15.87 | 78.65 | 56.88 | 2.24 | 6.49 | 3.37 | 0.80 | 0.25 | 1.65 | 0.26 |
| 5042 | 12.78 | 0.09 | 77.84 | 0.33 | 2.22 | 0.04 | 0.60 | 0.82 | 0.27 | 1.70 | 0.28 |
| 2865 | 12.37 | 22.18 | 75.33 | 79.48 | 2.15 | 9.07 | 2.54 | 0.94 | 0.51 | 1.95 | 0.53 |
| 4264 | 12.27 | 4.51 | 74.69 | 16.17 | 2.13 | 1.84 | −0.88 | 2.07 | 2.54 | 4.29 | 2.62 |
| 964 | 11.88 | 8.67 | 72.35 | 31.08 | 2.06 | 3.55 | 1.73 | 1.03 | 1.54 | 2.13 | 1.59 |
| 1268 | 11.85 | 22.35 | 72.18 | 80.12 | 2.06 | 9.14 | 3.33 | 0.95 | 0.91 | 1.97 | 0.94 |
| 5065 | 11.85 | 5.41 | 72.16 | 19.38 | 2.06 | 2.21 | −0.20 | 1.06 | 0.81 | 2.21 | 0.84 |
| 706 | 11.55 | 13.76 | 70.31 | 49.31 | 2.00 | 5.63 | 2.86 | 0.78 | 0.31 | 1.61 | 0.32 |
| 4704 | 11.23 | 2.07 | 68.39 | 7.40 | 1.95 | 0.84 | −0.08 | 1.13 | 0.72 | 2.35 | 0.74 |
| 569 | 11.17 | 81.65 | 68.00 | 292.66 | 1.94 | 33.40 | 4.36 | 0.74 | 0.78 | 1.53 | 0.80 |
| 2994 | 11.01 | 0.10 | 67.06 | 0.35 | 1.91 | 0.04 | 0.62 | 2.01 | 1.32 | 4.16 | 1.36 |
| 829 | 10.81 | 0.53 | 65.82 | 1.90 | 1.88 | 0.22 | 0.96 | 0.99 | 0.64 | 2.05 | 0.66 |
| 1171 | 10.78 | 9.90 | 65.68 | 35.48 | 1.87 | 4.05 | 2.66 | 1.09 | 1.09 | 2.26 | 1.13 |
| 1041 | 10.60 | 0.16 | 64.53 | 0.56 | 1.84 | 0.06 | 0.31 | 0.85 | 0.73 | 1.76 | 0.76 |
| 5070 | 10.34 | 3.89 | 62.97 | 13.95 | 1.80 | 1.59 | −0.20 | 1.26 | 0.56 | 2.61 | 0.58 |
| 139 | 10.29 | 3.51 | 62.66 | 12.57 | 1.79 | 1.43 | −0.44 | 0.97 | 0.69 | 2.01 | 0.72 |
| 3304 | 10.15 | 0.12 | 61.81 | 0.44 | 1.76 | 0.05 | −0.20 | 1.30 | 0.92 | 2.70 | 0.95 |
| 2431 | 9.87 | 2.16 | 60.13 | 7.73 | 1.71 | 0.88 | 0.73 | 1.11 | 0.84 | 2.31 | 0.87 |
| 4288 | 9.87 | 14.80 | 60.10 | 53.06 | 1.71 | 6.05 | −1.21 | 1.57 | 1.21 | 3.25 | 1.25 |
| 5795 | 9.85 | 0.24 | 59.96 | 0.86 | 1.71 | 0.10 | 1.01 | 0.91 | 0.56 | 1.89 | 0.58 |
| 1300 | 9.79 | 8.49 | 59.64 | 30.45 | 1.70 | 3.47 | 0.49 | 0.86 | 0.23 | 1.78 | 0.23 |
| 724 | 9.78 | 15.72 | 59.58 | 56.33 | 1.70 | 6.43 | 3.70 | 0.74 | 0.17 | 1.53 | 0.17 |
| 1770 | 9.77 | 0.07 | 59.47 | 0.26 | 1.70 | 0.03 | 0.37 | 1.43 | 0.49 | 2.96 | 0.50 |
| 2830 | 9.63 | 3.28 | 58.63 | 11.77 | 1.67 | 1.34 | 0.44 | 1.26 | 0.91 | 2.60 | 0.94 |
| 1972 | 9.56 | 3.95 | 58.19 | 14.16 | 1.66 | 1.62 | 0.18 | 0.95 | 0.36 | 1.96 | 0.37 |
| 1649 | 9.53 | 0.26 | 58.02 | 0.93 | 1.65 | 0.11 | −0.19 | 1.86 | 2.25 | 3.86 | 2.33 |
| 2515 | 9.50 | 0.05 | 57.88 | 0.20 | 1.65 | 0.02 | 0.42 | 0.76 | 0.87 | 1.58 | 0.89 |
| 2849 | 9.40 | 0.05 | 57.27 | 0.17 | 1.63 | 0.02 | −0.06 | 0.90 | 0.63 | 1.86 | 0.65 |
| 3796 | 9.34 | 0.05 | 56.90 | 0.17 | 1.62 | 0.02 | 0.20 | 1.13 | 0.43 | 2.35 | 0.44 |
| 5815 | 9.33 | 0.42 | 56.83 | 1.50 | 1.62 | 0.17 | −0.48 | 2.37 | 1.72 | 4.90 | 1.78 |
| 118 | 9.27 | 0.33 | 56.48 | 1.19 | 1.61 | 0.14 | 1.79 | 0.99 | 0.23 | 2.05 | 0.24 |
| 4766 | 9.22 | 0.12 | 56.16 | 0.44 | 1.60 | 0.05 | 0.82 | 1.12 | 1.62 | 2.32 | 1.67 |
| 770 | 9.17 | 0.19 | 55.84 | 0.67 | 1.59 | 0.08 | 0.56 | 1.50 | 0.15 | 3.11 | 0.16 |
| 1069 | 9.14 | 7.40 | 55.67 | 26.53 | 1.59 | 3.03 | 2.12 | 1.07 | 2.73 | 2.22 | 2.82 |
| 3061 | 8.90 | 0.36 | 54.18 | 1.29 | 1.54 | 0.15 | 0.67 | 1.17 | 0.42 | 2.41 | 0.43 |
| 4290 | 8.86 | 3.51 | 53.97 | 12.58 | 1.54 | 1.44 | −1.31 | 0.95 | 0.59 | 1.98 | 0.61 |
| 4261 | 8.83 | 0.15 | 53.75 | 0.52 | 1.53 | 0.06 | 0.65 | 0.72 | 0.24 | 1.48 | 0.25 |
| 4239 | 8.78 | 0.47 | 53.44 | 1.68 | 1.52 | 0.19 | 0.00 | 1.11 | 0.72 | 2.31 | 0.75 |
| 3478 | 8.68 | 5.43 | 52.83 | 19.45 | 1.51 | 2.22 | −0.31 | 1.32 | 1.07 | 2.73 | 1.11 |
| 1887 | 8.65 | 39.87 | 52.71 | 142.90 | 1.50 | 16.31 | 3.43 | 1.39 | 0.89 | 2.88 | 0.92 |
| 2671 | 8.57 | 4.68 | 52.17 | 16.79 | 1.49 | 1.92 | 0.67 | 1.06 | 0.60 | 2.19 | 0.62 |
| 256 | 8.38 | 3.78 | 51.03 | 13.55 | 1.45 | 1.55 | −0.26 | 1.33 | 0.59 | 2.76 | 0.61 |
| 1256 | 8.35 | 6.91 | 50.87 | 24.78 | 1.45 | 2.83 | −0.63 | 1.82 | 1.94 | 3.77 | 2.00 |
| 719 | 8.31 | 71.34 | 50.61 | 255.71 | 1.44 | 29.18 | 3.02 | 1.02 | 0.93 | 2.11 | 0.96 |
| 1448 | 8.27 | 0.14 | 50.38 | 0.49 | 1.44 | 0.06 | 0.43 | 0.85 | 0.85 | 1.76 | 0.87 |
| 280 | 8.25 | 4.62 | 50.23 | 16.57 | 1.43 | 1.89 | 0.22 | 1.06 | 0.41 | 2.20 | 0.42 |
| 4338 | 8.21 | 13.98 | 49.98 | 50.11 | 1.42 | 5.72 | −0.64 | 1.17 | 0.68 | 2.43 | 0.71 |
| 5037 | 8.09 | 3.86 | 49.29 | 13.83 | 1.41 | 1.58 | −0.89 | 1.29 | 0.43 | 2.66 | 0.44 |
| 1901 | 8.01 | 0.69 | 48.80 | 2.46 | 1.39 | 0.28 | −0.82 | 1.42 | 1.05 | 2.93 | 1.09 |
| 438 | 7.99 | 6.30 | 48.67 | 22.59 | 1.39 | 2.58 | −0.31 | 1.10 | 0.77 | 2.27 | 0.79 |
| 2834 | 7.96 | 9.98 | 48.50 | 35.77 | 1.38 | 4.08 | 1.10 | 1.98 | 0.92 | 4.09 | 0.95 |
| 5491 | 7.96 | 1.42 | 48.45 | 5.10 | 1.38 | 0.58 | −0.32 | 3.00 | 3.06 | 6.22 | 3.16 |
| 4591 | 7.94 | 11.88 | 48.35 | 42.57 | 1.38 | 4.86 | −0.26 | 1.17 | 0.92 | 2.43 | 0.95 |
| 4936 | 7.94 | 1.98 | 48.33 | 7.08 | 1.38 | 0.81 | −0.42 | 1.23 | 0.91 | 2.54 | 0.94 |

As shown in TABLE 9, “DNA FC Input” represents the fold change of counts per million (CPM) values, normalized by Trimmed Mean of M-values (TMM) of capsid DNA relative to viral input in CNS tissue. “RNA FC Input” represents the fold change of CPM values (normalized by TMM) of capsid RNA relative to viral input in CNS tissue. “DNA FC AAV9” represents the fold change of CPM values (normalized by TMM and to viral input) of capsid DNA relative to wild-type AAV9 in CNS tissue. “RNA FC AAV9” represents the fold change of CPM values (normalized by TMM and to viral input) of capsid RNA relative to wild-type AAV9 in CNS tissue. “DNA FC AAV5” represents the fold change of CPM values (normalized by TMM and to viral input) of capsid DNA relative to wild-type AAV5 in CNS tissue. “RNA FC AAV5” represents the fold change of CPM values (normalized by TMM and to viral input) of capsid RNA relative to wild-type AAV5 in CNS tissue. “Z. Ave.” represents the average of z-scores calculated from the enrichment values of 12 distinct differential expression analyses comparing the tissue of interest to all other tissues. “Liver DNA FC AAV5” represents the fold change of CPM values (normalized by TMM and to viral input) of capsid DNA relative to wild-type AAV5 in the liver. “Liver RNA FC AAV5” represents the fold change of CPM values (normalized by TMM and to viral input) of capsid RNA relative to wild-type AAV5 in the liver. “Liver DNA FC AAV9” represents the fold change of CPM values (normalized by TMM and to viral input) of capsid DNA relative to wild-type AAV9 in the liver. “Liver RNA FC AAV9” represents the fold change of CPM values (normalized by TMM and to viral input) of capsid RNA relative to wild-type AAV9 in the liver.

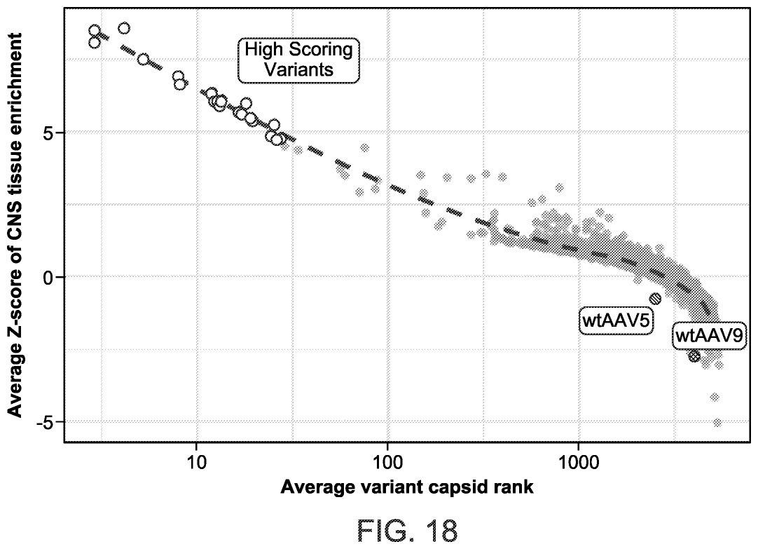

The average Z-score of CNS tissue enrichment for the screened capsid variants, plotted in order of capsid rank, is shown in FIG. 18. High scoring variants corresponding to SEQ ID NO: 18, SEQ ID NO: 2028, SEQ ID NO: 424, SEQ ID NO: 3306, SEQ ID NO: 3472, SEQ ID NO: 415, SEQ ID NO: 709, SEQ ID NO: 1971, SEQ ID NO: 2791, SEQ ID NO: 1956, SEQ ID NO: 262, SEQ ID NO: 425, SEQ ID NO: 2536, SEQ ID NO: 708, SEQ ID NO: 54, SEQ ID NO: 3283, SEQ ID NO: 430, SEQ ID NO: 3297, SEQ ID NO: 2661, SEQ ID NO: 428, SEQ ID NO: 885, SEQ ID NO: 429, SEQ ID NO: 569, SEQ ID NO: 724, SEQ ID NO: 1576, SEQ ID NO: 4545, SEQ ID NO: 426, SEQ ID NO: 1887, SEQ ID NO: 3906, and SEQ ID NO: 3935 are emphasized. The high scoring variant capsid, corresponding to the outlined points in the upper left quadrant, were ranked consistently highly across multiple analysis strategies and ranked consistently higher than wild type AAV5 (wtAAV5) and wild type AAV9 (wtAAV9).

### Example 7

### Secondary Screen Results to Identify Cardiac Tissue-Tropic Recombinant AAV Capsids

This example describes secondary screen results to identify cardiac tissue-tropic recombinant AAV capsids. The secondary screen and enrichment analysis were performed as described in EXAMPLE 5. Recombinant AAV capsids containing variant 581-589 regions that were enriched in cardiac tissue (including heart tissue and aorta tissue) and exhibited functional transduction in cardiac tissue were identified. TABLE 10 provides enrichment data for the 581-589 region sequences of the top 100 cardiac tissue-tropic AAV capsids, as identified in the secondary screen, and the 15 wild type spike-in controls. Superior capsids were selected based on parameters including relative DNA and RNA abundance in cardiac tissue, as well as additional metrics shown in TABLE 10, including liver DNA fold change over AAV5, liver RNA fold change over AAV5, liver DNA fold change over AAV9, and liver RNA fold change over AAV9.

TABLE 10
Secondary Screen Results for Cardiac Tissue-Tropic Capsids

| | DNA | RNA | DNA | RNA | DNA | RNA | Liver | Liver | Liver |
|---|---|---|---|---|---|---|---|---|---|
| SEQ | FC | FC | FC | FC | FC | FC | DNA FC | RNA FC | DNA FC |
| ID NO | Input | Input | AAV9 | AAV9 | AAV5 | AAV5 | AAV5 | AAV5 | AAV9 |
| 348 | 9.32 | 10.61 | 62.21 | 47.20 | 12.56 | 14.17 | 2.85 | 7.14 | 2.94 |
| 2536 | 7.74 | 2.24 | 51.67 | 9.97 | 10.43 | 2.99 | 0.24 | 2.16 | 0.25 |
| 5607 | 6.03 | 2.46 | 40.23 | 10.95 | 8.12 | 3.29 | 18.66 | 32.87 | 19.25 |
| 1576 | 5.53 | 1.38 | 36.89 | 6.15 | 7.45 | 1.84 | 0.99 | 2.91 | 1.02 |
| 4955 | 5.37 | 2.20 | 35.86 | 9.78 | 7.24 | 2.93 | 0.38 | 2.70 | 0.39 |
| 5017 | 5.22 | 2.68 | 34.87 | 11.94 | 7.04 | 3.58 | 1.94 | 2.76 | 2.00 |
| 4349 | 5.12 | 2.31 | 34.20 | 10.28 | 6.90 | 3.08 | 0.46 | 2.40 | 0.47 |
| 4964 | 4.95 | 2.55 | 33.04 | 11.35 | 6.67 | 3.41 | 1.28 | 3.69 | 1.32 |
| 293 | 4.92 | 4.10 | 32.83 | 18.24 | 6.63 | 5.48 | 0.79 | 3.06 | 0.81 |
| 4314 | 4.78 | 2.66 | 31.91 | 11.83 | 6.44 | 3.55 | 0.49 | 2.21 | 0.50 |
| 4995 | 4.74 | 3.17 | 31.66 | 14.08 | 6.39 | 4.23 | 0.73 | 1.95 | 0.76 |
| 4366 | 4.50 | 1.99 | 30.03 | 8.83 | 6.06 | 2.65 | 1.20 | 2.86 | 1.24 |
| 4961 | 4.47 | 1.64 | 29.86 | 7.30 | 6.03 | 2.19 | 1.02 | 2.25 | 1.05 |
| 4952 | 4.42 | 4.11 | 29.52 | 18.29 | 5.96 | 5.49 | 0.96 | 2.80 | 0.99 |
| 5075 | 4.41 | 3.45 | 29.42 | 15.34 | 5.94 | 4.60 | 1.52 | 2.70 | 1.57 |
| 4354 | 4.40 | 2.03 | 29.39 | 9.02 | 5.93 | 2.71 | 1.91 | 1.81 | 1.97 |
| 5060 | 4.39 | 2.88 | 29.29 | 12.82 | 5.91 | 3.85 | 0.41 | 2.62 | 0.42 |
| 5138 | 4.32 | 5.91 | 28.86 | 26.27 | 5.82 | 7.89 | 0.57 | 2.04 | 0.59 |
| 5206 | 4.27 | 3.04 | 28.50 | 13.53 | 5.75 | 4.06 | 0.63 | 2.58 | 0.65 |
| 4288 | 4.23 | 3.18 | 28.25 | 14.14 | 5.70 | 4.24 | 1.21 | 3.25 | 1.25 |
| 5283 | 4.20 | 2.02 | 28.00 | 8.98 | 5.65 | 2.70 | 5.79 | 11.85 | 5.97 |
| 5092 | 4.18 | 1.66 | 27.89 | 7.39 | 5.63 | 2.22 | 0.36 | 1.88 | 0.37 |
| 4059 | 4.12 | 1.97 | 27.50 | 8.76 | 5.55 | 2.63 | 0.72 | 2.34 | 0.75 |
| 4295 | 4.07 | 2.17 | 27.15 | 9.66 | 5.48 | 2.90 | 1.19 | 2.89 | 1.23 |
| 5030 | 4.04 | 3.17 | 26.99 | 14.08 | 5.45 | 4.23 | 3.24 | 8.80 | 3.34 |
| 4938 | 4.03 | 2.25 | 26.92 | 10.02 | 5.43 | 3.01 | 0.68 | 2.83 | 0.70 |
| 2661 | 4.02 | 1.99 | 26.86 | 8.85 | 5.42 | 2.66 | 1.24 | 4.51 | 1.27 |
| 5215 | 4.02 | 2.68 | 26.82 | 11.94 | 5.41 | 3.58 | 1.53 | 5.08 | 1.58 |
| 4960 | 4.00 | 1.79 | 26.69 | 7.97 | 5.39 | 2.39 | 0.59 | 2.26 | 0.61 |
| 4969 | 3.95 | 1.63 | 26.38 | 7.26 | 5.33 | 2.18 | 0.29 | 2.49 | 0.30 |
| 5023 | 3.93 | 1.43 | 26.22 | 6.36 | 5.29 | 1.91 | 0.46 | 2.39 | 0.48 |
| 4934 | 3.92 | 1.92 | 26.15 | 8.54 | 5.28 | 2.56 | 0.85 | 2.00 | 0.88 |
| 4545 | 3.89 | 1.82 | 26.00 | 8.12 | 5.25 | 2.44 | 0.43 | 2.03 | 0.45 |
| 4963 | 3.89 | 1.47 | 25.95 | 6.53 | 5.24 | 1.96 | 3.75 | 10.01 | 3.87 |
| 4363 | 3.88 | 2.40 | 25.93 | 10.67 | 5.23 | 3.20 | 0.45 | 2.20 | 0.46 |
| 5159 | 3.79 | 2.96 | 25.30 | 13.15 | 5.11 | 3.95 | 1.03 | 2.57 | 1.06 |
| 4973 | 3.77 | 1.86 | 25.17 | 8.28 | 5.08 | 2.48 | 0.59 | 2.63 | 0.61 |
| 5157 | 3.68 | 1.60 | 24.58 | 7.13 | 4.96 | 2.14 | 5.00 | 13.10 | 5.15 |
| 4972 | 3.63 | 1.62 | 24.24 | 7.21 | 4.89 | 2.16 | 0.56 | 2.57 | 0.58 |
| 4345 | 3.63 | 1.68 | 24.21 | 7.46 | 4.89 | 2.24 | 1.11 | 2.15 | 1.14 |
| 5039 | 3.60 | 1.83 | 24.04 | 8.13 | 4.85 | 2.44 | 0.97 | 2.22 | 1.00 |
| 5163 | 3.59 | 4.67 | 23.94 | 20.78 | 4.83 | 6.24 | 0.78 | 2.10 | 0.80 |
| 5040 | 3.57 | 1.27 | 23.86 | 5.64 | 4.81 | 1.69 | 0.94 | 3.36 | 0.96 |
| 4304 | 3.55 | 2.44 | 23.71 | 10.87 | 4.79 | 3.26 | 0.49 | 2.44 | 0.50 |
| 18 | 3.52 | 2.41 | 23.50 | 10.73 | 4.74 | 3.22 | 1.22 | 2.78 | 1.25 |
| 5193 | 3.52 | 6.22 | 23.48 | 27.69 | 4.74 | 8.31 | 1.00 | 2.21 | 1.03 |
| 5077 | 3.51 | 1.71 | 23.42 | 7.59 | 4.73 | 2.28 | 2.91 | 1.91 | 3.01 |
| 5814 | 3.51 | 1.79 | 23.42 | 7.97 | 4.73 | 2.39 | 0.88 | 2.44 | 0.91 |
| 386 | 3.51 | 3.26 | 23.40 | 14.49 | 4.72 | 4.35 | 0.77 | 2.30 | 0.80 |
| 5081 | 3.49 | 1.81 | 23.30 | 8.04 | 4.70 | 2.41 | 0.86 | 2.79 | 0.89 |
| 308 | 3.48 | 1.69 | 23.25 | 7.50 | 4.69 | 2.25 | 0.38 | 2.37 | 0.39 |
| 22 | 3.46 | 1.30 | 23.12 | 5.76 | 4.67 | 1.73 | 0.90 | 3.65 | 0.93 |
| 4338 | 3.42 | 3.00 | 22.83 | 13.34 | 4.61 | 4.00 | 0.68 | 2.43 | 0.71 |
| 3846 | 3.41 | 1.25 | 22.79 | 5.58 | 4.60 | 1.68 | 0.63 | 2.50 | 0.65 |
| 278 | 3.40 | 2.04 | 22.68 | 9.07 | 4.58 | 2.72 | 0.92 | 2.90 | 0.95 |
| 5127 | 3.39 | 1.55 | 22.66 | 6.91 | 4.57 | 2.07 | 0.70 | 2.45 | 0.72 |
| 5029 | 3.39 | 1.80 | 22.62 | 8.00 | 4.57 | 2.40 | 0.42 | 2.51 | 0.43 |
| 5037 | 3.38 | 1.50 | 22.54 | 6.67 | 4.55 | 2.00 | 0.43 | 2.66 | 0.44 |
| 5143 | 3.33 | 1.67 | 22.21 | 7.42 | 4.48 | 2.23 | 1.65 | 3.58 | 1.70 |
| 4983 | 3.31 | 1.90 | 22.09 | 8.46 | 4.46 | 2.54 | 0.60 | 3.28 | 0.62 |
| 4361 | 3.31 | 1.50 | 22.08 | 6.67 | 4.46 | 2.00 | 0.86 | 1.63 | 0.88 |
| 4317 | 3.30 | 3.82 | 22.00 | 17.01 | 4.44 | 5.10 | 1.15 | 2.41 | 1.19 |
| 5080 | 3.27 | 2.62 | 21.80 | 11.67 | 4.40 | 3.50 | 1.05 | 3.25 | 1.09 |
| 23 | 3.26 | 0.85 | 21.76 | 3.78 | 4.39 | 1.14 | 0.79 | 3.25 | 0.82 |
| 4343 | 3.25 | 1.60 | 21.67 | 7.11 | 4.37 | 2.13 | 0.91 | 2.77 | 0.94 |
| 4986 | 3.25 | 3.11 | 21.66 | 13.85 | 4.37 | 4.16 | 0.92 | 1.96 | 0.94 |
| 4311 | 3.24 | 1.76 | 21.65 | 7.85 | 4.37 | 2.36 | 2.50 | 6.05 | 2.58 |
| 5102 | 3.22 | 1.90 | 21.49 | 8.44 | 4.34 | 2.53 | 0.65 | 2.60 | 0.67 |
| 5280 | 3.21 | 3.51 | 21.45 | 15.61 | 4.33 | 4.68 | 2.00 | 6.31 | 2.06 |
| 5185 | 3.19 | 1.80 | 21.32 | 8.00 | 4.30 | 2.40 | 0.47 | 2.63 | 0.49 |
| 964 | 3.18 | 1.42 | 21.26 | 6.33 | 4.29 | 1.90 | 1.54 | 2.13 | 1.59 |
| 4935 | 3.17 | 1.99 | 21.18 | 8.85 | 4.27 | 2.65 | 1.00 | 2.81 | 1.03 |
| 4379 | 3.15 | 2.48 | 21.00 | 11.02 | 4.24 | 3.31 | 0.61 | 1.85 | 0.63 |
| 5052 | 3.13 | 6.63 | 20.92 | 29.49 | 4.22 | 8.85 | 1.14 | 2.00 | 1.17 |
| 5027 | 3.13 | 2.34 | 20.91 | 10.40 | 4.22 | 3.12 | 0.93 | 2.41 | 0.96 |
| 5173 | 3.12 | 1.46 | 20.86 | 6.51 | 4.21 | 1.95 | 2.28 | 5.66 | 2.35 |
| 5055 | 3.12 | 2.16 | 20.81 | 9.61 | 4.20 | 2.88 | 1.05 | 2.09 | 1.08 |
| 5123 | 3.11 | 1.56 | 20.77 | 6.96 | 4.19 | 2.09 | 1.05 | 2.46 | 1.08 |
| 4335 | 3.09 | 1.86 | 20.65 | 8.29 | 4.17 | 2.49 | 1.06 | 2.53 | 1.10 |
| 4936 | 3.09 | 1.58 | 20.64 | 7.03 | 4.17 | 2.11 | 0.91 | 2.54 | 0.94 |
| 5208 | 3.09 | 2.28 | 20.60 | 10.12 | 4.16 | 3.04 | 0.43 | 2.13 | 0.44 |
| 4633 | 3.07 | 1.31 | 20.52 | 5.83 | 4.14 | 1.75 | 0.70 | 1.81 | 0.72 |
| 4359 | 3.06 | 1.64 | 20.42 | 7.30 | 4.12 | 2.19 | 1.00 | 2.44 | 1.03 |
| 289 | 3.05 | 1.43 | 20.39 | 6.35 | 4.11 | 1.90 | 0.60 | 1.93 | 0.62 |
| 4353 | 3.02 | 1.67 | 20.17 | 7.44 | 4.07 | 2.23 | 0.24 | 2.17 | 0.25 |
| 4316 | 3.02 | 1.57 | 20.13 | 7.00 | 4.06 | 2.10 | 0.12 | 1.93 | 0.12 |
| 5210 | 3.01 | 1.56 | 20.12 | 6.95 | 4.06 | 2.09 | 0.99 | 2.26 | 1.02 |
| 4949 | 3.00 | 2.82 | 20.03 | 12.56 | 4.04 | 3.77 | 0.84 | 1.89 | 0.87 |
| 4947 | 2.99 | 1.57 | 19.94 | 7.00 | 4.02 | 2.10 | 0.83 | 2.52 | 0.86 |
| 2838 | 2.98 | 1.35 | 19.87 | 6.01 | 4.01 | 1.80 | 0.69 | 2.52 | 0.71 |
| 378 | 2.97 | 1.75 | 19.81 | 7.77 | 4.00 | 2.33 | 0.57 | 2.70 | 0.58 |
| 5013 | 2.96 | 2.12 | 19.77 | 9.45 | 3.99 | 2.84 | 0.92 | 1.87 | 0.94 |
| 5278 | 2.96 | 1.90 | 19.73 | 8.47 | 3.98 | 2.54 | 0.98 | 3.14 | 1.01 |
| 1471 | 2.95 | 0.98 | 19.71 | 4.37 | 3.98 | 1.31 | 0.86 | 2.15 | 0.89 |
| 4346 | 2.94 | 2.93 | 19.63 | 13.01 | 3.96 | 3.91 | 0.59 | 2.13 | 0.61 |
| 297 | 2.94 | 1.15 | 19.61 | 5.12 | 3.96 | 1.54 | 0.36 | 2.22 | 0.37 |
| 4993 | 2.93 | 1.85 | 19.58 | 8.24 | 3.95 | 2.47 | 0.54 | 2.63 | 0.56 |
| 384 | 2.91 | 1.23 | 19.46 | 5.49 | 3.93 | 1.65 | 1.35 | 3.15 | 1.40 |
| 5062 | 2.91 | 1.51 | 19.41 | 6.73 | 3.92 | 2.02 | 0.94 | 2.22 | 0.96 |
| 4945 | 2.90 | 1.50 | 19.39 | 6.67 | 3.91 | 2.00 | 0.96 | 2.42 | 0.99 |

As shown in TABLE 10, “DNA FC Input” represents the fold change of counts per million (CPM) values, normalized by Trimmed Mean of M-values (TMM) of capsid DNA relative to viral input in cardiac muscle tissue. “RNA FC Input” represents the fold change of CPM values (normalized by TMM) of capsid RNA relative to viral input in cardiac muscle tissue. “DNA FC AAV9” represents the fold change of CPM values (normalized by TMM and to viral input) of capsid DNA relative to wild-type AAV9 in cardiac muscle tissue. “RNA FC AAV9” represents the fold change of CPM values (normalized by TMM and to viral input) of capsid RNA relative to wild-type AAV9 in cardiac muscle tissue. “DNA FC AAV5” represents the fold change of CPM values (normalized by TMM and to viral input) of capsid DNA relative to wild-type AAV5 in cardiac muscle tissue. “RNA FC AAV5” represents the fold change of CPM values (normalized by TMM and to viral input) of capsid RNA relative to wild-type AAV5 in cardiac muscle tissue. “Liver DNA FC AAV5” represents the fold change of CPM values (normalized by TMM and to viral input) of capsid DNA relative to wild-type AAV5 in the liver. “Liver RNA FC AAV5” represents the fold change of CPM values (normalized by TMM and to viral input) of capsid RNA relative to wild-type AAV5 in the liver. “Liver DNA FC AAV9” represents the fold change of CPM values (normalized by TMM and to viral input) of capsid DNA relative to wild-type AAV9 in the liver. “Liver RNA FC AAV9” represents the fold change of CPM values (normalized by TMM and to viral input) of capsid RNA relative to wild-type AAV9 in the liver.

### Example 8

### Secondary Screen Results to Identify Skeletal Muscle Tissue-Tropic Recombinant AAV Capsids

This example describes secondary screen results to identify skeletal muscle tissue-tropic recombinant AAV capsids. The secondary screen and enrichment analysis were performed as described in EXAMPLE 5. Recombinant AAV capsids containing variant 581-589 regions that were enriched in skeletal muscle tissue and exhibited functional transduction in skeletal muscle tissue were identified. TABLE 11 provides enrichment data for the 581-589 region sequences of the top 100 skeletal muscle tissue-tropic AAV capsids, as identified in the secondary screen, and the 15 wild type spike-in controls. Superior capsids were selected based on parameters including relative DNA and RNA abundance in skeletal muscle tissue, as well as additional metrics shown in TABLE 11, including liver DNA fold change over AAV5, liver RNA fold change over AAV5, liver DNA fold change over AAV9, and liver RNA fold change over AAV9.

TABLE 11
Secondary Screen Results for Skeletal Muscle Tissue-Tropic Capsids

| | DNA | RNA | DNA | RNA | DNA | RNA | Liver | Liver | Liver |
|---|---|---|---|---|---|---|---|---|---|
| SEQ | FC | FC | FC | FC | FC | FC | DNA FC | RNA FC | DNA FC |
| ID NO | Input | Input | AAV9 | AAV9 | AAV5 | AAV5 | AAV5 | AAV5 | AAV9 |
| 3472 | 7.59 | 29.12 | 34.42 | 48.42 | 8.24 | 4.67 | 0.62 | 2.70 | 0.64 |
| 3297 | 6.62 | 0.29 | 30.01 | 0.49 | 7.18 | 0.05 | 0.77 | 2.41 | 0.79 |
| 2661 | 5.62 | 0.14 | 25.49 | 0.24 | 6.10 | 0.02 | 1.24 | 4.51 | 1.27 |
| 4545 | 5.05 | 0.27 | 22.90 | 0.45 | 5.48 | 0.04 | 0.43 | 2.03 | 0.45 |
| 348 | 4.77 | 0.15 | 21.66 | 0.25 | 5.18 | 0.02 | 2.85 | 7.14 | 2.94 |
| 18 | 4.37 | 0.23 | 19.82 | 0.38 | 4.74 | 0.04 | 1.22 | 2.78 | 1.25 |
| 4349 | 4.18 | 0.14 | 18.97 | 0.23 | 4.54 | 0.02 | 0.46 | 2.40 | 0.47 |
| 293 | 4.15 | 0.55 | 18.81 | 0.91 | 4.50 | 0.09 | 0.79 | 3.06 | 0.81 |
| 5017 | 4.14 | 0.19 | 18.76 | 0.32 | 4.49 | 0.03 | 1.94 | 2.76 | 2.00 |
| 4955 | 4.09 | 0.14 | 18.54 | 0.24 | 4.44 | 0.02 | 0.38 | 2.70 | 0.39 |
| 4366 | 3.80 | 0.23 | 17.24 | 0.39 | 4.13 | 0.04 | 1.20 | 2.86 | 1.24 |
| 5092 | 3.67 | 0.28 | 16.63 | 0.47 | 3.98 | 0.05 | 0.36 | 1.88 | 0.37 |
| 210 | 3.61 | 0.57 | 16.38 | 0.95 | 3.92 | 0.09 | 1.40 | 3.81 | 1.44 |
| 4059 | 3.52 | 0.13 | 15.95 | 0.22 | 3.82 | 0.02 | 0.72 | 2.34 | 0.75 |
| 2536 | 3.49 | 0.23 | 15.85 | 0.37 | 3.80 | 0.04 | 0.24 | 2.16 | 0.25 |
| 5206 | 3.43 | 0.32 | 15.55 | 0.53 | 3.72 | 0.05 | 0.63 | 2.58 | 0.65 |
| 1971 | 3.31 | 0.68 | 15.00 | 1.13 | 3.59 | 0.11 | 0.62 | 1.84 | 0.64 |
| 5607 | 3.26 | 0.32 | 14.79 | 0.54 | 3.54 | 0.05 | 18.66 | 32.87 | 19.25 |
| 262 | 3.24 | 0.24 | 14.70 | 0.40 | 3.52 | 0.04 | 1.01 | 4.45 | 1.04 |
| 4343 | 3.20 | 1.16 | 14.50 | 1.93 | 3.47 | 0.19 | 0.91 | 2.77 | 0.94 |
| 4995 | 3.16 | 0.27 | 14.33 | 0.45 | 3.43 | 0.04 | 0.73 | 1.95 | 0.76 |
| 4009 | 3.12 | 0.56 | 14.15 | 0.93 | 3.39 | 0.09 | 0.71 | 2.51 | 0.74 |
| 5190 | 3.09 | 0.30 | 14.02 | 0.50 | 3.36 | 0.05 | 1.15 | 2.90 | 1.18 |
| 724 | 3.08 | 0.19 | 13.99 | 0.32 | 3.35 | 0.03 | 0.17 | 1.53 | 0.17 |
| 4288 | 3.08 | 0.09 | 13.96 | 0.16 | 3.34 | 0.02 | 1.21 | 3.25 | 1.25 |
| 4973 | 3.07 | 0.79 | 13.94 | 1.31 | 3.34 | 0.13 | 0.59 | 2.63 | 0.61 |
| 4314 | 3.06 | 0.18 | 13.88 | 0.30 | 3.32 | 0.03 | 0.49 | 2.21 | 0.50 |
| 1561 | 3.05 | 0.60 | 13.82 | 1.00 | 3.31 | 0.10 | 1.56 | 3.81 | 1.61 |
| 5147 | 3.01 | 0.15 | 13.68 | 0.25 | 3.27 | 0.02 | 0.54 | 2.71 | 0.56 |
| 4238 | 3.00 | 0.10 | 13.61 | 0.17 | 3.26 | 0.02 | 0.15 | 1.86 | 0.16 |
| 4961 | 2.99 | 0.64 | 13.57 | 1.06 | 3.25 | 0.10 | 1.02 | 2.25 | 1.05 |
| 4964 | 2.97 | 0.23 | 13.49 | 0.39 | 3.23 | 0.04 | 1.28 | 3.69 | 1.32 |
| 5527 | 2.96 | 0.87 | 13.45 | 1.45 | 3.22 | 0.14 | 1.35 | 3.25 | 1.39 |
| 3283 | 2.92 | 23.44 | 13.24 | 38.97 | 3.17 | 3.76 | 0.29 | 1.84 | 0.30 |
| 5075 | 2.86 | 0.12 | 12.97 | 0.20 | 3.11 | 0.02 | 1.52 | 2.70 | 1.57 |
| 4355 | 2.86 | 0.24 | 12.97 | 0.39 | 3.10 | 0.04 | 1.05 | 2.51 | 1.08 |
| 5157 | 2.85 | 0.19 | 12.93 | 0.32 | 3.09 | 0.03 | 5.00 | 13.10 | 5.15 |
| 5159 | 2.85 | 0.33 | 12.92 | 0.54 | 3.09 | 0.05 | 1.03 | 2.57 | 1.06 |
| 4363 | 2.82 | 0.53 | 12.81 | 0.88 | 3.07 | 0.08 | 0.45 | 2.20 | 0.46 |
| 4317 | 2.78 | 0.54 | 12.62 | 0.89 | 3.02 | 0.09 | 1.15 | 2.41 | 1.19 |
| 4963 | 2.77 | 0.18 | 12.58 | 0.30 | 3.01 | 0.03 | 3.75 | 10.01 | 3.87 |
| 5055 | 2.76 | 0.21 | 12.53 | 0.34 | 3.00 | 0.03 | 1.05 | 2.09 | 1.08 |
| 5125 | 2.73 | 0.25 | 12.38 | 0.42 | 2.96 | 0.04 | 0.42 | 2.88 | 0.43 |
| 4108 | 2.72 | 0.43 | 12.33 | 0.71 | 2.95 | 0.07 | 0.89 | 3.87 | 0.92 |
| 308 | 2.69 | 0.60 | 12.22 | 1.00 | 2.92 | 0.10 | 0.38 | 2.37 | 0.39 |
| 4311 | 2.68 | 0.14 | 12.17 | 0.23 | 2.91 | 0.02 | 2.50 | 6.05 | 2.58 |
| 5060 | 2.66 | 0.39 | 12.06 | 0.64 | 2.89 | 0.06 | 0.41 | 2.62 | 0.42 |
| 2948 | 2.65 | 0.55 | 12.01 | 0.91 | 2.88 | 0.09 | 1.46 | 4.76 | 1.51 |
| 3821 | 2.65 | 0.36 | 12.00 | 0.60 | 2.87 | 0.06 | 1.08 | 2.52 | 1.12 |
| 5138 | 2.64 | 422.81 | 11.96 | 702.96 | 2.86 | 67.77 | 0.57 | 2.04 | 0.59 |
| 4354 | 2.64 | 0.33 | 11.96 | 0.54 | 2.86 | 0.05 | 1.91 | 1.81 | 1.97 |
| 4353 | 2.63 | 0.37 | 11.93 | 0.62 | 2.86 | 0.06 | 0.24 | 2.17 | 0.25 |
| 5030 | 2.62 | 0.09 | 11.88 | 0.15 | 2.84 | 0.01 | 3.24 | 8.80 | 3.34 |
| 4346 | 2.59 | 0.27 | 11.77 | 0.46 | 2.82 | 0.04 | 0.59 | 2.13 | 0.61 |
| 5283 | 2.57 | 0.09 | 11.65 | 0.15 | 2.79 | 0.01 | 5.79 | 11.85 | 5.97 |
| 4632 | 2.55 | 0.19 | 11.55 | 0.32 | 2.76 | 0.03 | 0.57 | 2.33 | 0.59 |
| 294 | 2.53 | 0.38 | 11.48 | 0.63 | 2.75 | 0.06 | 0.92 | 1.86 | 0.95 |
| 386 | 2.50 | 0.20 | 11.35 | 0.33 | 2.72 | 0.03 | 0.77 | 2.30 | 0.80 |
| 4943 | 2.49 | 0.35 | 11.31 | 0.59 | 2.71 | 0.06 | 0.51 | 2.85 | 0.52 |
| 1391 | 2.49 | 2.22 | 11.31 | 3.70 | 2.71 | 0.36 | 0.58 | 3.88 | 0.60 |
| 5078 | 2.48 | 0.43 | 11.26 | 0.71 | 2.69 | 0.07 | 0.79 | 2.10 | 0.81 |
| 3299 | 2.48 | 0.87 | 11.26 | 1.44 | 2.69 | 0.14 | 0.34 | 2.91 | 0.35 |
| 2897 | 2.47 | 0.55 | 11.21 | 0.92 | 2.68 | 0.09 | 1.23 | 3.90 | 1.27 |
| 1537 | 2.46 | 1.24 | 11.16 | 2.06 | 2.67 | 0.20 | 0.40 | 3.00 | 0.41 |
| 5255 | 2.46 | 0.36 | 11.15 | 0.60 | 2.67 | 0.06 | 1.68 | 4.90 | 1.73 |
| 141 | 2.45 | 0.63 | 11.14 | 1.04 | 2.67 | 0.10 | 1.27 | 3.85 | 1.31 |
| 4345 | 2.45 | 0.12 | 11.12 | 0.19 | 2.66 | 0.02 | 1.11 | 2.15 | 1.14 |
| 5038 | 2.44 | 0.43 | 11.06 | 0.72 | 2.65 | 0.07 | 0.45 | 2.31 | 0.47 |
| 5153 | 2.44 | 0.17 | 11.06 | 0.29 | 2.65 | 0.03 | 0.63 | 3.87 | 0.65 |
| 1181 | 2.43 | 0.54 | 11.03 | 0.90 | 2.64 | 0.09 | 0.57 | 3.51 | 0.58 |
| 307 | 2.42 | 0.20 | 10.98 | 0.33 | 2.63 | 0.03 | 0.95 | 3.49 | 0.98 |
| 5039 | 2.42 | 0.19 | 10.98 | 0.31 | 2.63 | 0.03 | 0.97 | 2.22 | 1.00 |
| 1204 | 2.41 | 0.37 | 10.92 | 0.61 | 2.61 | 0.06 | 1.13 | 2.72 | 1.16 |
| 4404 | 2.41 | 0.22 | 10.92 | 0.36 | 2.61 | 0.04 | 0.25 | 1.64 | 0.26 |
| 5106 | 2.40 | 0.28 | 10.89 | 0.47 | 2.61 | 0.04 | 0.59 | 2.31 | 0.60 |
| 2779 | 2.40 | 0.51 | 10.89 | 0.85 | 2.61 | 0.08 | 1.56 | 4.50 | 1.61 |
| 4952 | 2.40 | 0.09 | 10.89 | 0.15 | 2.61 | 0.01 | 0.96 | 2.80 | 0.99 |
| 278 | 2.40 | 6.97 | 10.87 | 11.60 | 2.60 | 1.12 | 0.92 | 2.90 | 0.95 |
| 1824 | 2.40 | 1.12 | 10.87 | 1.86 | 2.60 | 0.18 | 1.76 | 5.93 | 1.82 |
| 5116 | 2.39 | 0.23 | 10.83 | 0.39 | 2.59 | 0.04 | 0.48 | 2.64 | 0.49 |
| 4304 | 2.37 | 0.16 | 10.75 | 0.26 | 2.57 | 0.02 | 0.49 | 2.44 | 0.50 |
| 4295 | 2.36 | 0.06 | 10.73 | 0.09 | 2.57 | 0.01 | 1.19 | 2.89 | 1.23 |
| 3179 | 2.36 | 0.41 | 10.69 | 0.68 | 2.56 | 0.07 | 0.82 | 2.94 | 0.85 |
| 3306 | 2.34 | 0.48 | 10.64 | 0.80 | 2.55 | 0.08 | 0.49 | 2.24 | 0.51 |
| 98 | 2.34 | 0.46 | 10.64 | 0.76 | 2.55 | 0.07 | 1.50 | 3.46 | 1.54 |
| 1872 | 2.34 | 0.50 | 10.63 | 0.84 | 2.55 | 0.08 | 0.54 | 2.47 | 0.56 |
| 5037 | 2.34 | 0.26 | 10.62 | 0.43 | 2.54 | 0.04 | 0.43 | 2.66 | 0.44 |
| 1268 | 2.34 | 0.81 | 10.60 | 1.35 | 2.54 | 0.13 | 0.91 | 1.97 | 0.94 |
| 1634 | 2.34 | 2.46 | 10.60 | 4.09 | 2.54 | 0.39 | 1.55 | 3.50 | 1.60 |
| 1060 | 2.33 | 0.23 | 10.57 | 0.38 | 2.53 | 0.04 | 1.55 | 2.63 | 1.59 |
| 4941 | 2.33 | 0.27 | 10.55 | 0.44 | 2.53 | 0.04 | 1.00 | 3.40 | 1.04 |
| 4361 | 2.32 | 0.27 | 10.53 | 0.44 | 2.52 | 0.04 | 0.86 | 1.63 | 0.88 |
| 4981 | 2.31 | 0.37 | 10.49 | 0.61 | 2.51 | 0.06 | 1.97 | 4.47 | 2.03 |
| 4359 | 2.31 | 0.22 | 10.47 | 0.37 | 2.51 | 0.04 | 1.00 | 2.44 | 1.03 |
| 5274 | 2.31 | 0.09 | 10.47 | 0.15 | 2.51 | 0.01 | 2.68 | 5.94 | 2.76 |
| 5816 | 2.30 | 0.80 | 10.44 | 1.33 | 2.50 | 0.13 | 2.09 | 5.41 | 2.16 |
| 2842 | 2.30 | 0.28 | 10.44 | 0.47 | 2.50 | 0.05 | 0.63 | 2.36 | 0.65 |
| 1934 | 2.30 | 28.74 | 10.43 | 47.78 | 2.50 | 4.61 | 1.26 | 2.95 | 1.30 |
| 5817 | 2.30 | 10.17 | 10.43 | 16.91 | 2.50 | 1.63 | 2.49 | 5.43 | 2.57 |
| 3998 | 2.29 | 0.66 | 10.41 | 1.10 | 2.49 | 0.11 | 2.54 | 5.64 | 2.62 |

As shown in TABLE 11, “DNA FC Input” represents the fold change of counts per million (CPM) values, normalized by Trimmed Mean of M-values (TMM) of capsid DNA relative to viral input in skeletal muscle tissue. “RNA FC Input” represents the fold change of CPM values (normalized by TMMN/) of capsid RNA relative to viral input in skeletal muscle tissue. “DNA FC AAV9” represents the fold change of CPM values (normalized by TMM and to viral input) of capsid DNA relative to wild-type AAV9 in skeletal muscle tissue. “RNA FC AAV9” represents the fold change of CPM values (normalized by TMM and to viral input) of capsid RNA relative to wild-type AAV9 in skeletal muscle tissue. “DNA FC AAV5” represents the fold change of CPM values (normalized by TMVM and to viral input) of capsid DNA relative to wild-type AAV5 in skeletal muscle tissue. “RNA FC AAV5” represents the fold change of CPM values (normalized by TMMN and to viral input) of capsid RNA relative to wild-type AAV5 in skeletal muscle tissue. “Liver DNA FC AAV5” represents the fold change of CPM values (normalized by TMMN and to viral input) of capsid DNA relative to wild-type AAV5 in the liver. “Liver RNA FC AAV5” represents the fold change of CPM values (normalized by TMMN and to viral input) of capsid RNA relative to wild-type AAV5 in the liver. “Liver DNA FC AAV9” represents the fold change of CPM values (normalized by TMM and to viral input) of capsid DNA relative to wild-type AAV9 in the liver. “Liver RNA FC AAV9” represents the fold change of CPM values (normalized by TMM and to viral input) of capsid RNA relative to wild-type AAV9 in the liver.

### Example 9

### Secondary Screen Results to Identify Muscle Tissue-Tropic Recombinant AAV Capsids

This example describes secondary screen results to identify muscle tissue-tropic recombinant AAV capsids. The secondary screen and enrichment analysis were performed as described in EXAMPLE 5. Recombinant AAV capsids containing variant 581-589 regions that were enriched in muscle tissue (including cardiac tissue and skeletal muscle tissue) and exhibited functional transduction in muscle tissue were identified. TABLE 12 provides enrichment data for the 581-589 region sequences of the top 100 muscle tissue-tropic AAV capsids, as identified in the secondary screen, and the 15 wild type spike-in controls. Superior capsids were selected based on parameters including relative DNA and RNA abundance in muscle tissue, as well as additional metrics shown in TABLE 12, including liver DNA fold change over AAV5, liver RNA fold change over AAV5, liver DNA fold change over AAV9, and liver RNA fold change over AAV9.

TABLE 12
Secondary Screen Results for Muscle Tissue-Tropic Capsids

| | DNA | RNA | DNA | RNA | DNA | RNA | Liver | Liver | Liver |
|---|---|---|---|---|---|---|---|---|---|
| SEQ | FC | FC | FC | FC | FC | FC | DNA FC | RNA FC | DNA FC |
| ID NO | Input | Input | AAV9 | AAV9 | AAV5 | AAV5 | AAV5 | AAV5 | AAV9 |
| 348 | 8.14 | 7.62 | 48.43 | 22.93 | 10.32 | 3.29 | 2.85 | 7.14 | 2.94 |
| 2536 | 6.64 | 1.67 | 39.50 | 5.01 | 8.42 | 0.72 | 0.24 | 2.16 | 0.25 |
| 5607 | 5.31 | 1.85 | 31.58 | 5.57 | 6.73 | 0.80 | 18.66 | 32.87 | 19.25 |
| 4955 | 5.04 | 1.61 | 29.97 | 4.85 | 6.39 | 0.70 | 0.38 | 2.70 | 0.39 |
| 5017 | 4.94 | 1.97 | 29.39 | 5.93 | 6.27 | 0.85 | 1.94 | 2.76 | 2.00 |
| 4349 | 4.88 | 1.69 | 29.02 | 5.08 | 6.19 | 0.73 | 0.46 | 2.40 | 0.47 |
| 293 | 4.72 | 3.09 | 28.06 | 9.28 | 5.98 | 1.33 | 0.79 | 3.06 | 0.81 |
| 1576 | 4.62 | 1.14 | 27.51 | 3.43 | 5.86 | 0.49 | 0.99 | 2.91 | 1.02 |
| 2661 | 4.44 | 1.46 | 26.39 | 4.40 | 5.63 | 0.63 | 1.24 | 4.51 | 1.27 |
| 4964 | 4.44 | 1.89 | 26.39 | 5.68 | 5.63 | 0.82 | 1.28 | 3.69 | 1.32 |
| 4314 | 4.33 | 1.95 | 25.78 | 5.87 | 5.50 | 0.84 | 0.49 | 2.21 | 0.50 |
| 4995 | 4.33 | 2.34 | 25.77 | 7.04 | 5.49 | 1.01 | 0.73 | 1.95 | 0.76 |
| 4366 | 4.32 | 1.48 | 25.68 | 4.47 | 5.48 | 0.64 | 1.20 | 2.86 | 1.24 |
| 4545 | 4.19 | 1.38 | 24.94 | 4.16 | 5.32 | 0.60 | 0.43 | 2.03 | 0.45 |
| 4961 | 4.09 | 1.35 | 24.32 | 4.07 | 5.19 | 0.58 | 1.02 | 2.25 | 1.05 |
| 5206 | 4.05 | 2.26 | 24.10 | 6.81 | 5.14 | 0.98 | 0.63 | 2.58 | 0.65 |
| 5092 | 4.04 | 1.27 | 24.06 | 3.81 | 5.13 | 0.55 | 0.36 | 1.88 | 0.37 |
| 3472 | 4.03 | 9.18 | 23.96 | 27.61 | 5.11 | 3.96 | 0.62 | 2.70 | 0.64 |
| 5075 | 4.01 | 2.50 | 23.83 | 7.51 | 5.08 | 1.08 | 1.52 | 2.70 | 1.57 |
| 4059 | 3.96 | 1.44 | 23.57 | 4.35 | 5.03 | 0.62 | 0.72 | 2.34 | 0.75 |
| 4354 | 3.94 | 1.54 | 23.46 | 4.64 | 5.00 | 0.67 | 1.91 | 1.81 | 1.97 |
| 5060 | 3.94 | 2.17 | 23.43 | 6.53 | 5.00 | 0.94 | 0.41 | 2.62 | 0.42 |
| 4288 | 3.93 | 2.30 | 23.39 | 6.91 | 4.99 | 0.99 | 1.21 | 3.25 | 1.25 |
| 4952 | 3.90 | 2.96 | 23.19 | 8.91 | 4.94 | 1.28 | 0.96 | 2.80 | 0.99 |
| 5138 | 3.89 | 125.02 | 23.11 | 376.10 | 4.93 | 53.95 | 0.57 | 2.04 | 0.59 |
| 5283 | 3.77 | 1.47 | 22.45 | 4.41 | 4.79 | 0.63 | 5.79 | 11.85 | 5.97 |
| 18 | 3.74 | 1.79 | 22.25 | 5.38 | 4.74 | 0.77 | 1.22 | 2.78 | 1.25 |
| 5030 | 3.67 | 2.29 | 21.85 | 6.88 | 4.66 | 0.99 | 3.24 | 8.80 | 3.34 |
| 3297 | 3.67 | 1.54 | 21.84 | 4.63 | 4.66 | 0.66 | 0.77 | 2.41 | 0.79 |
| 4295 | 3.63 | 1.57 | 21.57 | 4.72 | 4.60 | 0.68 | 1.19 | 2.89 | 1.23 |
| 4363 | 3.61 | 1.86 | 21.47 | 5.61 | 4.58 | 0.80 | 0.45 | 2.20 | 0.46 |
| 4963 | 3.60 | 1.10 | 21.41 | 3.31 | 4.56 | 0.47 | 3.75 | 10.01 | 3.87 |
| 4973 | 3.59 | 1.55 | 21.35 | 4.67 | 4.55 | 0.67 | 0.59 | 2.63 | 0.61 |
| 5159 | 3.55 | 2.21 | 21.09 | 6.63 | 4.50 | 0.95 | 1.03 | 2.57 | 1.06 |
| 4960 | 3.50 | 1.50 | 20.83 | 4.52 | 4.44 | 0.65 | 0.59 | 2.26 | 0.61 |
| 4938 | 3.47 | 1.67 | 20.63 | 5.03 | 4.40 | 0.72 | 0.68 | 2.83 | 0.70 |
| 5157 | 3.47 | 1.20 | 20.62 | 3.61 | 4.40 | 0.52 | 5.00 | 13.10 | 5.15 |
| 5023 | 3.45 | 1.06 | 20.55 | 3.18 | 4.38 | 0.46 | 0.46 | 2.39 | 0.48 |
| 4969 | 3.40 | 1.20 | 20.21 | 3.61 | 4.31 | 0.52 | 0.29 | 2.49 | 0.30 |
| 5215 | 3.34 | 2.05 | 19.84 | 6.17 | 4.23 | 0.88 | 1.53 | 5.08 | 1.58 |
| 4345 | 3.32 | 1.23 | 19.76 | 3.70 | 4.21 | 0.53 | 1.11 | 2.15 | 1.14 |
| 5039 | 3.29 | 1.36 | 19.60 | 4.09 | 4.18 | 0.59 | 0.97 | 2.22 | 1.00 |
| 308 | 3.28 | 1.38 | 19.50 | 4.14 | 4.16 | 0.59 | 0.38 | 2.37 | 0.39 |
| 4304 | 3.25 | 1.79 | 19.31 | 5.38 | 4.12 | 0.77 | 0.49 | 2.44 | 0.50 |
| 386 | 3.25 | 2.38 | 19.30 | 7.17 | 4.12 | 1.03 | 0.77 | 2.30 | 0.80 |
| 4934 | 3.24 | 1.46 | 19.29 | 4.41 | 4.11 | 0.63 | 0.85 | 2.00 | 0.88 |
| 4343 | 3.23 | 1.47 | 19.23 | 4.43 | 4.10 | 0.64 | 0.91 | 2.77 | 0.94 |
| 5163 | 3.22 | 3.36 | 19.14 | 10.11 | 4.08 | 1.45 | 0.78 | 2.10 | 0.80 |
| 5081 | 3.17 | 1.40 | 18.86 | 4.22 | 4.02 | 0.61 | 0.86 | 2.79 | 0.89 |
| 4317 | 3.16 | 2.88 | 18.81 | 8.68 | 4.01 | 1.24 | 1.15 | 2.41 | 1.19 |
| 5814 | 3.15 | 1.40 | 18.72 | 4.23 | 3.99 | 0.61 | 0.88 | 2.44 | 0.91 |
| 278 | 3.14 | 3.45 | 18.66 | 10.37 | 3.98 | 1.49 | 0.92 | 2.90 | 0.95 |
| 5040 | 3.12 | 0.95 | 18.55 | 2.85 | 3.95 | 0.41 | 0.94 | 3.36 | 0.96 |
| 4972 | 3.11 | 1.47 | 18.50 | 4.41 | 3.94 | 0.63 | 0.56 | 2.57 | 0.58 |
| 5037 | 3.11 | 1.15 | 18.48 | 3.45 | 3.94 | 0.49 | 0.43 | 2.66 | 0.44 |
| 4311 | 3.10 | 1.30 | 18.43 | 3.91 | 3.93 | 0.56 | 2.50 | 6.05 | 2.58 |
| 5127 | 3.08 | 1.18 | 18.33 | 3.56 | 3.91 | 0.51 | 0.70 | 2.45 | 0.72 |
| 5193 | 3.07 | 4.58 | 18.28 | 13.79 | 3.90 | 1.98 | 1.00 | 2.21 | 1.03 |
| 4361 | 3.05 | 1.15 | 18.15 | 3.45 | 3.87 | 0.50 | 0.86 | 1.63 | 0.88 |
| 5077 | 3.04 | 1.24 | 18.11 | 3.73 | 3.86 | 0.54 | 2.91 | 1.91 | 3.01 |
| 5029 | 3.03 | 1.35 | 18.04 | 4.05 | 3.85 | 0.58 | 0.42 | 2.51 | 0.43 |
| 5055 | 3.03 | 1.60 | 18.00 | 4.82 | 3.84 | 0.69 | 1.05 | 2.09 | 1.08 |
| 5143 | 3.01 | 1.23 | 17.88 | 3.69 | 3.81 | 0.53 | 1.65 | 3.58 | 1.70 |
| 5080 | 2.98 | 1.91 | 17.72 | 5.74 | 3.78 | 0.82 | 1.05 | 3.25 | 1.09 |
| 4983 | 2.94 | 1.48 | 17.51 | 4.46 | 3.73 | 0.64 | 0.60 | 3.28 | 0.62 |
| 5190 | 2.93 | 1.19 | 17.45 | 3.59 | 3.72 | 0.51 | 1.15 | 2.90 | 1.18 |
| 4353 | 2.92 | 1.30 | 17.37 | 3.91 | 3.70 | 0.56 | 0.24 | 2.17 | 0.25 |
| 4338 | 2.92 | 2.18 | 17.35 | 6.56 | 3.70 | 0.94 | 0.68 | 2.43 | 0.71 |
| 4379 | 2.91 | 1.81 | 17.32 | 5.44 | 3.69 | 0.78 | 0.61 | 1.85 | 0.63 |
| 5173 | 2.89 | 1.10 | 17.19 | 3.32 | 3.66 | 0.48 | 2.28 | 5.66 | 2.35 |
| 5052 | 2.88 | 4.80 | 17.12 | 14.43 | 3.65 | 2.07 | 1.14 | 2.00 | 1.17 |
| 3846 | 2.87 | 0.98 | 17.09 | 2.94 | 3.64 | 0.42 | 0.63 | 2.50 | 0.65 |
| 4986 | 2.87 | 2.35 | 17.07 | 7.07 | 3.64 | 1.01 | 0.92 | 1.96 | 0.94 |
| 4935 | 2.87 | 1.47 | 17.06 | 4.42 | 3.64 | 0.63 | 1.00 | 2.81 | 1.03 |
| 4359 | 2.86 | 1.24 | 17.04 | 3.72 | 3.63 | 0.53 | 1.00 | 2.44 | 1.03 |
| 4346 | 2.85 | 2.17 | 16.96 | 6.52 | 3.62 | 0.94 | 0.59 | 2.13 | 0.61 |
| 262 | 2.85 | 0.84 | 16.95 | 2.53 | 3.61 | 0.36 | 1.01 | 4.45 | 1.04 |
| 289 | 2.83 | 1.16 | 16.84 | 3.50 | 3.59 | 0.50 | 0.60 | 1.93 | 0.62 |
| 22 | 2.82 | 1.05 | 16.79 | 3.16 | 3.58 | 0.45 | 0.90 | 3.65 | 0.93 |
| 5280 | 2.82 | 2.53 | 16.75 | 7.61 | 3.57 | 1.09 | 2.00 | 6.31 | 2.06 |
| 5210 | 2.82 | 1.27 | 16.75 | 3.82 | 3.57 | 0.55 | 0.99 | 2.26 | 1.02 |
| 5027 | 2.81 | 1.80 | 16.73 | 5.41 | 3.57 | 0.78 | 0.93 | 2.41 | 0.96 |
| 5185 | 2.80 | 1.33 | 16.66 | 3.99 | 3.55 | 0.57 | 0.47 | 2.63 | 0.49 |
| 4009 | 2.79 | 1.18 | 16.59 | 3.56 | 3.54 | 0.51 | 0.71 | 2.51 | 0.74 |
| 5102 | 2.78 | 1.42 | 16.54 | 4.26 | 3.53 | 0.61 | 0.65 | 2.60 | 0.67 |
| 5123 | 2.78 | 1.25 | 16.53 | 3.75 | 3.52 | 0.54 | 1.05 | 2.46 | 1.08 |
| 294 | 2.77 | 1.07 | 16.47 | 3.21 | 3.51 | 0.46 | 0.92 | 1.86 | 0.95 |
| 4949 | 2.76 | 2.20 | 16.42 | 6.62 | 3.50 | 0.95 | 0.84 | 1.89 | 0.87 |
| 5208 | 2.75 | 1.66 | 16.34 | 4.99 | 3.48 | 0.72 | 0.43 | 2.13 | 0.44 |
| 4633 | 2.75 | 0.97 | 16.33 | 2.93 | 3.48 | 0.42 | 0.70 | 1.81 | 0.72 |
| 4238 | 2.74 | 1.01 | 16.31 | 3.05 | 3.48 | 0.44 | 0.15 | 1.86 | 0.16 |
| 5026 | 2.70 | 4.30 | 16.07 | 12.94 | 3.43 | 1.86 | 1.22 | 3.16 | 1.26 |
| 23 | 2.70 | 0.84 | 16.04 | 2.52 | 3.42 | 0.36 | 0.79 | 3.25 | 0.82 |
| 378 | 2.69 | 1.40 | 16.03 | 4.22 | 3.42 | 0.61 | 0.57 | 2.70 | 0.58 |
| 4316 | 2.68 | 1.17 | 15.93 | 3.51 | 3.40 | 0.50 | 0.12 | 1.93 | 0.12 |
| 964 | 2.66 | 1.29 | 15.83 | 3.89 | 3.38 | 0.56 | 1.54 | 2.13 | 1.59 |
| 5062 | 2.66 | 1.14 | 15.83 | 3.43 | 3.37 | 0.49 | 0.94 | 2.22 | 0.96 |
| 4290 | 2.65 | 1.22 | 15.74 | 3.68 | 3.35 | 0.53 | 0.59 | 1.98 | 0.61 |
| 4943 | 2.64 | 3.00 | 15.68 | 9.04 | 3.34 | 1.30 | 0.51 | 2.85 | 0.52 |
| 4947 | 2.63 | 1.25 | 15.62 | 3.75 | 3.33 | 0.54 | 0.83 | 2.52 | 0.86 |

As shown in TABLE 12, “DNA FC Input” represents the fold change of counts per million (CPM) values, normalized by Trimmed Mean of M-values (TMM) of capsid DNA relative to viral input in muscle tissue. “RNA FC Input” represents the fold change of CPM values (normalized by TMM) of capsid RNA relative to viral input in muscle tissue. “DNA FC AAV9” represents the fold change of CPM values (normalized by TMM and to viral input) of capsid DNA relative to wild-type AAV9 in muscle tissue. “RNA FC AAV9” represents the fold change of CPM values (normalized by TMM and to viral input) of capsid RNA relative to wild-type AAV9 in muscle tissue. “DNA FC AAV5” represents the fold change of CPM values (normalized by TMM and to viral input) of capsid DNA relative to wild-type AAV5 in muscle tissue. “RNA FC AAV5” represents the fold change of CPM values (normalized by TMM and to viral input) of capsid RNA relative to wild-type AAV5 in muscle tissue. “Liver DNA FC AAV5” represents the fold change of CPM values (normalized by TMM and to viral input) of capsid DNA relative to wild-type AAV5 in the liver. “Liver RNA FC AAV5” represents the fold change of CPM values (normalized by TMM and to viral input) of capsid RNA relative to wild-type AAV5 in the liver. “Liver DNA FC AAV9” represents the fold change of CPM values (normalized by TMM and to viral input) of capsid DNA relative to wild-type AAV9 in the liver. “Liver RNA FC AAV9” represents the fold change of CPM values (normalized by TMM and to viral input) of capsid RNA relative to wild-type AAV9 in the liver.

### Example 10

### Screening of AAV5 Capsid Variants in Mice

This example describes screening of recombinant AAV capsid candidates with variant 581-589 regions in mice. The goal of this screen was to identify capsid candidates with corresponding tissue specificity in both mice and non-human primates (NHPs) for use as a research tool in mice. The 6,000 AAV capsid variants library using the methods described and administered to NHPs in EXAMPLE 5 was administered to mice. Fifteen wild type spike-in controls representing wild type AAV1, AAV2, AAV5, AAV9, and AAV PHP.B, each at 1×, 10×, and 100× concentrations, along with 100 AAV capsids with stop codons in the 581-589 region, were also administered. Tissue-specific accumulation of each capsid was evaluated by quantifying capsid DNA in heart and CNS tissues. Barcode sequences for each capsid were extracted from raw sequencing data after filtering based on sequencing quality (Q30) and exact match to the reference library of 48,090 variants representing the 6,000 variant capsid sequences and 15 wild type spike-in controls.

Enrichment of variant capsids relative to wild type AAVs was quantified for the barcodes associated with each capsid. Enrichment was determined for each capsid for the DNA fraction from each of the analyzed tissues, as well as for the input viral library injected into each primate and mouse. DNA counts were representative of capsid accumulation

From the secondary screen, 17 capsid variants were identified that exhibited cardiac muscle tissue tropism in both NHPs and in mice. Quantification of cardiac muscle tissue tropism for these 17 variants is provided in TABLE 13. As shown in TABLE 13, capsid variants having 581-589 region sequences of SEQ ID NO: 5027, SEQ ID NO: 4633, SEQ ID NO: 4943, SEQ TD NO: 386, SEQ ID NO: 334, SEQ ID NO: 4309, SEQ ID NO: 4288, SEQ ID NO: 4964, SEQ TD NO: 5017, SEQ ID NO: 5814 SEQ ID NO: 4335, SEQ ID NO:348, SEQ ID NO: 4936, SEQ TD NO: 5206, SEQ ID NO: 4238, SEQ ID NO: 5077, and SEQ ID NO: 5603 were enriched in heart tissue in both NUPs and mice. Additionally, the enrichment of these 17 variants was well correlated between NUPs and mice, as indicated by the median log 2 fold change calculated from the differential expression analysis comparing cardiac tissue to all other tissues. Rho values represent the likelihood that enrichment occurred by chance.

TABLE 13
AAV Capsid Variant Enrichment in Heart Tissue

| | | | NHP Median | | Mouse Median |
|---|---|---|---|---|---|
| SEQ ID | | | log2 Fold | Mouse | log2 Fold |
| NO | Capsid | NHP Rho | Change | Rho | Change |
| 5027 | CVIRSSDQE | 4.69E−08 | 4.47 | 0.03 | 3.69 |
| 4633 | MACWKNATE | 3.08E−07 | 4.49 | 0.02 | 5.66 |
| 4943 | CIVFYMKNQ | 3.93E−05 | 4.07 | 0.02 | 4.95 |
| 386 | CYQFWSQYS | 1.09E−07 | 4.73 | 0.00 | 5.75 |
| 334 | CSAAFQLQP | 1.78E−05 | 4.45 | 0.00 | 4.07 |
| 4309 | CIAFVKQCC | 3.94E−06 | 4.64 | 0.03 | 3.05 |
| 4288 | CACKVSMSG | 2.30E−07 | 4.49 | 0.00 | 4.34 |
| 4964 | CVGKYGGHS | 2.71E−11 | 4.95 | 0.01 | 3.41 |
| 5017 | CFYKIQHKG | 2.18E−08 | 4.82 | 0.00 | 4.46 |
| 5814 | MACKVHLQP | 3.33E−12 | 4.94 | 0.00 | 4.66 |
| 4335 | CPMKHIQDR | 8.53E−10 | 4.65 | 0.04 | 4.95 |
| 348 | CTASWMSWD | 1.28E−07 | 4.28 | 0.02 | 3.10 |
| 4936 | CVMPYSNHP | 4.73E−08 | 4.65 | 0.02 | 4.84 |
| 5206 | CFFYPMSMS | 4.34E−07 | 4.50 | 0.04 | 2.97 |
| 4238 | AACFKQLMQ | 2.12E−07 | 4.44 | 0.03 | 2.79 |
| 5077 | CVGSQLHAM | 2.43E−07 | 4.63 | 0.02 | 5.60 |
| 5603 | QANPYNHGI | 7.85E−05 | 3.82 | 0.03 | 5.26 |

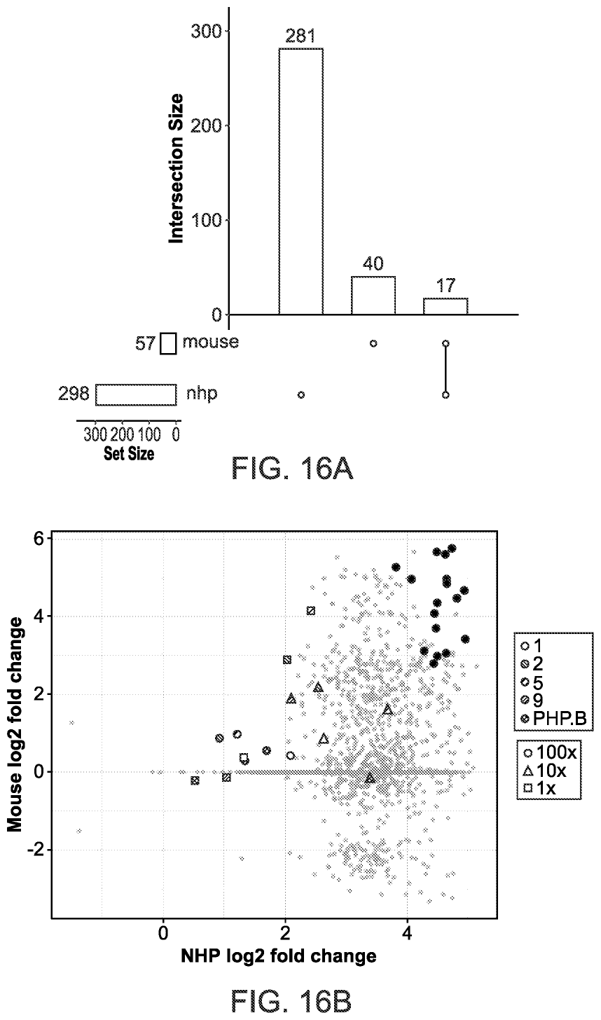

The number of capsids (“intersection size”) enriched in cardiac muscle tissue in NHPs, mice, or both NHPs and mice is shown in FIG. 16A. Seventeen capsid variants, corresponding to the capsid variants provided in TABLE 13, were enriched in cardiac tissue in both NHPs and mice. A differential expression analysis comparing cardiac muscle enrichment of the AAV capsid variants in mice and NHPs is shown in FIG. 16B. These data further demonstrated that these 17 variants (dark red circles) had significant enrichment in both NHPs (FDR <0.0001, log 2fc>1) and mice (FDR <0.05, log 2fc>1). Wild type spike-in controls are shown added at three different concentrations to the input library.

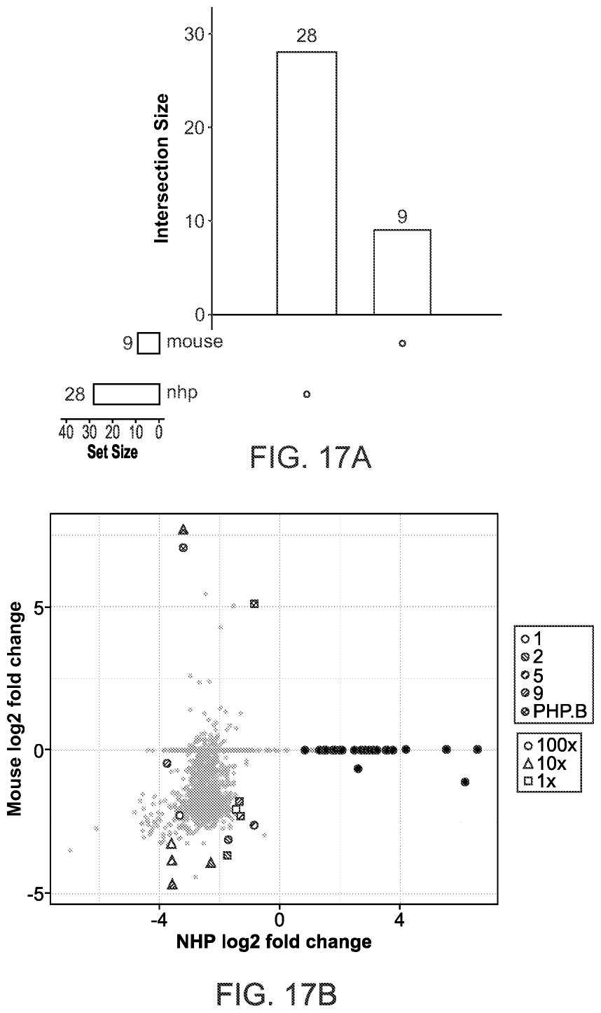

The number of capsids (“intersection size”) enriched in CNS tissue in NHPs or mice is shown in FIG. 17A. A differential expression analysis comparing CNS tissue enrichment of AAV capsid variants in mice and NHPs is shown in FIG. 17B. The 28 capsid variants enriched in NHP CNS tissue (dark gray circles) did not show enrichment in mouse CNS tissue.

### Example 11

### Treatment of a Neurological Disease or Condition with an AAV5-Derived Virion Encapsidating a Therapeutic Payload

This example describes treatment of a neurological disease or condition with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in the neurological disease or condition, or a transgene. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of neurological disease or condition is alleviated, or the subject is cured.

### Example 12

### Treatment of Alzheimer's Disease with an AAV5-Derived Virion Encapsidating a Therapeutic Payload

This example describes treatment of Alzheimer's disease with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. Each such variant is detargeted for all non-CNS tissues, including being detargeted for cardiac tissue. The therapeutic payload is a guide RNA targeting an mRNA encoded for by a gene implicated in Alzheimer's disease or a transgene. The mRNA targeted by the guide RNA is an mRNA encoded for by a gene encoding for amyloid precursor protein (APP), alpha-synuclein (SNCA), Tau protein (MAPT), or Apolipoprotein E (ApoE). The transgene is a gene encoding for amyloid precursor protein (APP), alpha-synuclein (SNCA), Tau protein (MAPT), or Apolipoprotein E (ApoE). The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Alzheimer's disease is alleviated, or the subject is cured.

### Example 13

### Treatment of Parkinson's Disease with an AAV5-Derived Virion Encapsidating a Therapeutic Payload

This example describes treatment of Parkinson's disease with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in Parkinson's disease or a transgene implicated in Parkinson's disease. The mRNA targeted by the guide RNA is an mRNA encoded for by a gene encoding for leucine-rich repeat kinase 2 (LRRK2). The transgene is LRRK2; GBA1; SNCA (alpha-synuclein); AADC; GDNF; Neurturin; GAD; NTN; hFOXG1; hKCNQ2; hFMR1; anti-Tau/miRNA; EPM2A or EPM2B. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Parkinson's disease is alleviated, or the subject is cured.

### Example 14

### Treatment of a Tauopathy with an AAV5-Derived Virion Encapsidating a Therapeutic Payload

This example describes treatment of a Tauopathy with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in the Tauopathy or a transgene. The mRNA targeted by the guide RNA is an mRNA encoded for by a gene (MAPT) encoding for Tau protein. The transgene is MAPT. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Tauopathy disease is alleviated or the subject is cured.

### Example 15

### Treatment of Frontotemporal Dementia with an AAV5-Derived Virion Encapsidating a Therapeutic Payload

This example describes treatment of frontotemporal dementia (FTD) with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in FTD or a transgene encoding a protein implicated in FTD. The gene or the protein implicated in FTD is MAPT (Tau), GRN (Granulin), or progranulin. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of FTD is alleviated, or the subject is cured.

### Example 16

### Treatment of Progressive Supranuclear Palsy with an AAV5-Derived Virion Encapsidating a Therapeutic Payload

This example describes treatment of progressive supranuclear palsy (PSP) with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in PSP or a transgene encoding a protein implicated in PSP. The gene or the protein implicated in PSP is MAPT (Tau). The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of PSP is alleviated, or the subject is cured.

### Example 17

### Treatment of Corticobasal Degeneration with an AAV5-Derived Virion Encapsidating a Therapeutic Payload

This example describes treatment of corticobasal degeneration (CBD) with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in CBD or a transgene encoding a protein implicated in CBD. The gene or the protein implicated in FTD is MAPT (Tau). The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of CBD is alleviated, or the subject is cured.

### Example 18

### Treatment of Chronic Traumatic Encephalopathy with an AAV5-derived Virion Encapsidating a Therapeutic Payload

This example describes treatment of chronic traumatic encephalopathy (CTE) with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in CTE or a transgene encoding a protein implicated in CTE. The gene or the protein implicated in CTE is MAPT (Tau). The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of CTE is alleviated, or the subject is cured.

### Example 19

### Treatment of a Synucleinopathy with an AAV5-derived Virion Encapsidating a Therapeutic Payload

This example describes treatment of a synucleinopathy with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in a synucleinopathy or a transgene encoding a protein implicated in a synucleinopathy. The gene or the protein implicated in the synucleinopathy is SNCA (α-synuclein). The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of the synucleinopathy is alleviated, or the subject is cured.

### Example 20

### Treatment of Rett Syndrome with an AAV5-derived Virion Encapsidating a Therapeutic Payload

This example describes treatment of Rett syndrome with a variant AAV5-derived virion having any one of the engineered CNS tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 12-SEQ ID NO: 3937 or SEQ ID NO: 3938-SEQ ID NO: 4237. The therapeutic payload is a guide RNA targeting an mRNA encoded for by a gene implicated in Rett syndrome, a suppressor tRNA targeting a premature termination codon (PTC) in a gene implicated in Rett syndrome, or a transgene. The mRNA targeted by the guide RNA is an mRNA encoded for by a gene encoding for MECP2. The suppressor tRNA targets a PTC in MECP2. The transgene is MECP2. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced CNS tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Rett syndrome is alleviated or the subject is cured.

### Example 21

### Treatment of a Cardiac Disorder with an AAV5-derived Virion Encapsidating a Therapeutic Payload

This example describes treatment of a cardiac disorder with a variant AAV5-derived virion having any one of the engineered muscle-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 4238-SEQ ID NO: 4933 or SEQ ID NO: 4934-SEQ ID NO: 5233. The therapeutic payload is a guide RNA targeting an mRNA encoded for by a gene implicated in the cardiac disorder or a transgene implicated in the cardiac disorder. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced muscle tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of the cardiac disorder is alleviated, or the subject is cured.

### Example 22

### Treatment of a Skeletal Muscle Disorder with an AAV5-derived Virion Encapsidating a Therapeutic Payload

This example describes treatment of a skeletal muscle disorder with a variant AAV5-derived virion having any one of the engineered muscle-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 4238-SEQ ID NO: 4933 or SEQ ID NO: 4934-SEQ ID NO: 5233. The therapeutic payload is a guide RNA targeting an mRNA encoded for by a gene implicated in the skeletal muscle disorder or a transgene implicated in the skeletal muscle disorder. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced muscle tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of the skeletal muscle disorder is alleviated, or the subject is cured.

### Example 23

### Treatment of Duchenne Muscular Dystrophy with an AAV5-derived Virion Encapsidating a Therapeutic Payload

This example describes treatment of Duchenne muscular dystrophy with a variant AAV5-derived virion having any one of the engineered muscle-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 4238-SEQ ID NO: 4933 or SEQ ID NO: 4934-SEQ ID NO: 5233. The therapeutic payload is a guide RNA targeting an mRNA encoded for by a gene implicated in Duchenne muscular dystrophy or a transgene Duchenne muscular dystrophy. The mRNA targeted by the guide RNA is an mRNA encoded for by a gene encoding for DMID. The transgene is DMID. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced muscle tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of Duchenne muscular dystrophy is alleviated, or the subject is cured.

### Example 24

### Treatment of Facioscapulohumeral Muscular Dystrophy with an AAV5-derived Virion Encapsidating a Therapeutic Payload

This example describes treatment of facioscapulohumeral muscular dystrophy-1 (FSHD) with a variant AAV5-derived virion having any one of the engineered muscle-tropic variant AAV5 VP capsid polypeptides disclosed herein, wherein the variant AAV5 virion encapsidates a therapeutic payload. Polynucleotide sequence encoding for AAV Rep, an AAV5-derived variant Cap and helper proteins and a therapeutic payload are transfected in cells to produce variant AAV5 virions, where the polynucleotide sequence encoding for the variant AAV5 Cap comprises at least one mutation in a 581-589 region, corresponding to residues 581 to 589 in the VP1 capsid polypeptide, and where the polynucleotide sequence encodes for a variant AAV5 Cap comprising a 581-589 region amino acid sequence of any one of SEQ ID NO: 4238-SEQ ID NO: 4933 or SEQ ID NO: 4934-SEQ ID NO: 5233. The therapeutic payload is a guide RNA targeting an mRNA encoded for by a gene implicated in FSHD or a transgene implicated in FSHD. The mRNA targeted by the guide RNA is an mRNA encoded for by a gene encoding for DUX4. The transgene is DUX4. The variant AAV5 virion encapsidating the payload is administered to a subject. The subject is a human or non-human animal. The route of administration is a systemic route of administration. The systemic route of administration is intravenous administration. Upon administration to the subject, the variant AAV5 virions encapsidating the therapeutic payload exhibit enhanced muscle tissue tropism as compared to wild type AAV5 virions encapsidating the therapeutic payload. Upon administration to the subject, a lower dose of the variant AAV5 virions encapsidating the therapeutic payload is administered as compared to wild type AAV5 virions encapsidating the therapeutic payload. Upon administration to the subject, at least one symptom of FSHD is alleviated, or the subject is cured.

### Example 25

### Tissue Abundance of Engineered AAV5 Variants by DNA Analysis

This example demonstrates the tissue abundance of engineered AAV5 variants of the present disclosure as compared to control serotypes by DNA analysis. The CNS-targeted secondary library of the present disclosure comprising engineered AAV5 variants was administered intravenously to four cynomolgus macaques. Four weeks post-injection, animals were euthanized, and a diverse set of tissues was collected for DNA (measuring total variant infection) and RNA (measuring functional transduction) analysis.

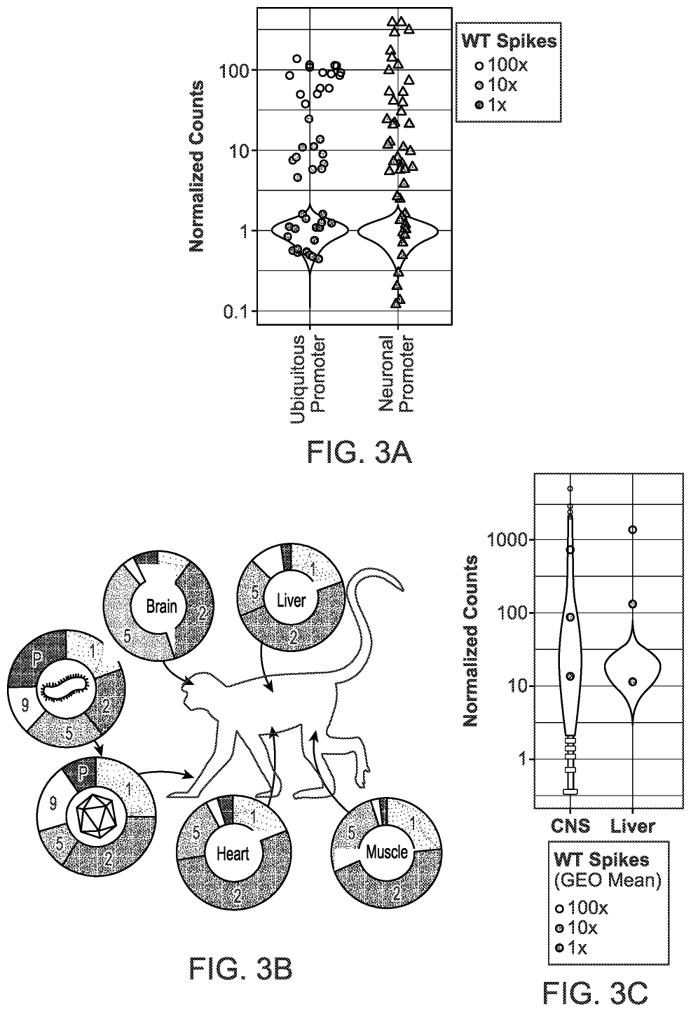

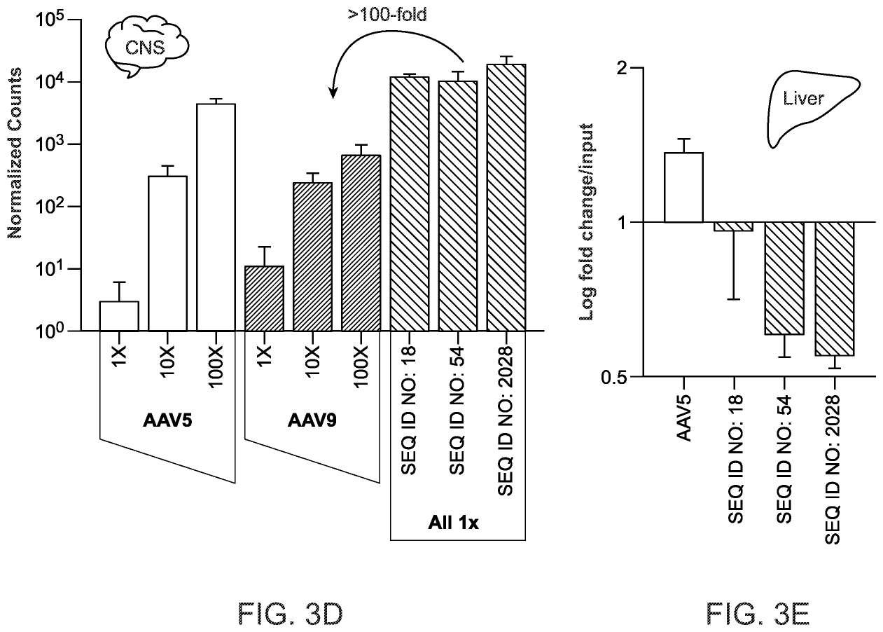

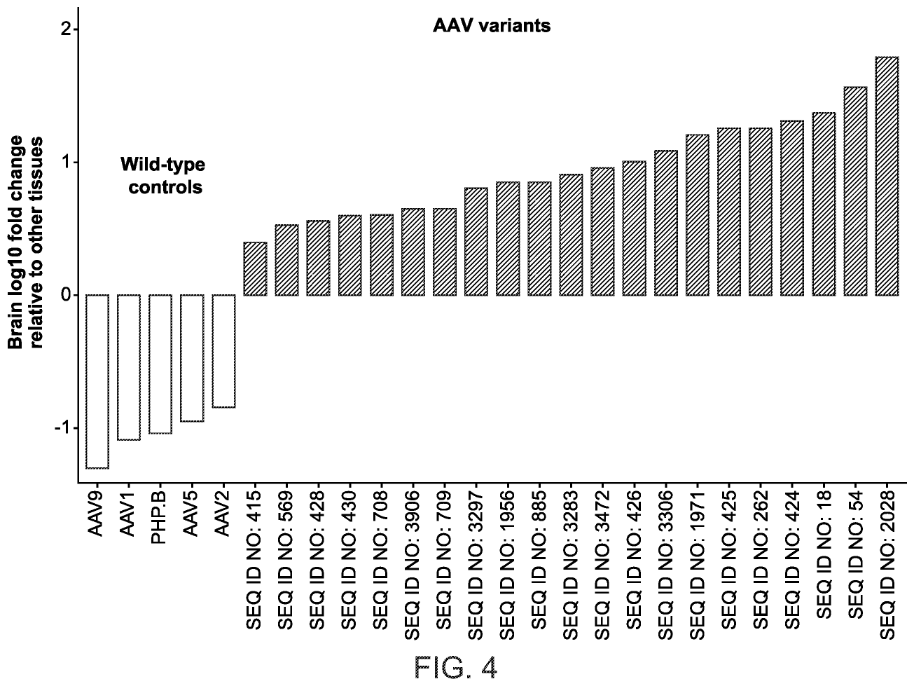

FIG. 3A shows a next generation sequencing (NGS) analysis of purified secondary screen library virus containing engineered AAV5 variants of the present disclosure and barcoded control serotypes (AAV1, AAV2, AAV5, AAV9, and AAV PHP.B) spiked in at 1×, 10×, and 100× stoichiometric ratios. FIG. 3B is a schematic highlighting identification of AAV spike-in control DNA in CNS, liver, heart, and skeletal muscle by NGS analysis of NHP tissues post-selection. FIG. 3C demonstrates that DNA from 94% of capsid variants predicted to target the CNS were found in the CNS. FIG. 3D demonstrates that in a comparison of DNA abundance in the CNS for three engineered AAV5 variants of the present disclosure (AAGRFNYFG (SEQ ID NO: 18), AEDYWDLGA (SEQ ID NO: 54), and MDDICEFYA (SEQ ID NO: 2028)) versus wild type AAV5 and AAV9 spike-in controls, the three engineered AAV5 variants show much greater infection than the highest (100×) controls. FIG. 3E shows that engineered AAV5 variants of the present disclosure exhibit decreased liver infection compared to wild type (parental) AAV5 control.

As demonstrated in FIG. 3A-FIG. 3E, select engineered AAV5 variants of the present disclosure exhibited greater than 100-fold infectivity for CNS and exhibited liver detuning as compared to control serotypes, including commonly used wild type AAVs.

### Example 26

### Functional Transduction of RNA Expression in CNS Tissue

This example demonstrates functional transduction of RNA expression in CNS tissue using CNS tissue-tropic variants. Variants of SEQ ID NO: 18 (AAGRFNYFG), SEQ ID NO: 424 (DAWCFISSY), and SEQ ID NO: 2028 (MDDICEFYA) were compared. A library of barcoded AAV variants was systemically administered to non-human primates. Wild type AAV9 was also administered for reference. Tissues were harvested from the non-human primates, and barcoded DNA was quantified to measure accumulation of the AAV variants in each tissue. RNA expressed from the DNA payload was also quantified to measure functional transduction.

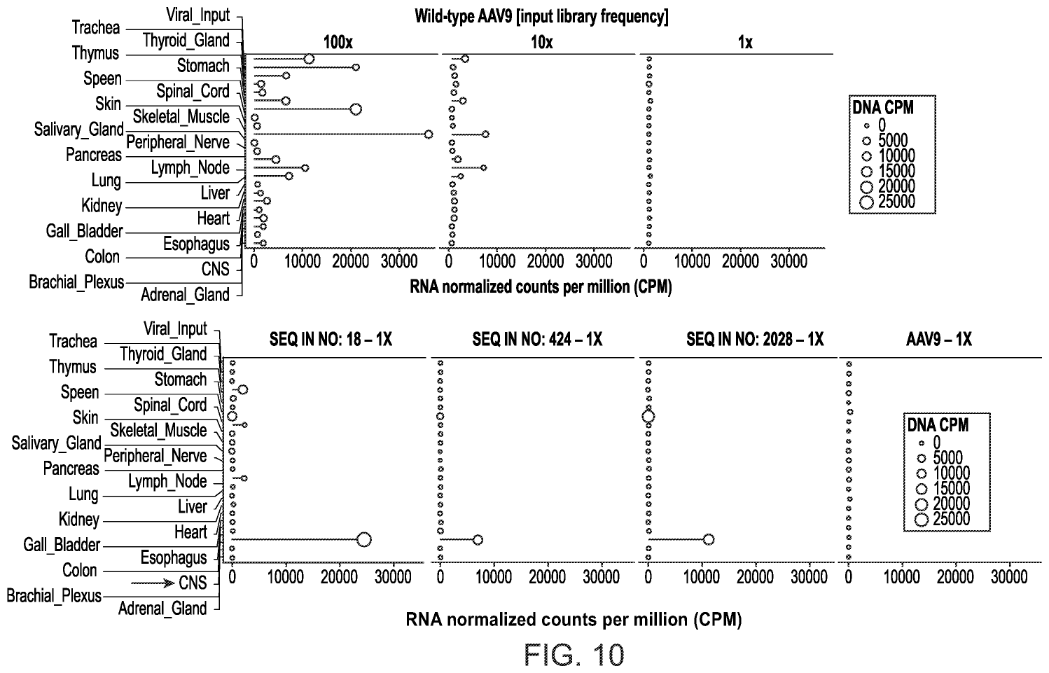

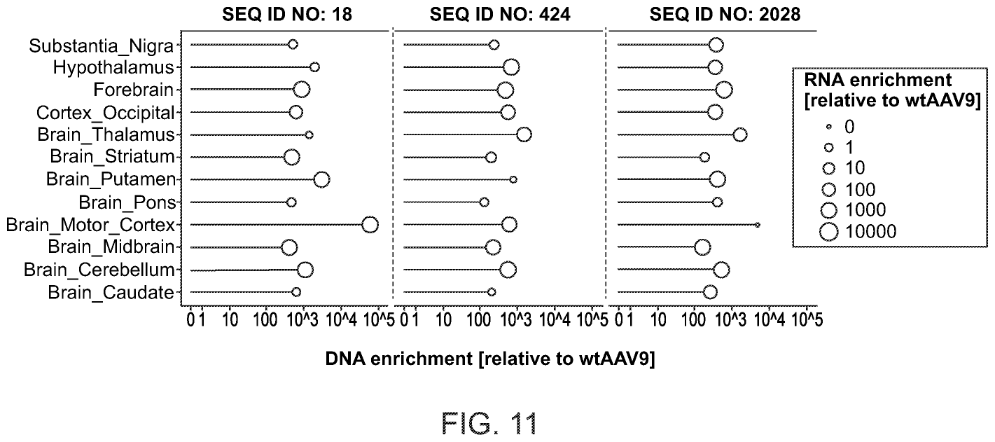

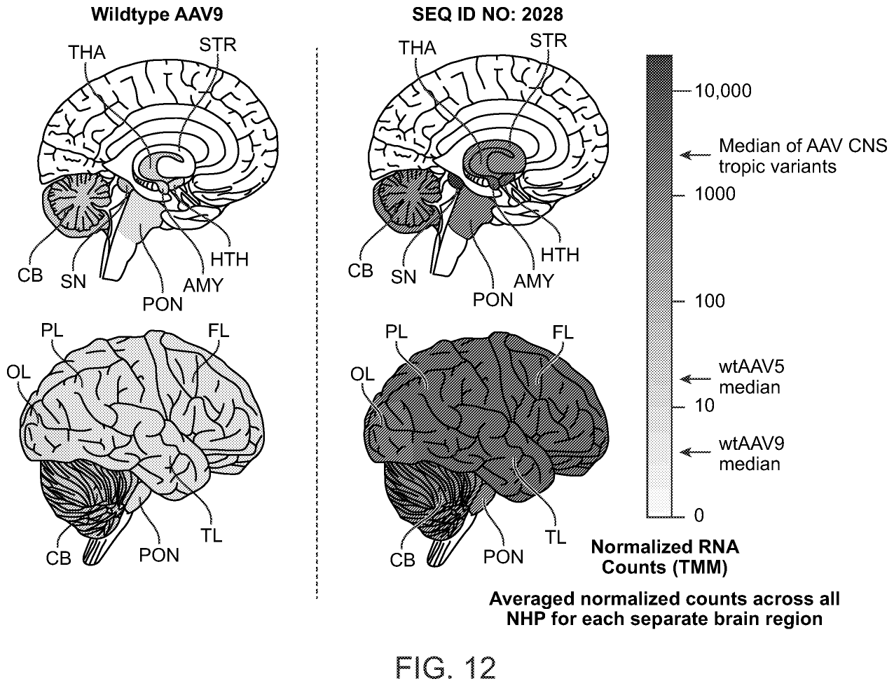

Relative accumulation of AAVs and functional transduction of RNA expression for wild type AAV9 and three CNS tissue-tropic AAV variants (SEQ ID NO: 18, SEQ ID NO: 424, and SEQ ID NO: 2028) is shown in FIG. 10. The three AAV variants exhibited increased CNS tissue tropism, as measured by DNA accumulation, compared to wild type AAV9. The three AAV variants also exhibited CNS tissue-specific transduction of RNA expression. The AAV variants also exhibited broad accumulation and functional transduction in various CNS tissues, including occipital cortex, motor cortex, forebrain, thalamus, striatum, hypothalamus, cerebellum, caudate, putamen, pons, midbrain, and substantia nigra, as shown in FIG. 11. The AAV variant comprising a sequence of SEQ ID NO: 2028 showed broad functional transduction in various CNS tissues compared to wild type AAV9, as shown in FIG. 12.

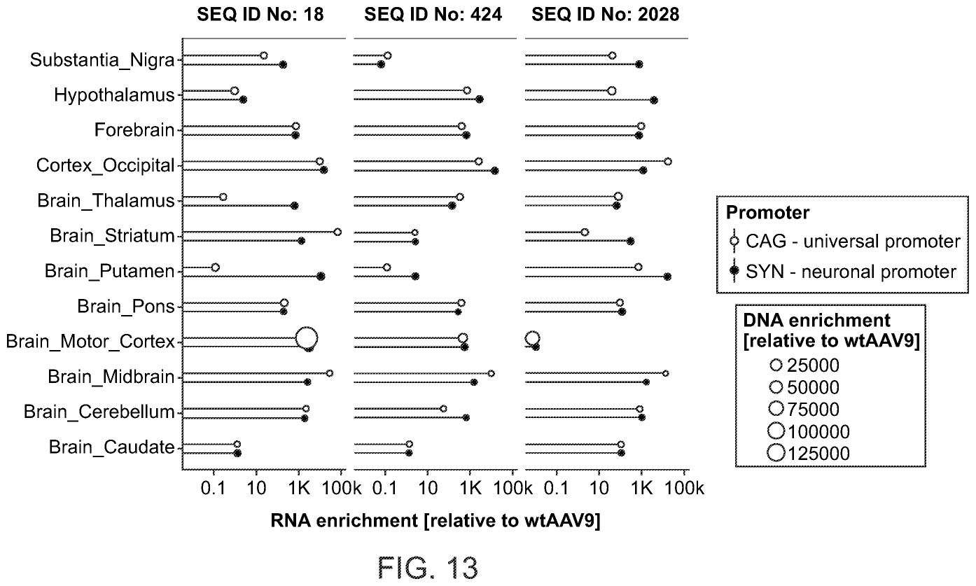

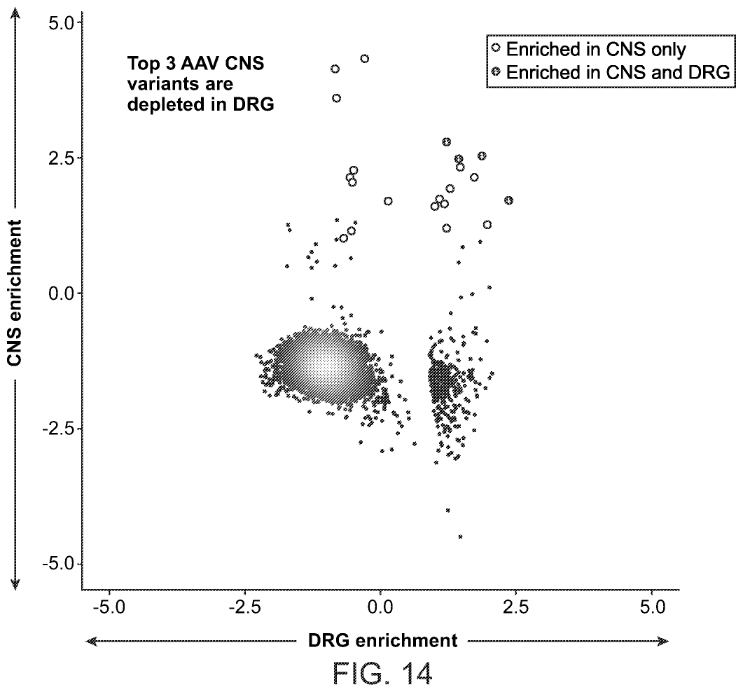

FIG. 13 shows that three AAV variants of the present disclosure (also shown in FIG. 11) functionally transduce neurons. CAG promoter-driven RNA expression highlights functional transduction of any cell type while SYN promoter-driven RNA expression highlights functional transduction of neurons. FIG. 14 shows that some AAV variants of the present disclosure that target CNS also demonstrate dorsal root ganglion (DRG) depletion.

### Example 27

### Functional Transduction of RNA Expression in CNS Tissue Versus Heart Muscle Tissues

This example demonstrates functional transduction of RNA expression in CNS tissue and heart muscle tissue. A library of barcoded AAV variants was systemically administered to non-human primates. Wild type AAV9 was also administered for reference. Tissues were harvested from the non-human primates, and DNA and RNA were quantified.

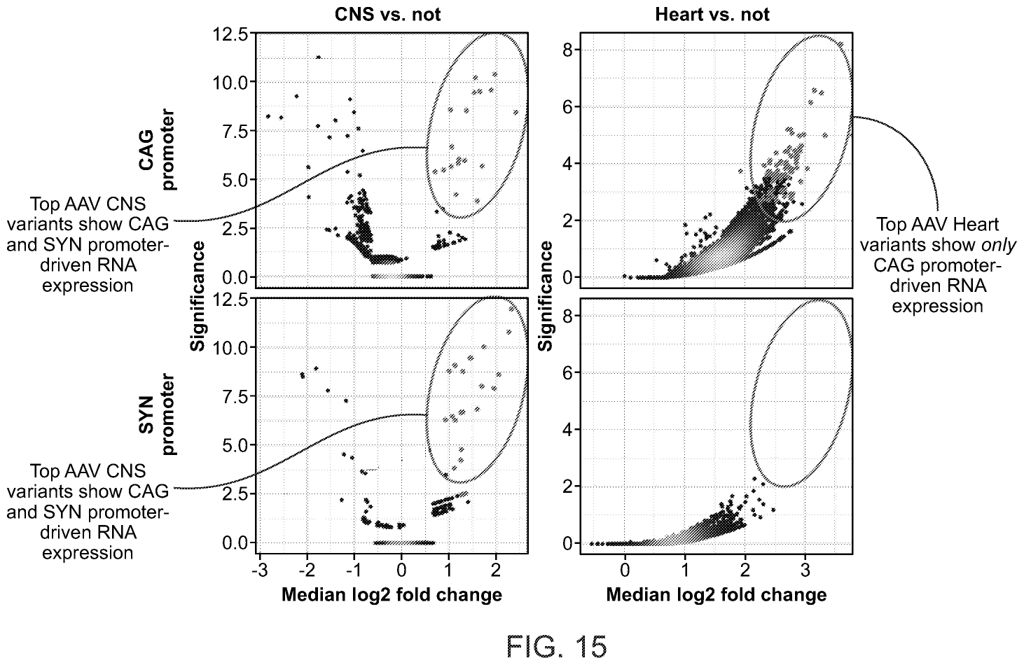

FIG. 15 shows that promoter usage differentiates CNS AAV variants and heart muscle AAV variants. For example, certain AAV CNS variants of the present disclosure shown CAG and SYN promoter-driven RNA expression, while certain AAV heart muscle variants show only CAG promoter-driven RNA expression.

### Example 28

### Singleplex Tertiary Screen to Validate CNS Tissue-Tropic Engineered AAV Capsids

This example describes a tertiary screen, in singleplex, to identify CNS tissue-tropic engineered AAV capsids. Prospective tissue-tropic capsids identified from an in vivo secondary screen, through machine learning, or both are introduced into an in vivo tertiary screen, in singleplex, to confirm tissue-tropic behavior of the identified capsids when individually dosed in NHP. Capsid variant sequences including SEQ ID NO: 2028, SEQ ID NO: 18, SEQ ID NO: 54, and SEQ ID NO: 424 are selected for singleplex screening. Recombinant AAVs of the selected variants encapsidate a model transgene. The transgene is GFP or a tagged frataxin protein. The tertiary screen capsid library is systemically administered to a non-human primate (NHP) by intravenous injection. Following injection, tissues, including CNS and liver, are isolated from the NHP, and the sequences are quantified. Prospective tissue-tropic capsids are compared to control, wild-type AAV to identify superior variant capsids of the present disclosure that target various CNS tissues using DNA and RNA analysis.

Preliminary DNA Enrichment Results. In a pilot study, four male NHPs were intravenously administered, in singleplex, wild-type AAV9 or a capsid variant of the present disclosure of having a 581-589 region of MDDICEFYA (SEQ ID NO: 2028), AAGRFNYFG (SEQ ID NO: 18), or AEDYWDLGA (SEQ ID NO: 54), each carrying a GFP transgene under control of a CMV promoter. Virus was administered at a dose of 3E13 viral genomes (VGs)/kg. Two weeks post-administration, NHPs were euthanized, and various tissues were harvested, including CNS and liver tissues. A kit was used to extract DNA from tissues, and primers against the CMV region of the transgene and GAPDH were used to track vector and genomic counts. DNA was quantitated via droplet digital PCR (ddPCR). In a preliminary analysis of an n=1 regional biopsy (4 mm tissue punches) from several CNS tissue regions (caudate; putamen; cerebellum; cortex, forebrain; cortex, occipital; cortex, temporal; midbrain; pons; substantia nigra; thalamus; motor cortex; Broca's area; hippocampus; hypothalamus; globus pallidus; Wernicke's area; and medulla) and liver tissue regions, the three capsid variants of the present disclosure each exhibited at least about a 7-fold average enrichment across brain regions and at least about a 4-fold average decrease in liver enrichment.

Further analyses are conducted to confirm the preliminary analysis and explore selectivity for CNS tissues as compared to liver and other tissues harvested from NHPs.

### Example 29

### Singleplex Tertiary Screen to Validate Muscle Tissue-Tropic Engineered AAV Capsids

This example describes a tertiary screen, in singleplex, to identify muscle tissue-tropic engineered AAV capsids. Prospective tissue-tropic capsids identified from an in vivo secondary screen, through machine learning, or both are introduced into an in vivo tertiary screen, in singleplex, to confirm tissue-tropic behavior of the identified capsids when individually dosed in NHP. Capsid variant sequences of the present disclosure are selected for singleplex screening. Recombinant AAVs of the selected variants encapsidate a model transgene. The tertiary screen capsid library is systemically administered to a non-human primate (NHP) by intravenous injection. Following injection, tissues, including muscle and liver, are isolated from the NHP, and the sequences are quantified. Prospective tissue-tropic capsids are compared to control, wild-type AAV to identify superior variant capsids of the present disclosure that target various muscle tissues using DNA and RNA analysis.

While the invention has been particularly shown and described with reference to a preferred embodiment and various alternate embodiments, it is understood by persons skilled in the relevant art that various changes in form and details can be made therein without departing from the spirit and scope of the invention.

1-139. (canceled)

140. A viral protein (VP) capsid polypeptide comprising a sequence that has at least 85% identity to any one of SEQ ID NO: 1971, SEQ ID NO: 3283, SEQ ID NO: 12-SEQ ID NO: 1970, SEQ ID NO: 1972-SEQ ID NO: 3282, or SEQ ID NO: 3284-SEQ ID NO: 4237.

141. The VP capsid polypeptide of claim 140, comprising the sequence of any one of SEQ ID NO: 1971, SEQ ID NO: 3283, SEQ ID NO: 12-SEQ ID NO: 1970, SEQ ID NO: 1972-SEQ ID NO: 3282, or SEQ ID NO: 3284-SEQ ID NO: 4237.

142. The VP capsid polypeptide of claim 140, wherein the VP capsid polypeptide comprises a 581-589 region corresponding to residues 581 to 589 of a VP1 polypeptide; wherein the 581-589 region has a sequence that is at least 85% identical to any one of any one SEQ ID NO: 1971, SEQ ID NO: 3283, SEQ ID NO: 12-SEQ ID NO: 1970, SEQ ID NO: 1972-SEQ ID NO: 3282, or SEQ ID NO: 3284-SEQ ID NO: 4237.

143. The VP capsid polypeptide of claim 142, wherein the VP capsid polypeptide comprises a 581-589 region corresponding to residues 581 to 589 of a VP1 polypeptide; wherein the 581-589 region has a sequence of any one of any one SEQ ID NO: 1971, SEQ ID NO: 3283, SEQ ID NO: 12-SEQ ID NO: 1970, SEQ ID NO: 1972-SEQ ID NO: 3282, or SEQ ID NO: 3284-SEQ ID NO: 4237.

144. The VP capsid polypeptide of claim 140, wherein the VP capsid polypeptide is capable of preferentially targeting a central nervous system (CNS) tissue at a DNA enrichment or RNA enrichment of at least 100-fold greater than a wild type AAV9.

145. The VP capsid polypeptide of claim 140, wherein the VP capsid polypeptide is capable of preferentially targeting a central nervous system (CNS) tissue at a DNA enrichment or RNA enrichment of at least 5-fold greater than a wild type AAV5.

146. The VP capsid polypeptide of claim 140, wherein the VP capsid polypeptide confers on a recombinant viral capsid an infection rate for central nervous system (CNS) tissue with at least 3-fold higher CNS tissue tropism than a wild type VP capsid polypeptide of SEQ ID NO: 1.

147. The VP capsid polypeptide of claim 146, wherein the CNS tissue is selected from forebrain cortex, occipital cortex, temporal cortex, thalamus, hypothalamus, substantia nigra, hippocampus DG, hippocampus CA1, hippocampus CA3, cerebellum, and any combination thereof.

148. A pharmaceutical composition comprising the VP capsid polypeptide of claim 140.

149. The pharmaceutical composition of claim 148, wherein the VP capsid polypeptide is assembled into a recombinant viral capsid.

150. A recombinant adeno-associated virus (rAAV), comprising the VP capsid polypeptide of claim 140, assembled into a recombinant viral capsid and a payload encapsidated by the recombinant viral capsid.

151. The rAAV of claim 150, wherein the payload encodes a therapeutic protein, therapeutic polynucleotide, a guide RNA, a tRNA, a suppressor tRNA, a siRNA, a miRNA, an mRNA, a shRNA, a circular RNA, an antisense oligonucleotide (ASO), a ribozyme, a DNAzyme, an aptamer, or any combination thereof.

152. The rAAV of claim 151, wherein the guide RNA is a CRISPR/Cas guide RNA or an ADAR guide RNA.

153. The rAAV of claim 151, wherein the therapeutic protein is selected from the group consisting of aromatic l-amino acid decarboxylase (AADC), amyloid precursor protein (APP), α-synuclein, microtubule associated protein tau (MAPT), ApoE, nerve growth factor (NGF), telomerase reverse transcriptase (TERT), CLN2, CLN3, CLN6, N-acetyl-α-glucosaminidase (NAGLU), granulin, glucosylceramidase 8 (GBA), aspartoacylase, GDNF, neurturin (NTN), glutamate decarboxylase (GAD), FOXG1, Kᵥ7.2, laforin, leucine rich repeat kinase 2 (LRRK2), hexosaminidase A (HEXA), hexosaminidase B (HEXB), huntingtin, cholesterol 24-hydroxylase, C9orf72, superoxide dismutase 1 (SOD1), dual specificity tyrosine-phosphorylation-regulated kinase 1A (DYRKA1), N-sulfoglucosamine sulfohydrolase (SGSH), surfeit locus protein 1 (SURF1), GABA transporter 1 (GAT1), and methyl-CpG binding protein 2 (MECP2).

154. The rAAV of claim 151, wherein the therapeutic polynucleotide targets an mRNA encoding a protein selected from the group consisting of aromatic l-amino acid decarboxylase (AADC), amyloid precursor protein (APP), α-synuclein, microtubule associated protein tau (MAPT), ApoE, nerve growth factor (NGF), telomerase reverse transcriptase (TERT), CLN2, CLN3, CLN6, N-acetyl-α-glucosaminidase (NAGLU), granulin, glucosylceramidase B (GBA), aspartoacylase, GDNF, neurturin (NTN), glutamate decarboxylase (GAD), FOXG1, Kᵥ7.2, laforin, leucine rich repeat kinase 2 (LRRK2), hexosaminidase A (HEXA), hexosaminidase B (HEXB), huntingtin, cholesterol 24-hydroxylase, C9orf72, superoxide dismutase 1 (SOD1), dual specificity tyrosine-phosphorylation-regulated kinase 1A (DYRKA1), N-sulfoglucosamine sulfohydrolase (SGSH), surfeit locus protein 1 (SURF1), GABA transporter 1 (GAT1), and methyl-CpG binding protein 2 (MECP2).

155. The rAAV of any one of claim 151, wherein the payload encodes a component of a CRISPR/Cas system, an adenosine deaminase acting on RNA (ADAR) enzyme, a transcriptional activator, or a transcriptional repressor.

156. The rAAV of claim 155, wherein the component of the CRISPR/Cas system comprises a Cas3, a Cas8, a Cas10, a Cas9, a Cas4, a Cas12, a Cas13, a guide RNA, or a combination thereof.

157. A method of delivering a payload to a central nervous system (CNS) tissue of a subject, the method comprising:
administering a recombinant adeno-associated virus (rAAV) to the subject, wherein the rAAV encapsidates the payload, and wherein the rAAV comprises the viral protein (VP) capsid polypeptide of claim 140;
infecting the CNS tissue with the rAAV; and
thereby delivering a payload to the CNS.

158. A method of transcribing a payload to a central nervous system (CNS) tissue of a subject, the method comprising:
administering a recombinant adeno-associated virus (rAAV) to the subject, wherein the rAAV encapsidates the payload, and wherein the rAAV comprises the viral protein (VP) capsid polypeptide of claim 140;
infecting the CNS tissue with the rAAV; and
preferentially transcribing the payload to the CNS tissue infected by the rAAV.

159. A method of transcribing a payload to a muscle tissue of a subject, the method comprising:
administering a recombinant adeno-associated virus (rAAV) to the subject, wherein the rAAV encapsidates the payload, and wherein the rAAV comprises the viral protein (VP) capsid polypeptide of claim 140;
infecting the muscle tissue with the rAAV; and
preferentially transcribing the payload to the muscle tissue infected by the rAAV.

160. The method of claim 159, wherein the muscle tissue is skeletal muscle tissue or cardiac muscle tissue.

161. A method of treating a condition in a subject, the method comprising:
administering a recombinant adeno-associated virus (rAAV) to the subject, wherein the rAAV encapsidates a payload, and wherein the rAAV comprises a viral protein (VP) capsid polypeptide comprising a sequence that has at least 85% identity to any one of SEQ ID NO: 1971, SEQ ID NO: 3283, SEQ ID NO: 12-SEQ ID NO: 1970, SEQ ID NO: 1972-SEQ ID NO: 3282, or SEQ ID NO: 3284-SEQ ID NO: 4237;
infecting a CNS tissue with the rAAV; and
delivering the payload to the CNS infected by the rAAV; and
producing a therapeutic effect in the CNS tissue, thereby treating the condition.

162. The method of claim 161, wherein the condition is a neurological condition, comprising an aromatic l-amino acid decarboxylase (AADC) deficiency, Alzheimer's disease, a tauopathy, a synucleinopathy, Batten disease, mucopolysaccharidosis type III, frontotemporal dementia, Parkinson's disease, corticobasal degeneration, progressive supranuclear palsy, chronic traumatic encephalopathy, Gaucher disease, Canavan disease, Tay-Sachs disease, Huntington's disease, Protocki-Lupski syndrome, amyotrophic lateral sclerosis, Down syndrome, Sanfilippo disease type A, Sanfilippo disease type B, or Rett syndrome.

163. The method of claim 161, comprising administering from 1×10⁵ to 5×10¹⁴ rAAVs per kg subject weight.

164. The method of claim 161, comprising systemically administering the rAAV to the subject.

165. The method of claim 161, comprising administering the rAAV via intravenous administration, intramuscular administration, intraperitoneal administration, or oral administration.
